(19) United States
(12) Patent Application Publication
(10) Pub. No.: US 2022/0305117 A1
(43) Pub. Date: Sep. 29, 2022

(54) COMBINATION OF HEPATITIS B VIRUS (HBV) VACCINES AND HBV-TARGETING RNAI

(71) Applicant: Janssen Sciences Ireland Unlimited Company, Co Cork (IE)

(72) Inventors: Helen HORTON, Beerse (BE); An DE CREUS, Beerse (BE); Jan BERKE, Beerse (BE)

(21) Appl. No.: 17/596,753

(22) PCT Filed: Jun. 18, 2020
(86) PCT No.: PCT/IB2020/055696

§ 371 Date: Dec. 17, 2021

Related U.S. Application Data
(60) Provisional application No. 62/862,754, filed on Jun. 18, 2019.

(51) Int. Cl. A61K 39/29; A61P 31/20; C12N 15/113; A61K 31/713

(52) U.S. Cl. CPC A61K 39/292; A61P 31/20; C12N 15/1131; A61K 31/713; C12N 2310/14; C12N 2320/31; A61K 2039/53

(57) ABSTRACT

Therapeutic combinations of hepatitis B virus (HBV) vaccines and an RNAi agent for inhibiting the expression of an HBV gene are described. Methods of inducing an immune response against HBV or treating an HBV-induced disease, particularly in individuals having chronic HBV infection, using the disclosed therapeutic combinations are also described.

### CROSS REFERENCE TO RELATED APPLICATION

This application claims priority to U.S. Provisional Application No. 62/862,754 filed on Jun. 18, 2019, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “065814_12WO1_Sequence_Listing” and a creation date of Jun. 15, 2020 and having a size of 47 kb. The sequence listing submitted via EFS-Web is part of the specification and is herein incorporated by reference in its entirety.

### BACKGROUND OF THE INVENTION

Hepatitis B virus (HBV) is a small 3.2-kb hepatotropic DNA virus that encodes four open reading frames and seven proteins. Approximately 240 million people have chronic hepatitis B infection (chronic HBV), characterized by persistent virus and subvirus particles in the blood for more than 6 months (Cohen et al. J. Viral Hepat. (2011) 18(6), 377-83). Persistent HBV infection leads to T-cell exhaustion in circulating and intrahepatic HBV-specific CD4+ and CD8+ T-cells through chronic stimulation of HBV-specific T-cell receptors with viral peptides and circulating antigens. As a result, T-cell polyfunctionality is decreased (i.e., decreased levels of IL-2, tumor necrosis factor (TNF)-α, IFN-γ, and lack of proliferation).

A safe and effective prophylactic vaccine against HBV infection has been available since the 1980s and is the mainstay of hepatitis B prevention (World Health Organization, Hepatitis B: Fact sheet No. 204 [Internet] 2015 March.). The World Health Organization recommends vaccination of all infants, and, in countries where there is low or intermediate hepatitis B endemicity, vaccination of all children and adolescents (<18 years of age), and of people of certain at risk population categories. Due to vaccination, worldwide infection rates have dropped dramatically. However, prophylactic vaccines do not cure established HBV infection.

Chronic HBV is currently treated with IFN-α and nucleoside or nucleotide analogs, but there is no ultimate cure due to the persistence in infected hepatocytes of an intracellular viral replication intermediate called covalently closed circular DNA (cccDNA), which plays a fundamental role as a template for viral RNAs, and thus new virions. It is thought that induced virus-specific T-cell and B-cell responses can effectively eliminate cccDNA-carrying hepatocytes. Current therapies targeting the HBV polymerase suppress viremia, but offer limited effect on cccDNA that resides in the nucleus and related production of circulating antigen. The most rigorous form of a cure may be elimination of HBV cccDNA from the organism, which has neither been observed as a naturally occurring outcome nor as a result of any therapeutic intervention. However, loss of HBV surface antigens (HBsAg) is a clinically credible equivalent of a cure, since disease relapse can occur only in cases of severe immunosuppression, which can then be prevented by prophylactic treatment. Thus, at least from a clinical standpoint, loss of HBsAg is associated with the most stringent form of immune reconstitution against HBV.

For example, immune modulation with pegylated interferon (pegIFN)-α has proven better in comparison to nucleoside or nucleotide therapy in terms of sustained off-treatment response with a finite treatment course. Besides a direct antiviral effect, IFN-α is reported to exert epigenetic suppression of cccDNA in cell culture and humanized mice, which leads to reduction of virion productivity and transcripts (Belloni et al. J. Clin. Invest. (2012) 122(2), 529-537). However, this therapy is still fraught with side-effects and overall responses are rather low, in part because IFN-α has only poor modulatory influences on HBV-specific T-cells. In particular, cure rates are low (<10%) and toxicity is high. Likewise, direct acting HBV antivirals, namely the HBV polymerase inhibitors entecavir and tenofovir, are effective as monotherapy in inducing viral suppression with a high genetic barrier to emergence of drug resistant mutants and consecutive prevention of liver disease progression. However, cure of chronic hepatitis B, defined by HBsAg loss or seroconversion, is rarely achieved with such HBV polymerase inhibitors. Therefore, these antivirals in theory need to be administered indefinitely to prevent reoccurrence of liver disease, similar to antiretroviral therapy for human immunodeficiency virus (HIV).

Therapeutic vaccination has the potential to eliminate HBV from chronically infected patients (Michel et al. J. Hepatol. (2011) 54(6), 1286-1296). Many strategies have been explored, but to date therapeutic vaccination has not proven successful.

### BRIEF SUMMARY OF THE INVENTION

Accordingly, there is an unmet medical need in the treatment of hepatitis B virus (HBV), particularly chronic HBV, for a finite well-tolerated treatment with a higher cure rate. The invention satisfies this need by providing therapeutic combinations or compositions and methods for inducing an immune response against hepatitis B viruses (HBV) infection. The immunogenic compositions/combinations and methods of the invention can be used to provide therapeutic immunity to a subject, such as a subject having chronic HBV infection.

In a general aspect, the application relates to therapeutic combinations or compositions comprising one or more HBV antigens, or one or more polynucleotides encoding the HBV antigens, and an RNAi agent for inhibiting the expression of an HBV gene, for use in treating an HBV infection in a subject in need thereof.

In one embodiment, the therapeutic combination comprises:

i) at least one of:
a) a truncated HBV core antigen consisting of an amino acid sequence that is at least 95%, such as at least 95%, 96%, 97%, 98%, 99% or 100%, identical to SEQ ID NO: 2,
b) a first non-naturally occurring nucleic acid molecule comprising a first polynucleotide sequence encoding the truncated HBV core antigen;
c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and

ii) an RNAi agent for inhibiting the expression of an HBV gene, such as those described herein.

In one embodiment, the truncated HBV core antigen consists of the amino acid sequence of SEQ ID NO: 2 or SEQ ID NO: 4, and the HBV polymerase antigen comprises the amino acid sequence of SEQ ID NO: 7.

In one embodiment, the therapeutic combination comprises at least one of the HBV polymerase antigen and the truncated HBV core antigen. In certain embodiments, the therapeutic combination comprises the HBV polymerase antigen and the truncated HBV core antigen.

In one embodiment, the therapeutic combination comprises at least one of the first non-naturally occurring nucleic acid molecule comprising the first polynucleotide sequence encoding the truncated HBV core antigen, and the second non-naturally occurring nucleic acid molecule comprising the second polynucleotide sequence encoding the HBV polymerase antigen. In certain embodiments, the first non-naturally occurring nucleic acid molecule further comprises a polynucleotide sequence encoding a signal sequence operably linked to the N-terminus of the truncated HBV core antigen, and the second non-naturally occurring nucleic acid molecule further comprises a polynucleotide sequence encoding a signal sequence operably linked to the N-terminus of the HBV polymerase antigen, preferably, the signal sequence independently comprises the amino acid sequence of SEQ ID NO: 9 or SEQ ID NO: 15, more preferably, the signal sequence is encoded by the polynucleotide sequence of SEQ ID NO: 8 or SEQ ID NO: 14, respectively.

In certain embodiments, the first polynucleotide sequence comprises the polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

In certain embodiments, the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

In certain embodiments, the RNAi agent for inhibiting the expression of an HBV gene useful for the invention, as well as related information such as its structure, production, biological activities, therapeutic applications, administration or delivery, etc., is described in US20130005793, WO2013003520 or WO2018027106, the contents of which are incorporated herein by reference in their entirety.

In an embodiment, a therapeutic combination comprises:
a) a first non-naturally occurring nucleic acid molecule comprising a first polynucleotide sequence encoding a truncated HBV core antigen consisting of an amino acid sequence that is at least 95%, such as at least 95%, 96%, 97%, 98%, 99% or 100%, identical to SEQ ID NO: 2;
b) a second non-naturally occurring nucleic acid molecule comprising a second polynucleotide sequence encoding an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity; and
c) an RNAi agent for inhibiting the expression of an HBV gene selected from the group consisting of:
   1) an RNAi agent having the core sense strand sequence and antisense strand sequence shown in Table 2;
   2) an RNAi agent having the sense strand sequence and antisense strand sequence shown in Table 3;
   3) an RNAi agent having the core sense strand sequence and antisense strand sequence shown in Table 4, preferably the RNAi having the modified sense strand sequence and antisense strand sequence shown in Table 4;
   4) an RNAi agent targeting a target sequence shown in Table 5;
   5) an RNAi agent having the core sense strand sequence and antisense strand sequence shown in Table 6;
   6) an RNAi agent having a core antisense sequence shown in Table 7 and a core sense strand sequence shown in Table 8, preferably the RNAi having the modified sense strand sequence shown in Table 7 and the modified antisense strand sequence shown in Table 8; and
   7) an RNAi agent having a duplex of an antisense strand and a sense strand shown in Table 9, preferably the RNAi agent comprises a duplex shown in Table 9.

In certain embodiments, an RNAi agent is delivered to a subject in need thereof by a lipid composition or a lipid nanoparticle. In other embodiment, an RNAi is delivered to a subject in need thereof by conjugating to a targeting ligand, such as a targeting ligand comprising N-acetyl-galactosamine. Preferably, the RNAi is delivered to a subject in need thereof by conjugating to a targeting ligand described herein, such as a targeting ligand comprising N-acetyl-galactosamine.

Preferably, the therapeutic combination comprises a) a first non-naturally occurring nucleic acid molecule comprising a first polynucleotide sequence encoding an truncated HBV core antigen consisting of the amino acid sequence of SEQ ID NO: 2 or SEQ ID NO: 4; b) a second non-naturally occurring nucleic acid molecule comprising a second polynucleotide sequence encoding an HBV polymerase antigen having the amino acid sequence of SEQ ID NO: 7, and (c) an RNAi agent for inhibiting the expression of an HBV gene described herein. Preferably, the RNAi agent comprises a duplex shown in Table 9. Each of the duplexes is preferably conjugated to a targeting ligand, preferably a targeting ligand comprising N-acetyl-galactosamine, more preferably a targeting ligand comprising a structure shown in Table 10.

Preferably, the therapeutic combination comprises a first non-naturally occurring nucleic acid molecule comprising a polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3, and a second non-naturally occurring nucleic acid molecule comprising the polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

More preferably, the therapeutic combination comprises a) a first non-naturally occurring nucleic acid molecule comprising a first polynucleotide sequence of SEQ ID NO: 1 or SEQ ID NO: 3; b) a second non-naturally occurring nucleic acid molecule comprising a second polynucleotide sequence of SEQ ID NO: 5 or 6; and c) an RNAi agent for inhibiting the expression of an HBV gene described herein.

In an embodiment, each of the first and the second non-naturally occurring nucleic acid molecules is a DNA molecule, preferably the DNA molecule is present on a plasmid or a viral vector.

In another embodiment, each of the first and the second non-naturally occurring nucleic acid molecules is an RNA molecule, preferably an mRNA or a self-replicating RNA molecule.

In some embodiments, each of the first and the second non-naturally occurring nucleic acid molecules is independently formulated with a lipid nanoparticle (LNP).

In another general aspect, the application relates to a kit comprising a therapeutic combination of the application.

The application also relates to a therapeutic combination or kit of the application for use in inducing an immune response against hepatitis B virus (HBV); and use of a therapeutic combination, composition or kit of the application in the manufacture of a medicament for inducing an immune response against hepatitis B virus (HBV). The use can further comprise a combination with another immunogenic or therapeutic agent, preferably another HBV antigen or another HBV therapy. Preferably, the subject has chronic HBV infection.

The application further relates to a therapeutic combination or kit of the application for use in treating an HBV-induced disease in a subject in need thereof; and use of a therapeutic combination or kit of the application in the manufacture of a medicament for treating an HBV-induced disease in a subject in need thereof. The use can further comprise a combination with another therapeutic agent, preferably another anti-HBV antigen. Preferably, the subject has chronic HBV infection, and the HBV-induced disease is selected from the group consisting of advanced fibrosis, cirrhosis, and hepatocellular carcinoma (HCC).

The application also relates to a method of inducing an immune response against an HBV or a method of treating an HBV infection or an HBV-induced disease, comprising administering to a subject in need thereof a therapeutic combination according to embodiments of the invention.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A and FIG. 1B show schematic representations of DNA plasmids according to embodiments of the application; FIG. 1A shows a DNA plasmid encoding an HBV core antigen according to an embodiment of the application; FIG. 1B shows a DNA plasmid encoding an HBV polymerase (pol) antigen according to an embodiment of the application; the HBV core and pol antigens are expressed under control of a CMV promoter with an N-terminal cystatin S signal peptide that is cleaved from the expressed antigen upon secretion from the cell; transcriptional regulatory elements of the plasmid include an enhancer sequence located between the CMV promoter and the polynucleotide sequence encoding the HBV antigen and a bGH polyadenylation sequence located downstream of the polynucleotide sequence encoding the HBV antigen; a second expression cassette is included in the plasmid in reverse orientation including a kanamycin resistance gene under control of an Ampr (bla) promoter; an origin of replication (pUC) is also included in reverse orientation;

FIG. 2A and FIG. 2B. show the schematic representations of the expression cassettes in adenoviral vectors according to embodiments of the application; FIG. 2A shows the expression cassette for a truncated HBV core antigen, which contains a CMV promoter, an intron (a fragment derived from the human ApoAI gene—GenBank accession X01038 base pairs 295-523, harboring the ApoAI second intron), a human immunoglobulin secretion signal, followed by a coding sequence for a truncated HBV core antigen and a SV40 polyadenylation signal; FIG. 2B shows the expression cassette for a fusion protein of a truncated HBV core antigen operably linked to an HBV polymerase antigen, which is otherwise identical to the expression cassette for the truncated HBV core antigen except the HBV antigen;

FIG. 3 shows ELISPOT responses of Balb/c mice immunized with different DNA plasmids expressing HBV core antigen or HBV pol antigen, as described in Example 3; peptide pools used to stimulate splenocytes isolated from the various vaccinated animal groups are indicated in gray scale; the number of responsive T-cells are indicated on the y-axis expressed as spot forming cells (SFC) per 10⁶ splenocytes;

FIG. 4 shows core sequences of RNAi agents targeting HBV genes useful for the invention, described in more detail in US20130005793;

FIG. 5 shows modified sequences of RNAi agents targeting HBV genes useful for the invention, described in more detail in US20130005793;

FIG. 6 shows core sequences of RNAi agents targeting HBV genes and their modified counterparts useful for the invention, described in more detail in US20130005793;

FIG. 7 shows example 19-mer HBV cDNA target sequences for HBV RNAi agents useful for the invention, taken from HBV subtype ADW2, genotype A, complete genome GenBank AM282986.1, described in more detail in WO2018027106;

FIG. 8 shows HBV RNAi agent antisense and sense strand core stretch sequences useful for the invention, described in more detail in WO2018027106;

FIG. 9 shows HBV RNAi agent antisense sequences useful for the invention, described in more detail in WO2018027106;

FIG. 10 shows HBV RNAi agent sense sequences useful for the invention, described in more detail in WO2018027106;

FIG. 11 shows examples of HBV RNAi agent duplexes useful for the invention, described in more detail in WO2018027106; and

FIG. 12 shows examples of targeting ligand useful for the invention, described in more detail in WO2018027106.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

The phrases “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul S F, et al (1997), Nucleic Acids Res. 25:3389-3402).

As used herein, the terms and phrases “in combination,” “in combination with,” “co-delivery,” and “administered together with” in the context of the administration of two or more therapies or components to a subject refers to simultaneous administration or subsequent administration of two or more therapies or components, such as two vectors, e.g., DNA plasmids, peptides, or a therapeutic combination and an adjuvant. “Simultaneous administration” can be administration of the two or more therapies or components at least within the same day. When two components are “administered together with” or “administered in combination with,” they can be administered in separate compositions sequentially within a short time period, such as 24, 20, 16, 12, 8 or 4 hours, or within 1 hour, or they can be administered in a single composition at the same time. “Subsequent administration” can be administration of the two or more therapies or components in the same day or on separate days. The use of the term “in combination with” does not restrict the order in which therapies or components are administered to a subject. For example, a first therapy or component (e.g. first DNA plasmid encoding an HBV antigen) can be administered prior to (e.g., 5 minutes to one hour before), concomitantly with or simultaneously with, or subsequent to (e.g., 5 minutes to one hour after) the administration of a second therapy or component (e.g., second DNA plasmid encoding an HBV antigen), and/or a third therapy or component (e.g., RNAi agent for inhibiting the expression of an HBV gene). In some embodiments, a first therapy or component (e.g. first DNA plasmid encoding an HBV antigen), a second therapy or component (e.g., second DNA plasmid encoding an HBV antigen), and a third therapy or component (e.g., RNAi agent for inhibiting the expression of an HBV gene) are administered in the same composition. In other embodiments, a first therapy or component (e.g. first DNA plasmid encoding an HBV antigen), a second therapy or component (e.g., second DNA plasmid encoding an HBV antigen), and a third therapy or component (e.g., RNAi agent for inhibiting the expression of an HBV gene) are administered in separate compositions, such as two or three separate compositions.

As used herein, a “non-naturally occurring” nucleic acid or polypeptide, refers to a nucleic acid or polypeptide that does not occur in nature. A “non-naturally occurring” nucleic acid or polypeptide can be synthesized, treated, fabricated, and/or otherwise manipulated in a laboratory and/or manufacturing setting. In some cases, a non-naturally occurring nucleic acid or polypeptide can comprise a naturally-occurring nucleic acid or polypeptide that is treated, processed, or manipulated to exhibit properties that were not present in the naturally-occurring nucleic acid or polypeptide, prior to treatment. As used herein, a “non-naturally occurring” nucleic acid or polypeptide can be a nucleic acid or polypeptide isolated or separated from the natural source in which it was discovered, and it lacks covalent bonds to sequences with which it was associated in the natural source. A “non-naturally occurring” nucleic acid or polypeptide can be made recombinantly or via other methods, such as chemical synthesis.

As used herein, “subject” means any animal, preferably a mammal, most preferably a human, to whom will be or has been treated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “operably linked” refers to a linkage or a juxtaposition wherein the components so described are in a relationship permitting them to function in their intended manner. For example, a regulatory sequence operably linked to a nucleic acid sequence of interest is capable of directing the transcription of the nucleic acid sequence of interest, or a signal sequence operably linked to an amino acid sequence of interest is capable of secreting or translocating the amino acid sequence of interest over a membrane.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. For example, while embodiments of HBV vectors of the application (e.g., plasmid DNA or viral vectors) described herein may contain particular components, including, but not limited to, certain promoter sequences, enhancer or regulatory sequences, signal peptides, coding sequence of an HBV antigen, polyadenylation signal sequences, etc. arranged in a particular order, those having ordinary skill in the art will appreciate that the concepts disclosed herein may equally apply to other components arranged in other orders that can be used in HBV vectors of the application. The application contemplates use of any of the applicable components in any combination having any sequence that can be used in HBV vectors of the application, whether or not a particular combination is expressly described. The invention generally relates to a therapeutic combination comprising one or more HBV antigens and at least one RNAi agent for inhibiting the expression of an HBV gene.

### Hepatitis B Virus (HBV)

As used herein “hepatitis B virus” or “HBV” refers to a virus of the hepadnaviridae family. HBV is a small (e.g., 3.2 kb) hepatotropic DNA virus that encodes four open reading frames and seven proteins. The seven proteins encoded by HBV include small (S), medium (M), and large (L) surface antigen (HBsAg) or envelope (Env) proteins, pre-Core protein, core protein, viral polymerase (Pol), and HBx protein. HBV expresses three surface antigens, or envelope proteins, L, M, and S, with S being the smallest and L being the largest. The extra domains in the M and L proteins are named Pre-S2 and Pre-S1, respectively. Core protein is the subunit of the viral nucleocapsid. Pol is needed for synthesis of viral DNA (reverse transcriptase, RNaseH, and primer), which takes place in nucleocapsids localized to the cytoplasm of infected hepatocytes. PreCore is the core protein with an N-terminal signal peptide and is proteolytically processed at its N and C termini before secretion from infected cells, as the so-called hepatitis B e-antigen (HBeAg). HBx protein is required for efficient transcription of covalently closed circular DNA (cccDNA). HBx is not a viral structural protein. All viral proteins of HBV have their own mRNA except for core and polymerase, which share an mRNA. With the exception of the protein pre-Core, none of the HBV viral proteins are subject to post-translational proteolytic processing.

The HBV virion contains a viral envelope, nucleocapsid, and single copy of the partially double-stranded DNA genome. The nucleocapsid comprises 120 dimers of core protein and is covered by a capsid membrane embedded with the S, M, and L viral envelope or surface antigen proteins. After entry into the cell, the virus is uncoated and the capsid-containing relaxed circular DNA (rcDNA) with covalently bound viral polymerase migrates to the nucleus. During that process, phosphorylation of the core protein induces structural changes, exposing a nuclear localization signal enabling interaction of the capsid with so-called importins. These importins mediate binding of the core protein to nuclear pore complexes upon which the capsid disassembles and polymerase/rcDNA complex is released into the nucleus. Within the nucleus the rcDNA becomes deproteinized (removal of polymerase) and is converted by host DNA repair machinery to a covalently closed circular DNA (cccDNA) genome from which overlapping transcripts encode for HBeAg, HBsAg, Core protein, viral polymerase and HBx protein. Core protein, viral polymerase, and pre-genomic RNA (pgRNA) associate in the cytoplasm and self-assemble into immature pgRNA-containing capsid particles, which further convert into mature rcDNA-capsids and function as a common intermediate that is either enveloped and secreted as infectious virus particles or transported back to the nucleus to replenish and maintain a stable cccDNA pool.

To date, HBV is divided into four serotypes (adr, adw, ayr, ayw) based on antigenic epitopes present on the envelope proteins, and into eight genotypes (A, B, C, D, E, F, G, and H) based on the sequence of the viral genome. The HBV genotypes are distributed over different geographic regions. For example, the most prevalent genotypes in Asia are genotypes B and C. Genotype D is dominant in Africa, the Middle East, and India, whereas genotype A is widespread in Northern Europe, sub-Saharan Africa, and West Africa.

### HBV Antigens

As used herein, the terms “HBV antigen,” “antigenic polypeptide of HBV,” “HBV antigenic polypeptide,” “HBV antigenic protein,” “HBV immunogenic polypeptide,” and “HBV immunogen” all refer to a polypeptide capable of inducing an immune response, e.g., a humoral and/or cellular mediated response, against an HBV in a subject. The HBV antigen can be a polypeptide of HBV, a fragment or epitope thereof, or a combination of multiple HBV polypeptides, portions or derivatives thereof. An HBV antigen is capable of raising in a host a protective immune response, e.g., inducing an immune response against a viral disease or infection, and/or producing an immunity (i.e., vaccinates) in a subject against a viral disease or infection, that protects the subject against the viral disease or infection. For example, an HBV antigen can comprise a polypeptide or immunogenic fragment(s) thereof from any HBV protein, such as HBeAg, pre-core protein, HBsAg (S, M, or L proteins), core protein, viral polymerase, or HBx protein derived from any HBV genotype, e.g., genotype A, B, C, D, E, F, G, and/or H, or combination thereof.

(1) HBV Core Antigen As used herein, each of the terms “HBV core antigen,” “HBc” and “core antigen” refers to an HBV antigen capable of inducing an immune response, e.g., a humoral and/or cellular mediated response, against an HBV core protein in a subject. Each of the terms “core,” “core polypeptide,” and “core protein” refers to the HBV viral core protein. Full-length core antigen is typically 183 amino acids in length and includes an assembly domain (amino acids 1 to 149) and a nucleic acid binding domain (amino acids 150 to 183). The 34-residue nucleic acid binding domain is required for pre-genomic RNA encapsidation. This domain also functions as a nuclear import signal. It comprises 17 arginine residues and is highly basic, consistent with its function. HBV core protein is dimeric in solution, with the dimers self-assembling into icosahedral capsids. Each dimer of core protein has four a-helix bundles flanked by an a-helix domain on either side. Truncated HBV core proteins lacking the nucleic acid binding domain are also capable of forming capsids.

In an embodiment of the application, an HBV antigen is a truncated HBV core antigen. As used herein, a “truncated HBV core antigen,” refers to an HBV antigen that does not contain the entire length of an HBV core protein, but is capable of inducing an immune response against the HBV core protein in a subject. For example, an HBV core antigen can be modified to delete one or more amino acids of the highly positively charged (arginine rich) C-terminal nucleic acid binding domain of the core antigen, which typically contains seventeen arginine (R) residues. A truncated HBV core antigen of the application is preferably a C-terminally truncated HBV core protein which does not comprise the HBV core nuclear import signal and/or a truncated HBV core protein from which the C-terminal HBV core nuclear import signal has been deleted. In an embodiment, a truncated HBV core antigen comprises a deletion in the C-terminal nucleic acid binding domain, such as a deletion of 1 to 34 amino acid residues of the C-terminal nucleic acid binding domain, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acid residues, preferably a deletion of all 34 amino acid residues. In a preferred embodiment, a truncated HBV core antigen comprises a deletion in the C-terminal nucleic acid binding domain, preferably a deletion of all 34 amino acid residues.

An HBV core antigen of the application can be a consensus sequence derived from multiple HBV genotypes (e.g., genotypes A, B, C, D, E, F, G, and H). As used herein, “consensus sequence” means an artificial sequence of amino acids based on an alignment of amino acid sequences of homologous proteins, e.g., as determined by an alignment (e.g., using Clustal Omega) of amino acid sequences of homologous proteins. It can be the calculated order of most frequent amino acid residues, found at each position in a sequence alignment, based upon sequences of HBV antigens (e.g., core, pol, etc.) from at least 100 natural HBV isolates. A consensus sequence can be non-naturally occurring and different from the native viral sequences. Consensus sequences can be designed by aligning multiple HBV antigen sequences from different sources using a multiple sequence alignment tool, and at variable alignment positions, selecting the most frequent amino acid. Preferably, a consensus sequence of an HBV antigen is derived from HBV genotypes B, C, and D. The term “consensus antigen” is used to refer to an antigen having a consensus sequence.

An exemplary truncated HBV core antigen according to the application lacks the nucleic acid binding function, and is capable of inducing an immune response in a mammal against at least two HBV genotypes. Preferably a truncated HBV core antigen is capable of inducing a T cell response in a mammal against at least HBV genotypes B, C and D. More preferably, a truncated HBV core antigen is capable of inducing a CD8 T cell response in a human subject against at least HBV genotypes A, B, C and D.

Preferably, an HBV core antigen of the application is a consensus antigen, preferably a consensus antigen derived from HBV genotypes B, C, and D, more preferably a truncated consensus antigen derived from HBV genotypes B, C, and D. An exemplary truncated HBV core consensus antigen according to the application consists of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4. SEQ ID NO: 2 and SEQ ID NO: 4 are core consensus antigens derived from HBV genotypes B, C, and D. SEQ ID NO: 2 and SEQ ID NO: 4 each contain a 34-amino acid C-terminal deletion of the highly positively charged (arginine rich) nucleic acid binding domain of the native core antigen.

In one embodiment of the application, an HBV core antigen is a truncated HBV antigen consisting of the amino acid sequence of SEQ ID NO: 2. In another embodiment, an HBV core antigen is a truncated HBV antigen consisting of the amino acid sequence of SEQ ID NO: 4. In another embodiment, an HBV core antigen further contains a signal sequence operably linked to the N-terminus of a mature HBV core antigen sequence, such as the amino acid sequence of SEQ ID NO: 2 or SEQ ID NO: 4. Preferably, the signal sequence has the amino acid sequence of SEQ ID NO: 9 or SEQ ID NO: 15.

(2) HBV Polymerase Antigen

As used herein, the term “HBV polymerase antigen,” “HBV Pol antigen” or “HBV pol antigen” refers to an HBV antigen capable of inducing an immune response, e.g., a humoral and/or cellular mediated response, against an HBV polymerase in a subject. Each of the terms “polymerase,” “polymerase polypeptide,” “Pol” and “pol” refers to the HBV viral DNA polymerase. The HBV viral DNA polymerase has four domains, including, from the N terminus to the C terminus, a terminal protein (TP) domain, which acts as a primer for minus-strand DNA synthesis; a spacer that is nonessential for the polymerase functions; a reverse transcriptase (RT) domain for transcription; and a RNase H domain.

In an embodiment of the application, an HBV antigen comprises an HBV Pol antigen, or any immunogenic fragment or combination thereof. An HBV Pol antigen can contain further modifications to improve immunogenicity of the antigen, such as by introducing mutations into the active sites of the polymerase and/or RNase domains to decrease or substantially eliminate certain enzymatic activities.

Preferably, an HBV Pol antigen of the application does not have reverse transcriptase activity and RNase H activity, and is capable of inducing an immune response in a mammal against at least two HBV genotypes. Preferably, an HBV Pol antigen is capable of inducing a T cell response in a mammal against at least HBV genotypes B, C and D. More preferably, an HBV Pol antigen is capable of inducing a CD8 T cell response in a human subject against at least HBV genotypes A, B, C and D.

Thus, in some embodiments, an HBV Pol antigen is an inactivated Pol antigen. In an embodiment, an inactivated HBV Pol antigen comprises one or more amino acid mutations in the active site of the polymerase domain. In another embodiment, an inactivated HBV Pol antigen comprises one or more amino acid mutations in the active site of the RNaseH domain. In a preferred embodiment, an inactivated HBV pol antigen comprises one or more amino acid mutations in the active site of both the polymerase domain and the RNaseH domain. For example, the “YXDD” motif in the polymerase domain of an HBV pol antigen that can be required for nucleotide/metal ion binding can be mutated, e.g., by replacing one or more of the aspartate residues (D) with asparagine residues (N), eliminating or reducing metal coordination function, thereby decreasing or substantially eliminating reverse transcriptase function. Alternatively, or in addition to mutation of the “YXDD” motif, the “DEDD” motif in the RNaseH domain of an HBV pol antigen required for Mg2+ coordination can be mutated, e.g., by replacing one or more aspartate residues (D) with asparagine residues (N) and/or replacing the glutamate residue (E) with glutamine (Q), thereby decreasing or substantially eliminating RNaseH function. In a particular embodiment, an HBV pol antigen is modified by (1) mutating the aspartate residues (D) to asparagine residues (N) in the “YXDD” motif of the polymerase domain; and (2) mutating the first aspartate residue (D) to an asparagine residue (N) and the first glutamate residue (E) to a glutamine residue (N) in the “DEDD” motif of the RNaseH domain, thereby decreasing or substantially eliminating both the reverse transcriptase and RNaseH functions of the pol antigen.

In a preferred embodiment of the application, an HBV pol antigen is a consensus antigen, preferably a consensus antigen derived from HBV genotypes B, C, and D, more preferably an inactivated consensus antigen derived from HBV genotypes B, C, and D. An exemplary HBV pol consensus antigen according to the application comprises an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to

SEQ ID NO: 7, preferably at least 98% identical to SEQ ID NO: 7, such as at least 98%, 98.5%, 99%, 99.1%, 99.2%, 99.3%, 99.4%, 99.5%, 99.6%, 99.7%, 99.8%, 99.9% or 100% identical to SEQ ID NO: 7. SEQ ID NO: 7 is a pol consensus antigen derived from HBV genotypes B, C, and D comprising four mutations located in the active sites of the polymerase and RNaseH domains. In particular, the four mutations include mutation of the aspartic acid residues (D) to asparagine residues (N) in the “YXDD” motif of the polymerase domain; and mutation of the first aspartate residue (D) to an asparagine residue (N) and mutation of the glutamate residue (E) to a glutamine residue (Q) in the “DEDD” motif of the RNaseH domain.

In a particular embodiment of the application, an HBV pol antigen comprises the amino acid sequence of SEQ ID NO: 7. In other embodiments of the application, an HBV pol antigen consists of the amino acid sequence of SEQ ID NO: 7. In a further embodiment, an HBV pol antigen further contains a signal sequence operably linked to the N-terminus of a mature HBV pol antigen sequence, such as the amino acid sequence of SEQ ID NO: 7. Preferably, the signal sequence has the amino acid sequence of SEQ ID NO: 9 or SEQ ID NO: 15.

(3) Fusion of HBV Core Antigen and HBV Polymerase Antigen

As used herein the term “fusion protein” or “fusion” refers to a single polypeptide chain having at least two polypeptide domains that are not normally present in a single, natural polypeptide.

In an embodiment of the application, an HBV antigen comprises a fusion protein comprising a truncated HBV core antigen operably linked to an HBV Pol antigen, or an HBV Pol antigen operably linked to a truncated HBV core antigen, preferably via a linker.

For example, in a fusion protein containing a first polypeptide and a second heterologous polypeptide, a linker serves primarily as a spacer between the first and second polypeptides. In an embodiment, a linker is made up of amino acids linked together by peptide bonds, preferably from 1 to 20 amino acids linked by peptide bonds, wherein the amino acids are selected from the 20 naturally occurring amino acids. In an embodiment, the 1 to 20 amino acids are selected from glycine, alanine, proline, asparagine, glutamine, and lysine. Preferably, a linker is made up of a majority of amino acids that are sterically unhindered, such as glycine and alanine. Exemplary linkers are polyglycines, particularly (Gly)5, (Gly)8; poly(Gly-Ala), and polyalanines. One exemplary suitable linker as shown in the Examples below is (AlaGly)n, wherein n is an integer of 2 to 5.

Preferably, a fusion protein of the application is capable of inducing an immune response in a mammal against HBV core and HBV Pol of at least two HBV genotypes. Preferably, a fusion protein is capable of inducing a T cell response in a mammal against at least HBV genotypes B, C and D. More preferably, the fusion protein is capable of inducing a CD8 T cell response in a human subject against at least HBV genotypes A, B, C and D.

In an embodiment of the application, a fusion protein comprises a truncated HBV core antigen having an amino acid sequence at least 90%, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4, a linker, and an HBV Pol antigen having an amino acid sequence at least 90%, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100%, identical to SEQ ID NO: 7.

In a preferred embodiment of the application, a fusion protein comprises a truncated HBV core antigen consisting of the amino acid sequence of SEQ ID NO: 2 or SEQ ID NO: 4, a linker comprising (AlaGly)n, wherein n is an integer of 2 to 5, and an HBV Pol antigen having the amino acid sequence of SEQ ID NO: 7. More preferably, a fusion protein according to an embodiment of the application comprises the amino acid sequence of SEQ ID NO: 16.

In one embodiment of the application, a fusion protein further comprises a signal sequence operably linked to the N-terminus of the fusion protein. Preferably, the signal sequence has the amino acid sequence of SEQ ID NO: 9 or SEQ ID NO: 15. In one embodiment, a fusion protein comprises the amino acid sequence of SEQ ID NO: 17.

Additional disclosure on HBV vaccines that can be used for the present invention are described in U.S. patent application Ser. No. 16/223,251, filed Dec. 18, 2018, the contents of the application, more preferably the examples of the application, are hereby incorporated by reference in their entireties.

### Polynucleotides and Vectors

In another general aspect, the application provides a non-naturally occurring nucleic acid molecule encoding an HBV antigen useful for an invention according to embodiments of the application, and vectors comprising the non-naturally occurring nucleic acid. A first or second non-naturally occurring nucleic acid molecule can comprise any polynucleotide sequence encoding an HBV antigen useful for the application, which can be made using methods known in the art in view of the present disclosure. Preferably, a first or second polynucleotide encodes at least one of a truncated HBV core antigen and an HBV polymerase antigen of the application. A polynucleotide can be in the form of RNA or in the form of DNA obtained by recombinant techniques (e.g., cloning) or produced synthetically (e.g., chemical synthesis). The DNA can be single-stranded or double-stranded, or can contain portions of both double-stranded and single-stranded sequence. The DNA can, for example, comprise genomic DNA, cDNA, or combinations thereof. The polynucleotide can also be a DNA/RNA hybrid. The polynucleotides and vectors of the application can be used for recombinant protein production, expression of the protein in host cell, or the production of viral particles. Preferably, a polynucleotide is DNA.

In an embodiment of the application, a first non-naturally occurring nucleic acid molecule comprises a first polynucleotide sequence encoding a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2, preferably 98%, 99% or 100% identical to SEQ ID NO: 2 or SEQ ID NO: 4. In a particular embodiment of the application, a first non-naturally occurring nucleic acid molecule comprises a first polynucleotide sequence encoding a truncated HBV core antigen consisting the amino acid sequence of SEQ ID NO: 2 or SEQ ID NO: 4.

Examples of polynucleotide sequences of the application encoding a truncated HBV core antigen consisting of the amino acid sequence of SEQ ID NO: 2 or SEQ ID NO: 4 include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3. Exemplary non-naturally occurring nucleic acid molecules encoding a truncated HBV core antigen have the polynucleotide sequence of SEQ ID NOs: 1 or 3.

In another embodiment, a first non-naturally occurring nucleic acid molecule further comprises a coding sequence for a signal sequence that is operably linked to the N-terminus of the HBV core antigen sequence. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In an embodiment of the application, a second non-naturally occurring nucleic acid molecule comprises a second polynucleotide sequence encoding an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 100% identical to SEQ ID NO: 7. In a particular embodiment of the application, a second non-naturally occurring nucleic acid molecule comprises a second polynucleotide sequence encoding an HBV polymerase antigen consisting of the amino acid sequence of SEQ ID NO: 7.

Examples of polynucleotide sequences of the application encoding an HBV Pol antigen comprising the amino acid sequence of at least 90% identical to SEQ ID NO: 7 include, but are not limited to, a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. Exemplary non-naturally occurring nucleic acid molecules encoding an HBV pol antigen have the polynucleotide sequence of SEQ ID NOs: 5 or 6.

In another embodiment, a second non-naturally occurring nucleic acid molecule further comprises a coding sequence for a signal sequence that is operably linked to the N-terminus of the HBV pol antigen sequence, such as the amino acid sequence of SEQ ID NO: 7. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In another embodiment of the application, a non-naturally occurring nucleic acid molecule encodes an HBV antigen fusion protein comprising a truncated HBV core antigen operably linked to an HBV Pol antigen, or an HBV Pol antigen operably linked to a truncated HBV core antigen. In a particular embodiment, a non-naturally occurring nucleic acid molecule of the application encodes a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2 or SEQ ID NO: 4, preferably 100% identical to SEQ ID NO: 2 or SEQ ID NO: 4, more preferably 100% identical to SEQ ID NO: 2 or SEQ ID NO:4; a linker; and an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 98%, 99% or 100% identical to SEQ ID NO: 7. In a particular embodiment of the application, a non-naturally occurring nucleic acid molecule encodes a fusion protein comprising a truncated HBV core antigen consisting of the amino acid sequence of SEQ ID NO: 2 or SEQ ID NO: 4, a linker comprising (AlaGly)n, wherein n is an integer of 2 to 5; and an HBV Pol antigen comprising the amino acid sequence of SEQ ID NO: 7. In a particular embodiment of the application, a non-naturally occurring nucleic acid molecule encodes an HBV antigen fusion protein comprising the amino acid sequence of SEQ ID NO: 16.

Examples of polynucleotide sequences of the application encoding an HBV antigen fusion protein include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, operably linked to a linker coding sequence at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 11, preferably 98%, 99% or 100% identical to SEQ ID NO: 11, which is further operably linked a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. In particular embodiments of the application, a non-naturally occurring nucleic acid molecule encoding an HBV antigen fusion protein comprises SEQ ID NO: 1 or SEQ ID NO: 3, operably linked to SEQ ID NO: 11, which is further operably linked to SEQ ID NO: 5 or SEQ ID NO: 6.

In another embodiment, a non-naturally occurring nucleic acid molecule encoding an HBV fusion further comprises a coding sequence for a signal sequence that is operably linked to the N-terminus of the HBV fusion sequence, such as the amino acid sequence of SEQ ID NO: 16. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14. In one embodiment, the encoded fusion protein with the signal sequence comprises the amino acid sequence of SEQ ID NO: 17.

The application also relates to a vector comprising the first and/or second non-naturally occurring nucleic acid molecules. As used herein, a “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. A vector can be a DNA vector or an RNA vector. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

Vectors of the application can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some embodiments of the application, a vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g. a linear covalently closed DNA such as linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of DNA plasmids suitable that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC® complete baculovirus expression system (Thermo Fisher Scientific), which can be used for production and/or expression in insect cells; pcDNATM or pcDNA3TM (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNATM, pcDNA3TM, pVAX, pVAX-1, ADVAX, NTC8454, etc. Preferably, an expression vector is based on pVAX-1, which can be further modified to optimize protein expression in mammalian cells. pVAX-1 is commonly used plasmid in DNA vaccines, and contains a strong human intermediate early cytomegalovirus (CMV-IE) promoter followed by the bovine growth hormone (bGH)-derived polyadenylation sequence (pA). pVAX-1 further contains a pUC origin of replication and kanamycin resistance gene driven by a small prokaryotic promoter that allows for bacterial plasmid propagation.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, etc. Examples of viral vectors that can be used include, but are not limited to, arenavirus viral vectors, replication-deficient arenavirus viral vectors or replication-competent arenavirus viral vectors, bi-segmented or tri-segmented arenavirus, infectious arenavirus viral vectors, nucleic acids which comprise an arenavirus genomic segment wherein one open reading frame of the genomic segment is deleted or functionally inactivated (and replaced by a nucleic acid encoding an HBV antigen as described herein), arenavirus such as lymphocytic choriomeningitidis virus (LCMV), e.g., clone 13 strain or MP strain, and arenavirus such as Junin virus e.g., Candid #1 strain. The vector can also be a non-viral vector.

Preferably, a viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g. rhAd51, rhAd52 or rhAd53.

The vector can also be a linear covalently closed double-stranded DNA vector. As used herein, a “linear covalently closed double-stranded DNA vector” refers to a closed linear deoxyribonucleic acid (DNA) that is structurally distinct from a plasmid DNA. It has many of the advantages of plasmid DNA as well as a minimal cassette size similar to RNA strategies. For example, it can be a vector cassette generally comprising an encoded antigenic sequence, a promoter, a polyadenylation sequence, and telomeric ends. The plasmid-free construct can be synthesized through an enzymatic process without the need for bacterial sequences. Examples of suitable linear covalently closed DNA vectors include, but are not limited to, commercially available expression vectors such as ‘Doggybone™ closed linear DNA’ (dbDNA™) (Touchlight Genetics Ltd.; London, England). See, e.g., Scott et al, Hum Vaccin Immunother. 2015 August; 11(8): 1972-1982, the entire content of which is incorporated herein by reference. Some examples of linear covalently closed double-stranded DNA vectors, compositions and methods to create and use such vectors for delivering DNA molecules, such as active molecules of this invention, are described in US2012/0282283, US2013/0216562, and US2018/0037943, the relevant content of each of which is hereby incorporated by reference in its entirety.

A recombinant vector useful for the application can be prepared using methods known in the art in view of the present disclosure. For example, in view of the degeneracy of the genetic code, several nucleic acid sequences can be designed that encode the same polypeptide. A polynucleotide encoding an HBV antigen of the application can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

A vector of the application, e.g., a DNA plasmid, a viral vector (particularly an adenoviral vector), an RNA vector (such as a self-replicating RNA replicon), or a linear covalently closed double-stranded DNA vector, can comprise any regulatory elements to establish conventional function(s) of the vector, including but not limited to replication and expression of the HBV antigen(s) encoded by the polynucleotide sequence of the vector. Regulatory elements include, but are not limited to, a promoter, an enhancer, a polyadenylation signal, translation stop codon, a ribosome binding element, a transcription terminator, selection markers, origin of replication, etc. A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest (e.g., HBV antigen) from a start codon to a stop codon. Regulatory elements of the expression cassette can be operably linked to a polynucleotide sequence encoding an HBV antigen of interest. As used herein, the term “operably linked” is to be taken in its broadest reasonable context, and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

A vector can comprise a promoter sequence, preferably within an expression cassette, to control expression of an HBV antigen of interest. The term “promoter” is used in its conventional sense, and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding an HBV antigen within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metalothionein. A promoter can also be a tissue specific promoter, such as a muscle or skin specific promoter, natural or synthetic.

Preferably, a promoter is a strong eukaryotic promoter, preferably a cytomegalovirus immediate early (CMV-IE) promoter. A nucleotide sequence of an exemplary CMV-IE promoter is shown in SEQ ID NO: 18 or SEQ ID NO: 19.

A vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., an HBV antigen) within an expression cassette of the vector. Enhancer sequences are regulatory DNA sequences that, when bound by transcription factors, enhance the transcription of an associated gene. An enhancer sequence is preferably located upstream of the polynucleotide sequence encoding an HBV antigen, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. Preferably, a polyadenylation signal is a bovine growth hormone (bGH) polyadenylation signal or a SV40 polyadenylation signal. A nucleotide sequence of an exemplary bGH polyadenylation signal is shown in SEQ ID NO: 20. A nucleotide sequence of an exemplary SV40 polyadenylation signal is shown in SEQ ID NO: 13.

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein Al precursor (ApoAI), untranslated R-U5 domain of the human T-cell leukemia virus type 1 (HTLV-1) long terminal repeat (LTR), a splicing enhancer, a synthetic rabbit β-globin intron, or any combination thereof. Preferably, an enhancer sequence is a composite sequence of three consecutive elements of the untranslated R-U5 domain of HTLV-1 LTR, rabbit β-globin intron, and a splicing enhancer, which is referred to herein as “a triple enhancer sequence.” A nucleotide sequence of an exemplary triple enhancer sequence is shown in SEQ ID NO: 10. Another exemplary enhancer sequence is an ApoAI gene fragment shown in SEQ ID NO: 12.

A vector can comprise a polynucleotide sequence encoding a signal peptide sequence. Preferably, the polynucleotide sequence encoding the signal peptide sequence is located upstream of the polynucleotide sequence encoding an HBV antigen. Signal peptides typically direct localization of a protein, facilitate secretion of the protein from the cell in which it is produced, and/or improve antigen expression and cross-presentation to antigen-presenting cells. A signal peptide can be present at the N-terminus of an HBV antigen when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

Preferably, a signal peptide sequence is a cystatin S signal peptide. Exemplary nucleic acid and amino acid sequences of a cystatin S signal peptide are shown in SEQ ID NOs: 8 and 9, respectively. Exemplary nucleic acid and amino acid sequences of an immunoglobulin secretion signal are shown in SEQ ID NOs: 14 and 15, respectively.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding an HBV antigen, or in the opposite (reverse) orientation. An origin of replication (ORI) is a sequence at which replication is initiated, enabling a plasmid to reproduce and survive within cells. Examples of ORIS suitable for use in the application include, but are not limited to ColE1, pMB1, pUC, pSC101, R6K, and 15A, preferably pUC. An exemplary nucleotide sequence of a pUC ORI is shown in SEQ ID NO: 21.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., HBV antigen. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kanr), ampicillin resistance gene (Ampr), and tetracycline resistance gene (Tetr), as well as genes conferring resistance to chloramphenicol, bleomycin, spectinomycin, carbenicillin, etc.

Preferably, an antibiotic resistance gene in the antibiotic expression cassette of a vector is a kanamycin resistance gene (Kanr). The sequence of Kanr gene is shown in SEQ ID NO: 22. Preferably, the Kanr gene is codon optimized. An exemplary nucleic acid sequence of a codon optimized Kanr gene is shown in SEQ ID NO: 23. The Kanr can be operably linked to its native promoter, or the Kanr gene can be linked to a heterologous promoter. In a particular embodiment, the Kanr gene is operably linked to the ampicillin resistance gene (Ampr) promoter, known as the bla promoter. An exemplary nucleotide sequence of a bla promoter is shown in SEQ ID NO: 24.

In a particular embodiment of the application, a vector is a DNA plasmid comprising an expression cassette including a polynucleotide encoding at least one of an HBV antigen selected from the group consisting of an HBV pol antigen comprising an amino acid sequence at least 90%, such as 90%, 91%, 92%, 93%, 94%, 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 7, and a truncated HBV core antigen consisting of the amino acid sequence at least 95%, such as 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical of SEQ ID NO: 2 or SEQ ID NO: 4; an upstream sequence operably linked to the polynucleotide encoding the HBV antigen comprising, from 5′ end to 3′ end, a promoter sequence, preferably a CMV promoter sequence of SEQ ID NO: 18, an enhancer sequence, preferably a triple enhancer sequence of SEQ ID NO: 10, and a polynucleotide sequence encoding a signal peptide sequence, preferably a cystatin S signal peptide having the amino acid sequence of SEQ ID NO: 9; and a downstream sequence operably linked to the polynucleotide encoding the HBV antigen comprising a polyadenylation signal, preferably a bGH polyadenylation signal of SEQ ID NO: 20. Such vector further comprises an antibiotic resistance expression cassette including a polynucleotide encoding an antibiotic resistance gene, preferably a Kanr gene, more preferably a codon optimized Kanr gene of at least 90% identical to SEQ ID NO: 23, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 23, preferably 100% identical to SEQ ID NO: 23, operably linked to an Ampr (bla) promoter of SEQ ID NO: 24, upstream of and operably linked to the polynucleotide encoding the antibiotic resistance gene; and an origin of replication, preferably a pUC ori of SEQ ID NO: 21. Preferably, the antibiotic resistance cassette and the origin of replication are present in the plasmid in the reverse orientation relative to the HBV antigen expression cassette.

In another particular embodiment of the application, a vector is a viral vector, preferably an adenoviral vector, more preferably an Ad26 or Ad35 vector, comprising an expression cassette including a polynucleotide encoding at least one of an HBV antigen selected from the group consisting of an HBV pol antigen comprising an amino acid sequence at least 90%, such as 90%, 91%, 92%, 93%, 94%, 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 7, and a truncated HBV core antigen consisting of the amino acid sequence at least 95%, such as 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical of SEQ ID NO: 2 or SEQ ID NO: 4; an upstream sequence operably linked to the polynucleotide encoding the HBV antigen comprising, from 5′ end to 3′ end, a promoter sequence, preferably a CMV promoter sequence of SEQ ID NO: 19, an enhancer sequence, preferably an ApoAl gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

In an embodiment of the application, a vector, such as a plasmid DNA vector or a viral vector (preferably an adenoviral vector, more preferably an Ad26 or Ad35 vector), encodes an HBV Pol antigen having the amino acid sequence of SEQ ID NO: 7. Preferably, the vector comprises a coding sequence for the HBV Pol antigen that is at least 90% identical to the polynucleotide sequence of SEQ ID NO: 5 or 6, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or 6, preferably 100% identical to SEQ ID NO: 5 or 6.

In an embodiment of the application, a vector, such as a plasmid DNA vector or a viral vector (preferably an adenoviral vector, more preferably an Ad26 or Ad35 vector), encodes a truncated HBV core antigen consisting of the amino acid sequence of SEQ ID NO: 2 or SEQ ID NO: 4. Preferably, the vector comprises a coding sequence for the truncated HBV core antigen that is at least 90% identical to the polynucleotide sequence of SEQ ID NO: 1 or SEQ ID NO: 3, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 100% identical to SEQ ID NO: 1 or SEQ ID NO: 3.

In yet another embodiment of the application, a vector, such as a plasmid DNA vector or a viral vector (preferably an adenoviral vector, more preferably an Ad26 or Ad35 vector), encodes a fusion protein comprising an HBV Pol antigen having the amino acid sequence of SEQ ID NO: 7 and a truncated HBV core antigen consisting of the amino acid sequence of SEQ ID NO: 1 or SEQ ID NO: 3. Preferably, the vector comprises a coding sequence for the fusion, which contains a coding sequence for the truncated HBV core antigen at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, more preferably SEQ ID NO: 1 or SEQ ID NO: 3, operably linked to a coding sequence for the HBV Pol antigen at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6, more preferably SEQ ID NO: 5 or SEQ ID NO: 6. Preferably, the coding sequence for the truncated HBV core antigen is operably linked to the coding sequence for the HBV Pol antigen via a coding sequence for a linker at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 11, preferably 98%, 99% or 100% identical to SEQ ID NO: 11. In particular embodiments of the application, a vector comprises a coding sequence for the fusion having SEQ ID NO: 1 or SEQ ID NO: 3 operably linked to SEQ ID NO: 11, which is further operably linked to SEQ ID NO: 5 or SEQ ID NO: 6.

The polynucleotides and expression vectors encoding the HBV antigens of the application can be made by any method known in the art in view of the present disclosure. For example, a polynucleotide encoding an HBV antigen can be introduced or “cloned” into an expression vector using standard molecular biology techniques, e.g., polymerase chain reaction (PCR), etc., which are well known to those skilled in the art.

### Cells, Polypeptides and Antibodies

The application also provides cells, preferably isolated cells, comprising any of the polynucleotides and vectors described herein. The cells can, for instance, be used for recombinant protein production, or for the production of viral particles.

Embodiments of the application thus also relate to a method of making an HBV antigen of the application. The method comprises transfecting a host cell with an expression vector comprising a polynucleotide encoding an HBV antigen of the application operably linked to a promoter, growing the transfected cell under conditions suitable for expression of the HBV antigen, and optionally purifying or isolating the HBV antigen expressed in the cell. The HBV antigen can be isolated or collected from the cell by any method known in the art including affinity chromatography, size exclusion chromatography, etc. Techniques used for recombinant protein expression will be well known to one of ordinary skill in the art in view of the present disclosure. The expressed HBV antigens can also be studied without purifying or isolating the expressed protein, e.g., by analyzing the supernatant of cells transfected with an expression vector encoding the HBV antigen and grown under conditions suitable for expression of the HBV antigen.

Thus, also provided are non-naturally occurring or recombinant polypeptides comprising an amino acid sequence that is at least 90% identical to the amino acid sequence of SEQ ID NO: 2, SEQ ID NO: 4, or SEQ ID NO: 7. As described above and below, isolated nucleic acid molecules encoding these sequences, vectors comprising these sequences operably linked to a promoter, and compositions comprising the polypeptide, polynucleotide, or vector are also contemplated by the application.

In an embodiment of the application, a recombinant polypeptide comprises an amino acid sequence that is at least 90% identical to the amino acid sequence of SEQ ID NO: 2, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2. Preferably, a non-naturally occurring or recombinant polypeptide consists of SEQ ID NO: 2.

In another embodiment of the application, a non-naturally occurring or recombinant polypeptide comprises an amino acid sequence that is at least 90% identical to the amino acid sequence of SEQ ID NO: 4, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 4. Preferably, a non-naturally occurring or recombinant polypeptide comprises SEQ ID NO: 4.

In another embodiment of the application, a non-naturally occurring or recombinant polypeptide comprises an amino acid sequence that is at least 90% identical to the amino acid sequence of SEQ ID NO: 7, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7. Preferably, a non-naturally occurring or recombinant polypeptide consists of SEQ ID NO: 7.

Also provided are antibodies or antigen-binding fragments thereof that specifically bind to a non-naturally occurring polypeptide of the application. In an embodiment of the application, an antibody specific to a non-naturally occurring HBV antigen of the application does not bind specifically to another HBV antigen. For example, an antibody of the application that binds specifically to an HBV Pol antigen having the amino acid sequence of SEQ ID NO: 7 will not bind specifically to an HBV Pol antigen not having the amino acid sequence of SEQ ID NO: 7.

As used herein, the term “antibody” includes polyclonal, monoclonal, chimeric, humanized, Fv, Fab and F(ab')2; bifunctional hybrid (e.g., Lanzavecchia et al., Eur. J. Immunol. 17:105, 1987), single-chain (Huston et al., Proc. Natl. Acad. Sci. USA 85:5879, 1988; Bird et al., Science 242:423, 1988); and antibodies with altered constant regions (e.g., U.S. Pat. No. 5,624,821).

As used herein, an antibody that “specifically binds to” an antigen refers to an antibody that binds to the antigen with a KD of 1×10⁻⁷ M or less. Preferably, an antibody that “specifically binds to” an antigen binds to the antigen with a KD of 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as a Octet RED96 system.

The smaller the value of the KD of an antibody, the higher affinity that the antibody binds to a target antigen.

### RNAi Agents

The application also relates to therapeutic applications of RNAi agents for inhibiting the expression of an HBV gene, also referred to herein as “HBV RNAi molecules” or “HBV RNAi agents”.

RNAi agents for inhibiting the expression of an HBV gene are known in the art. For example, RNAi agents for inhibiting the expression of an HBV gene include, but are not limited to, those described in US20130005793, WO2013003520 and WO2018027106, the content of each of which is incorporated herein in its entirety.

Each HBV RNAi agent comprises a sense strand and an antisense strand. The sense strand and the antisense strand each can be 16 to 30 nucleotides in length. In some embodiments, the sense and antisense strands each can be 17 to 26 nucleotides in length. The sense and antisense strands can be either the same length or they can be different lengths. In some embodiments, the sense and antisense strands are each independently 17 to 26 nucleotides in length. In some embodiments, the sense and antisense strands are each independently 1 7-21 nucleotides in length. In some embodiments, both the sense and antisense strands are each 21-26 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, both the sense and antisense strands are each 26 nucleotides in length. In some embodiments, the RNAi agent sense and antisense strands are each independently 1 7, 18, 19, 20, 21, 22, 23, 24, 25, or 26 nucleotides in length. In some embodiments, a double-stranded RNAi agent has a duplex length of about 16, 17. 18, 19, 20, 21. 22, 23 or 24 nucleotides. This region of perfect or substantial complementarity between the sense strand and the antisense strand is typically 15-25 (e.g., 15, 16, 17, 18, 19, 20, 21, 22, 23, 24, or 25) nucleotides in length and occurs at or near the 5′ end of the antisense strand (e.g., this region may be separated from the 5′ end of the antisense strand by 0, 1, 2, 3, or 4 nucleotides that are not perfectly or substantially complementary).

The sense strand and antisense strand each contain a core stretch sequence that is 16 to 23 nucleobases in length. An antisense strand core stretch sequence is 100% (perfectly) complementary or at least about 85% (substantially) complementary to a nucleotide sequence (sometimes referred to, e.g., as a target sequence) present in the HBV mRNA target. A sense strand core stretch sequence is 100% (perfectly) complementary or at least about 85% (substantially) complementary to a core stretch sequence in the antisense strand, and thus the sense strand core stretch sequence is perfectly identical or at least about 85% identical to a nucleotide sequence (target sequence) present in the HBV mRNA target. A sense strand core stretch sequence can be the same length as a corresponding antisense core sequence or it can be a different length. In some embodiments, the antisense strand core stretch sequence is 16, 17, 18, 19, 20, 21, 22, or 23 nucleotides in length. In some embodiments, the sense strand core stretch sequence is 16, 17, 18, 19, 20, 21, 22, or 23 nucleotides in length.

As used herein, an “RNA interference agent,” “RNAi agent,” “RNA interference molecule” or “RNAi molecule” means a composition that contains an RNA or RNA-hke (e.g., chemically modified RNA) oligonucleotide molecule that is capable of degrading or inhibiting translation of messenger RNA (mRNA) transcripts of a target mRNA in a sequence specific manner. As used herein, RNAi agents can operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short interfering RNAs (siRNAs), double-stranded RNAs (dsRNA), micro RNAs (miRNAs), short hairpin RNAs (shRNA), and dicer substrates. RNAi agents of the application are preferably dsRNAs. The antisense strand of the RNAi agents described herein is at least partially complementary to the mRNA being targeted. RNAi agents can be comprised of modified nucleotides and/or one or more non-phosphodiester linkages.

The term “double-stranded RNA”, “dsRNA molecule”, or “dsRNA”, as used herein, refers to a ribonucleic acid molecule, or complex of ribonucleic acid molecules, having a duplex structure comprising two anti-parallel and substantially complementary nucleic acid strands. The two strands forming the duplex structure can be different portions of one larger RNA molecule, or they can be separate RNA molecules. Where the two strands are part of one larger molecule, and therefore are connected by an uninterrupted chain of nucleotides between the 3-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands can have the same or a different number of nucleotides. In addition to the duplex structure, a dsRNA can comprise one or more nucleotide overhangs or can be blunt ended.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein or protein subunit translated from the mRNA in a cell, group of cells, tissue, organ, or subject in which the gene is transcribed, is reduced when the cell, group of cells, tissue, organ, or subject is treated with oligomeric compounds, such as RNAi agents, described herein as compared to a second cell, group of cells, tissue, organ, or subject that has not or have not been so treated.

The term “Hepatitis B Virus gene” as used herein relates to the genes necessary for replication and pathogenesis of Hepatitis B Vims, in particular to the genes that encode core protein, viral polymerase, surface antigen, c-antigen and the X protein and the genes that encode the functional fragments of the same. The term “Hepatitis B Virus gene/sequence” does not only relate to (the) wild-type sequence(s) but also to mutations and alterations which can be comprised in said gene/sequence. Accordingly, the present application is not limited to the specific RNAi agents provided herein. The application also relates to RN Ai agents that comprise an antisense strand that is at least 85% complementary to the corresponding nucleotide stretch of an RNA transcript of a Hepatitis B Virus gene that comprises such mutations/alterations.

As used herein, the term “consensus sequence” refers to at least 13 contiguous nucleotides, preferably at least 17 contiguous nucleotides, most preferably at least 19 contiguous nucleotides, which is highly conserved among the Hepatitis B Virus genomic sequences of genotype A, B, C and D.

As used herein, “target sequence” refers to a contiguous portion of the nucleotide sequence of an mRNA molecule formed during the transcription of a Hepatitis B Virus gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature. However, as detailed herein, such a “strand comprising a sequence” can also comprise modifications, like modified nucleotides.

RNAi agents are capable of inhibiting the expression of a Hepatitis B Virus by at least about 60%, preferably by at least 70%, most preferably by at least 80% in in vitro assays, i.e. In vitro. The term “in vitro” as used herein includes but is not limited to cell culture assays. The person skilled in the art can readily determine such an inhibition rate and related effects, in particular in light of the assays provided herein. The term “off target” as used herein refers to all non-target mRNAs of the transcriptome that are predicted by in silico methods to hybridize to the described RNAi agents based on sequence complementarity. The RNAi agents of the present application preferably specifically inhibit the expression of Hepatitis B Virus gene, i.e. do not inhibit the expression of any off-target.

RNAi agents of the application can contain one or more mismatches to the target sequence. In a preferred embodiment, RNAi agents of the application contains no more than 13 mismatches. If the antisense strand of the RNAi agent contains mismatches to a target sequence, it is preferable that the area of mismatch not be located within nucleotides 2-7 of the 5′ terminus of the antisense strand. In another embodiment, it is preferable that the area of mismatch not be located within nucleotides 2-9 of the 5′ terminus of the antisense strand.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence. “Complementary” sequences, as used herein, can also include, or be formed entirely from, non-Watson-Crick base pairs and/or base pairs formed from non-natural and modified nucleotides, in as far as the above requirements with respect to their ability to hybridize are fulfilled.

The term “antisense strand” refers to the strand of a dsRNA which includes a region that is substantially complementary to a target sequence. As used herein, the term “region of complementarity” refers to the region on the anti sense strand that is substantially complementary to a sequence, for example a target sequence. Where the region of complementarity is not fully complementary to the target sequence, the mismatches are most tolerated outside nucleotides 2-7 of the 5′ terminus of the antisense strand.

The term “sense strand,” as used herein, refers to the strand of a dsRNA that includes a region that is substantially complementary to a region of the antisense strand. “Substantially complementary” means preferably at least 85% of the overlapping nucleotides in sense and antisense strand are complementary.

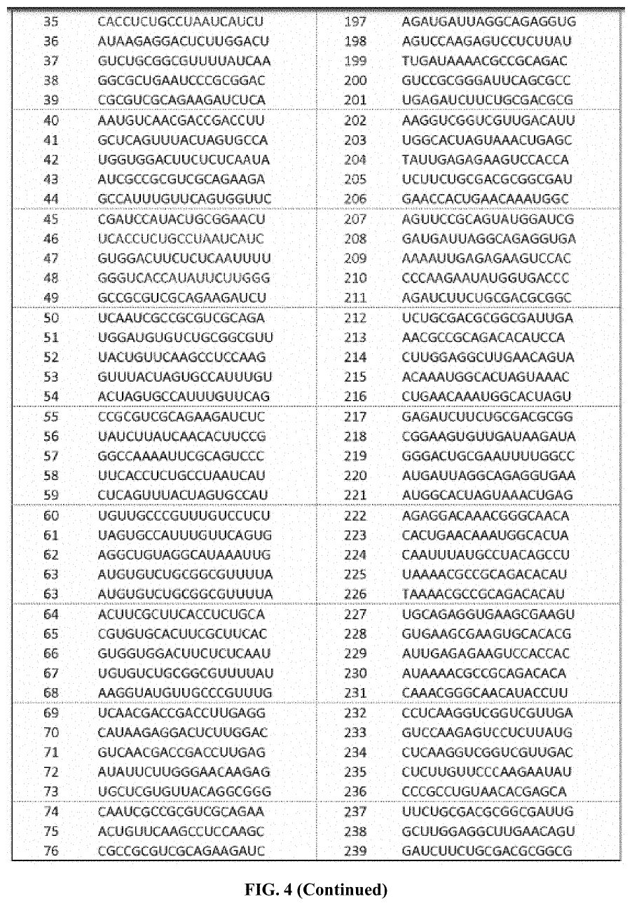

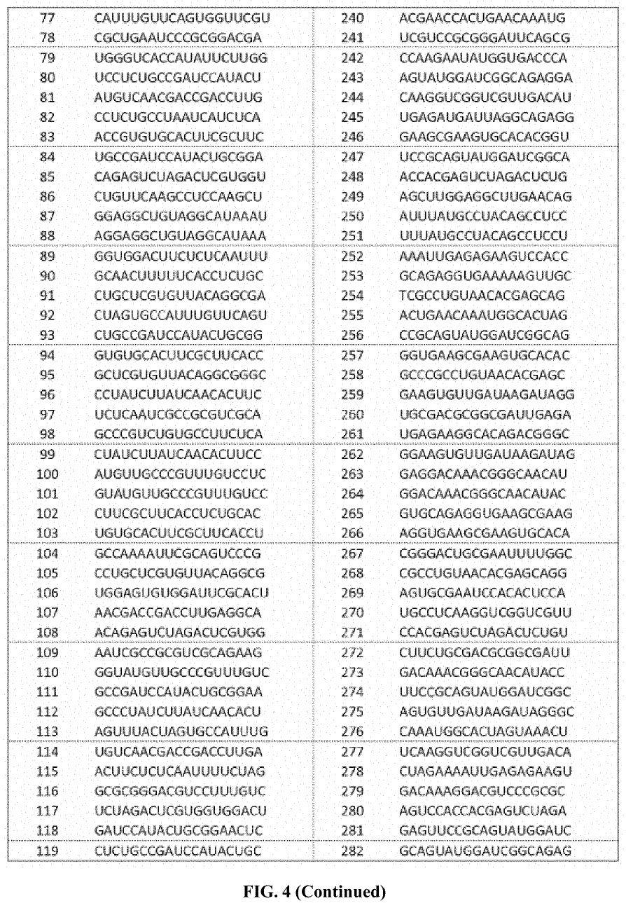

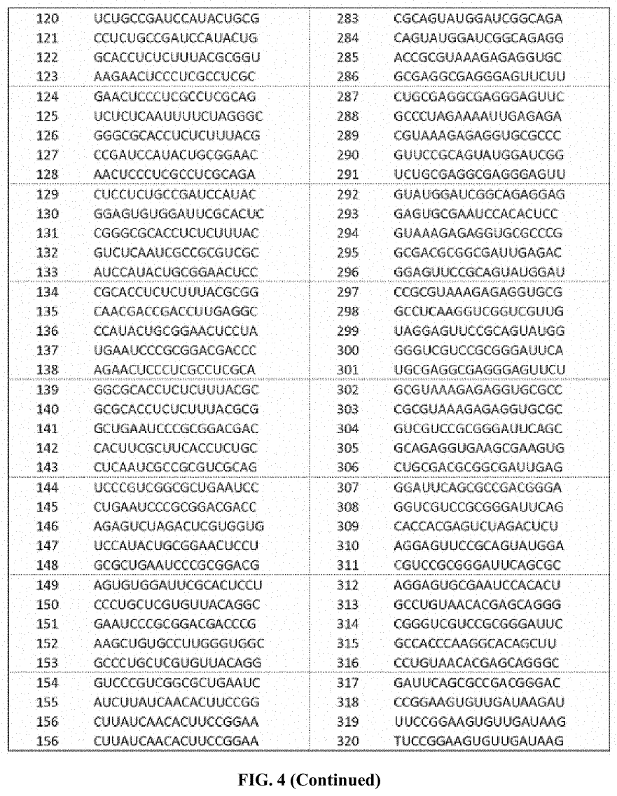

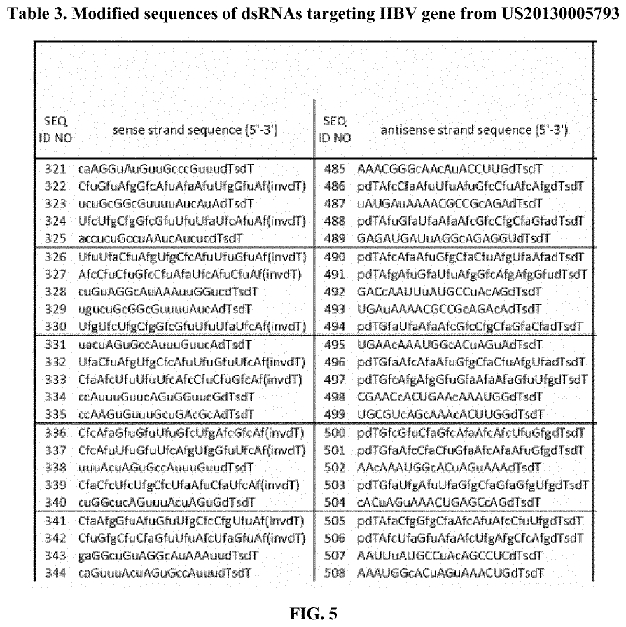

Examples of sense and antisense strand nucleotide sequences used in forming HBV RNAi agents are provided in FIGS. 4-6 and 8-10, reproduced from US20130005793 and WO2018027106, the content of which are incorporated herein in their entirety.

The HBV RNAi agent sense and antisense strands anneal to form a duplex. A sense strand and an antisense strand of an HBV RNAi agent can be partially, substantially, or fully complementary to each other. Within the complementary duplex region, the sense strand core stretch sequence is at least about 85% complementary or 100% complementary to the anti sense core stretch sequence. In some embodiments, the sense strand core stretch sequence contains a sequence of at least 16, at least 17, at least 18, at least 19, at least 20, or at least 21 nucleotides that is at least about 85% or 100% complementary to a corresponding 16, 17, 18, 19, 20, or 21 nucleotide sequence of the antisense strand core stretch sequence (i.e., the sense strand and antisense core stretch sequences of an HBV RNAi agent have a region of at least 16, at least 17, at least 18, at least 19, at least 20, or at least 21 nucleotides that is at least 85% base paired or 100% base paired).

In some embodiments, the antisense strand of an HBV RNAi agent disclosed. herein differs by 0, 1, 2, or 3 nucleotides from any of the antisense strand sequences described herein. In some embodiments, the sense strand of an HBV RNAi agent disclosed herein differs by 0, 1, 2, or 3 nucleotides from any of the sense strand sequences described herein.

The length of the HBV RNAi agent sense and anti sense strands described herein are independently 16 to 30 nucleotides in length. In some embodiments, the sense and antisense strands are independently 17 to 26 nucleotides in length. In some embodiments, the sense and antisense strands are 19-26 nucleotides in length. In some embodiments, the described RNAi agent sense and antisense strands are independently 17, 18, 19, 20, 21, 22, 23, 24, 25, or 26 nucleotides in length. The sense and antisense strands can be either the same length or they can be different lengths. In some embodiments, a sense strand and an antisense strand are each 26 nucleotides in length. In some embodiments, a sense strand is 23 nucleotides in length and an antisense strand is 21 nucleotides in length. In some embodiments, a sense strand is 22 nucleotides in length and an antisense strand is 21 nucleotides in length. In some embodiments, a sense strand is 21 nucleotides in length and an anti sense strand is 21 nucleotides in length. In some embodiments, a sense strand is 19 nucleotides in length and an antisense strand is 21 nucleotides in length.

The sense strand and/or the antisense strand can optionally and independently contain an additional 1, 2, 3, 4, 5, or 6 nucleotides (extension) at the 3′ end, the 5′ end, or both the 3′ and 5′ ends of the core sequences. The antisense strand additional nucleotides, if present, may or may not be complementary to the corresponding sequence in an HBV mRNA. The sense strand additional nucleotides, if present, may or may not be identical to the corresponding sequence in an HBV mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch sequence nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand, In other embodiments, one or more of 3′ extension nucleotides of one strand do not base pair with one or more 5′ extension nucleotides of the other strand. In some embodiments, an HBV RNAi agent has an antisense strand having a 3′ extension and a sense strand having a 5′ extension. In some embodiments, an HBV RNAi agent comprises an antisense strand having a 3′ extension of 1, 2, 3, 4, 5, or 6 nucleotides in length. In other embodiments, an HBV RNAi agent comprises an antisense strand having a 3′ extension of 1, 2, or 3 nucleotides in length, In some embodiments, one or more of the antisense strand extension nucleotides comprise uracil or thymidine nucleotides or nucleotides which are complementary to a corresponding HBV mRNA sequence. In some embodiments, a 3′ antisense strand extension includes or consists of, but is not limited to: AUA, UGCUU, CUG, UG, UGCC, CUGCC, CGU, CUU, UGCCUA, CUGCCU, UGCCU, UGAUU, GCCUAU, T, TT, U, UU (each listed 5′ to 3′). In some embodiments, the 3′ end of the antisense strand can include additional abasic nucleosides (Ab). In some embodiments, Ab or AbAb can be added to the 3′ end of the antisense strand.

In some embodiments, an HBV RNAi agent comprises an antisense strand having a 5′ extension of 1, 2, 3, 4, or 5 nucleotides in length. In other embodiments, an HBV RNAi agent comprises an antisense strand having a 5′ extension of 1 or 2 nucleotides in length. In some embodiments, one or more of the antisense strand extension nucleotides comprises uracil or thymidine nucleotides or nucleotides which are complementary to a corresponding HBV mRNA sequence. In some embodiments, the 5′ antisense strand extension includes or consists of, but is no limited to, UA, TU, U, T, UU, TT, CUC (each listed 5′ to 3′). An antisense strand can have any of the 3′ extensions described above in combination with any of the 5′ antisense strand extensions described, if present.

In some embodiments, an HBV RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides which correspond to nucleotides in the HBV mRNA sequence. In some embodiments, the 3′ sense strand extension includes or consists of, but is not limited to: T, UT, TT, UU, UUT, TTT, or TTTT (each listed 5′ to 3′).

In some embodiments, the 3′ end of the sense strand can include additional abasic nucleosides. In some embodiments, UUAb, UAb, or Ab can be added to the 3′ end of the sense strand. In some embodiments, the one or more abasic nucleosides added to the 3′ end of the sense strand can be inverted (invAb). In some embodiments, one or more inverted abasic nucleosides can be inserted between the targeting ligand and the nucleohase sequence of the sense strand of the RNAi agent. In some embodiments, the inclusion of one or more inverted abasic nucleosides at or near the terminal end or terminal ends of the sense strand of an RNAi agent can allow for enhanced activity or other desired properties of an RNAi agent. In some embodiments, an HBV RNAi agent comprises a sense strand having a 5′ extension of 1, 2, 3, 4, 5, or 6 nucleotides in length. In some embodiments, one or more of the sense strand extension nucleotides comprise uracil or adenosine nucleotides or nucleotides which correspond to nucleotides in the HBV mRNA sequence. In some embodiments, the sense strand 5′ extension can be, but is not limited to: CA, AUAGGC, AUAGG, AUAG, AUA, A, AA, AC, GCA, GGCA, GGC, UAUCA, UAUC, UCA, UAU, U, UU (each listed 5′ to 3′). A sense strand can have a 3′ extension and/or a 5′ extension.

In some embodiments, the 5′ end of the sense strand can include an additional abasic nucleoside (Ab) or nucleosides (AbAb). In some embodiments, the one or more abasic nucleosides added to the 5′ end of the sense strand can be inverted (invAb). In some embodiments, one or more inverted abasic nucleosides can be inserted between the targeting ligand and the nucleobase sequence of the sense strand of the RNAi agent. In some embodiments, the inclusion of one or more inverted abasic nucleosides at or near the terminal end or terminal ends of the sense strand of an RNAi agent can allow for enhanced activity or other desired properties of an RNAi agent.

Examples of nucleotide sequences used in forming HBV RNAi agents are provided in FIGS. 4-6 and 8-10, reproduced from US20130005793 and WO2018027106. In some embodiments, an HBV RNAi agent antisense strand includes a nucleotide sequence of any of the sequences in FIG. 4-6, 8 or 9. In some embodiments, an BBV RNAi agent antisense strand includes the sequence of nucleotides 1-17, 2-17, 1-18, 2-18, 1-19, 2-19, 1-20, 2-20, 1-21, 2-21, 1-22, 2-22, 1-23, 2-23, 1-24, 2-24, 1-25, 2-25, 1-26, or 2-26 of any of the sequences in FIG. 4-6, 8 or 9. In some embodiments, an HBV RNAi agent sense strand includes the nucleotide sequence of any of the sequences in FIG. 4-6, 8 or 10. In some embodiments, an HB RNAi agent sense strand includes the sequence of nucleotides 1-18, 1-19, 1-20, 1-21, 1-22. 1-23, 1-24, 1-25, 1-26, 2-19, 2-20, 2-21, 2-22, 2-23, 2-24, 2-25, 2-26, 3-20, 3-21, 3-22, 3-23, 3-24, 3-25, 3-26, 4-21, 4-22, 4-23, 4-24, 4-25, 4-26, 5-22, 5-23, 5-24, 5-25, 5-26, 6-23, 6-24, 6-25, 6-26, 7-24, 7-25, 7-25, 8-25, 8-26 of any of the sequences in FIG. 4-6, 8 or 10. In some embodiments, the sense and antisense strands of the RNAi agents described herein contain the same number of nucleotides. In some embodiments, the sense and anti sense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a blunt end refers to an end of a double stranded. RNAi agent in which the terminal nucleotides of the two annealed strands are complementary (form a complementary base-pair). In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands from a pair (i.e. do not form an overhang) but are not complementary (i.e. form a non-complementary pair). As used herein, an overhang is a stretch of one or more unpaired nucleotides at the end of one strand of a double stranded RNAi agent. The unpaired nucleotides can be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends.

A nucleotide base (or nucleobase) is a heterocyclic pyrimidine or purine compound which is a constituent of all nucleic acids and includes adenine (A), guanine (G), cytosine (C), thymine (T), and uracil (U). As used herein, the term “nucleotide” can include a modified nucleotide (such as, for example, a nucleotide mimic, abasic site (Ab), or a surrogate replacement moiety). Modified nucleotides, when used in various polynucleotide or oligonucleotide constructs, can preserve activity of the compound in cells while at the same time increasing the serum stability of these compounds, and can also minimize the possibility of activating interferon activity in humans upon administering of the polynucleotide or oligonucleotide construct.

In some embodiments, an HBV RNAi agent is prepared or provided as a salt, mixed salt, or a free-acid. In some embodiments, an HBV RNAi agent is prepared as a sodium salt. Such forms are within the scope of the application disclosed herein.

“Introducing into a cell”, when referring to RNAi agents, means facilitating uptake or absorption into the cell, as is understood by those skilled in the art. Absorption or uptake of RNAi agents can occur through unaided diffusive or active cellular processes, or by auxiliary agents or devices. The meaning of this term is not limited to cells in vitro; RNAi agents can also be “introduced into a cell”, wherein the cell is part of a living organism. In such instance, introduction into the cell will include the delivery to the organism. For example, for in vivo delivery, RNAi agents can be injected into a tissue site or administered systemically. It is, for example envisaged that the RNAi agents of this application be administered to a subject in need of medical intervention. Such an administration can comprise the injection of the RNAi agents, the vector or a cell of this application into a diseased site in said subject, for example into liver tissue/cells or into cancerous tissues/cells, like liver cancer tissue. In addition, the injection is preferably in close proximity to the diseased tissue envisaged. In vitro introduction into a cell includes methods known in the art such as electroporation and lipofection.

The term “half-life” as used herein is a measure of stability of a compound or molecule and can be assessed by methods known to a person skilled in the art, especially in light of the assays provided herein. The term “non-immunostimulatory” as used herein refers to the absence of any induction of an immune response by the described RNAi agents. Methods to determine immune responses are well known to a person skilled in the art, for example by assessing the release of cytokines, as described in the examples section.

### Modified Nucleotides

In some embodiments, an HBV RNAi agent contains one or more modified nucleotides. The nucleic acids of the application can be synthesized and/or modified by methods well established in the art. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides (represented herein as Ab), 2′-modified nucleotides. 3′ to 3′ linkages (inverted) nucleotides (represented herein as invdN, invN, invn, invAb), non-natural base-comprising nucleotides, bridged nucleotides, peptide nucleic acids (PNAs), 2′,3′-seco nucleotide mimics (unlocked nucleobase analogues, represented herein as NUNA), locked nucleotides (represented herein as NLNA), 3′-0-methoxy (2′ internucleoside linked) nucleotides (represented herein as 3′-OMen), 2′-F-Arabino nucleotides (represented herein as NfANA), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e. a nucleotide with a group other than a hydroxyl group at the 2′ position of the five-membered sugar ring) include, but are not limited to, 2′-0-methyl nucleotides (represented herein as a lower case letter ‘n’ in a nucleotide sequence), 2′-deoxy-2′-fluoro nucleotides (represented herein as Nf, also represented herein as 2′-fluoro nucleotide), 2′-deoxy nucleotides (represented herein as dN), 2′-methoxy ethyl (2′-0-2-methoxylethyl) nucleotides (represented herein as NM or 2′-MOE), 2′-amino nucleotides, and 2′-alkyl nucleotides. It is not necessary for all positions in a given compound to be uniformly modified. Conversely, more than one modification can be incorporated in a single HBV RNAi agent or even in a single nucleotide thereof. The HBV RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidmes, 6-azapyrimi dines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, -uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoiOinethyl, and other 5-substituted uracils and cytosines, 7-methylguanine and 7-methyladenine, 8-azaguanine and 8-azaadenine, 7-deazaguanine, 7-dea/aadenine, 3-deazaguanine, and 3-deazaadenine. In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides. As used herein, a sense strand wherein substantially all of the nucleotides present are modified nucleotides is a sense strand having two or fewer (i.e., 0, 1, or 2) nucleotides in the sense strand being ribonucleotides. As used herein, an antisense sense strand wherein substantially all of the nucleotides present are modified nucleotides is an antisense strand having two or fewer (i.e., 0, 1, or 2) nucleotides in the sense strand being ribonucleotides. In some embodiments, one or more nucleotides of an RNAi agent is a ribonucleotide.

As used herein, the term “sugar substituent group” or “2′-substituent group” includes groups attached to the 2′-position of the ribofuranosyl moiety with or without an oxygen atom. Sugar substituent groups include, but are not limited to, fluoro, O-alkyl, O-alkylamino, O-alkylalkoxy, protected O-alkylamino, O-alkylaminoalkyl, O-alkyl imidazole and poly ethers of the formula (O-alkyl)m, wherein m is 1 to about 10. Preferred among these polyethers are linear and cyclic polyethylene glycols (PEGs), and (PEG)-containing groups, such as crown ethers and, inter glia, those which are disclosed by Delgardo et. al. (Critical Reviews in Therapeutic Drug Carrier Systems (1992) 9:249). Further sugar modifications are disclosed by Cook (Anti-fibrosis Drug Design, (1991) 6:585-607). Fluoro, O-alkyl, O-alkylamino, O-alkyl imidazole, O-alkylaminoalkyl, and alkyl amino substitution is described in U.S. Pat. No. 6,166,197, entitled “Oligomeric Compounds having Pyrimidine, Nucleotide(s) with 2′ and 5′ Substitutions.” hereby incorporated by reference in its entirety.

Additional sugar substituent groups amenable to the application include 2′-SR and 2′-NR₂ groups, wherein each R is, independently, hydrogen, a protecting group or substituted or unsubstituted alkyl, alkenyl, or alkynyl. 2′-SR Nucleosides are disclosed in U.S. Pat. No. 5,670,633, hereby incorporated by reference in its entirety. The incorporation of 2′-SR monomer synthons is disclosed by Hamm et al. (J. Org. Chem., (1997) 62:3415-3420). 2′-NR nucleosides are disclosed by Thomson J B, J. Org. Chem., (1996) 61:6273-6281; and Polushin et al., Tetrahedron Lett., (1996) 37:3227-3230. Further representative 2′-substituent groups amenable to the application include those having one of formula I or II:

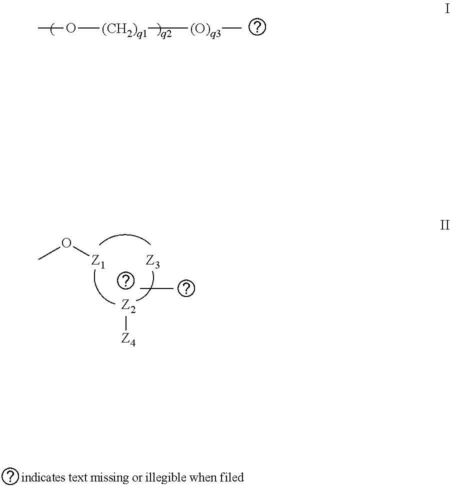

wherein

E is C₁-C₁₀ alkyl, N(Q3)(Q4) or C(Q3)(Q4); each Q3 and Q4 is, independently, H, C₁-C₁₀ alkyl, dialkylaminoalkyl, a nitrogen protecting group, a tethered or untethered conjugate group, a linker to a solid support; or Q3 and Q4, together, form a nitrogen protecting group or a ring stnicture optionally including at least one additional heteroatom selected from N and O;

q1 is an integer from 1 to 10;

q2 is an integer from 1 to 10;

q3 is 0 or 1;

q4 is 0, 1 or 2;

each Z1, Z2, and Z3 is, independently, C₄-C₇ cycloalkyl, C₅-C₁₄ aryl or C₃-C₁₅ heterocyclyl, wherein the heteroatom in said heterocyclyl group is selected from oxygen, nitrogen and sulfur;

Z4 is OM1, SMI, or N(M1)₂; each Ml is, independently, H, C₁-C₈ alkyl, C₁-C₈ haloalkyl, C(═NH)N(H)M2, C(═O)N(H)M2 or OC(═O)N(H)M2; M2 is H or C₁-C₈ alkyl; and

Z5 is C₁-C₁₀ alkyl, C₁-C₀ haloalkyl, C₂-C₁₀ alkenyl, C₂-C₁₀ alkynyl, C₆-C₁₄ aryl, N(Q3)(Q4), OQ3, halo, SQ3 or CN.

Representative 2′-O-sugar substituent groups of formula I are disclosed in U.S. Pat. No. 6,172,209, entitled “Capped 2′-Oxyethoxy Oligonucleotides,” hereby incorporated by reference in its entirety. Representative cyclic 2′-O-sugar substituent groups of formula II are disclosed in U.S. Pat. No. 6,271,358, entitled “RNA Targeted 2′-Modified Oligonucleotides that are Conformationally Preorganized,” hereby incorporated by reference in its entirety.

Sugars having O-substitutions on the ribosyl ring are also amenable to the application. Representative substitutions for ring O include, but are not limited to, S, CH₂, CHF, and CF₂.
Oligonucleotides can also have sugar mimetics, such as cyclobutyl moieties, in place of the pentofuranosyl sugar. Representative United States patents relating to the preparation of such modified sugars include, but are not limited to, U.S. Pat. No. 5,359,044, U.S. Pat. No. 5,466,786, U.S. Pat. No. 5,519,134, U.S. Pat. No. 5,591,722, U.S. Pat. No. 5,597,909, U.S. Pat. No. 5,646,265, and U.S. Pat. No. 5,700,920, all of which are hereby incorporated by reference. Modified internucleoside Linkages

In some embodiments, one or more nucleotides of an HBV RNAi agent are linked by nonstandard linkages or backbones (i.e., modified internucleoside linkages or modified backbones). In some embodiments, a modified internucleoside linkage is a non-phosphate-containing covalent internucleoside linkage. Modified internucleoside linkages or backbones include, but are not limited to, 5′-phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates. phosphinates, phosphorami dates (e.g., 3′-amino phosphoramidate, aminoalkylphosphoramidates, or thionophosphoramidates), thionoalkyl-phosphonates, thionoalkylphosphotriesters, morpholine linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphoms atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, 0, S, and CH2 components.

In some embodiments, a sense strand of an HBV RNAi agent can contain 1, 2, 3, 4, 5, or 6 phosphorothioate linkages, an antisense strand of an HBV RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an HBV RNAi agent can contain 1, 2, 3, or 4 phosphorothioate linkages, an antisense strand of an HBV RNAi agent can contain 1, 2, 3, or 4 phosphorothioate linkages, or both the sense strand and the antisense strand independently can contain 1, 2, 3, or 4 phosphorothioate linkages. In some embodiments, an HBV RNAi agent sense strand contains at least two phosphorothioate internucleoside linkages. In some embodiments, the at least two phosphorothioate internucleoside linkages are between the nucleotides at positions 1-3 from the 3′ end of the sense strand. In some embodiments, the at least two phosphorothioate internucleoside linkages are between the nucleotides at positions 1-3, 2-4, 3-5, 4-6, 4-5, or 6-8 from the 5′ end of the sense strand. In some embodiments, an HBV RNAi agent antisense strand contains four phosphorothioate internucleoside linkages. In some embodiments, the four phosphorothioate internucleoside linkages are between the nucleotides at positions 1-3 from the 5′ end of the sense strand and between the nucleotides at positions 19-21, 20-22, 21-23, 22-24, 23-25, or 24-26 from the 5′ end. In some embodiments, an HBV RNAi agent contains at least two phosphorothioate internucleoside linkages in the sense strand and three or four

In some embodiments, an HBV RNAi agent contains one or more modified nucleotides and one or more modified internucleoside linkages. In some embodiments, a 2′-modified nucleoside is combined with modified internucleoside linkage.

### Chemical Modifications

RNAi agents of the present application can also be chemically modified to enhance stability. The nucleic acids of the application can be synthesized and/or modified by methods well established in the art. Chemical modifications can include, but are not limited to 2′ modifications, introduction of non-natural bases, covalent attachment to a ligand, and replacement of phosphate linkages with thiophosphate linkages, inverted deoxythymidines. In this embodiment, the integrity of the duplex structure is strengthened by at least one, and preferably two, chemical linkages. Chemical linking can be achieved by any of a variety of well-known techniques, for example by introducing covalent, ionic or hydrogen bonds; hydrophobic interactions, van der Waals or stacking interactions; by means of metal-ion coordination, or through use of purine analogues. Preferably, the chemical groups that can be used to modify the RNAi agents include, without limitation, methylene blue; bifunctional groups, preferably bis-(2-chloroethyl)amine; -acetyl-N′-(p-glyoxylbenzoyl)cystamine; 4-thiouracil; and psoralen. In one preferred embodiment, the linker is a hexa-ethylene glycol linker. In this case, the RNAi agents are produced by solid phase synthesis and the hexa-ethylene glycol linker is incorporated according to standard methods (e.g., Williams D J and Hall K B, Biochem. (1996) 35: 14665-14670). In a particular embodiment, the 5′-end of the antisense strand and the 3′-end of the sense strand are chemically linked via a hexaethylene glycol linker. In another embodiment, at least one nucleotide of the RNAi agent comprises a phosphorothioate or phosphorodithioate groups. The chemical bond at the ends of the RNAi agent is preferably formed by triple-helix bonds.

### HBV RNAi Agents

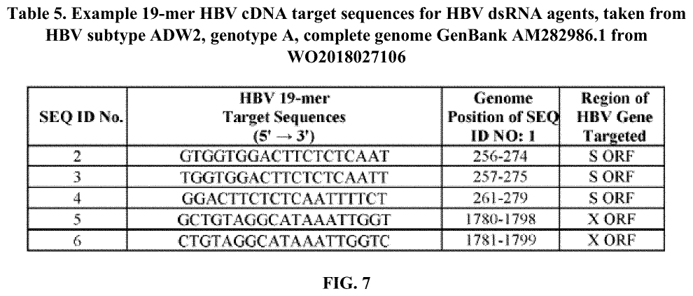

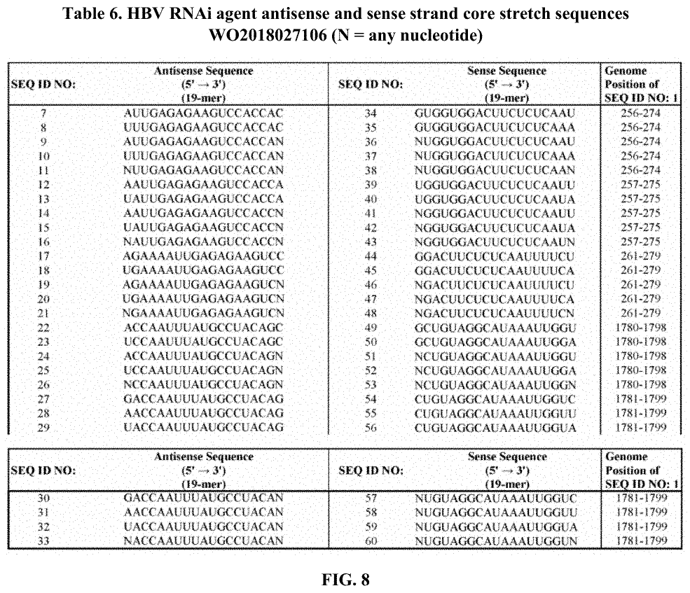

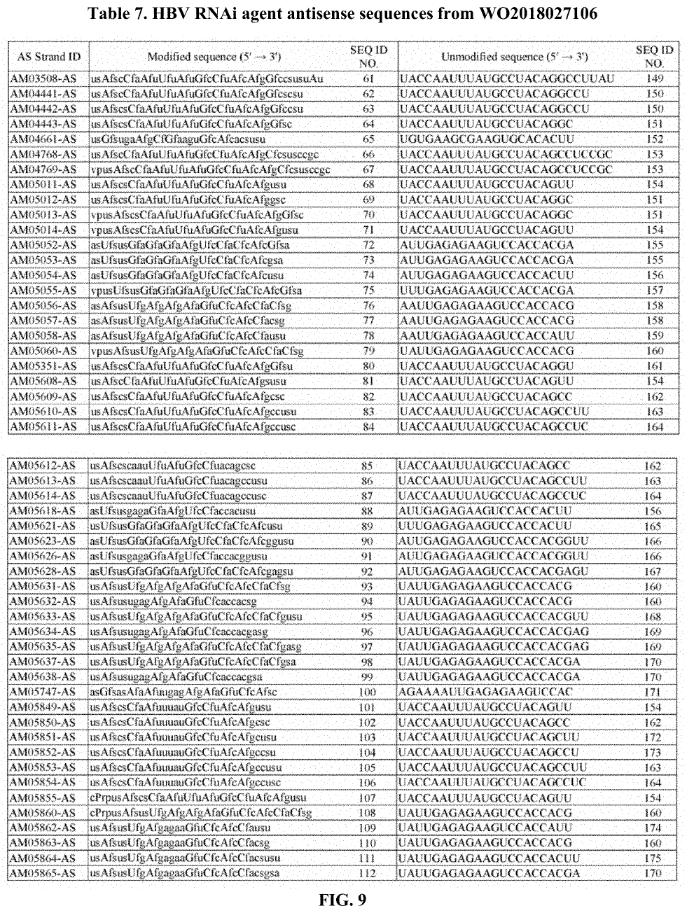

In some embodiments, the HBV RNAi agents disclosed herein target an HBV gene at or near the positions of the HBV genome shown in FIG. 7. In some embodiments, the antisense strand of an HBV RNAi agent disclosed herein includes a core stretch sequence that is fully, substantially, or at least partially complementary to a target HBV 19-mer sequence disclosed in FIG. 7.

In some embodiments, an HBV RNAi agent includes an antisense strand wherein position 19 of the antisense strand (5′→3′) is capable of forming a base pair with position 1 of a 19-mer target sequence disclosed in FIG. 7. In some embodiments, an HBV RNAi agent includes an antisense strand wherein position 1 of the antisense strand (5′→3′) is capable of forming a base pair with position 19 of the 19-mer target sequence disclosed in FIG. 7. In some embodiments, an HBV RNAi agent includes an antisense strand wherein position 2 of the antisense strand (5′→3′) is capable of forming a base pair with position 18 of the 19-mer target sequence disclosed in FIG. 7. In some embodiments, an HBV RNAi agent includes an antisense strand wherein positions 2 through 18 of the antisense strand (5→3′) are capable of forming base pairs with each of the respective complementary bases located at positions 18 through 2 of the 19-mer target sequence disclosed in FIG. 7.

In some embodiments, the HBV RNAi agents include core 19-mer nucleotide sequences shown in FIG. 4-6 or 8. The HBV RNAi agent sense strands and antisense strands that comprise or consist of the nucleotide sequences in FIG. 4-6 or 8 can be modified nucleotides or unmodified nucleotides. In some embodiments, the HBV RNAi agents having the sense and antisense strand sequences that comprise or consist of the nucleotide sequences in FIG. 4-6 or 8 are all or substantially all modified nucleotides. In some embodiments, the antisense strand of an HBV RNAi agent disclosed herein differs by 0, 1, 2, or 3 nucleotides from any of the antisense strand sequences in FIG. 4-6 or 8. In some embodiments, the sense strand of an HBV RNAi agent disclosed herein differs by 0, 1, 2, or 3 nucleotides from any of the sense strand sequences in FIG. 4-6 or 8.

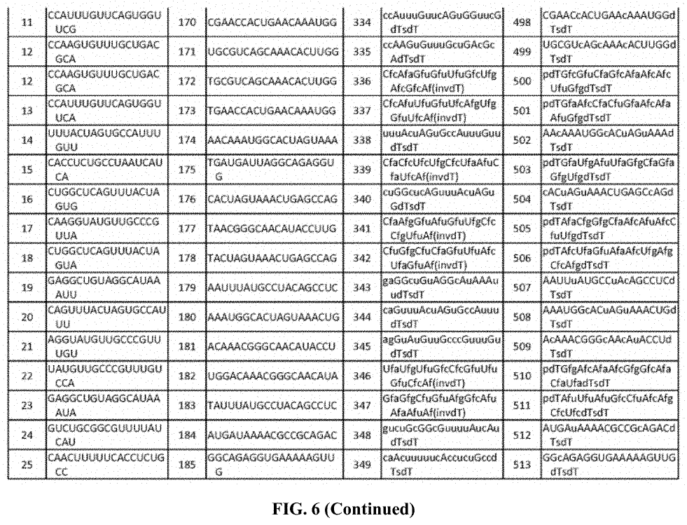

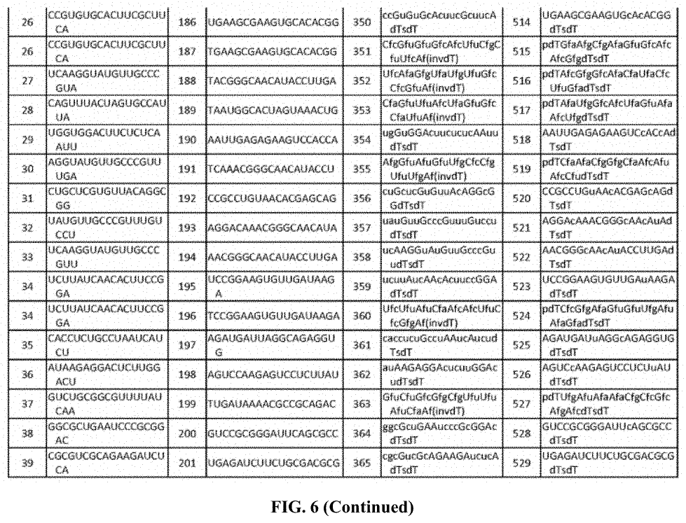

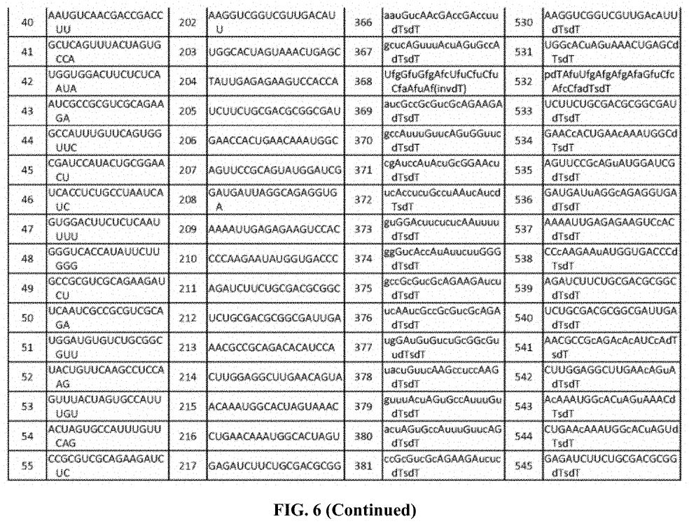

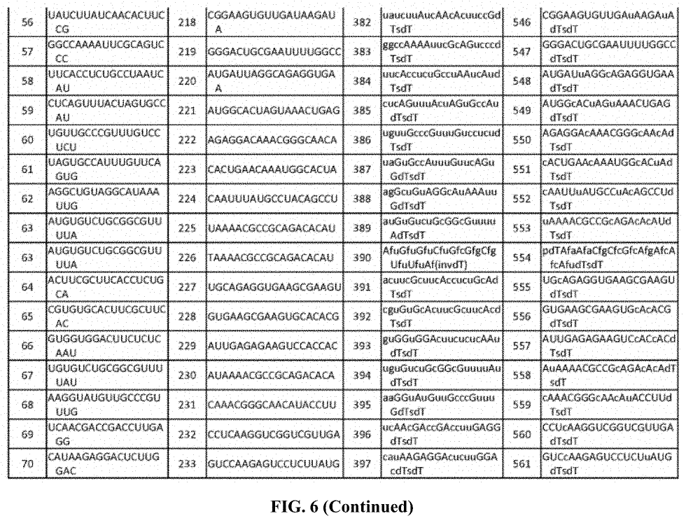

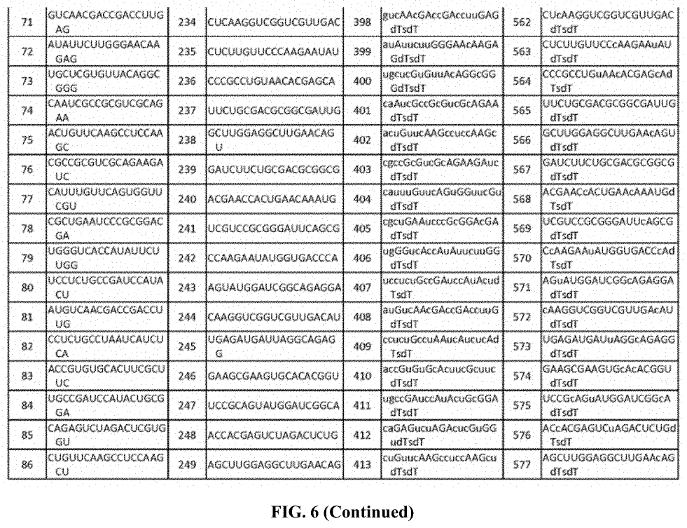

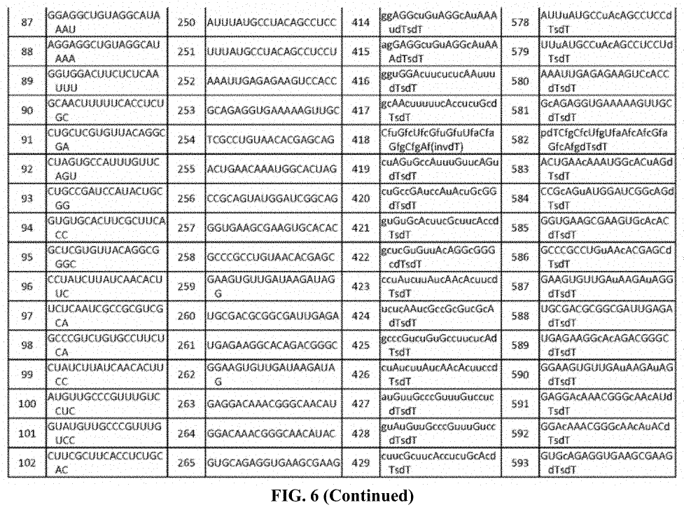

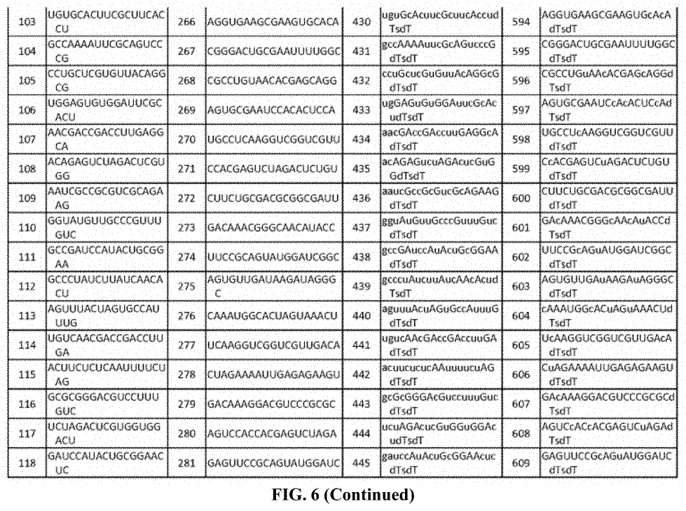

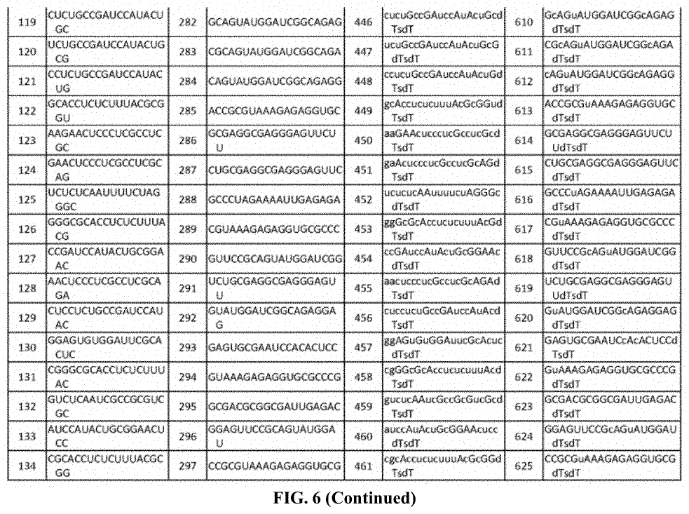

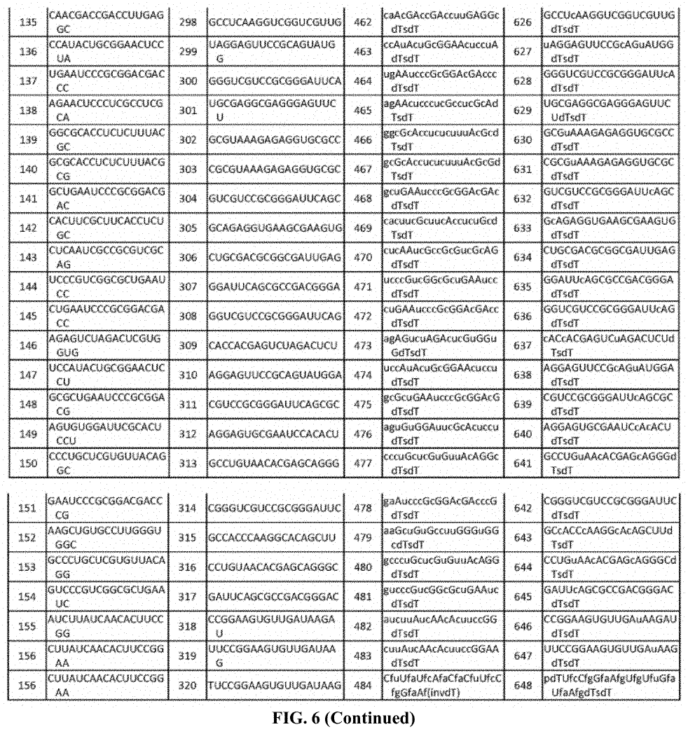

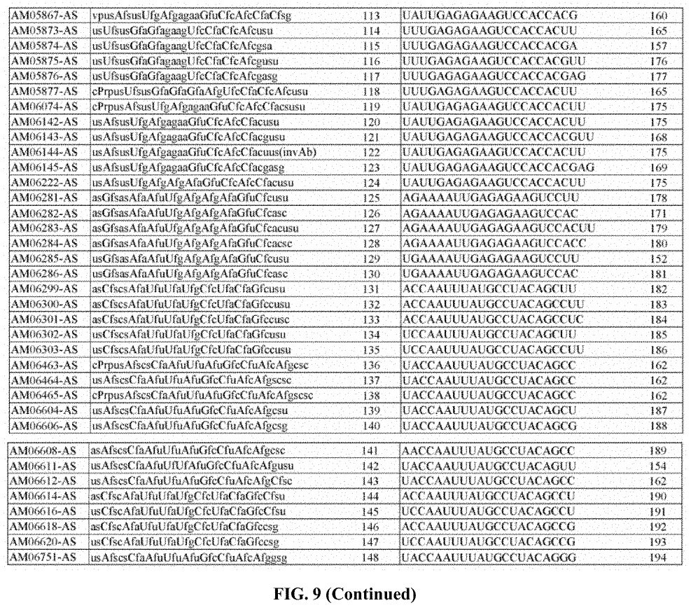

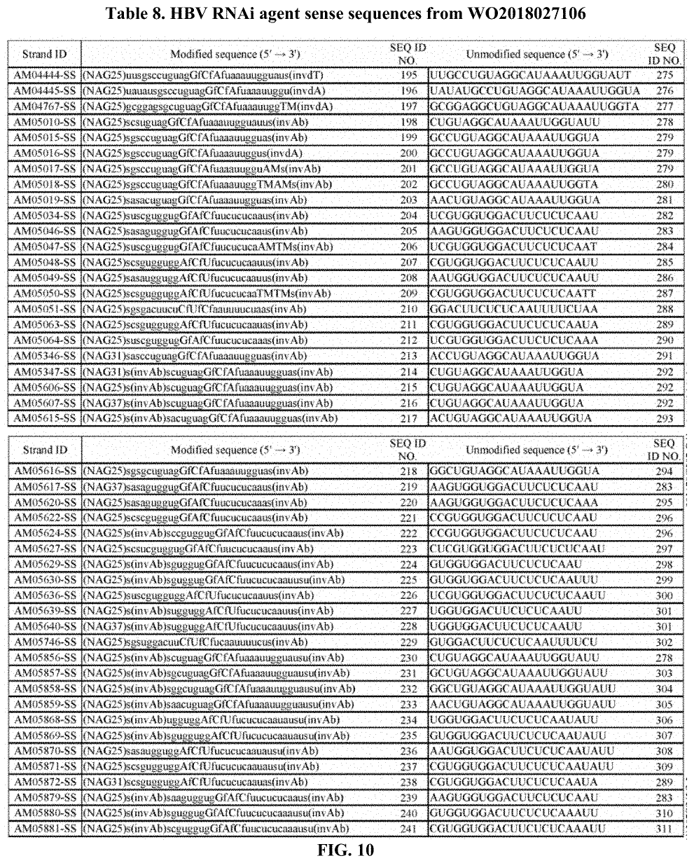

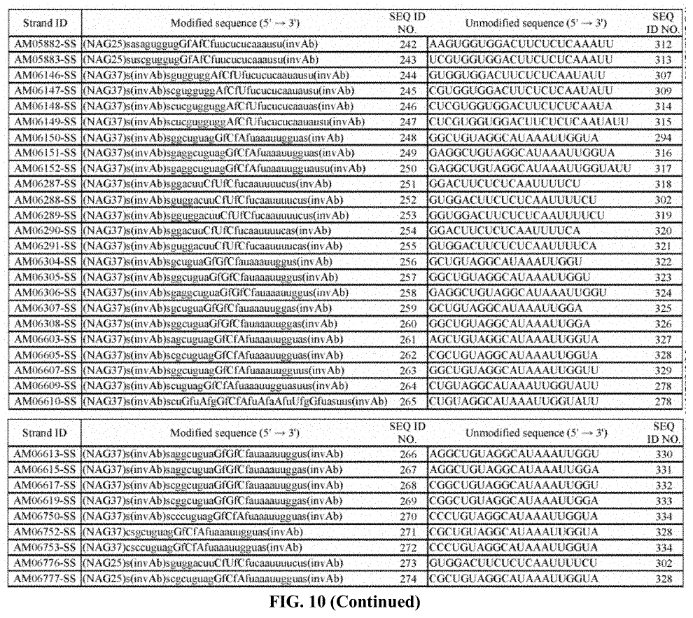

Modified HBV RNAi agent antisense strand sequences, as well as their underlying unmodified sequences, are provided in FIGS. 6 and 9. Modified HBV RNAi agent sense strands, as well as their underlying unmodified sequences, are provided in FIGS. 6 and 10. In forming HBV RNAi agents, each of the nucleotides in each of the unmodified sequences listed in FIGS. 6 and 9-10 can be a modified nucleotide.

As used herein (including in FIGS. 9-10), the following notations are used to indicate modified nucleotides, targeting groups, and linking groups. As the person of ordinary skill in the art would readily understand, unless otherwise indicated by the sequence, that when present in an oligonucleotide, the monomers are mutually linked by 5′-3′-phosphodiester bonds:

A=adenosine-3′-phosphate;

C=cytidine-3′-phosphate;

G=guanosine-3′-phosphate;

U=uridine-3′-phosphate

n=any 2′-OMe modified nucleotide

a=2′-0-methyladenosine-3′-phosphate

as=2′-0-methyladenosine-3′-phosphorothioate

c=2′-0-methylcytidine-3′-phosphate

cs=2′-0-methylcytidine-3′-phosphorothioate

g=2′-0-methylguanosine-3′-phosphate

gs=2′-0-methylguanosine-3′-phosphorothioate

t=2′-0-methyl-5-methyluridine-3′-phosphate

ts=2′-0-methyl-5-methyluridine-3′-phosphorothioate

u=2′-0-methyluridine-3′-phosphate

us=2′-0-methyluridine-3′-phosphorothioate Nf=any 2′-fluoro modified nucleotide

Af=2′-fluoroadenosine-3′-phosphate

Afs=2′-fluoroadenosine-3′-phosporothioate

Cf=2′-fluorocytidine-3′-phosphate

Cfs=2′-fluorocytidine-3′-phosphorothioate

Gf=2′-fluoroguanosine-3′-phosphate

Gfs=2′-fluoroguanosine-3′-phosphorothioate

Tf=2′-fluoro-5′-methyluridine-3′-phosphate

Tfs=2′-fluoro-5′-methyluridine-3′-phosphorothioate

Uf=2′-fluorouridine-3′-phosphate

Ufs=2′-fluorouridine-3′-phosphorothioate

dN=any 2′-deoxyribonucleotide

dT=2′-deoxythymidine-3′-phosphate

NuNA=2′,3′-seco nucleotide mimics (unlocked nucleobase analogs)

NLNA=locked nucleotide

NfANA=2′-F-Arabino nucleotide

NM=2′-methoxyethyl nucleotide

AM=2′-methoxyethyladenosine-3′-phosphate

AMs=2′-methoxyethyladenosine-3′-phosphorothioate

TM=2′-methoxyethylthymidine-3′-phosphate

TMs=2′-methoxyethylthymidine-3′-phosphorothioate

R=ribitol

(invdN)=any inverted deoxyribonucleotide (3′-3′ linked nucleotide)

(invAb)=inverted (3′-3′ linked) abasic deoxyribonucleotide, see Table

(invAb)s=inverted (3′-3′ linked) abasic deoxyribonucleotide-5′-phosphorothioate, see Table 6

(invn)=any inverted 2′-OMe nucleotide (3′-3′ linked nucleotide) s=phosphorothioate linkage

vpdN=vinyl phosphonate deoxyribonucleotide

(5Me-Nf)=5′-Me, 2′-fluoro nucleotide

cPrp=cyclopropyl phosphonate, see Table 6 of WO2018027106

epTcPr=see Table 6 of WO2018027106

epTM=see Table 6 of WO2018027106

The person or ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo.

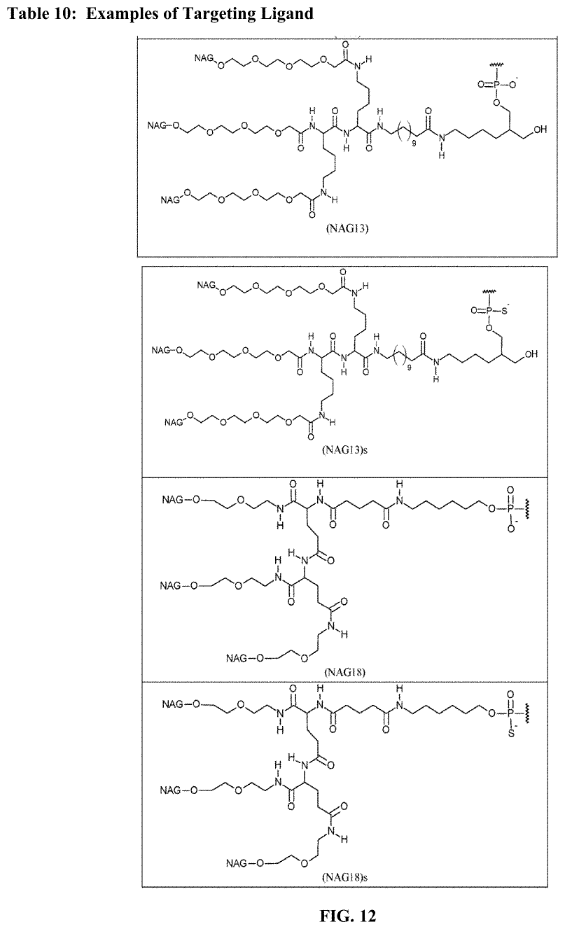

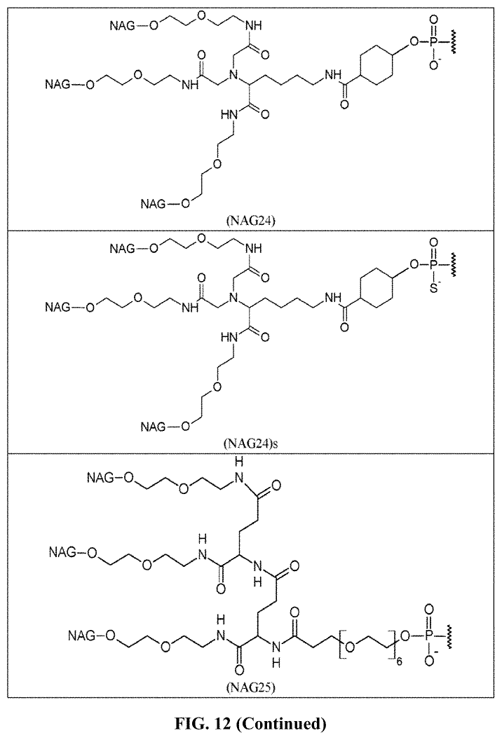

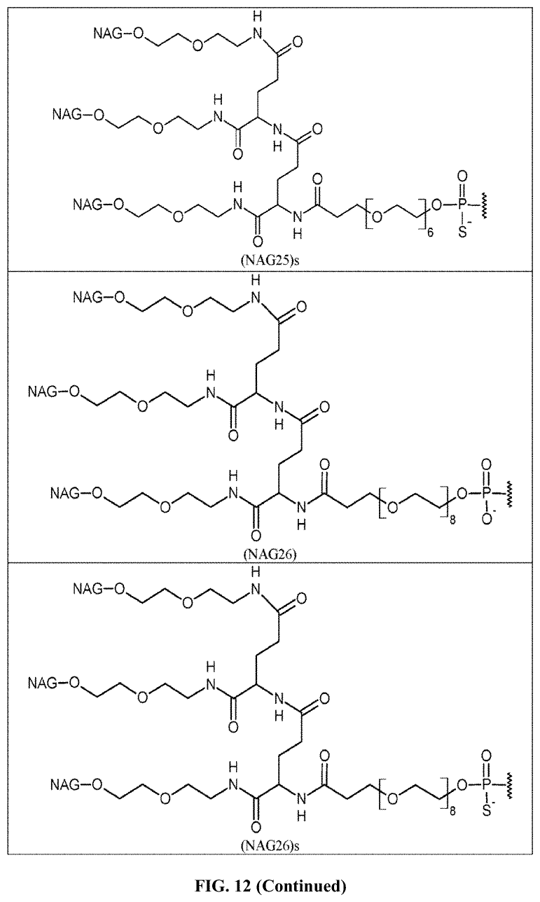

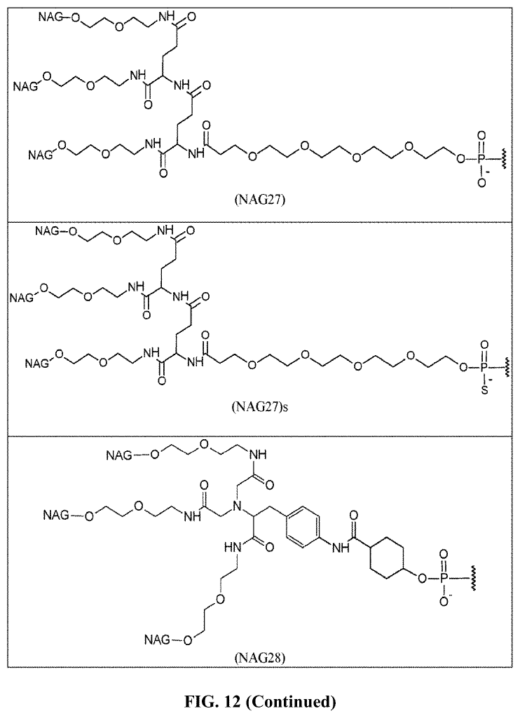

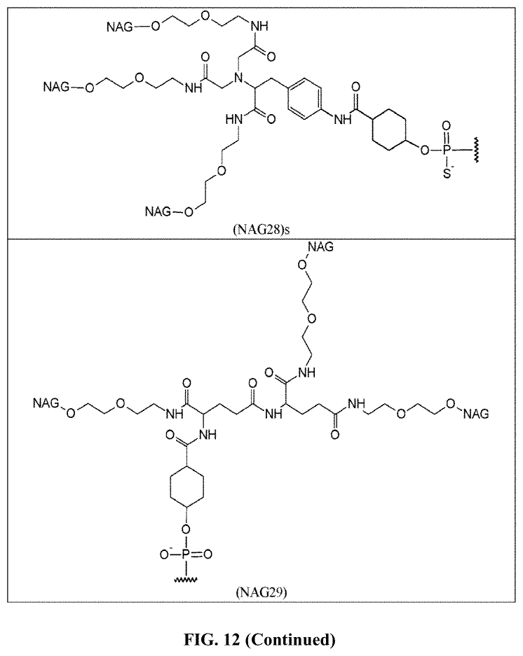

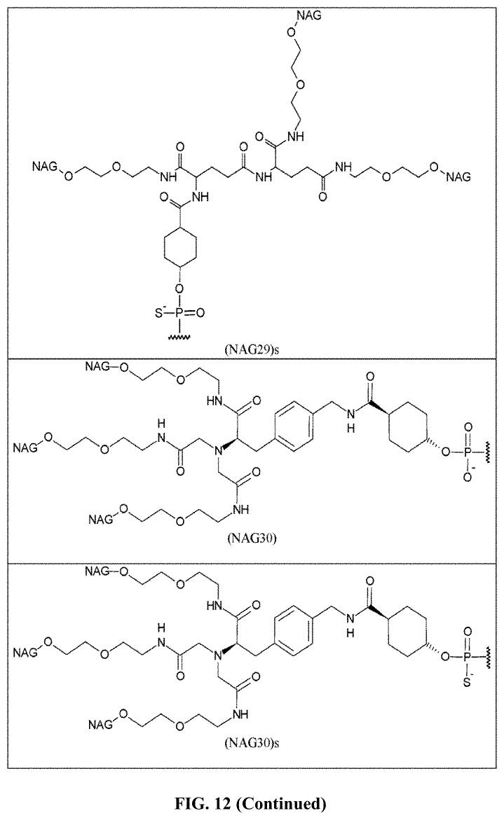

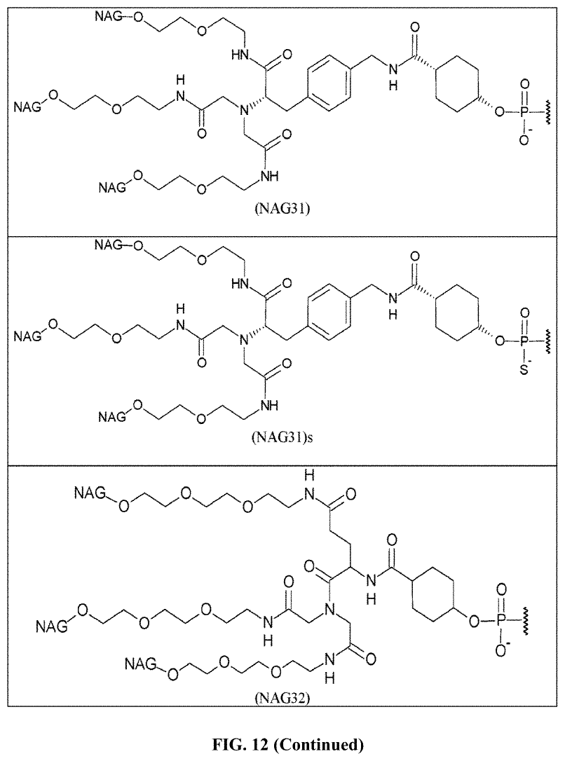

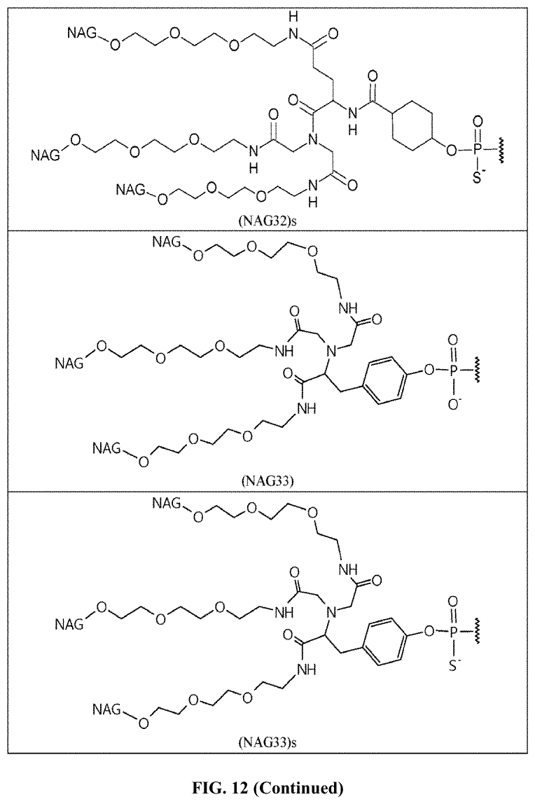

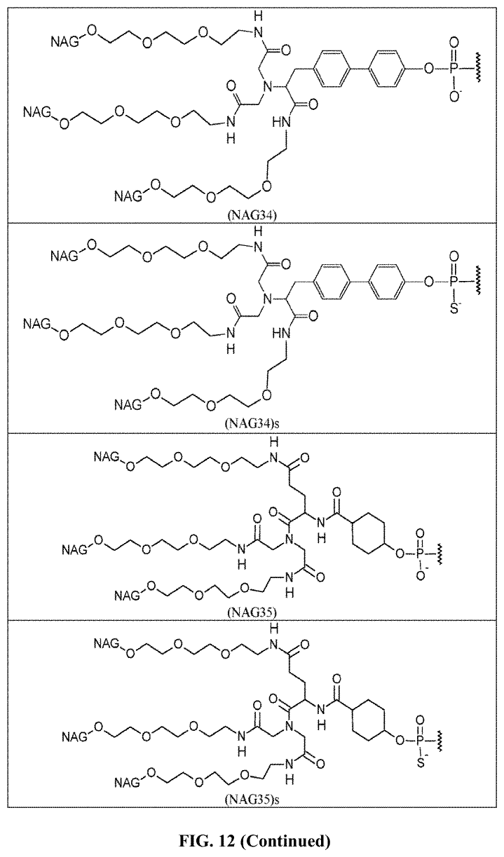

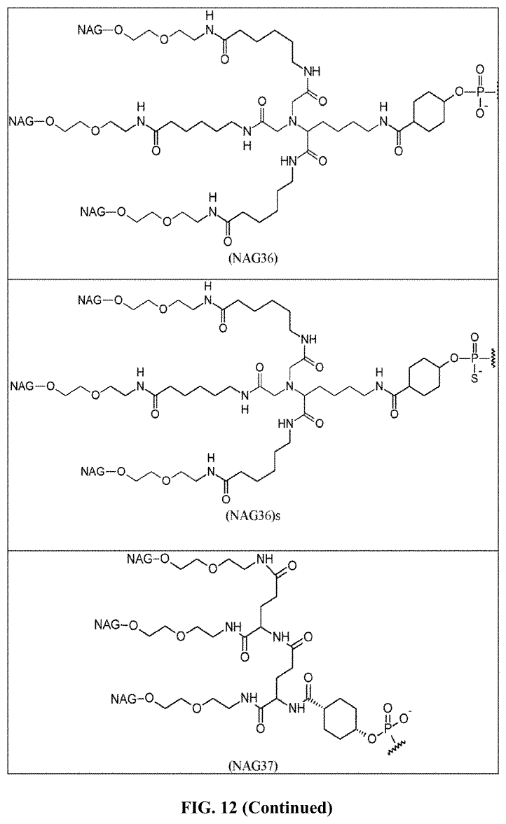

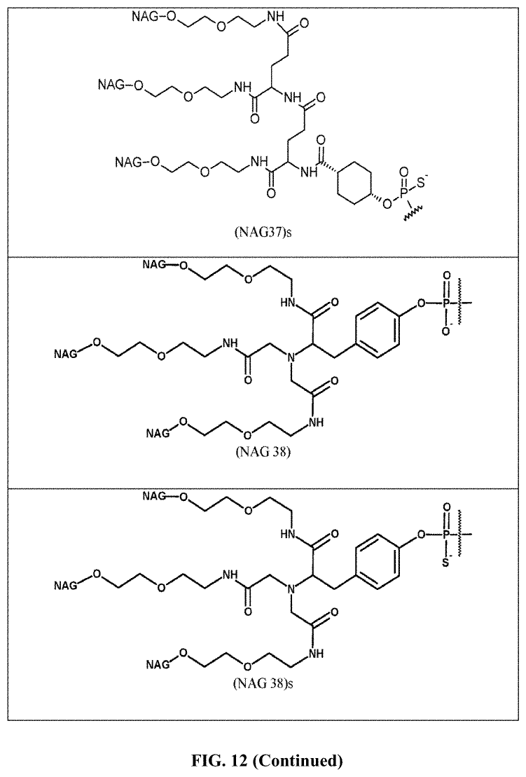

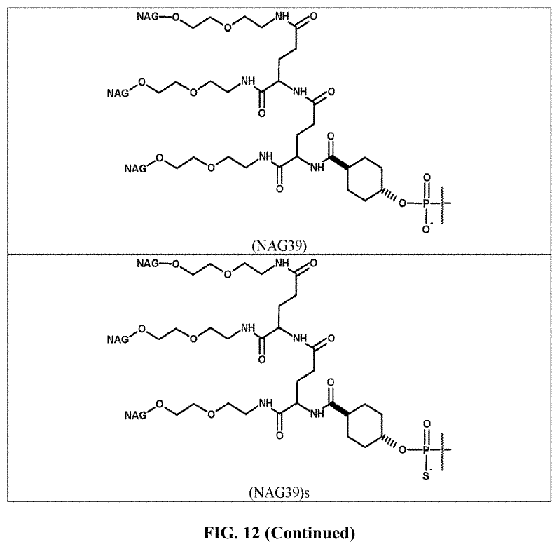

Targeting groups and linking groups include the following, for which their chemical structures are provided below in Table 6 of WO2018027106 and some of which are depicted in Table 10 (FIG. 12): (PAZ), (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), (NAG39)s. Each sense strand and/or antisense strand can have any targeting groups or linking groups listed above, as well as other targeting or linking groups, conjugated to the 5′ and/or 3′ end of the sequence.

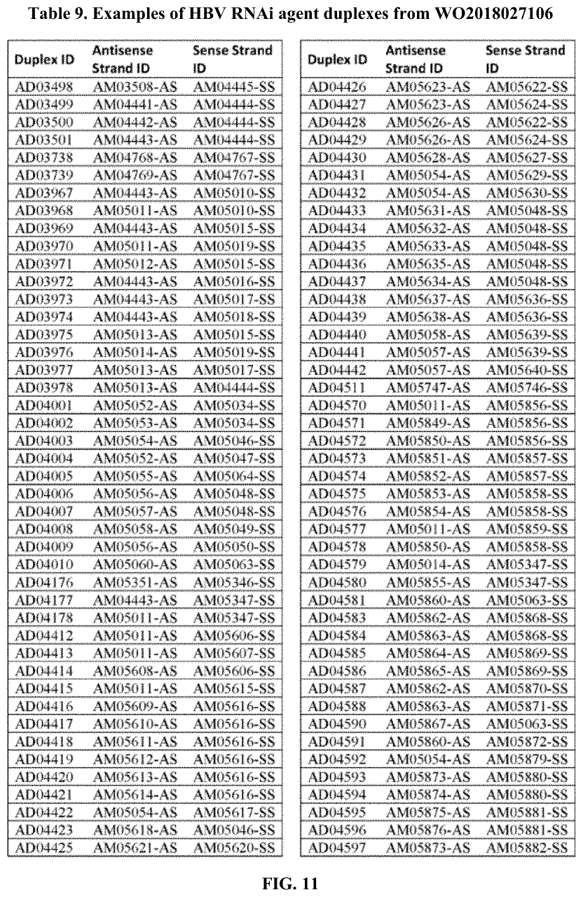

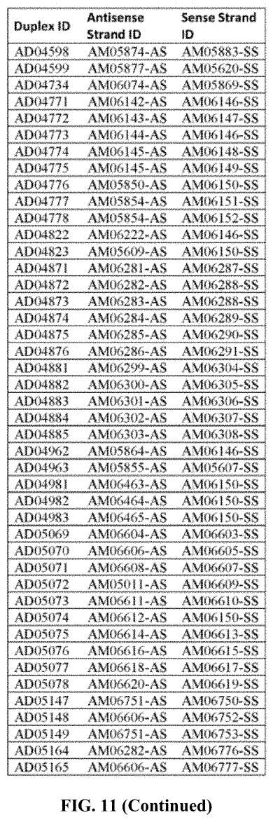

The HBV RNAi agents described herein are formed by annealing an antisense strand with a sense strand. Representative sequence pairings are exemplified by the Duplex ID Nos. shown in FIG. 11.

For the HBV RNAi agents disclosed herein, the nucleotide at position 1 of the antisense strand (from 5′ end 3′ end) can be perfectly complementary to an HBV gene, or can be non-complementary to an HBV gene. In some embodiments, the nucleotide at position 1 of the antisense strand (from 5′ end 3′ end) is a U, A, or dT. In some embodiments, the nucleotide at position 1 of the antisense strand (from 5′ end→3′ end) forms an A: U or U: A base pair with the sense strand.

In some embodiments, an HBV RNAi agent comprises an antisense strand and a sense strand having the modified nucleotide sequences of any of the antisense strand and/or sense strand nucleotide sequences of any of the duplexes described herein, and further comprises an asialoglycoprotein receptor ligand targeting group.

RNAi agents for inhibiting the expression of an HBV gene are known in the art. For example, RNAi agents for inhibiting the expression of an HBV gene include, but are not limited to, RNAi agents for inhibiting the expression of an HBV gene described in US20130005793, WO2013003520, and WO2018027106, the contents of which are incorporated herein in their entirety.

Examples of RNAi agents for inhibiting the expression of an HBV gene include, e.g., RNAi agents comprising one of the sequences in Tables 1, 2 and 4 of US20130005793 (reproduced herein as Tables 2-4 (FIGS. 4-6) or in Tables 1-5 of WO2018027106 (reproduced herein as Tables 5-9 (FIGS. 7-11).

Examples of RNAi agents for inhibiting the expression of an HBV gene include, e.g., RNAi agents comprising a duplex shown in Table 9. According to particular embodiments, the RNAi agent comprises at least one of the duplexes AD04872 (SEQ ID NOs: 25-26 herein) (AM06282-AS (SEQ ID NOs: 126 and 171) and AM06288-SS (SEQ ID NOs: 252 and 302) of WO2018027106) and AD05070 (SEQ ID NOs: 27-28 herein) (AM06606-AS (SEQ ID NOs: 140 and 188) and AM06605-SS (SEQ ID NOs: 262 and 328) of WO2018027106), each of which is conjugated to a targeting ligand, such as one of those having a structure depicted in Table 10, for example, NAG37.

### Targeting Groups, Linking Groups and Delivery Vehicles

In some embodiments, an HBV RNAi agent is conjugated to one or more non-nucleotide groups including, but not limited to a targeting group, linking group, delivery polymer, or a delivery vehicle. The non-nucleotide group can enhance targeting, delivery or attachment of the RNAi agent. Examples of targeting groups and linking groups are provided in Table 6 of WO2018027106. The non-nucleotide group can be covalently linked to the 3′ and/or 5′ end of either the sense strand and/or the antisense strand. In some embodiments, an HBV RNAi agent contains a non-nucleotide group linked to the 3′ and/or 5′ end of the sense strand. In some embodiments, a non-nucleotide group is linked to the 5′ end of an HBV RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting moieties enhance the pharmacokinetic or biodistribution properties of a conjugate to which they are attached to improve cell-specific distribution and cell-specific uptake of the conjugate. A targeting group can be monovalent, divalent, trivalent, tetravalent, or have higher valency. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) groups. In some embodiments, a targeting group comprises a galactose derivative cluster. The HBV RNAi agents described herein can be synthesized having a reactive group, such as an amine group, at the 5′-terminus. The reactive group can be used to subsequently attach a targeting moiety using methods typical in the art. In some embodiments, a targeting group comprises an asialoglycoprotein receptor ligand. In some embodiments, an asialoglycoprotein receptor ligand includes or consists of one or more galactose derivatives. As used herein, the term galactose derivative includes both galactose and derivatives of galactose having affinity for the asialoglycoprotein receptor that is equal to or greater than that of galactose. Galactose derivatives include, but are not limited to: galactose, galactosamine, N-formylgalactosamine, N-acetyl-galactosamine, N-propionyl-galactosamine, N-n-butanoyl-galactosamine, and N-iso-butanoylgalactos-amine (see for example: Iobst, S. T. and Drickamer, K. J. B. C. 1996, 277, 6686). Galactose derivatives, and clusters of galactose derivatives, that are useful for in vivo targeting of oligonucleotides and other molecules to the liver are known in the art (see, for example, Baenziger and Fiete, 1980, Cell, 22, 611-620; Connolly et al., 1982, J. Biol. Chem, 257, 939-945). Galactose derivatives have been used to target molecules to hepatocvtes in vivo through their binding to the asialoglycoprotein receptor (ASGPr) expressed on the surface of hepatocytes. Binding of ASGPr ligands to the ASGPr(s) facilitates cell-specific targeting to hepatocytes and endocytosis of the molecule into hepatocytes. ASGPr ligands can be monomeric (e.g., having a single galactose derivative) or multimeric (e.g., having multiple galactose derivatives). The galactose derivative or galactose derivative cluster can be attached to the 3 Or 5′ end of the RNAi polynucleotide using methods known in the art. The preparation of targeting groups, such as galactose derivative clusters, is described in, for example, US20180064819 and US20170253875, the contents of both of which are incorporated herein in their entirety.

As used herein, a galactose derivative cluster comprises a molecule having two to four terminal galactose derivatives. A terminal galactose derivative is attached to a molecule through its C-1 carbon. In some embodiments, the galactose derivative cluster is a galactose derivative trimer (also referred to as tri-antennary galactose derivative or tri-valent galactose derivative). In some embodiments, the galactose derivative cluster comprises N-acetyl-galactosamines. In some embodiments, the galactose derivative cluster comprises three N-acetyl-galactosamines. In some embodiments, the galactose derivative cluster is a galactose derivative tetramer (also referred to as tetra-antennary galactose derivative or tetra-valent galactose derivative). In some embodiments, the galactose derivative cluster comprises four N-acetyl-galactosamines.

As used herein, a galactose derivative trimer contains three galactose derivatives, each linked to a central branch point. As used herein, a galactose derivative tetramer contains four galactose derivatives, each linked to a central branch point. The galactose derivatives can be attached to the central branch point through the C-l carbons of the saccharides. In some embodiments, the galactose derivatives are linked to the branch point via linkers or spacers. In some embodiments, the linker or spacer is a flexible hydrophilic spacer, such as a PEG group (see, for example, U.S. Pat. No. 5,885,968; Biessen et al, J. Med. Chem. 1995 Vol. 39 p. 1538-1546). In some embodiments, the PEG spacer is a PEG3 spacer. The branch point can be any small molecule which permits attachment of three galactose derivatives and further permits attachment of the branch point to the RNAi agent. An example of branch point group is a di-lysine or di-glutamate. Attachment of the branch point to the RNAi agent can occur through a linker or spacer. In some embodiments, the linker or spacer comprises a flexible hydrophilic spacer, such as, but not limited to, a PEG spacer. In some embodiments, the linker comprises a rigid linker, such as a cyclic group. In some embodiments, a galactose derivative comprises or consists of N-acetyl-galactosamine. In some embodiments, the galactose derivative duster is comprised of a galactose derivative tetramer, which can be, for example, an N-acetyl-galactosamine tetramer.

In some embodiments, a linking group is conjugated to the RNAi agent. The linking group facilitates covalent linkage of the agent to a targeting group or delivery polymer or delivery vehicle. The linking group can be linked to the 3 or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include, but are not limited to: reactive groups such a primary amines and alkynes, alkyl groups, abasic nucleosides, ribitol (abasic ribose), and/or PEG groups.

A linker or linking group is a connection between two atoms that links one chemical group (such as an RNAi agent) or segment of interest to another chemical group (such as a targeting group or delivery polymer) or segment of interest via one or more covalent bonds. A labile linkage contains a labile bond. A linkage can optionally include a spacer that increases the distance between the two joined atoms. A spacer can further add flexibility and/or length to the linkage. Spacers can include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

### Delivery Vehicles

In some embodiments, a delivery vehicle can be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active poly amine.

In some embodiments, the RNAi agents can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/0022309, WO 2011/104169, and WO 2012/083185, WO 2013/032829, WO 2013/158141, each of which is incorporated herein by reference), or other delivery systems available in the art.

Other lipophilic compounds that have been conjugated to oligonucleotides include 1-pyrene butyric acid, l,3-bis-O-(hexadecyl)glycerol, and menthol. One example of a ligand for receptor-mediated endocytosis is folic acid. Folic acid enters the cell by folate-receptor-mediated endocytosis. RNAi agents bearing folic acid would be efficiently transported into the cell via the folate-receptor-mediated endocytosis. Attachment of folic acid to the 3′-terminus of an oligonucleotide results in increased cellular uptake of the oligonucleotide (Li S, Deshmukh H M, and Huang L, Pharm. Res. (1998) 15: 1540). Other ligands that have been conjugated to oligonucleotides include polyethylene glycols, carbohydrate clusters, cross-linking agents, porphyrin conjugates, and delivery peptides. In certain instances, conjugation of a cationic ligand to oligonucleotides often results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed throughout the oligonucleotide. See Manoharan M, Antisense & Nucleic Acid Drug Development (2002) 12: 103 and references therein.

Additional modifications can also be made at other positions on the oligonucleotide, particularly the 3′ position of the sugar on the 3′ terminal nucleotide. For example, one additional modification of the ligand-conjugated oligonucleotides of the application involves chemically linking to the oligonucleotide one or more additional non-ligand moieties or conjugates which enhance the activity, cellular distribution or cellular uptake of the oligonucleotide. Such moieties include but are not limited to lipid moieties, such as a cholesterol moiety (Letsinger et al, Proc. Natl. Acad. Sci. USA, (1989) 86:6553), cholic acid (Manoharan et al, Bioorg. Med. Chem. Lett., (1994) 4: 1053), a thioether, e.g., hexyl-S-tritylthiol (Manoharan et al., Ann. N Y. Acad. Sci., (1992) 660:306; Manoharan et al, Bioorg. Med. Chem. Let., (1993) 3:2765), a fhiochoiestero (Oberhauser et al., Nucl Acids Res., (1992) 20:533), an aliphatic chain, e.g., dodecandiol or undecyl residues (Saison-Behmoaras et al., EMBO J., (1991) 10: 1 1 1; Kabanov et al, FEBS Lett., (1990) 259:327; Svinarchuk et al, Biochimie, (1993) 75:49), a phospholipid, e.g., di-hexadecyl-rac-glycerol or triethylammonium 1,2-di-O-hexadecyl-rac-glycero-3-H-phosphonate (Manoharan et al, Tetrahedron Lett., (1995) 36:3651; Shea et al, Nucl Acids Res., (1990) 18:3777), a polyamine or a polyethylene glycol chain (Manoharan et al., Nucleosides & Nucleotides, (1995) 14:969), or adamantane acetic acid (Manoharan et al., Tetrahedron Lett., (1995) 36:3651), a palmityl moiety (Mishra et al., Biochim. Biophys. Acta, (1995) 1264:229), or an octadecylamine or hexylamino-carbonyl-oxycholesterol moiety (Crooke et al., J. Pharmacol. Exp. Ther., (1996) 277:923).

Additional modifications can also he made at other positions on the oligonucleotide, particularly the 3′ position of the sugar on the 3′ terminal nucleotide. For example, one additional modification of the ligand-conjugated oligonucleotides of the application involves chemically linking to the oligonucleotide one or more additional non-ligand moieties or conjugates which enhance the activity, cellular distribution or cellular uptake of the oligonucleotide. Such moieties include but are not limited to lipid moieties, such as a cholesterol moiety (Letsinger et al, Proc. Natl. Acad. Sci. USA, (1989) 86:6553), cholic acid (Manoharan et al, Bioorg. Med. Chem. Lett., (1994) 4: 1053), a thioether, e.g., hexyl-S-tritylthiol (Manoharan et al., Ann. N Y. Acad. Sci., (1992) 660:306; Manoharan et al, Bioorg. Med. Chem. Let., (1993) 3:2765), a fhiochoiesterol (Oberhauser et al., Nucl Acids Res., (1992) 20:533), an aliphatic chain, e.g., dodecandiol or undecyl residues (Saison-Behmoaras et al., EMBO J., (1991) 10: 1 1 1; Kabanov et al, FEBS Lett., (1990) 259:327; Svinarchuk et al, Biochimie, (1993) 75:49), a phospholipid, e.g., di-hexadecyl-rac-glycerol or triethylammonium 1,2-di-O-hexadecyl-rac-glycero-3-H-phosphonate (Manoharan et al, Tetrahedron Lett., (1995) 36:3651; Shea et al, Nucl Acids Res., (1990) 18:3777), a polyamine or a polyethylene glycol chain (Manoharan et al., Nucleosides & Nucleotides, (1995) 14:969), or adamantane acetic acid (Manoharan et al., Tetrahedron Lett., (1995) 36:3651), a palmityl moiety (Mishra et al., Biochim. Biophys. Acta, (1995) 1264:229), or an octadecylamine or hexylamino-carbonyl-oxycholesterol moiety (Crooke et al., J. Pharmacol. Exp. Ther., (1996) 277:923).

The application also includes compositions employing oligonucleotides that are substantially chirally pure with regard to particular positions within the oligonucleotides.

Examples of substantially chirally pure oligonucleotides include, but are not limited to, those having phosphorothioatc linkages that are at least 75% Sp or Rp (Cook et al., U.S. Pat. No. 5,587,361) and those having substantially chirally pure (Sp or Rp) alkylphosphonate, phosphoramidate or phosphotriester linkages (Cook, U.S. Pat. No. 5,212,295 and U.S. Pat. No. 5,521,302).

In certain instances, the oligonucleotide can be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Letsinger et al., Proc. Natl. Acad. Sci. USA, (1989, 86:6553), cholic acid (Manoharan et al., Bioorg. Med. Chem. Lett., (1994, 4: 1053), a thioether, e.g., hexyl-S-tritylthiol (Manoharan et al, Ann. N. Y. Acad. Sci (1992, 660:306; Manoharan et al., Bioorg. Med. Chem. Let., (1993, 3:2765), a thiocholesterol (Oberhauser et al, Nucl. Acids Res., (1992, 20:533), an aliphatic chain, e.g., dodecandiol or undecyl residues (Saison-Behmoaras et al., EMBO J., (1991) 10: 1 1 1; Kabanov et al., FEBS Lett, (1990) 259:327; Svinarchuk et al, Biochimie, (1993) 75:49), a phospholipid, e.g., di-hexadecyl-rac-glycerol or triethylammonium 1,2-di-O-hexadecyl-rac-glycero-3-H-phosphonate (Manoharan et al, Tetrahedron Lett., (1995) 36:3651; Shea et al., Nucl. Acids Res., (1990) 18:3777), a polyamine or a polyethylene glycol chain (Manoharan et al, Nucleosides & Nucleotides, (1995) 14:969), or adamantane acetic acid (Manoharan et al., Tetrahedron Lett., (1995) 36:3651), a palmityl moiety (Mishra et al., Biochim. Biophys. Acta, (1995) 1264:229), or an octadecylamine or hexylamino-carbonyl-oxycholesterol moiety (Crooke et al, J. Pharmacol. Exp. Ther., (1996) 277:923). Typical conjugation protocols involve the synthesis of oligonucleotides bearing an aminolinker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide in solution phase. Purification of the oligonucleotide conjugate by HPLC typically affords the pure conjugate.

Alternatively, the molecule being conjugated can be converted into a building block, such as a phosphoramidite, via an alcohol group present in the molecule or by attachment of a linker bearing an alcohol group that can be phosphorylated. Importantly, each of these approaches can be used for the synthesis of ligand conjugated oligonucleotides. Amino linked oligonucleotides can be coupled directly with ligand via the use of coupling reagents or following activation of the ligand as an NHS or pentfluorophenolate ester. Ligand phosphoramidites can be synthesized via the attachment of an aminohexanol linker to one of the carboxyl groups followed by phosphity ation of the terminal alcohol functionality. Other linkers, such as cysteamine, can also be utilized for conjugation to a chloroacetyl linker present on a synthesized oligonucleotide.

The person skilled in the art is readily aware of methods to introduce the molecules of this application into cells, tissues or organisms. Corresponding examples have also been provided in the detailed description of the application above. For example, the nucleic acid molecules or the vectors of this application, encoding for at least one strand of the described RNAi agents can be introduced into cells or tissues by methods known in the art, like transfections etc.

Also for the introduction of RNAi agents, means and methods have been provided. For example, targeted delivery by glycosylated and folate-modified molecules, including the use of polymeric carriers with ligands, such as galactose and lactose or the attachment of folic acid to various macromolecules allows the binding of molecules to be delivered to folate receptors. Targeted delivery by peptides and proteins other than antibodies, for example, including RGD-modified nanoparticics to deliver siRNA in vivo or multicomponent (nonviral) delivery systems including short cyclodextrins, adamantine-PEG are known. Yet, also the targeted delivery using antibodies or antibody fragments, including (monovalent) Fab-fragments of an antibody (or other fragments of such an antibody) or single-chain antibodies are envisaged. Injection approaches for target directed delivery comprise, inter alia, hydrodynamic i.v. injection. Also, cholesterol conjugates of RNAi agents can be used for targeted delivery, whereby the conjugation to lipophilic groups enhances cell uptake and improve pharmacokinetics and tissue biodistribution of oligonucleotides. Also, cationic delivery systems are known, whereby synthetic vectors with net positive (cationic) charge to facilitate the complex formation with the polyanionic nucleic acid and interaction with the negatively charged cell membrane. Such cationic delivery systems comprise also cationic liposomal delivery systems, cationic polymer and peptide delivery systems. Other delivery systems for the cellular uptake of dsRNA/siRNA are aptamer-ds/si RNA. Also, gene therapy approaches can be used to deliver the described RNAi agents or nucleic acid molecules encoding the same. Such systems comprise the use of non-pathogenic virus, modified viral vectors, as well as deliveries with nanoparticles or liposomes. Other delivery methods for the cellular uptake of RNAi agents are extracorporeal, for example ex vivo treatments of cells, organs or tissues. Certain of these technologies are described and summarized in publications, like Akhtar, Journal of Clinical Investigation (2007) 1 17:3623-3632, Nguyen et al, Current Opinion in Molecular Therapeutics (2008) 10: 158-167, Zambon i, Clin Cancer Res (2005) 1 1:8230-8234 or Ikeda et al, Pharmaceutical Research (2006) 23:1631-1640.

Methods of making and using RNAi agents and conjugates thereof are known in the art. Any such known methods can be used in the context of the present application to make and use RNAi agents and conjugates thereof for inhibiting the expression of an HBV gene. Methods of making and using RNAi agents and conjugates thereof are described, e.g., in US20130005793, WO2013003520, WO2018027106, U.S. Pat. No. 5,218,105, U.S. Pat. No. 5,541,307, U.S. Pat. No. 5,521,302, U.S. Pat. No. 5,539,082, U.S. Pat. No. 5,554,746, U.S. Pat. No. 5,571,902, U.S. Pat. No. 5,578,718, U.S. Pat. No. 5,587,361, U.S. Pat. No. 5,506,351, U.S. Pat. No. 5,587,469, U.S. Pat. No. 5,587,470, U.S. Pat. No. 5,608,046, U.S. Pat. No. 5,610,289, U.S. Pat. No. 6,262,241, WO9307883, all of which are incorporated herein by reference in their entirety.

### Compositions, Therapeutic Combinations, and Vaccines

The application also relates to compositions, therapeutic combinations, more particularly kits, and vaccines comprising one or more HBV antigens, polynucleotides, and/or vectors encoding one or more HBV antigens according to the application, and/or one or more RNAi agent for inhibiting the expression of an HBV gene. Any of the HBV antigens, polynucleotides (including RNA and DNA), and/or vectors of the application described herein, and any of the RNAi agents for inhibiting the expression of an HBV gene of the application described herein, can be used in the compositions, therapeutic combinations or kits, and vaccines of the application.

In an embodiment of the application, a composition comprises an isolated or non-naturally occurring nucleic acid molecule (DNA or RNA) comprising polynucleotide sequence encoding a truncated HBV core antigen consisting of an amino acid sequence that is at least 90% identical to SEQ ID NO: 2 or SEQ ID NO: 4, or an HBV polymerase antigen comprising an amino acid sequence that is at least 90% identical to SEQ ID NO: 7, a vector comprising the isolated or non-naturally occurring nucleic acid molecule, and/or an isolated or non-naturally occurring polypeptide encoded by the isolated or non-naturally occurring nucleic acid molecule.

In an embodiment of the application, a composition comprises an isolated or non-naturally occurring nucleic acid molecule (DNA or RNA) comprising a polynucleotide sequence encoding an HBV Pol antigen comprising an amino acid sequence that is at least 90% identical to SEQ ID NO: 7, preferably 100% identical to SEQ ID NO: 7.

In an embodiment of the application, a composition comprises an isolated or non-naturally occurring nucleic acid molecule (DNA or RNA) encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises an isolated or non-naturally occurring nucleic acid molecule (DNA or RNA) comprising a polynucleotide sequence encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and an isolated or non-naturally occurring nucleic acid molecule (DNA or RNA) comprising a polynucleotide sequence encoding an HBV Pol antigen comprising an amino acid sequence that is at least 90% identical to SEQ ID NO: 7, preferably 100% identical to SEQ ID NO: 7. The coding sequences for the truncated HBV core antigen and the HBV Pol antigen can be present in the same isolated or non-naturally occurring nucleic acid molecule (DNA or RNA), or in two different isolated or non-naturally occurring nucleic acid molecules (DNA or RNA).

In an embodiment of the application, a composition comprises a vector, preferably a DNA plasmid or a viral vector (such as an adenoviral vector) comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises a vector, preferably a DNA plasmid or a viral vector (such as an adenoviral vector), comprising a polynucleotide encoding an HBV Pol antigen comprising an amino acid sequence that is at least 90% identical to SEQ ID NO: 7, preferably 100% identical to SEQ ID NO: 7.

In an embodiment of the application, a composition comprises a vector, preferably a DNA plasmid or a viral vector (such as an adenoviral vector), comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and a vector, preferably a DNA plasmid or a viral vector (such as an adenoviral vector), comprising a polynucleotide encoding an HBV Pol antigen comprising an amino acid sequence that is at least 90% identical to SEQ ID NO: 7, preferably 100% identical to SEQ ID NO: 7. The vector comprising the coding sequence for the truncated HBV core antigen and the vector comprising the coding sequence for the HBV Pol antigen can be the same vector, or two different vectors.

In an embodiment of the application, a composition comprises a vector, preferably a DNA plasmid or a viral vector (such as an adenoviral vector), comprising a polynucleotide encoding a fusion protein comprising a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4, operably linked to an HBV Pol antigen comprising an amino acid sequence that is at least 90% identical to SEQ ID NO: 7, preferably 100% identical to SEQ ID NO: 7, or vice versa. Preferably, the fusion protein further comprises a linker that operably links the truncated HBV core antigen to the HBV Pol antigen, or vice versa. Preferably, the linker has the amino acid sequence of (AlaGly)n, wherein n is an integer of 2 to 5.

In an embodiment of the application, a composition comprises an isolated or non-naturally occurring truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises an isolated or non-naturally occurring HBV Pol antigen comprising an amino acid sequence that is at least 90% identical to SEQ ID NO: 7, preferably 100% identical to SEQ ID NO: 7.

In an embodiment of the application, a composition comprises an isolated or non-naturally occurring truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and an isolated or non-naturally occurring HBV Pol antigen comprising an amino acid sequence that is at least 90% identical to SEQ ID NO: 7, preferably 100% identical to SEQ ID NO: 7.

In an embodiment of the application, a composition comprises an isolated or non-naturally occurring fusion protein comprising a truncated HBV core antigen consisting of an amino acid sequence that is at least 90% identical to SEQ ID NO: 2 or SEQ ID NO: 14, preferably 100% identical to SEQ ID NO: 2 or SEQ ID NO: 4, operably linked to an HBV Pol antigen comprising an amino acid sequence that is at least 90% identical to SEQ ID NO: 7, preferably 100% identical to SEQ ID NO: 7, or vice versa. Preferably, the fusion protein further comprises a linker that operably links the truncated HBV core antigen to the HBV Pol antigen, or vice versa. Preferably, the linker has the amino acid sequence of (AlaGly)n, wherein n is an integer of 2 to 5.

In an embodiment of the application, a composition comprises an RNAi agent for inhibiting the expression of an HBV gene, such as those described in US20130005793, WO2013003520 or WO2018027106.

The application also relates to a therapeutic combination or a kit comprising polynucleotides expressing a truncated HBV core antigen and an HBV pol antigen according to embodiments of the application and/or RNAi agents for inhibiting the expression of an HBV gene according to embodiments of the application. Any polynucleotides and/or vectors encoding HBV core and pol antigens of the application described herein can be used in the therapeutic combinations or kits of the application and any RNAi agents for inhibiting the expression of an HBV gene of the application described herein can be used in the therapeutic combinations or kits of the application.

According to embodiments of the application, a therapeutic combination or kit for use in treating an HBV infection in a subject in need thereof, comprises:

i) at least one of:
a) a truncated HBV core antigen consisting of an amino acid sequence that is at least 95% identical to SEQ ID NO: 2, and
b) a first non-naturally occurring nucleic acid molecule comprising a first polynucleotide sequence encoding the truncated HBV core antigen
c) an HBV polymerase antigen having an amino acid sequence that is at least 90% identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and

ii) an RNAi agent for inhibiting the expression of an HBV gene, such as those describe herein.

In a particular embodiment of the application, a therapeutic combination or kit comprises: i) a first non-naturally occurring nucleic acid molecule comprising a first polynucleotide sequence encoding a truncated HBV core antigen consisting of an amino acid sequence that is at least 95% identical to SEQ ID NO: 2; ii) a second non-naturally occurring nucleic acid molecule comprising a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity; and iii) an RNAi agent for inhibiting the expression of an HBV gene, preferably the RNAi agent comprises a duplex shown in Table 9, more preferably the RNAi agent comprises at least one of the duplexes AD04872 (SEQ ID NOs: 25-26) and AD05070 (SEQ ID NOs: 27-28), each of which is conjugated to a targeting ligand, such as that having a structure depicted in Table 10, for example, NAG37.

According to embodiments of the application, the polynucleotides in a vaccine combination or kit can be linked or separate, such that the HBV antigens expressed from such polynucleotides are fused together or produced as separate proteins, whether expressed from the same or different polynucleotides. In an embodiment, the first and second polynucleotides are present in separate vectors, e.g., DNA plasmids or viral vectors, used in combination either in the same or separate compositions, such that the expressed proteins are also separate proteins, but used in combination. In another embodiment, the HBV antigens encoded by the first and second polynucleotides can be expressed from the same vector, such that an HBV core-pol fusion antigen is produced. Optionally, the core and pol antigens can be joined or fused together by a short linker. Alternatively, the HBV antigens encoded by the first and second polynucleotides can be expressed independently from a single vector using a using a ribosomal slippage site (also known as cis-hydrolase site) between the core and pol antigen coding sequences. This strategy results in a bicistronic expression vector in which individual core and pol antigens are produced from a single mRNA transcript. The core and pol antigens produced from such a bicistronic expression vector can have additional N or C-terminal residues, depending upon the ordering of the coding sequences on the mRNA transcript. Examples of ribosomal slippage sites that can be used for this purpose include, but are not limited to, the FA2 slippage site from foot-and-mouth disease virus (FMDV). Another possibility is that the HBV antigens encoded by the first and second polynucleotides can be expressed independently from two separate vectors, one encoding the HBV core antigen and one encoding the HBV pol antigen.

In a preferred embodiment, the first and second polynucleotides are present in separate vectors, e.g., DNA plasmids or viral vectors. Preferably, the separate vectors are present in the same composition.

According to preferred embodiments of the application, a therapeutic combination or kit comprises a first polynucleotide present in a first vector, a second polynucleotide present in a second vector. The first and second vectors can be the same or different. Preferably the vectors are DNA plasmids.

In a particular embodiment of the application, the first vector is a first DNA plasmid, the second vector is a second DNA plasmid. Each of the first and second DNA plasmids comprises an origin of replication, preferably pUC ORI of SEQ ID NO: 21, and an antibiotic resistance cassette, preferably comprising a codon optimized Kanr gene having a polynucleotide sequence that is at least 90% identical to SEQ ID NO: 23, preferably under control of a bla promoter, for instance the bla promoter shown in SEQ ID NO: 24. Each of the first and second DNA plasmids independently further comprises at least one of a promoter sequence, enhancer sequence, and a polynucleotide sequence encoding a signal peptide sequence operably linked to the first polynucleotide sequence or the second polynucleotide sequence. Preferably, each of the first and second DNA plasmids comprises an upstream sequence operably linked to the first polynucleotide or the second polynucleotide, wherein the upstream sequence comprises, from 5′ end to 3′ end, a promoter sequence of SEQ ID NO: 18 or 19, an enhancer sequence, and a polynucleotide sequence encoding a signal peptide sequence having the amino acid sequence of SEQ ID NO: 9 or 15. Each of the first and second DNA plasmids can also comprise a polyadenylation signal located downstream of the coding sequence of the HBV antigen, such as the bGH polyadenylation signal of SEQ ID NO: 20.

In one particular embodiment of the application, the first vector is a viral vector and the second vector is a viral vector. Preferably, each of the viral vectors is an adenoviral vector, more preferably an Ad26 or Ad35 vector, comprising an expression cassette including the polynucleotide encoding an HBV pol antigen or an truncated HBV core antigen of the application; an upstream sequence operably linked to the polynucleotide encoding the HBV antigen comprising, from 5′ end to 3′ end, a promoter sequence, preferably a CMV promoter sequence of SEQ ID NO: 19, an enhancer sequence, preferably an ApoAI gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

In another preferred embodiment, the first and second polynucleotides are present in a single vector, e.g., DNA plasmid or viral vector. Preferably, the single vector is an adenoviral vector, more preferably an Ad26 vector, comprising an expression cassette including a polynucleotide encoding an HBV pol antigen and a truncated HBV core antigen of the application, preferably encoding an HBV pol antigen and a truncated HBV core antigen of the application as a fusion protein; an upstream sequence operably linked to the polynucleotide encoding the HBV pol and truncated core antigens comprising, from 5′ end to 3′ end, a promoter sequence, preferably a CMV promoter sequence of SEQ ID NO: 19, an enhancer sequence, preferably an ApoAI gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

When a therapeutic combination of the application comprises a first vector, such as a DNA plasmid or viral vector, and a second vector, such as a DNA plasmid or viral vector, the amount of each of the first and second vectors is not particularly limited. For example, the first DNA plasmid and the second DNA plasmid can be present in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second DNA plasmids are present in a ratio of 1:1, by weight. The therapeutic combination of the application can further comprise a third vector encoding a third active agent useful for treating an HBV infection.

Compositions and therapeutic combinations of the application can comprise additional polynucleotides or vectors encoding additional HBV antigens and/or additional HBV antigens or immunogenic fragments thereof, such as an HBsAg, an HBV L protein or HBV envelope protein, or a polynucleotide sequence encoding thereof or RNAi agent for inhibiting the expression of an HBV gene according to embodiments of the application. However, in particular embodiments, the compositions and therapeutic combinations of the application do not comprise certain antigens.

In a particular embodiment, a composition or therapeutic combination or kit of the application does not comprise a HBsAg or a polynucleotide sequence encoding the HBsAg.

In another particular embodiment, a composition or therapeutic combination or kit of the application does not comprise an HBV L protein or a polynucleotide sequence encoding the HBV L protein.

In yet another particular embodiment of the application, a composition or therapeutic combination of the application does not comprise an HBV envelope protein or a polynucleotide sequence encoding the HBV envelope protein.

Compositions and therapeutic combinations of the application can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. Pharmaceutically acceptable carriers can include vehicles, such as lipid nanoparticles (LNPs). The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions and therapeutic combinations of the application can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions of the application can also be formulated for other routes of administration including transmucosal, ocular, rectal, long acting implantation, sublingual administration, under the tongue, from oral mucosa bypassing the portal circulation, inhalation, or intranasal.

In a preferred embodiment of the application, compositions and therapeutic combinations of the application are formulated for parental injection, preferably subcutaneous, intradermal injection, or intramuscular injection, more preferably intramuscular injection.

According to embodiments of the application, compositions and therapeutic combinations for administration will typically comprise a buffered solution in a pharmaceutically acceptable carrier, e.g., an aqueous carrier such as buffered saline and the like, e.g., phosphate buffered saline (PBS). The compositions and therapeutic combinations can also contain pharmaceutically acceptable substances as required to approximate physiological conditions such as pH adjusting and buffering agents. For example, a composition or therapeutic combination of the application comprising plasmid DNA can contain phosphate buffered saline (PBS) as the pharmaceutically acceptable carrier. The plasmid DNA can be present in a concentration of, e.g., 0.5 mg/mL to 5 mg/mL, such as 0.5 mg/mL 1, mg/mL, 2 mg/mL, 3 mg/mL, 4 mg/mL, or 5 mg/mL, preferably at 1 mg/mL.

Compositions and therapeutic combinations of the application can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

In a particular embodiment of the application, a composition or therapeutic combination is a DNA vaccine. DNA vaccines typically comprise bacterial plasmids containing a polynucleotide encoding an antigen of interest under control of a strong eukaryotic promoter. Once the plasmids are delivered to the cell cytoplasm of the host, the encoded antigen is produced and processed endogenously. The resulting antigen typically induces both humoral and cell-medicated immune responses. DNA vaccines are advantageous at least because they offer improved safety, are temperature stable, can be easily adapted to express antigenic variants, and are simple to produce. Any of the DNA plasmids of the application can be used to prepare such a DNA vaccine.

In other particular embodiments of the application, a composition or therapeutic combination is an RNA vaccine. RNA vaccines typically comprise at least one single-stranded RNA molecule encoding an antigen of interest, e.g., a fusion protein or HBV antigen according to the application. Once the RNA is delivered to the cell cytoplasm of the host, the encoded antigen is produced and processed endogenously, inducing both humoral and cell-mediated immune responses, similar to a DNA vaccine. The RNA sequence can be codon optimized to improve translation efficiency. The RNA molecule can be modified by any method known in the art in view of the present disclosure to enhance stability and/or translation, such by adding a polyA tail, e.g., of at least 30 adenosine residues; and/or capping the 5-end with a modified ribonucleotide, e.g., 7-methylguanosine cap, which can be incorporated during RNA synthesis or enzymatically engineered after RNA transcription. An RNA vaccine can also be self-replicating RNA vaccine developed from an alphavirus expression vector. Self-replicating RNA vaccines comprise a replicase RNA molecule derived from a virus belonging to the alphavirus family with a subgenomic promoter that controls replication of the fusion protein or HBV antigen RNA followed by an artificial poly A tail located downstream of the replicase.

In certain embodiments, a further adjuvant can be included in a composition or therapeutic combination of the application, or co-administered with a composition or therapeutic combination of the application. Use of another adjuvant is optional, and can further enhance immune responses when the composition is used for vaccination purposes. Other adjuvants suitable for co-administration or inclusion in compositions in accordance with the application should preferably be ones that are potentially safe, well tolerated and effective in humans. An adjuvant can be a small molecule or antibody including, but not limited to, immune checkpoint inhibitors (e.g., anti-PD1, anti-TIM-3, etc.), toll-like receptor agonists (e.g., TLR7 agonists and/or TLR8 agonists), RIG-1 agonists, IL-15 superagonists (Altor Bioscience), mutant IRF3 and IRF7 genetic adjuvants, STING agonists (Aduro), FLT3L genetic adjuvant, and IL-7-hyFc. For example, adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, CTLA-4 inhibitors; Agonists of co-stimulatory receptors that are expressed on immune cells (more particularly T cells), such as CD27 and CD28; BTK inhibitors; Other drugs for treating HBV; IDO inhibitors; Arginase inhibitors; and KDM5 inhibitors.

In certain embodiments, each of the first and second non-naturally occurring nucleic acid molecules is independently formulated with a lipid nanoparticle (LNP).

The application also provides methods of making compositions and therapeutic combinations of the application. A method of producing a composition or therapeutic combination comprises mixing an isolated polynucleotide encoding an HBV antigen, vector, and/or polypeptide of the application with one or more pharmaceutically acceptable carriers. One of ordinary skill in the art will be familiar with conventional techniques used to prepare such compositions.

### Methods of Inducing an Immune Response or Treating an HBV Infection

The application also provides methods of inducing an immune response against hepatitis B virus (HBV) in a subject in need thereof, comprising administering to the subject an immunogenically effective amount of a composition or immunogenic composition of the application. Any of the compositions and therapeutic combinations of the application described herein can be used in the methods of the application.

As used herein, the term “infection” refers to the invasion of a host by a disease causing agent. A disease causing agent is considered to be “infectious” when it is capable of invading a host, and replicating or propagating within the host. Examples of infectious agents include viruses, e.g., HBV and certain species of adenovirus, prions, bacteria, fungi, protozoa and the like. “HBV infection” specifically refers to invasion of a host organism, such as cells and tissues of the host organism, by HBV.

The phrase “inducing an immune response” when used with reference to the methods described herein encompasses causing a desired immune response or effect in a subject in need thereof against an infection, e.g., an HBV infection. “Inducing an immune response” also encompasses providing a therapeutic immunity for treating against a pathogenic agent, e.g., HBV. As used herein, the term “therapeutic immunity” or “therapeutic immune response” means that the vaccinated subject is able to control an infection with the pathogenic agent against which the vaccination was done, for instance immunity against HBV infection conferred by vaccination with HBV vaccine. In an embodiment, “inducing an immune response” means producing an immunity in a subject in need thereof, e.g., to provide a therapeutic effect against a disease, such as HBV infection. In certain embodiments, “inducing an immune response” refers to causing or improving cellular immunity, e.g., T cell response, against HBV infection. In certain embodiments, “inducing an immune response” refers to causing or improving a humoral immune response against HBV infection. In certain embodiments, “inducing an immune response” refers to causing or improving a cellular and a humoral immune response against HBV infection.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with said agent.

Typically, the administration of compositions and therapeutic combinations of the application will have a therapeutic aim to generate an immune response against HBV after HBV infection or development of symptoms characteristic of HBV infection, e.g., for therapeutic vaccination.

As used herein, “an immunogenically effective amount” or “immunologically effective amount” means an amount of a composition, polynucleotide, vector, or antigen sufficient to induce a desired immune effect or immune response in a subject in need thereof. An immunogenically effective amount can be an amount sufficient to induce an immune response in a subject in need thereof. An immunogenically effective amount can be an amount sufficient to produce immunity in a subject in need thereof, e.g., provide a therapeutic effect against a disease such as HBV infection. An immunogenically effective amount can vary depending upon a variety of factors, such as the physical condition of the subject, age, weight, health, etc.; the particular application, e.g., providing protective immunity or therapeutic immunity; and the particular disease, e.g., viral infection, for which immunity is desired. An immunogenically effective amount can readily be determined by one of ordinary skill in the art in view of the present disclosure.

In particular embodiments of the application, an immunogenically effective amount refers to the amount of a composition or therapeutic combination which is sufficient to achieve one, two, three, four, or more of the following effects: (i) reduce or ameliorate the severity of an HBV infection or a symptom associated therewith; (ii) reduce the duration of an HBV infection or symptom associated therewith; (iii) prevent the progression of an HBV infection or symptom associated therewith; (iv) cause regression of an HBV infection or symptom associated therewith; (v) prevent the development or onset of an HBV infection, or symptom associated therewith; (vi) prevent the recurrence of an HBV infection or symptom associated therewith; (vii) reduce hospitalization of a subject having an HBV infection; (viii) reduce hospitalization length of a subject having an HBV infection; (ix) increase the survival of a subject with an HBV infection; (x) eliminate an HBV infection in a subject; (xi) inhibit or reduce HBV replication in a subject; and/or (xii) enhance or improve the prophylactic or therapeutic effect(s) of another therapy.

An immunogenically effective amount can also be an amount sufficient to reduce HBsAg levels consistent with evolution to clinical seroconversion; achieve sustained HBsAg clearance associated with reduction of infected hepatocytes by a subject's immune system; induce HBV-antigen specific activated T-cell populations; and/or achieve persistent loss of HBsAg within 12 months. Examples of a target index include lower HBsAg below a threshold of 500 copies of HBsAg international units (IU) and/or higher CD8 counts.

As general guidance, an immunogenically effective amount when used with reference to a DNA plasmid can range from about 0.1 mg/mL to 10 mg/mL of DNA plasmid total, such as 0.1 mg/mL, 0.25 mg/mL, 0.5 mg/mL. 0.75 mg/mL 1 mg/mL, 1.5 mg/mL, 2 mg/mL, 3 mg/mL, 4 mg/mL, 5 mg/mL, 6 mg/mL, 7 mg/mL, 8 mg/mL, 9 mg/mL, or 10 mg/mL. Preferably, an immunogenically effective amount of DNA plasmid is less than 8 mg/mL, more preferably less than 6 mg/mL, even more preferably 3-4 mg/mL. An immunogenically effective amount can be from one vector or plasmid, or from multiple vectors or plasmids. As further general guidance, an immunogenically effective amount when used with reference to a peptide can range from about 10 μg to 1 mg per administration, such as 10, 20, 50, 100, 200, 300, 400, 500, 600, 700, 800, 9000, or 1000 μg per administration. An immunogenically effective amount can be administered in a single composition, or in multiple compositions, such as 1, 2, 3, 4, 5, 6, 7, 8, 9, or 10 compositions (e.g., tablets, capsules or injectables, or any composition adapted to intradermal delivery, e.g., to intradermal delivery using an intradermal delivery patch), wherein the administration of the multiple capsules or injections collectively provides a subject with an immunogenically effective amount. For example, when two DNA plasmids are used, an immunogenically effective amount can be 3-4 mg/mL, with 1.5-2 mg/mL of each plasmid. It is also possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

A therapeutic combination comprising two DNA plasmids, e.g., a first DNA plasmid encoding an HBV core antigen and second DNA plasmid encoding an HBV pol antigen, can be administered to a subject by mixing both plasmids and delivering the mixture to a single anatomic site. Alternatively, two separate immunizations each delivering a single expression plasmid can be performed. In such embodiments, whether both plasmids are administered in a single immunization as a mixture of in two separate immunizations, the first DNA plasmid and the second DNA plasmid can be administered in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second DNA plasmids are administered in a ratio of 1:1, by weight.

As general guidance, an immunogenically effective amount when used with reference to an RNAi agent can range from about 0.05 mg/kg to about 5 mg/kg, e.g. about 0,05 mg to about 4 mg/kg or about 1 mg/kg to about 3 mg/kg, or for example about 0.05, 0.1, 0.2, 0.3, 0.4, 0.5, 1, 1.5, 2, 2.5, 3, 3.5, 4, 4.5 or 5 mg/kg, but can even higher, for example about 10, 11, 12, 13, 14, 15, 16, 17, 18, 19, 20, 21, 22, 23, 24, 25, 30, 40, 50, 60, 70, 80, 90 or 100 mg/kg. A fixed unit dose can also be given, for example, 50, 100, 200, 500 or 1000 mg, or the dose can be based on the patient's surface area, e.g., 500, 400, 300, 250, 200, or 100 mg/m2. Usually between 1 and 8 doses, (e.g., 1, 2, 3, 4, 5, 6, 7 or 8) can be administered to treat the patient, but 9, 10, 11, 12, 13, 14, 15, 16, 17, 18, 19, 20 or more doses can be given.

Administration of RNAi agents of the application can be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration can be at the same dose or at a different dose. For example, RNAi agents of the application can be provided as a daily dosage in an amount of about 0.05-5 mg/kg, such as 0.05, 0.1, 0.2, 0.3, 0.4, 0.5, 1, 1.5, 2, 2.5, 3, 3.5, 4, 4.5 or 5 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

In some embodiments, the ratio of AD04872 to AD05070 administered to a subject in need thereof is about 2:1. In some embodiments, the ratio of AD04872 to AD05070 administered to a subject in need thereof is about 3:1. In some embodiments, the ratio of AD04872 to AD05070 administered to a subject in need thereof is about 1:1. In some embodiments, the ratio of AD04872 to AD05070 administered to a subject in need thereof is about 4:1. In some embodiments, the ratio of AD04872 to AD05070 administered to a subject in need thereof is about 5:1. In some embodiments, the ratio of AD04872 to AD05070 administered to a subject in need thereof is about 1:2.

Preferably, a subject to be treated according to the methods of the application is an HBV-infected subject, particular a subject having chronic HBV infection. Acute HBV infection is characterized by an efficient activation of the innate immune system complemented with a subsequent broad adaptive response (e.g., HBV-specific T-cells, neutralizing antibodies), which usually results in successful suppression of replication or removal of infected hepatocytes. In contrast, such responses are impaired or diminished due to high viral and antigen load, e.g., HBV envelope proteins are produced in abundance and can be released in sub-viral particles in 1,000-fold excess to infectious virus.

Chronic HBV infection is described in phases characterized by viral load, liver enzyme levels (necroinflammatory activity), HBeAg, or HBsAg load or presence of antibodies to these antigens. cccDNA levels stay relatively constant at approximately 10 to 50 copies per cell, even though viremia can vary considerably. The persistence of the cccDNA species leads to chronicity. More specifically, the phases of chronic HBV infection include: (i) the immune-tolerant phase characterized by high viral load and normal or minimally elevated liver enzymes; (ii) the immune activation HBeAg-positive phase in which lower or declining levels of viral replication with significantly elevated liver enzymes are observed; (iii) the inactive HBsAg carrier phase, which is a low replicative state with low viral loads and normal liver enzyme levels in the serum that may follow HBeAg seroconversion; and (iv) the HBeAg-negative phase in which viral replication occurs periodically (reactivation) with concomitant fluctuations in liver enzyme levels, mutations in the pre-core and/or basal core promoter are common, such that HBeAg is not produced by the infected cell.

As used herein, “chronic HBV infection” refers to a subject having the detectable presence of HBV for more than 6 months. A subject having a chronic HBV infection can be in any phase of chronic HBV infection. Chronic HBV infection is understood in accordance with its ordinary meaning in the field. Chronic HBV infection can for example be characterized by the persistence of HBsAg for 6 months or more after acute HBV infection. For example, a chronic HBV infection referred to herein follows the definition published by the Centers for Disease Control and Prevention (CDC), according to which a chronic HBV infection can be characterized by laboratory criteria such as: (i) negative for IgM antibodies to hepatitis B core antigen (IgM anti-HBc) and positive for hepatitis B surface antigen (HBsAg), hepatitis B e antigen (HBeAg), or nucleic acid test for hepatitis B virus DNA, or (ii) positive for HBsAg or nucleic acid test for HBV DNA, or positive for HBeAg two times at least 6 months apart.

Preferably, an immunogenically effective amount refers to the amount of a composition or therapeutic combination of the application which is sufficient to treat chronic HBV infection.

In some embodiments, a subject having chronic HBV infection is undergoing nucleoside analog (NUC) treatment, and is NUC-suppressed. As used herein, “NUC-suppressed” refers to a subject having an undetectable viral level of HBV and stable alanine aminotransferase (ALT) levels for at least six months. Examples of nucleoside/nucleotide analog treatment include HBV polymerase inhibitors, such as entacavir and tenofovir. Preferably, a subject having chronic HBV infection does not have advanced hepatic fibrosis or cirrhosis. Such subject would typically have a METAVIR score of less than 3 for fibrosis and a fibroscan result of less than 9 kPa. The METAVIR score is a scoring system that is commonly used to assess the extent of inflammation and fibrosis by histopathological evaluation in a liver biopsy of patients with hepatitis B. The scoring system assigns two standardized numbers: one reflecting the degree of inflammation and one reflecting the degree of fibrosis.

It is believed that elimination or reduction of chronic HBV may allow early disease interception of severe liver disease, including virus-induced cirrhosis and hepatocellular carcinoma. Thus, the methods of the application can also be used as therapy to treat HBV-induced diseases. Examples of HBV-induced diseases include, but are not limited to cirrhosis, cancer (e.g., hepatocellular carcinoma), and fibrosis, particularly advanced fibrosis characterized by a METAVIR score of 3 or higher for fibrosis. In such embodiments, an immunogenically effective amount is an amount sufficient to achieve persistent loss of HBsAg within 12 months and significant decrease in clinical disease (e.g., cirrhosis, hepatocellular carcinoma, etc.).

Methods according to embodiments of the application further comprises administering to the subject in need thereof another immunogenic agent (such as another HBV antigen or other antigen) or another anti-HBV agent (such as a nucleoside analog or other anti-HBV agent) in combination with a composition of the application. For example, another anti-HBV agent or immunogenic agent can be a small molecule or antibody including, but not limited to, immune checkpoint inhibitors (e.g., anti-PD1, anti-TIM-3, etc.), toll-like receptor agonists (e.g., TLR7 agonists and/oror TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, IL-7-hyFc; CAR-T which bind HBV env (S-CAR cells); capsid assembly modulators;

cccDNA inhibitors, HBV polymerase inhibitors (e.g., entecavir and tenofovir). The one or other anti-HBV active agents can be, for example, a small molecule, an antibody or antigen-binding fragment thereof, a polypeptide, protein, or nucleic acid. The one or other anti-HBV agents can e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDMS inhibitors.

### Methods of Delivery

Compositions and therapeutic combinations of the application can be administered to a subject by any method known in the art in view of the present disclosure, including, but not limited to, parenteral administration (e.g., intramuscular, subcutaneous, intravenous, or intradermal injection), oral administration, transdermal administration, and nasal administration. Preferably, compositions and therapeutic combinations are administered parenterally (e.g., by intramuscular injection or intradermal injection) or transdermally.

In some embodiments of the application in which a composition or therapeutic combination comprises one or more DNA plasmids, administration can be by injection through the skin, e.g., intramuscular or intradermal injection, preferably intramuscular injection. Intramuscular injection can be combined with electroporation, i.e., application of an electric field to facilitate delivery of the DNA plasmids to cells. As used herein, the term “electroporation” refers to the use of a transmembrane electric field pulse to induce microscopic pathways (pores) in a bio-membrane. During in vivo electroporation, electrical fields of appropriate magnitude and duration are applied to cells, inducing a transient state of enhanced cell membrane permeability, thus enabling the cellular uptake of molecules unable to cross cell membranes on their own. Creation of such pores by electroporation facilitates passage of biomolecules, such as plasmids, oligonucleotides, siRNAs, drugs, etc., from one side of a cellular membrane to the other. In vivo electroporation for the delivery of DNA vaccines has been shown to significantly increase plasmid uptake by host cells, while also leading to mild-to-moderate inflammation at the injection site. As a result, transfection efficiency and immune response are significantly improved (e.g., up to 1,000 fold and 100 fold respectively) with intradermal or intramuscular electroporation, in comparison to conventional injection.

In a typical embodiment, electroporation is combined with intramuscular injection. However, it is also possible to combine electroporation with other forms of parenteral administration, e.g., intradermal injection, subcutaneous injection, etc.

Administration of a composition, therapeutic combination or vaccine of the application via electroporation can be accomplished using electroporation devices that can be configured to deliver to a desired tissue of a mammal a pulse of energy effective to cause reversible pores to form in cell membranes. The electroporation device can include an electroporation component and an electrode assembly or handle assembly. The electroporation component can include one or more of the following components of electroporation devices: controller, current waveform generator, impedance tester, waveform logger, input element, status reporting element, communication port, memory component, power source, and power switch. Electroporation can be accomplished using an in vivo electroporation device. Examples of electroporation devices and electroporation methods that can facilitate delivery of compositions and therapeutic combinations of the application, particularly those comprising DNA plasmids, include CELLECTRA® (Inovio Pharmaceuticals, Blue Bell, Pa.), Elgen electroporator (Inovio Pharmaceuticals, Inc.) Tri-Grid™ delivery system (Ichor Medical Systems, Inc., San Diego, Calif. 92121) and those described in U.S. Pat. No. 7,664,545, U.S. Pat. No. 8,209,006, U.S. Pat. No. 9,452,285, U.S. Pat. No. 5,273,525, U.S. Pat. No. 6,110,161, U.S. Pat. No. 6,261,281, U.S. Pat. No. 6,958,060, and U.S. Pat. No. 6,939,862, U.S. Pat. No. 7,328,064, U.S. Pat. No. 6,041,252, U.S. Pat. No. 5,873,849, U.S. Pat. No. 6,278,895, U.S. Pat. No. 6,319,901, U.S. Pat. No. 6,912,417, U.S. Pat. No. 8,187,249, U.S. Pat. No. 9,364,664, U.S. Pat. No. 9,802,035, U.S. Pat. No. 6,117,660, and International Patent Application Publication WO2017172838, all of which are herein incorporated by reference in their entireties. Other examples of in vivo electroporation devices are described in International Patent Application entitled “Method and Apparatus for the Delivery of Hepatitis B Virus (HBV) Vaccines,” filed on the same day as this application with the Attorney Docket Number 688097-405WO, the contents of which are hereby incorporated by reference in their entireties. Also contemplated by the application for delivery of the compositions and therapeutic combinations of the application are use of a pulsed electric field, for instance as described in, e.g., U.S. Pat. No. 6,697,669, which is herein incorporated by reference in its entirety.

In other embodiments of the application in which a composition or therapeutic combination comprises one or more DNA plasmids, the method of administration is transdermal. Transdermal administration can be combined with epidermal skin abrasion to facilitate delivery of the DNA plasmids to cells. For example, a dermatological patch can be used for epidermal skin abrasion. Upon removal of the dermatological patch, the composition or therapeutic combination can be deposited on the abraised skin.

Methods of delivery are not limited to the above described embodiments, and any means for intracellular delivery can be used. Other methods of intracellular delivery contemplated by the methods of the application include, but are not limited to, liposome encapsulation, lipid nanoparticles (LNPs), etc.

In certain embodiments of the application, the method of administration is a lipid composition, such as a lipid nanoparticle (LNP). Lipid compositions, preferably lipid nanoparticles, that can be used to deliver a therapeutic product (such as one or more nucleic acid molecules of the invention), include, but are not limited to, liposomes or lipid vesicles, wherein an aqueous volume is encapsulated by amphipathic lipid bilayers, or wherein the lipids coat an interior that comprises a therapeutic product; or lipid aggregates or micelles, wherein the lipid-encapsulated therapeutic product is contained within a relatively disordered lipid mixture.

In particular embodiments, the LNPs comprise a cationic lipid to encapsulate and/or enhance the delivery of a nucleic acid molecule, such as a DNA or RNA molecule of the invention, into the target cell. The cationic lipid can be any lipid species that carries a net positive charge at a selected pH, such as physiological pH. The lipid nanoparticles can be prepared by including multi-component lipid mixtures of varying ratios employing one or more cationic lipids, non-cationic lipids and polyethylene glycol (PEG)-modified lipids. Several cationic lipids have been described in the literature, many of which are commercially available. For example, suitable cationic lipids for use in the compositions and methods of the invention include 1,2-dioleoyl-3-trimethylammonium-propane (DOTAP).

The LNP formulations can include anionic lipids. The anionic lipids can be any lipid species that carries a net negative charge at a selected pH, such as physiological pH. The anionic lipids, when combined with cationic lipids, are used to reduce the overall surface charge of LNPs and to introduce pH-dependent disruption of the LNP bilayer structure, facilitating nucleotide release. Several anionic lipids have been described in the literature, many of which are commercially available. For example, suitable anionic lipids for use in the compositions and methods of the invention include 1,2-dioleoyl-sn-glycero-3-phosphoethanolamine (DOPE).

LNPs can be prepared using methods well known in the art in view of the present disclosure. For example, the LNPs can be prepared using ethanol injection or dilution, thin film hydration, freeze-thaw, French press or membrane extrusion, diafiltration, sonication, detergent dialysis, ether infusion, and reverse phase evaporation.

Some examples of lipids, lipid compositions, and methods to create lipid carriers for delivering active nucleic acid molecules, such as those of this invention, are described in: US2017/0190661, US2006/0008910, US2015/0064242, US2005/0064595, WO/2019/036030, US2019/0022247, WO/2019/036028, WO/2019/036008, WO/2019/036000, US2016/0376224, US2017/0119904, WO/2018/200943, WO/2018/191657, US2014/0255472, and US2013/0195968, the relevant content of each of which is hereby incorporated by reference in its entirety.

A pharmaceutical composition comprising RNAi agents of the application comprises a pharmacologically effective amount of at least one kind of RNAi and a pharmaceutically acceptable carrier. However, such a “pharmaceutical composition” can also comprise individual strands of such RNAi agents or vector(s) comprising a regulatory sequence operably linked to a nucleotide sequence that encodes at least one strand of a sense or an antisense strand comprised in the RNAi's of this application. It is also envisaged that cells, tissues or isolated organs that express or comprise the herein defined RNAi can be used as “pharmaceutical compositions”.

RNAi agents for inhibiting the expression of an HBV gene of the application can be administered to a subject by any suitable route, for example parentally by intravenous (i.v.) infusion or bolus injection, intramuscularly or subcutaneously or intraperitoneally. Intravenous infusion can be given over for example 15, 30, 60, 90, 120, 180, or 240 minutes, or from 1, 2, 3, 4, 5, 6, 7, 8, 9, 10, 11 or 12 hours.

For intramuscular, subcutaneous and intravenous use, the pharmaceutical compositions comprising RNAi agents of the application will generally be provided in sterile aqueous solutions or suspensions, buffered to an appropriate pH and isotonicity. In a preferred embodiment, the carrier consists exclusively of an aqueous buffer. In this context, “exclusively” means no auxiliary agents or encapsulating substances are present which might affect or mediate uptake of dsRNA in the cells that express a Hepatitis B Virus gene. Aqueous suspensions according to the application can include suspending agents such as cellulose derivatives, sodium alginate, polyvinylpyrrolidone and gum tragaeanth, and a wetting agent such as lecithin. Suitable preservatives for aqueous suspensions include ethyl and n-propyl p-hydroxybenzoate. The pharmaceutical compositions comprising RNAi agents useful according to the application also include encapsulated formulations to protect the RN Ai agents against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions and bi-specific antibodies can also be used as pharmaceutically acceptable carriers. These can be prepared according to methods known to those skilled in the art, for example, as described in PCT publication WO91/06309 and WO 2011/003780 which are incorporated by reference in their entirety herein.

### Adjuvants

In some embodiments of the application, a method of inducing an immune response against HBV further comprises administering an adjuvant. The terms “adjuvant” and “immune stimulant” are used interchangeably herein, and are defined as one or more substances that cause stimulation of the immune system. In this context, an adjuvant is used to enhance an immune response to HBV antigens and antigenic HBV polypeptides of the application.

According to embodiments of the application, an adjuvant can be present in a therapeutic combination or composition of the application, or administered in a separate composition. An adjuvant can be, e.g., a small molecule or an antibody. Examples of adjuvants suitable for use in the application include, but are not limited to, immune checkpoint inhibitors (e.g., anti-PD1, anti-TIM-3, etc.), toll-like receptor agonists (e.g., TLR7 and/or TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, and IL-7-hyFc. Examples of adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDMS inhibitors.

Compositions and therapeutic combinations of the application can also be administered in combination with at least one other anti-HBV agent. Examples of anti-HBV agents suitable for use with the application include, but are not limited to small molecules, antibodies, and/or CAR-T therapies which bind HBV env (S-CAR cells), capsid assembly modulators, TLR agonists (e.g., TLR7 and/or TLR8 agonists), cccDNA inhibitors, HBV polymerase inhibitors (e.g., entecavir and tenofovir), and/or immune checkpoint inhibitors, etc.

The at least one anti-HBV agent can e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors. Such anti-HBV agents can be administered with the compositions and therapeutic combinations of the application simultaneously or sequentially.

### Methods of Prime/Boost Immunization

Embodiments of the application also contemplate administering an immunogenically effective amount of a composition or therapeutic combination to a subject, and subsequently administering another dose of an immunogenically effective amount of a composition or therapeutic combination to the same subject, in a so-called prime-boost regimen Thus, in an embodiment, a composition or therapeutic combination of the application is a primer vaccine used for priming an immune response. In another embodiment, a composition or therapeutic combination of the application is a booster vaccine used for boosting an immune response. The priming and boosting vaccines of the application can be used in the methods of the application described herein. This general concept of a prime-boost regimen is well known to the skilled person in the vaccine field. Any of the compositions and therapeutic combinations of the application described herein can be used as priming and/or boosting vaccines for priming and/or boosting an immune response against HBV.

In some embodiments of the application, a composition or therapeutic combination of the application can be administered for priming immunization. The composition or therapeutic combination can be re-administered for boosting immunization. Further booster administrations of the composition or vaccine combination can optionally be added to the regimen, as needed. An adjuvant can be present in a composition of the application used for boosting immunization, present in a separate composition to be administered together with the composition or therapeutic combination of the application for the boosting immunization, or administered on its own as the boosting immunization. In those embodiments in which an adjuvant is included in the regimen, the adjuvant is preferably used for boosting immunization.

An illustrative and non-limiting example of a prime-boost regimen includes administering a single dose of an immunogenically effective amount of a composition or therapeutic combination of the application to a subject to prime the immune response; and subsequently administering another dose of an immunogenically effective amount of a composition or therapeutic combination of the application to boost the immune response, wherein the boosting immunization is first administered about two to six weeks, preferably four weeks after the priming immunization is initially administered. Optionally, about 10 to 14 weeks, preferably 12 weeks, after the priming immunization is initially administered, a further boosting immunization of the composition or therapeutic combination, or other adjuvant, is administered.

### Kits

Also provided herein is a kit comprising a therapeutic combination of the application. A kit can comprise the first polynucleotide, the second polynucleotide, and the RNAi agent for inhibiting the expression of an HBV genein one or more separate compositions, or a kit can comprise the first polynucleotide, the second polynucleotide, and the RNAi agent for inhibiting the expression of an HBV gene in a single composition. A kit can further comprise one or more adjuvants or immune stimulants, and/or other anti-HBV agents.

The ability to induce or stimulate an anti-HBV immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELISPOT), by determination of the activation status of immune effector cells (e.g. T cell proliferation assays by a classical [3H] thymidine uptake or flow cytometry-based assays), by assaying for antigen-specific T lymphocytes in a sensitized subject (e.g. peptide-specific lysis in a cytotoxicity assay, etc.).

The ability to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

### Embodiments

The invention provides also the following non-limiting embodiments.

Embodiment 1 is a therapeutic combination for use in treating a hepatitis B virus (HBV) infection in a subject in need thereof, comprising:

i) at least one of:
a) a truncated HBV core antigen consisting of an amino acid sequence that is at least 95%, such as at least 95%, 96%, 97%, 98%, 99% or 100%, identical to SEQ ID NO: 2,
b) a first non-naturally occurring nucleic acid molecule comprising a first polynucleotide sequence encoding the truncated HBV core antigen
c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and

ii) an RNAi agent for inhibiting the expression of an HBV gene, such as those described in US20130005793, WO2013003520 or WO2018027106, the contents of which are incorporated herein by reference in their entirety.

Embodiment 2 is the therapeutic combination of embodiment 1, comprising at least one of the HBV polymerase antigen and the truncated HBV core antigen.

Embodiment 3 is the therapeutic combination of embodiment 2, comprising the HBV polymerase antigen and the truncated HBV core antigen.

Embodiment 4 is the therapeutic combination of embodiment 1, comprising at least one of the first non-naturally occurring nucleic acid molecule comprising the first polynucleotide sequence encoding the truncated HBV core antigen, and the second non-naturally occurring nucleic acid molecule comprising the second polynucleotide sequence encoding the HBV polymerase antigen.

Embodiment 5 is a therapeutic combination for use in treating a hepatitis B virus (HBV) infection in a subject in need thereof, comprising

i) a first non-naturally occurring nucleic acid molecule comprising a first polynucleotide sequence encoding a truncated HBV core antigen consisting of an amino acid sequence that is at least 95% identical to SEQ ID NO: 2; and

ii) a second non-naturally occurring nucleic acid molecule comprising a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity; and

iii) an RNAi agent for inhibiting the expression of an HBV gene, such as those described in US20130005793, WO2013003520 or WO2018027106, the contents of which are incorporated herein by reference in their entirety.

Embodiment 6 is the therapeutic combination of embodiment 4 or 5, wherein the first non-naturally occurring nucleic acid molecule further comprises a polynucleotide sequence encoding a signal sequence operably linked to the N-terminus of the truncated HBV core antigen.

Embodiment 6a is the therapeutic combination of any one of embodiments 4 to 6, wherein the second non-naturally occurring nucleic acid molecule further comprises a polynucleotide sequence encoding a signal sequence operably linked to the N-terminus of the HBV polymerase antigen.

Embodiment 6b is the therapeutic combination of embodiment 6 or 6a, wherein the signal sequence independently comprises the amino acid sequence of SEQ ID NO: 9 or SEQ ID NO: 15.

Embodiment 6c is the therapeutic combination of embodiment 6 or 6a, wherein the signal sequence is independently encoded by the polynucleotide sequence of SEQ ID NO: 8 or SEQ ID NO: 14.

Embodiment 7 is the therapeutic combination of any one of embodiments 1-6c, wherein the HBV polymerase antigen comprises an amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 7.

Embodiment 7a is the therapeutic combination of embodiment 7, wherein the HBV polymerase antigen comprises the amino acid sequence of SEQ ID NO: 7.

Embodiment 7b is the therapeutic combination of any one of embodiments 1 to 7a, wherein and the truncated HBV core antigen consists of the amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 2.

Embodiment 7c is the therapeutic combination of embodiment 7b, wherein the truncated HBV antigen consists of the amino acid sequence of SEQ ID NO: 2 or SEQ ID NO: 4.

Embodiment 8 is the therapeutic combination of any one of embodiments 1-7c, wherein each of the first and second non-naturally occurring nucleic acid molecules is a DNA molecule.

Embodiment 8a is the therapeutic combination of embodiment 8, wherein the DNA molecule is present on a DNA vector.

Embodiment 8b is the therapeutic combination of embodiment 8a, wherein the DNA vector is selected from the group consisting of DNA plasmids, bacterial artificial chromosomes, yeast artificial chromosomes, and closed linear deoxyribonucleic acid.

Embodiment 8c is the therapeutic combination of embodiment 8, wherein the DNA molecule is present on a viral vector.

Embodiment 8d is the therapeutic combination of embodiment 8c, wherein the viral vector is selected from the group consisting of bacteriophages, animal viruses, and plant viruses.

Embodiment 8e is the therapeutic combination of any one of embodiments 1-7c, wherein each of the first and second non-naturally occurring nucleic acid molecules is an RNA molecule.

Embodiment 8f is the therapeutic combination of embodiment 8e, wherein the RNA molecule is an RNA replicon, preferably a self-replicating RNA replicon, an mRNA replicon, a modified mRNA replicon, or self-amplifying mRNA.

Embodiment 8g is the therapeutic combination of any one of embodiments 1 to 8f, wherein each of the first and second non-naturally occurring nucleic acid molecules is independently formulated with a lipid composition, preferably a lipid nanoparticle (LNP).

Embodiment 9 is the therapeutic combination of any one of embodiments 4-8g, comprising the first non-naturally occurring nucleic acid molecule and the second non-naturally occurring nucleic acid molecule in the same non-naturally occurring nucleic acid molecule.

Embodiment 10 is the therapeutic combination of any one of embodiments 4-8g, comprising the first non-naturally occurring nucleic acid molecule and the second non-naturally occurring nucleic acid molecule in two different non-naturally occurring nucleic acid molecules.

Embodiment 11 is the therapeutic combination of any one of embodiments 4-10, wherein the first polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

Embodiment 11a is the therapeutic combination of embodiment 11, wherein the first polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 1 or SEQ ID NO: 3.

Embodiment 12 is the therapeutic combination of embodiment 11a, wherein the first polynucleotide sequence comprises the polynucleotide sequence of SEQ ID NO: 1 or SEQ ID NO: 3.

Embodiment 13 the therapeutic combination of any one of embodiments 4 to 12, wherein the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

Embodiment 13a the therapeutic combination of embodiment 13, wherein the second polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 5 or SEQ ID NO: 6.

Embodiment 14 is the therapeutic combination of embodiment 13a, wherein the second polynucleotide sequence comprises the polynucleotide sequence of SEQ ID NO: 5 or SEQ ID NO: 6.

Embodiment 15 is the therapeutic combination of any one of embodiments 1 to 14, wherein the RNAi agent has the core sense strand sequence and antisense strand sequence shown in Table 2.

Embodiment 15a is the therapeutic combination of any one of embodiments 1 to 14, wherein the RNAi agent has the sense strand sequence and antisense strand sequence shown in Table 3.

Embodiment 15b is the therapeutic combination of any one of embodiments 1 to 14, wherein the RNAi agent has the core sense strand sequence and antisense strand sequence shown in Table 4.

Embodiment 15c is the therapeutic combination of embodiment 15b, wherein the RNAi agent has the modified sense strand sequence and antisense strand sequence shown in Table 4.

Embodiment 15d is the therapeutic combination of any one of embodiments 1 to 14, wherein the RNAi agent targets a target sequence shown in Table 5.

Embodiment 15e is the therapeutic combination of any one of embodiments 1 to 14, wherein the RNAi agent has the core sense strand sequence and antisense strand sequence shown in Table 6.

Embodiment 15f is the therapeutic combination of any one of embodiments 1 to 14, wherein the RNAi agent has a core antisense sequence shown in Table 7 and a core sense strand sequence shown in Table 8.

Embodiment 15g is the therapeutic combination of embodiment 15f, wherein the RNAi agent has the modified sense strand sequence shown in Table 7 and the modified antisense strand sequence shown in Table 8.

Embodiment 15h is the therapeutic combination of any one of embodiments 1 to 14, wherein the RNAi agent has a duplex of an antisense strand and a sense strand shown in Table 9.

Embodiment 15i is the therapeutic combination of embodiment 15h, wherein the RNAi agent has the duplex structure of AD04580; AD04585; AD04776; AD04872; AD04962; AD04963; AD04982; or AD05070 shown in Table 9.

Embodiment 15j is the therapeutic combination of any one of embodiments 1 to 14, wherein the therapeutic combination comprises a first RNAi agent targeting the S open reading frame (ORF) of an HBV gene, and a second RNAi agent targeting the X open reading frame (ORF) of an HBV gene.

Embodiment 15k is the therapeutic combination of embodiment 15j, wherein the first RNAi agent is selected from the group consisting of: AD04001; AD04002; AD04003; AD04004; AD04005; AD04006; AD04007; AD04008; AD04009; AD04010; AD04422; AD04423; AD04425; AD04426; AD04427; AD04428; AD04429; AD04430; AD04431; AD04432; AD04433; AD04434; AD04435; AD04436; AD04437; AD04438; AD04439; AD04440; AD04441; AD04442; AD04511; AD04581; AD04583; AD04584; AD04585; AD04586; AD04587; AD04588; AD04590; AD04591; AD04592; AD04593; AD04594; AD04595; AD04596; AD04597; AD04598; AD04599; AD04734; AD04771; AD04772; AD04773; AD04774; AD04775; AD04822; AD04871; AD04872; AD04873; AD04874; AD04875; AD04876; AD04962; and AD05164; and the second RNAi agent is selected from the group consisting of: AD03498; AD03499; AD03500; AD03501; AD03738; AD03739; AD03967; AD03968; AD03969; AD03970; AD03971; AD03972; AD03973; AD03974; AD03975; AD03976; AD03977; AD03978; AD04176; AD04177; AD04178; AD04412; AD04413; AD04414; AD04415; AD04416; AD04417; AD04418; AD04419; AD04420; AD04421; AD04570; AD04571; AD04572; AD04573; AD04574; AD04575; AD04576; AD04577; AD04578; AD04579; AD04580; AD04776; AD04777; AD04778; AD04823; AD04881; AD04882; AD04883; AD04884; AD04885; AD04963; AD04981; AD04982; AD04983; AD05069; AD05070; AD05071; AD05072; AD05073; AD05074; AD05075; AD05076; AD05077; AD05078; AD05147; AD05148; AD05149; and AD05165, each of which is described in WO2018027106 and the disclosure of which is incorporated herein by reference in its entirety.

Embodiment 15l is the therapeutic combination of embodiment 15k, wherein the first RNAi agent is AD04872, which comprises a duplex having the sequences of SEQ ID NOs: 25-26, and the second RNAi agent is AD05070, which comprises a duplex having the sequences of SEQ ID NOs: 27-28.

Embodiment 15m is the therapeutic combination of embodiment 15k, wherein the first RNAi agent is AD04872 and the second RNAi agent is AD04982.

Embodiment 15n is the therapeutic combination of embodiment 15k, wherein the first RNAi agent is AD04872 and the second RNAi agent is AD04776.

Embodiment 15o is the therapeutic combination of embodiment 15k, wherein the first RNAi agent is AD04585 and the second RNAi agent is AD04580.

Embodiment 15p is the therapeutic combination of any one of embodiments 1 to 15o, wherein the RNAi agent is formulated in a lipid composition, preferably a lipid nanoparticle.

Embodiment 15p is the therapeutic combination of any one of embodiments 1 to 15o, wherein the RNA agent is conjugated to a targeting ligand.

Embodiment 15q is the therapeutic combination of embodiment 15p, wherein the targeting ligand comprises N-acetyl-galactosamine.

Embodiment 15r is the therapeutic combination of embodiment 15p, wherein the targeting ligand is (NAG13), (NAG13)s, (NAG18), (NAG18)s, (NAG24), (NAG24)s, (NAG25), (NAG25)s, (NAG26), (NAG26)s, (NAG27), (NAG27)s, (NAG28), (NAG28)s, (NAG29), (NAG29)s, (NAG30), (NAG30)s, (NAG31), (NAG31)s, (NAG32), (NAG32)s, (NAG33), (NAG33)s, (NAG34), (NAG34)s, (NAG35), (NAG35)s, (NAG36), (NAG36)s, (NAG37), (NAG37)s, (NAG38), (NAG38)s, (NAG39), or (NAG39) depicted in Table 10, each of which is described in more detail in WO2018027106 and the disclosure of which is incorporated herein by reference in its entirety.

Embodiment 15s is the therapeutic combination of embodiment 15p, wherein the targeting ligand is (NAG34), (NAG34)s, (NAG35), (NAG35)s, (NAG36), (NAG36)s, (NAG37), (NAG37)s, (NAG38), (NAG38)s (NAG39), or (NAG39)s, more preferably (NAG37) or (NAG37)s.

Embodiment 15t is the therapeutic combination of any one of embodiments 15p to 15s, wherein the targeting ligand is conjugated to the sense strand of the RNAi agent.

Embodiment 16 is a kit comprising the therapeutic combination of any one of embodiments 1 to 15t, and instructions for using the therapeutic combination in treating a hepatitis B virus (HBV) infection in a subject in need thereof.

Embodiment 17 is a method of treating a hepatitis B virus (HBV) infection in a subject in need thereof, comprising administering to the subject the therapeutic combination of any one of embodiments 1 to 15t.

Embodiment 17a is the method of embodiment 17, wherein the treatment induces an immune response against a hepatitis B virus in a subject in need thereof, preferably the subject has chronic HBV infection.

Embodiment 17b is the method of embodiment 17 or 17a, wherein the subject has chronic HBV infection.

Embodiment 17c is the method of any one of embodiments 17 to 17b, wherein the subject is in need of a treatment of an HBV-induced disease selected from the group consisting of advanced fibrosis, cirrhosis and hepatocellular carcinoma (HCC).

Embodiment 18 is the method of any one of embodiments 17-17c, wherein the therapeutic combination is administered by injection through the skin, e.g., intramuscular or intradermal injection, preferably intramuscular injection.

Embodiment 19 is the method of embodiment 18, wherein the therapeutic combination comprises at least one of the first and second non-naturally occurring nucleic acid molecules.

Embodiment 19a is the method of embodiment 19, wherein the therapeutic combination comprises the first and second non-naturally occurring nucleic acid molecules.

Embodiment 20 is the method of embodiment 19 or 19a, wherein the non-naturally occurring nucleic acid molecules are administered to the subject by intramuscular injection in combination with electroporation.

Embodiment 21 is the method of embodiment 19 or 19a, wherein the non-naturally occurring nucleic acid molecules are administered to the subject by a lipid composition, preferably by a lipid nanoparticle.

### EXAMPLES

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Example 1

### HBV Core Plasmid & HBV Pol Plasmid

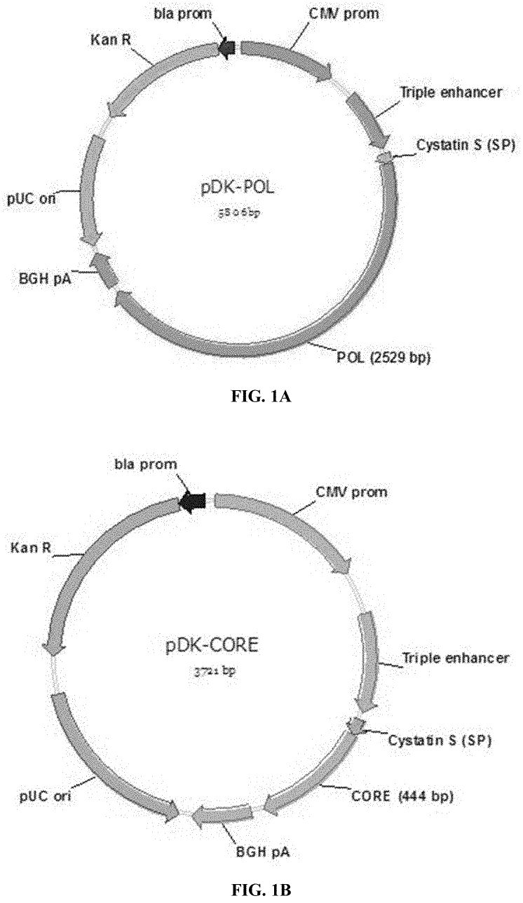

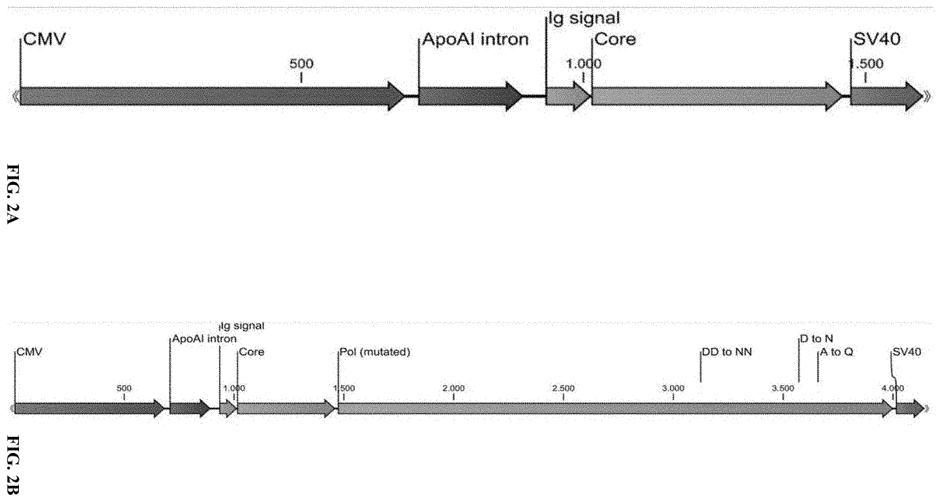

A schematic representation of the pDK-pol and pDK-core vectors is shown in FIGS. 1A and 1B, respectively. An HBV core or pol antigen optimized expression cassette containing a CMV promoter (SEQ ID NO: 18), a splicing enhancer (triple composite sequence) (SEQ ID NO: 10), Cystatin S precursor signal peptide SPCS (NP_0018901.1) (SEQ ID NO: 9), and pol (SEQ ID NO: 5) or core (SEQ ID NO: 2) gene was introduced into a pDK plasmid backbone, using standard molecular biology techniques.

The plasmids were tested in vitro for core and pol antigen expression by Western blot analysis using core and pol specific antibodies, and were shown to provide consistent expression profile for cellular and secreted core and pol antigens (data not shown).

### Example 2

### Generation of Adenoviral Vectors Expressing a Fusion of Truncated HBV Core Antigen with HBV Pol Antigen

The creation of an adenovirus vector has been designed as a fusion protein expressed from a single open reading frame. Additional configurations for the expression of the two proteins, e.g. using two separate expression cassettes, or using a 2A-like sequence to separate the two sequences, can also be envisaged.

### Design of Expression Cassettes for Adenoviral Vectors

The expression cassettes (diagrammed in FIG. 2A and FIG. 2B) are comprised of the CMV promoter (SEQ ID NO: 19), an intron (SEQ ID NO:12) (a fragment derived from the human ApoAI gene—GenBank accession X01038 base pairs 295-523, harboring the ApoAI second intron), followed by the optimized coding sequence—either core alone or the core and polymerase fusion protein preceded by a human immunoglobulin secretion signal coding sequence (SEQ ID NO: 14), and followed by the SV40 polyadenylation signal (SEQ ID NO: 13).

A secretion signal was included because of past experience showing improvement in the manufacturability of some adenoviral vectors harboring secreted transgenes, without influencing the elicited T-cell response (mouse experiments).

The last two residues of the Core protein (VV) and the first two residues of the Polymerase protein (MP) if fused results in a junction sequence (VVMP) that is present on the human dopamine receptor protein (D3 isoform), along with flanking homologies.

The interjection of an AGAG linker between the core and the polymerase sequences eliminates this homology and returned no further hits in a Blast of the human proteome.

### Example 3

### In Vivo Immunogenicity Study of DNA Vaccine in Mice

An immunotherapeutic DNA vaccine containing DNA plasmids encoding an HBV core antigen or HBV polymerase antigen was tested in mice. The purpose of the study was designed to detect T-cell responses induced by the vaccine after intramuscular delivery via electroporation into BALB/c mice. Initial immunogenicity studies focused on determining the cellular immune responses that would be elicited by the introduced HBV antigens.

In particular, the plasmids tested included a pDK-Pol plasmid and pDK-Core plasmid, as shown in FIGS. 1A and 1B, respectively, and as described above in Example 1. The pDK-Pol plasmid encoded a polymerase antigen having the amino acid sequence of SEQ ID NO: 7, and the pDK-Core plasmid encoding a Core antigen having the amino acid sequence of SEQ ID NO: 2. First, T-cell responses induced by each plasmid individually were tested. The DNA plasmid (pDNA) vaccine was intramuscularly delivered via electroporation to Balb/c mice using a commercially available TriGrid™ delivery system-intramuscular (TDS-IM) adapted for application in the mouse model in cranialis tibialis. See International Patent Application Publication WO2017172838, and U.S. Patent Application No. 62/607,430, entitled “Method and Apparatus for the Delivery of Hepatitis B Virus (HBV) Vaccines,” filed on Dec. 19, 2017 for additional description on methods and devices for intramuscular delivery of DNA to mice by electroporation, the disclosures of which are hereby incorporated by reference in their entireties. In particular, the TDS-IM array of a TDS-IM v1.0 device having an electrode array with a 2.5 mm spacing between the electrodes and an electrode diameter of 0.030 inch was inserted percutaneously into the selected muscle, with a conductive length of 3.2 mm and an effective penetration depth of 3.2 mm, and with the major axis of the diamond configuration of the electrodes oriented in parallel with the muscle fibers. Following electrode insertion, the injection was initiated to distribute DNA (e.g., 0.020 ml) in the muscle. Following completion of the IM injection, a 250 V/cm electrical field (applied voltage of 59.4-65.6 V, applied current limits of less than 4 A, 0.16 A/sec) was locally applied for a total duration of about 400 ms at a 10% duty cycle (i.e., voltage is actively applied for a total of about 40 ms of the about 400 ms duration) with 6 total pulses. Once the electroporation procedure was completed, the TriGrid™ array was removed and the animals were recovered. High-dose (20 μg) administration to BALB/c mice was performed as summarized in Table 1. Six mice were administered plasmid DNA encoding the HBV core antigen (pDK-core; Group 1), six mice were administered plasmid DNA encoding the HBV pol antigen (pDK-pol; Group 2), and two mice received empty vector as the negative control. Animals received two DNA immunizations two weeks apart and splenocytes were collected one week after the last immunization.

TABLE 1
Mouse immunization experimental design of the pilot study.

| | | | Unilateral | | | | Endpoint |
|---|---|---|---|---|---|---|---|
| | | | Admin Site | | | | (spleen |
| | | | (alternate | | | Admin | harvest) |
| Group | N | pDNA | sides) | Dose | Vol | Days | Day |
| 1 | 6 | Core | CT + EP | 20 μg | 20 μL | 0, 14 | 21 |
| 2 | 6 | Pol | CT + EP | 20 μg | 20 μL | 0, 14 | 21 |
| 3 | 2 | Empty | CT + EP | 20 μg | 20 μL | 0, 14 | 21 |
| | | Vector | | | | | |
| | | (neg | | | | | |
| | | control) | | | | | |

CT, cranialis tibialis muscle; EP, electroporation.

Antigen-specific responses were analyzed and quantified by IFN-γ enzyme-linked immunospot (ELISPOT). In this assay, isolated splenocytes of immunized animals were incubated overnight with peptide pools covering the Core protein, the Pol protein, or the small peptide leader and junction sequence (2 μg/ml of each peptide). These pools consisted of 15 mer peptides that overlap by 11 residues matching the Genotypes BCD consensus sequence of the Core and Pol vaccine vectors. The large 94 kDan HBV Pol protein was split in the middle into two peptide pools. Antigen-specific T cells were stimulated with the homologous peptide pools and IFN-γ-positive T cells were assessed using the ELISPOT assay. IFN-γ release by a single antigen-specific T cell was visualized by appropriate antibodies and subsequent chromogenic detection as a colored spot on the microplate referred to as spot-forming cell (SFC).

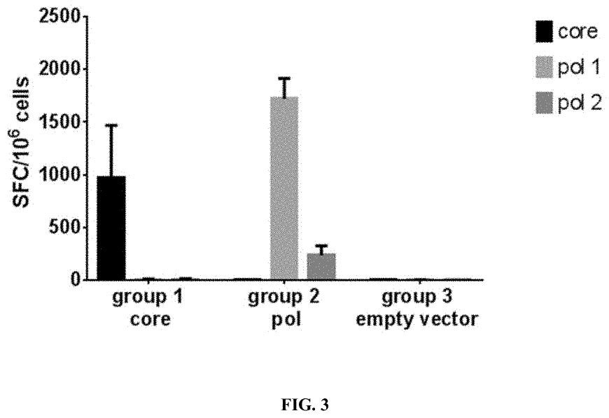

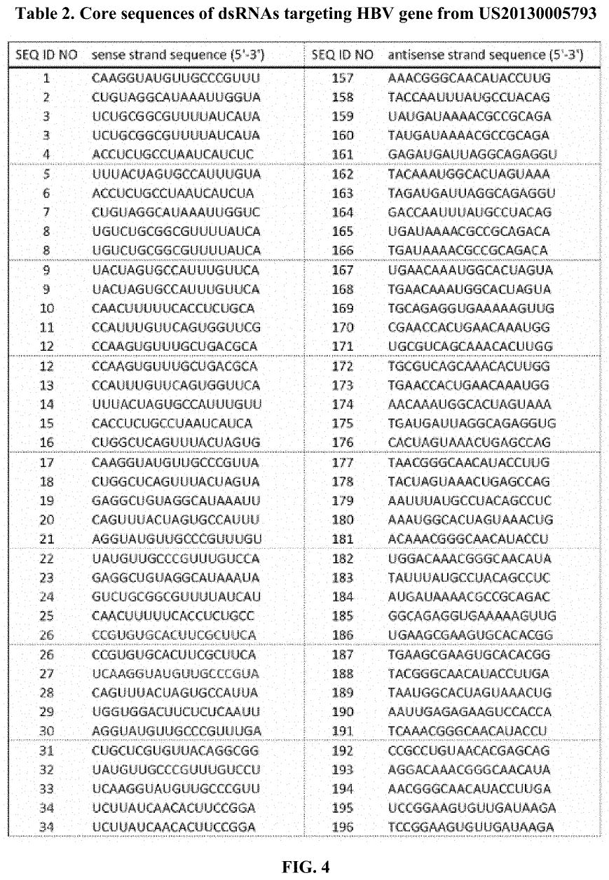

Substantial T-cell responses against HBV Core were achieved in mice immunized with the DNA vaccine plasmid pDK-Core (Group 1) reaching 1,000 SFCs per 10⁶ cells (FIG. 3). Pol T-cell responses towards the Pol 1 peptide pool were strong (˜1,000 SFCs per 10⁶ cells). The weak Pol-2-directed anti-Pol cellular responses were likely due to the limited MHC diversity in mice, a phenomenon called T-cell immunodominance defined as unequal recognition of different epitopes from one antigen. A confirmatory study was performed confirming the results obtained in this study (data not shown).

The above results demonstrate that vaccination with a DNA plasmid vaccine encoding HBV antigens induces cellular immune responses against the administered HBV antigens in mice. Similar results were also obtained with non-human primates (data not shown).

### Example 4

### In Vivo Immunogenicity Study of DNA Vaccine in Combination with HBV siRNA in Mice

C57BL/6 male mice (6-8 wks old; Janvier, France) are infected via tail vein injection with 1×10¹¹ vg AAV-HBV (FivePlus MMI, China) diluted in 1×PBS. Infection is allowed to establish for 28 days before treatment commencement. Mice (n=8/group) are then put into 6 separate groups to explore siRNA alone or therapeutic vaccine (Tx Vx) alone, or in combination (Table 2). TxVx is a 1:1 mixture of the pDK-Pol plasmid and the pDK-Core plasmid of Example 1 above (see also FIGS. 1A and 1B, respectively). siRNA is as described in WO 2018 027106 (e.g., claim 54 of WO 2018 027106), more particularly is a mixture of two RNAi agents AD04872+AD5070, AD04872+AD04982, AD04872+AD04776, or AD04585+AD04580 as described in WO 2018 027106. Dose and timing of dose for both the siRNA and Tx Vx is given in Table 2. The first day of treatment is designated DO and is after an infection establishment period of 28 days.

TABLE 2
Outline of treatment regimen for each of the study groups

| | Mice/ | | Tx Vx | | siRNA |
|---|---|---|---|---|---|
| Group | Group | Tx Vx | Dose time | siRNA | Dose time |
| 1 | 8 | vehicle | D 0, D 21 | vehicle | D 0, D 21 |
| 2 | 8 | vehicle | — | 10 mpk | D 0, D 21 |
| 3 | 8 | 10 ug pol/ | D 0, D 21 | vehicle | — |
| | | 10 ug core | | | |
| 4 | 8 | 10 ug pol/ | D 0, D 21 | 10 mpk | D 0, D 21 |
| | | 10 ug core | | | |
| 5 | 8 | 10 ug pol/ | D 21, D 42 | vehicle | — |
| | | 10 ug core | | | |
| 6 | 8 | 10 ug pol/ | D 21, D 42 | 10 mpk | D 0, D 21 |
| | | 10 ug core | | | |
| 7 | 8 | 10 ug pol/ | D 42, D 63 | 10 mpk | D 0, D 21 |
| | | 10 ug core | | | |
| 8 | 8 | 10 ug pol/ | D 42, D 63 | vehicle | D 0, D 21 |
| | | 10 ug core | | | |

— designates no treatment

The Tx Vx is diluted in 1×PBS at the concentrations designated in Table 2 and administered via electroporation in the tibialis muscle (Ichor, USA). siRNA is delivered via a subcutaneous injection on the back of neck at a concentration of 10 mpk in 1×PBS. The siRNA and Tx Vx combination in groups 4 and 6 are administered together (Group 4) or staggered so that the siRNA is administered 3 weeks before the first Tx Vx dose (Group 6) or 3 weeks after the last siRNA treatment (Group 7). All end points are 3 weeks after the last drug administration which corresponds to Day 42 for Groups 1-4, Day 63 for groups 5 and 6, and Day 84 for groups 7 and 8.

Blood samples are taken weekly to measure viral parameters (HBeAg, HBsAg and HBV DNA) and liver ALT in serum. Spleen is taken at end points, and immunogenicity is assessed in all groups by IFNy ELISPOT after ex vivo stimulation with HBV peptide pools covering both Tx Vx core and pol sequences. All endpoints are 3 weeks after last therapeutic dose.

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

1-16. (canceled)

17. A therapeutic combination for treating a hepatitis B virus (HBV) infection in a subject in need thereof, comprising:
i) a non-naturally occurring polynucleotide sequence encoding a HBV polymerase antigen consisting of an amino acid sequence that is at least 90% identical to SEQ ID NO: 7, wherein the HBV polymerase antigen does not have reverse transcriptase activity and RNase H activity and is capable of inducing a T cell response against at least HBV genotypes B, C and D; and
ii) an RNAi agent for inhibiting the expression of an HBV gene.

18. The therapeutic combination of claim 17, further comprising a non-naturally occurring polynucleotide sequence encoding a truncated HBV core antigen consisting of an amino acid sequence that is at least 95% identical to SEQ ID NO: 2.

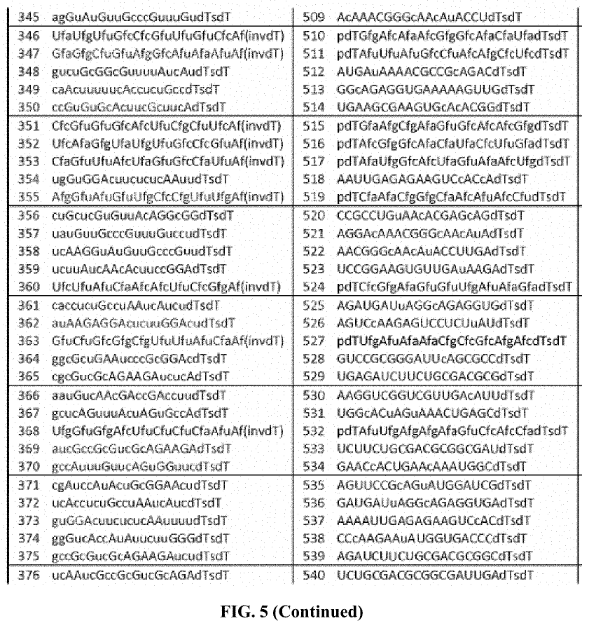

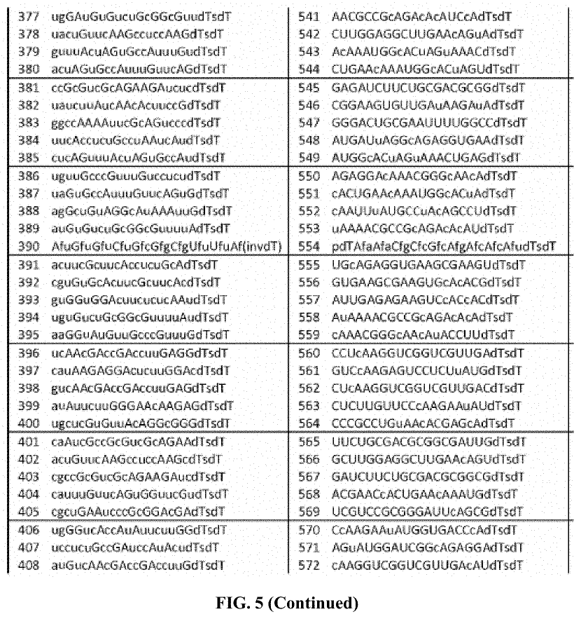

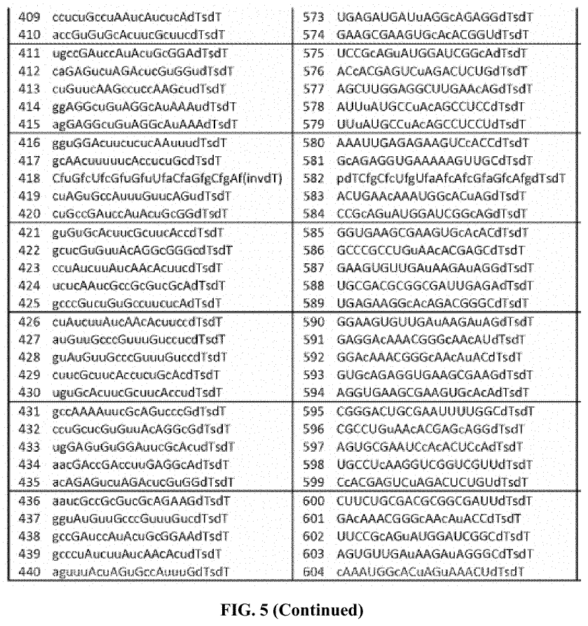

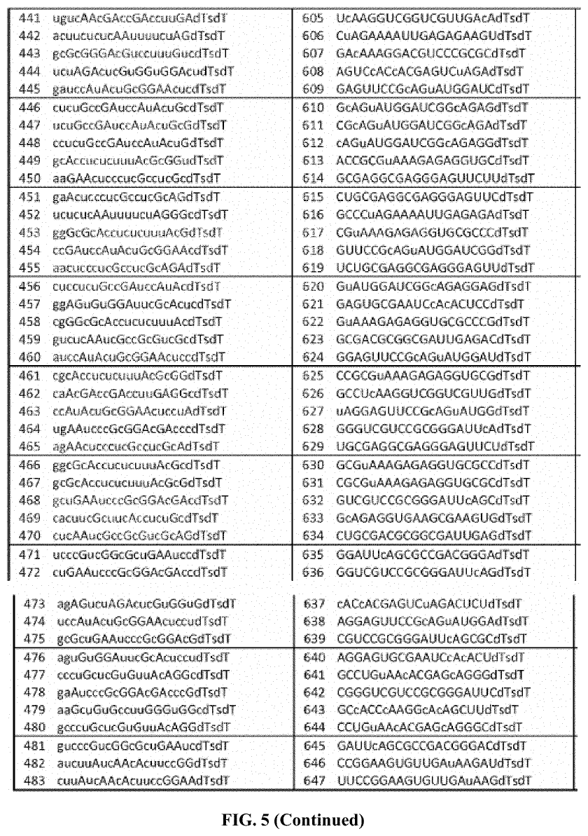

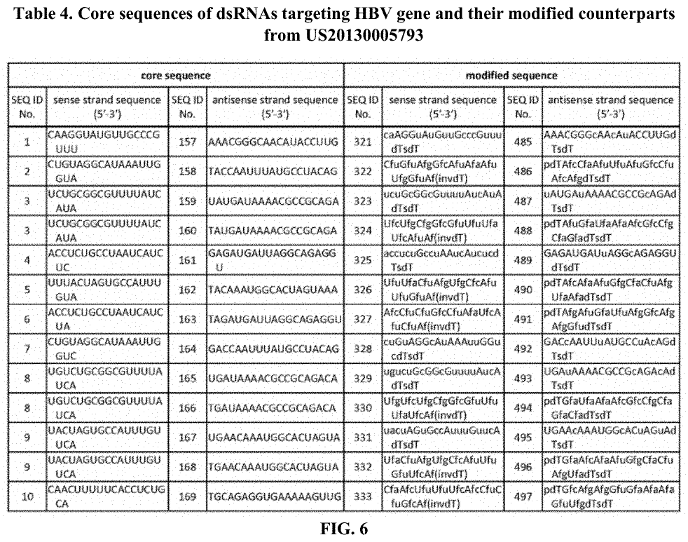

19. A therapeutic combination for treating a hepatitis B virus (HBV) infection in a subject in need thereof, comprising:
i) a non-naturally occurring polynucleotide sequence encoding a HBV polymerase antigen consisting of an amino acid sequence that is at least 90% identical to SEQ ID NO: 7, wherein the HBV polymerase antigen does not have reverse transcriptase activity and RNase H activity and is capable of inducing a T cell response against at least HBV genotypes B, C and D; and
ii) an RNAi agent for inhibiting the expression of an HBV gene, wherein the RNAi agent is selected from the group consisting of:
1) an RNAi agent having a core sense strand sequence and the corresponding antisense strand sequence shown in Table 2 of FIG. 4;
2) an RNAi agent having a sense strand sequence and the corresponding antisense strand sequence shown in Table 3 of FIG. 5;
3) an RNAi agent having a core sense strand sequence and the corresponding antisense strand sequence shown in Table 4 of FIG. 6;
4) an RNAi agent targeting a target sequence shown in Table 5 of FIG. 7;
5) an RNAi agent having a core sense strand sequence and the corresponding antisense strand sequence shown in Table 6 of FIG. 8;
6) an RNAi agent having an antisense sequence and the corresponding sense strand sequence shown in Table 7 of FIG. 9;
7) an RNAi agent having an antisense sequence and the corresponding sense strand sequence shown in Table 8 of FIG. 10; and
8) an RNAi agent having a duplex of an antisense strand and a sense strand shown in Table 9 of FIG. 11.

20. The therapeutic combination of claim 19, further comprising a non-naturally occurring polynucleotide sequence encoding a truncated HBV core antigen consisting of an amino acid sequence that is at least 95% identical to SEQ ID NO: 2

21. The therapeutic combination of claim 17, further comprising a polynucleotide sequence encoding a signal sequence operably linked to the N-terminus of the HBV polymerase antigen.

22. The therapeutic combination of claim 18, wherein
a) the truncated HBV core antigen consists of the amino acid sequence of SEQ ID NO: 2 or SEQ ID NO: 4; and
b) the HBV polymerase antigen comprises the amino acid sequence of SEQ ID NO: 7.

23. The therapeutic combination of claim 17, wherein the non-naturally occurring polynucleotide sequence is a DNA sequence.

24. The therapeutic combination of claim 18, comprising a non-naturally occurring nucleic acid molecule encoding both the HBV polymerase antigen and the HBV core antigen.

25. The therapeutic combination of claim 18, comprising a first non-naturally occurring nucleic acid molecule encoding the HBV polymerase antigen and a second, different non-naturally occurring nucleic acid molecule encoding the HBV core antigen.

26. The therapeutic combination of claim 18, wherein the polynucleotide sequence encoding the HBV core antigen comprises a polynucleotide sequence having at least 90% sequence identity to SEQ ID NO: 1 or SEQ ID NO: 3.

27. The therapeutic combination of claim 26, wherein the polynucleotide sequence encoding the HBV core antigen comprises the polynucleotide sequence of SEQ ID NO:
1 or SEQ ID NO: 3.

28. The therapeutic combination of claim 17, wherein the polynucleotide sequence encoding the HBV polymerase antigen comprises a polynucleotide sequence having at least 90% sequence identity to SEQ ID NO: 5 or SEQ ID NO: 6.

29. The therapeutic combination of claim 28, wherein the polynucleotide sequence encoding the HBV polymerase antigen comprises the polynucleotide sequence of SEQ ID NO: 5 or SEQ ID NO: 6.

30. The therapeutic combination of claim 17, wherein the RNAi agent has the duplex structure of AD04580; AD04585; AD04776; AD04872; AD04962; AD04963; AD04982; or AD05070 shown in Table 9.

31. The therapeutic combination of claim 17, wherein the non-naturally occurring polynucleotide sequence encodes the HBV polymerase antigen consisting of an amino acid sequence that is at least 98% identical to SEQ ID NO: 7.

32. A therapeutic combination for treating a hepatitis B virus (HBV) infection in a subject in need thereof, comprising:
i) a non-naturally occurring polynucleotide sequence encoding a HBV polymerase antigen consisting of an amino acid sequence that is at least 98% identical to SEQ ID NO: 7, wherein the HBV polymerase antigen does not have reverse transcriptase activity and RNase H activity and is capable of inducing a T cell response against at least HBV genotypes B, C and D; and
ii) an RNAi agent for inhibiting the expression of an HBV gene, wherein the RNAi agent is selected from the group consisting of:
1) an RNAi agent having a core sense strand sequence and the corresponding antisense strand sequence shown in Table 2 of FIG. 4;
2) an RNAi agent having a sense strand sequence and the corresponding antisense strand sequence shown in Table 3 of FIG. 5;
3) an RNAi agent having a core sense strand sequence and the corresponding antisense strand sequence shown in Table 4 of FIG. 6;
4) an RNAi agent targeting a target sequence shown in Table 5 of FIG. 7;
5) an RNAi agent having a core sense strand sequence and the corresponding antisense strand sequence shown in Table 6 of FIG. 8;
6) an RNAi agent having an antisense sequence and the corresponding sense strand sequence shown in Table 7 of FIG. 9;
7) an RNAi agent having an antisense sequence and the corresponding sense strand sequence shown in Table 8 of FIG. 10; and
8) an RNAi agent having a duplex of an antisense strand and a sense strand shown in Table 9 of FIG. 11.

33. The therapeutic combination of claim 32, further comprising a non-naturally occurring polynucleotide sequence encoding a truncated HBV core antigen consisting of an amino acid sequence that is at least 95% identical to SEQ ID NO 2.

34. A method of treating a hepatitis B virus (HBV) infection or an HBV-induced disease in a subject in need thereof, comprising administering to the subject thereof the therapeutic combination of claim 17.


SEQUENCE LISTING (1009 sequences)

SEQ ID NO: 1 - DNA, 444 bp, Artificial Sequence
  HBV truncated core antigen gene
gacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtc

SEQ ID NO: 2 - protein, 148 aa, Artificial Sequence
  HBV truncated core antigen
DIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 3 - DNA, 447 bp, Artificial Sequence
  HBV truncated core antigen gene
atggacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatccaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggatccgcactccccctgcttatcgaccacccaacgcaccaattctgtccaccctgcccgagaccacagtggtc

SEQ ID NO: 4 - protein, 149 aa, Artificial Sequence
  HBV truncated core antigen
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 5 - DNA, 2529 bp, Artificial Sequence
  HBV pol antigen gene
atgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 6 - DNA, 2529 bp, Artificial Sequence
  HBV pol antigen gene
atgcccctgtcttaccagcactttagaaagctgctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 7 - protein, 843 aa, Artificial Sequence
  HBV pol antigen
MPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 8 - DNA, 63 bp, Artificial Sequence
  Cystatin S signal peptide coding sequence
atggctcgacctctgtgtaccctgctactcctgatggctaccctggctggagctctggccagc

SEQ ID NO: 9 - protein, 21 aa, Artificial Sequence
  Cystatin S signal peptide sequence
MARPLCTLLLLMATLAGALAS

SEQ ID NO: 10 - DNA, 378 bp, Artificial Sequence
  triple enhancer regulatory sequence
ggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagac

SEQ ID NO: 11 - DNA, 12 bp, Artificial Sequence
  linker coding sequence
gccggagctggc

SEQ ID NO: 12 - DNA, 248 bp, Artificial Sequence
  ApoAI gene fragment
ttggccgtgctcttcctgacgggtaggtgtcccctaacctagggagccaaccatcggggggccttctccctaaatccccgtggcccaccctcctgggcagaggcagcaggtttctcactggccccctctcccccacctccaagcttggcctttcggctcagatctcagcccacagctggcctgatctgggtctcccctcccaccctcagggagccaggctcggcatttcgtcgacaagcttagccacc

SEQ ID NO: 13 - DNA, 130 bp, Artificial Sequence
  SV40 polyadenylation signal sequence
aacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 14 - DNA, 81 bp, Artificial Sequence
  immunoglobulin secretion signal coding sequence
atggagttcggcctgtcttgggtctttctggtggcaatcctgaagggcgtgcagtgtgaagtgcagctgctggagtctgga

SEQ ID NO: 15 - protein, 27 aa, Artificial Sequence
  immunoglobulin secretion signal sequence
MEFGLSWVFLVAILKGVQCEVQLLESG

SEQ ID NO: 16 - protein, 996 aa, Artificial Sequence
  HBV core-pol fusion antigen sequence
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 17 - protein, 1023 aa, Artificial Sequence
  HBV core-pol fusion antigen sequence with Ig signal sequence
MEFGLSWVFLVAILKGVQCEVQLLESGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 18 - DNA, 584 bp, Artificial Sequence
  hCMV promoter
tgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagc

SEQ ID NO: 19 - DNA, 684 bp, Artificial Sequence
  hCMV promoter sequence
accgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgg

SEQ ID NO: 20 - DNA, 225 bp, Artificial Sequence
  bGH polyA signal
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 671 bp, Artificial Sequence
  pUC ORI
cccgtagaaaagatcaaaggatcttcttgagatcctttttttctgcgcgtaatctgctgcttgcaaacaaaaaaaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 22 - DNA, 795 bp, Artificial Sequence
  Kanr
atgattgagcaagatggtcttcacgctggctcgccagctgcgtgggtggaacgcctgtttggttatgattgggcgcagcagactattggatgttccgacgcggctgtatttcggctgtctgctcagggtcgccccgtgctgtttgtgaagacggatttgtctggcgcattaaatgagttacaggacgaggcggctcgtctgagttggttggccaccaccggcgtgccctgcgccgcagtgctggatgtcgtgacagaagcaggccgcgattggctccttctcggcgaagtgccgggccaggacctgctcagcagccacttggcaccggcagaaaaagtttctatcatggccgacgccatgcgtcgtcttcacactctcgatccggccacgtgcccctttgaccaccaggccaagcatcgtattgaacgtgcgcgtactcggatggaagcaggtttagtagaccaggacgatttggatgaggaacatcaaggcctggccccggctgaactgtttgcgcgcttaaaagcgtcgatgccagatggcgaagatttggtagtcacccatggagatgcgtgtttgccaaacatcatggttgaaaatggccgcttctcaggctttattgactgtgggcgcctgggtgttgccgaccgctatcaagatattgcgctcgcaactcgtgacatcgctgaagagctgggcggagaatgggctgaccgtttcctggtactgtatggcattgcagcgcccgattcccaacgcatcgcattttatcgtctgctggatgagtttttctaa

SEQ ID NO: 23 - protein, 264 aa, Artificial Sequence
  Codon optimized Kanr
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 24 - DNA, 99 bp, Artificial Sequence
  bla promoter
acccctatttgtttatttttctaaatacattcaaatatgtatccgctcatgagacaataaccctgataaatgcttcaataatattgaaaaaggaagagt

SEQ ID NO: 25 - RNA, 21 nt, Artificial sequence
  HBV RNAi Agent antisense strand modified sequence
agaaaauugagagaaguccac

SEQ ID NO: 26 - RNA, 21 nt, Artificial sequence
  HBV RNAi Agent sense strand modified sequence
guggacuucucucaauuuucu

SEQ ID NO: 27 - RNA, 21 nt, Artificial sequence
  HBV RNAi Agent antisense strand modified sequence
uaccaauuuaugccuacagcg

SEQ ID NO: 28 - RNA, 21 nt, Artificial sequence
  HBV RNAi Agent sense strand modified sequence
cgcuguaggcauaaauuggua

SEQ ID NO: 29 - RNA, 19 nt, Artificial sequence
  Description of the artificial sequence: sense strand of dsRNA
  modified_base 1: /mod_base = “nucleoside: lacks 5′-phosphate group”
caagguauguugcccguuu

SEQ ID NO: 30 - RNA, 19 nt, Artificial sequence
  Description of the artificial sequence: sense strand of dsRNA
  modified_base 1: /mod_base = “nucleoside: lacks 5′-phosphate group”
cuguaggcauaaauuggua

SEQ ID NO: 31 - RNA, 19 nt, Artificial sequence
  Description of the artificial sequence: sense strand of dsRNA
  modified_base 1: /mod_base = “nucleoside: lacks 5′-phosphate group”
ucugcggcguuuuaucaua

SEQ ID NO: 32 - RNA, 19 nt, Artificial sequence
  Description of the artificial sequence: sense strand of dsRNA
  modified_base 1: /mod_base = “nucleoside: lacks 5′-phosphate group”
accucugccuaaucaucuc

SEQ ID NO: 33 - RNA, 19 nt, Artificial sequence
  Description of the artificial sequence: sense strand of dsRNA
  modified_base 1: /mod_base = “nucleoside: lacks 5′-phosphate group”
uuuacuagugccauuugua

SEQ ID NO: 34 - RNA, 19 nt, Artificial sequence
  Description of the artificial sequence: sense strand of dsRNA
  modified_base 1: /mod_base = “nucleoside: lacks 5′-phosphate group”
accucugccuaaucaucua

SEQ ID NO: 35 - RNA, 19 nt, Artificial sequence
  Description of the artificial sequence: sense strand of dsRNA
  modified_base 1: /mod_base = “nucleoside: lacks 5′-phosphate group”
cuguaggcauaaauugguc

SEQ ID NO: 36 - RNA, 19 nt, Artificial sequence
  Description of the artificial sequence: sense strand of dsRNA
  modified_base 1: /mod_base = “nucleoside: lacks 5′-phosphate group”
ugucugcggcguuuuauca

SEQ ID NO: 37 - RNA, 19 nt, Artificial sequence
  Description of the artificial sequence: sense strand of dsRNA
  modified_base 1: /mod_base = “nucleoside: lacks 5′-phosphate group”
uacuagugccauuuguuca

SEQ ID NO: 38 - RNA, 19 nt, Artificial sequence
  Description of the artificial sequence: sense strand of dsRNA
  modified_base 1: /mod_base = “nucleoside: lacks 5′-phosphate group”
caacuuuuucaccucugca

SEQ ID NO: 39 - RNA, 19 nt, Artificial sequence
  Description of the artificial sequence: sense strand of dsRNA
  modified_base 1: /mod_base = “nucleoside: lacks 5′-phosphate group”
ccauuuguucagugguucg

SEQ ID NO: 40 - RNA, 19 nt, Artificial sequence
  Description of the artificial sequence: sense strand of dsRNA
  modified_base 1: /mod_base = “nucleoside: lacks 5′-phosphate group”
ccaaguguuugcugacgca

SEQ ID NO: 41 - RNA, 19 nt, Artificial sequence
  Description of the artificial sequence: sense strand of dsRNA
  modified_base 1: /mod_base = “nucleoside: lacks 5′-phosphate group”
ccauuuguucagugguuca

SEQ ID NO: 42 - RNA, 19 nt, Artificial sequence
  Description of the artificial sequence: sense strand of dsRNA
  modified_base 1: /mod_base = “nucleoside: lacks 5′-phosphate group”
uuuacuagugccauuuguu

SEQ ID NO: 43 - RNA, 19 nt, Artificial sequence
  Description of the artificial sequence: sense strand of dsRNA
  modified_base 1: /mod_base = “nucleoside: lacks 5′-phosphate group”
caccucugccuaaucauca

SEQ ID NO: 44 - RNA, 19 nt, Artificial sequence
  Description of the artificial sequence: sense strand of dsRNA
  modified_base 1: /mod_base = “nucleoside: lacks 5′-phosphate group”
cuggcucaguuuacuagug

SEQ ID NO: 45 - RNA, 19 nt, Artificial sequence
  Description of the artificial sequence: sense strand of dsRNA
  modified_base 1: /mod_base = “nucleoside: lacks 5′-phosphate group”
caagguauguugcccguua

SEQ ID NO: 46 - RNA, 19 nt, Artificial sequence
  Description of the artificial sequence: sense strand of dsRNA
  modified_base 1: /mod_base = “nucleoside: lacks 5′-phosphate group”
cuggcucaguuuacuagua

SEQ ID NO: 47 - RNA, 19 nt, Artificial sequence
  Description of the artificial sequence: sense strand of dsRNA
  modified_base 1: /mod_base = “nucleoside: lacks 5′-phosphate group”
gaggcuguaggcauaaauu

SEQ ID NO: 48 - RNA, 19 nt, Artificial sequence
  Description of the artificial sequence: sense strand of dsRNA
  modified_base 1: /mod_base = “nucleoside: lacks 5′-phosphate group”
caguuuacuagugccauuu

SEQ ID NO: 49 - RNA, 19 nt, Artificial sequence
  Description of the artificial sequence: sense strand of dsRNA
  modified_base 1: /mod_base = “nucleoside: lacks 5′-phosphate group”
agguauguugcccguuugu

SEQ ID NO: 50 - RNA, 19 nt, Artificial sequence
  Description of the artificial sequence: sense strand of dsRNA
  modified_base 1: /mod_base = “nucleoside: lacks 5′-phosphate group”
uauguugcccguuugucca

SEQ ID NO: 51 - RNA, 19 nt, Artificial sequence
  Description of the artificial sequence: sense strand of dsRNA
  modified_base 1: /mod_base = “nucleoside: lacks 5′-phosphate group”
gaggcuguaggcauaaaua

SEQ ID NO: 52 - RNA, 19 nt, Artificial sequence
  Description of the artificial sequence: sense strand of dsRNA
  modified_base 1: /mod_base = “nucleoside: lacks 5′-phosphate group”
gucugcggcguuuuaucau

SEQ ID NO: 53 - RNA, 19 nt, Artificial sequence
  Description of the artificial sequence: sense strand of dsRNA
  modified_base 1: /mod_base = “nucleoside: lacks 5′-phosphate group”
caacuuuuucaccucugcc

SEQ ID NO: 54 - RNA, 19 nt, Artificial sequence
  Description of the artificial sequence: sense strand of dsRNA
  modified_base 1: /mod_base = “nucleoside: lacks 5′-phosphate group”
ccgugugcacuucgcuuca

SEQ ID NO: 55 - RNA, 19 nt, Artificial sequence
  Description of the artificial sequence: sense strand of dsRNA
  modified_base 1: /mod_base = “nucleoside: lacks 5′-phosphate group”
ucaagguauguugcccgua

SEQ ID NO: 56 - RNA, 19 nt, Artificial sequence
  Description of the artificial sequence: sense strand of dsRNA
  modified_base 1: /mod_base = “nucleoside: lacks 5′-phosphate group”
caguuuacuagugccauua

SEQ ID NO: 57 - RNA, 19 nt, Artificial sequence
  Description of the artificial sequence: sense strand of dsRNA
  modified_base 1: /mod_base = “nucleoside: lacks 5′-phosphate group”
ugguggacuucucucaauu

SEQ ID NO: 58 - RNA, 19 nt, Artificial sequence
  Description of the artificial sequence: sense strand of dsRNA
  modified_base 1: /mod_base = “nucleoside: lacks 5′-phosphate group”
agguauguugcccguuuga

SEQ ID NO: 59 - RNA, 19 nt, Artificial sequence
  Description of the artificial sequence: sense strand of dsRNA
  modified_base 1: /mod_base = “nucleoside: lacks 5′-phosphate group”
cugcucguguuacaggcgg

SEQ ID NO: 60 - RNA, 19 nt, Artificial sequence
  Description of the artificial sequence: sense strand of dsRNA
  modified_base 1: /mod_base = “nucleoside: lacks 5′-phosphate group”
uauguugcccguuuguccu

SEQ ID NO: 61 - RNA, 19 nt, Artificial sequence
  Description of the artificial sequence: sense strand of dsRNA
  modified_base 1: /mod_base = “nucleoside: lacks 5′-phosphate group”
ucaagguauguugcccguu

SEQ ID NO: 62 - RNA, 19 nt, Artificial sequence
  Description of the artificial sequence: sense strand of dsRNA
  modified_base 1: /mod_base = “nucleoside: lacks 5′-phosphate group”
ucuuaucaacacuuccgga

SEQ ID NO: 63 - RNA, 19 nt, Artificial sequence
  Description of the artificial sequence: sense strand of dsRNA
  modified_base 1: /mod_base = “nucleoside: lacks 5′-phosphate group”
caccucugccuaaucaucu

SEQ ID NO: 64 - RNA, 19 nt, Artificial sequence
  Description of the artificial sequence: sense strand of dsRNA
  modified_base 1: /mod_base = “nucleoside: lacks 5′-phosphate group”
auaagaggacucuuggacu

SEQ ID NO: 65 - RNA, 19 nt, Artificial sequence
  Description of the artificial sequence: sense strand of dsRNA
  modified_base 1: /mod_base = “nucleoside: lacks 5′-phosphate group”
gucugcggcguuuuaucaa

SEQ ID NO: 66 - RNA, 19 nt, Artificial sequence
  Description of the artificial sequence: sense strand of dsRNA
  modified_base 1: /mod_base = “nucleoside: lacks 5′-phosphate group”
ggcgcugaaucccgcggac

SEQ ID NO: 67 - RNA, 19 nt, Artificial sequence
  Description of the artificial sequence: sense strand of dsRNA
  modified_base 1: /mod_base = “nucleoside: lacks 5′-phosphate group”
cgcgucgcagaagaucuca

SEQ ID NO: 68 - RNA, 19 nt, Artificial sequence
  Description of the artificial sequence: sense strand of dsRNA
  modified_base 1: /mod_base = “nucleoside: lacks 5′-phosphate group”
aaugucaacgaccgaccuu

SEQ ID NO: 69 - RNA, 19 nt, Artificial sequence
  Description of the artificial sequence: sense strand of dsRNA
  modified_base 1: /mod_base = “nucleoside: lacks 5′-phosphate group”
gcucaguuuacuagugcca

SEQ ID NO: 70 - RNA, 19 nt, Artificial sequence
  Description of the artificial sequence: sense strand of dsRNA
  modified_base 1: /mod_base = “nucleoside: lacks 5′-phosphate group”
ugguggacuucucucaaua

SEQ ID NO: 71 - RNA, 19 nt, Artificial sequence
  Description of the artificial sequence: sense strand of dsRNA
  modified_base 1: /mod_base = “nucleoside: lacks 5′-phosphate group”
aucgccgcgucgcagaaga

SEQ ID NO: 72 - RNA, 19 nt, Artificial sequence
  Description of the artificial sequence: sense strand of dsRNA
  modified_base 1: /mod_base = “nucleoside: lacks 5′-phosphate group”
gccauuuguucagugguuc

SEQ ID NO: 73 - RNA, 19 nt, Artificial sequence
  Description of the artificial sequence: sense strand of dsRNA
  modified_base 1: /mod_base = “nucleoside: lacks 5′-phosphate group”
cgauccauacugcggaacu

SEQ ID NO: 74 - RNA, 19 nt, Artificial sequence
  Description of the artificial sequence: sense strand of dsRNA
  modified_base 1: /mod_base = “nucleoside: lacks 5′-phosphate group”
ucaccucugccuaaucauc

SEQ ID NO: 75 - RNA, 19 nt, Artificial sequence
  Description of the artificial sequence: sense strand of dsRNA
  modified_base 1: /mod_base = “nucleoside: lacks 5′-phosphate group”
guggacuucucucaauuuu

SEQ ID NO: 76 - RNA, 19 nt, Artificial sequence
  Description of the artificial sequence: sense strand of dsRNA
  modified_base 1: /mod_base = “nucleoside: lacks 5′-phosphate group”
gggucaccauauucuuggg

SEQ ID NO: 77 - RNA, 19 nt, Artificial sequence
  Description of the artificial sequence: sense strand of dsRNA
  modified_base 1: /mod_base = “nucleoside: lacks 5′-phosphate group”
gccgcgucgcagaagaucu

SEQ ID NO: 78 - RNA, 19 nt, Artificial sequence
  Description of the artificial sequence: sense strand of dsRNA
  modified_base 1: /mod_base = “nucleoside: lacks 5′-phosphate group”
ucaaucgccgcgucgcaga

SEQ ID NO: 79 - RNA, 19 nt, Artificial sequence
  Description of the artificial sequence: sense strand of dsRNA
  modified_base 1: /mod_base = “nucleoside: lacks 5′-phosphate group”
uggaugugucugcggcguu

SEQ ID NO: 80 - RNA, 19 nt, Artificial sequence
  Description of the artificial sequence: sense strand of dsRNA
  modified_base 1: /mod_base = “nucleoside: lacks 5′-phosphate group”
uacuguucaagccuccaag

SEQ ID NO: 81 - RNA, 19 nt, Artificial sequence
  Description of the artificial sequence: sense strand of dsRNA
  modified_base 1: /mod_base = “nucleoside: lacks 5′-phosphate group”
guuuacuagugccauuugu

SEQ ID NO: 82 - RNA, 19 nt, Artificial sequence
  Description of the artificial sequence: sense strand of dsRNA
  modified_base 1: /mod_base = “nucleoside: lacks 5′-phosphate group”
acuagugccauuuguucag

SEQ ID NO: 83 - RNA, 19 nt, Artificial sequence
  Description of the artificial sequence: sense strand of dsRNA
  modified_base 1: /mod_base = “nucleoside: lacks 5′-phosphate group”
ccgcgucgcagaagaucuc

SEQ ID NO: 84 - RNA, 19 nt, Artificial sequence
  Description of the artificial sequence: sense strand of dsRNA
  modified_base 1: /mod_base = “nucleoside: lacks 5′-phosphate group”
uaucuuaucaacacuuccg

SEQ ID NO: 85 - RNA, 19 nt, Artificial sequence
  Description of the artificial sequence: sense strand of dsRNA
  modified_base 1: /mod_base = “nucleoside: lacks 5′-phosphate group”
ggccaaaauucgcaguccc

SEQ ID NO: 86 - RNA, 19 nt, Artificial sequence
  Description of the artificial sequence: sense strand of dsRNA
  modified_base 1: /mod_base = “nucleoside: lacks 5′-phosphate group”
uucaccucugccuaaucau

SEQ ID NO: 87 - RNA, 19 nt, Artificial sequence
  Description of the artificial sequence: sense strand of dsRNA
  modified_base 1: /mod_base = “nucleoside: lacks 5′-phosphate group”
cucaguuuacuagugccau

SEQ ID NO: 88 - RNA, 19 nt, Artificial sequence
  Description of the artificial sequence: sense strand of dsRNA
  modified_base 1: /mod_base = “nucleoside: lacks 5′-phosphate group”
uguugcccguuuguccucu

SEQ ID NO: 89 - RNA, 19 nt, Artificial sequence
  Description of the artificial sequence: sense strand of dsRNA
  modified_base 1: /mod_base = “nucleoside: lacks 5′-phosphate group”
uagugccauuuguucagug

SEQ ID NO: 90 - RNA, 19 nt, Artificial sequence
  Description of the artificial sequence: sense strand of dsRNA
  modified_base 1: /mod_base = “nucleoside: lacks 5′-phosphate group”
aggcuguaggcauaaauug

SEQ ID NO: 91 - RNA, 19 nt, Artificial sequence
  Description of the artificial sequence: sense strand of dsRNA
  modified_base 1: /mod_base = “nucleoside: lacks 5′-phosphate group”
augugucugcggcguuuua

SEQ ID NO: 92 - RNA, 19 nt, Artificial sequence
  Description of the artificial sequence: sense strand of dsRNA
  modified_base 1: /mod_base = “nucleoside: lacks 5′-phosphate group”
acuucgcuucaccucugca

SEQ ID NO: 93 - RNA, 19 nt, Artificial sequence
  Description of the artificial sequence: sense strand of dsRNA
  modified_base 1: /mod_base = “nucleoside: lacks 5′-phosphate group”
cgugugcacuucgcuucac

SEQ ID NO: 94 - RNA, 19 nt, Artificial sequence
  Description of the artificial sequence: sense strand of dsRNA
  modified_base 1: /mod_base = “nucleoside: lacks 5′-phosphate group”
gugguggacuucucucaau

SEQ ID NO: 95 - RNA, 19 nt, Artificial sequence
  Description of the artificial sequence: sense strand of dsRNA
  modified_base 1: /mod_base = “nucleoside: lacks 5′-phosphate group”
ugugucugcggcguuuuau

SEQ ID NO: 96 - RNA, 19 nt, Artificial sequence
  Description of the artificial sequence: sense strand of dsRNA
  modified_base 1: /mod_base = “nucleoside: lacks 5′-phosphate group”
aagguauguugcccguuug

SEQ ID NO: 97 - RNA, 19 nt, Artificial sequence
  Description of the artificial sequence: sense strand of dsRNA
  modified_base 1: /mod_base = “nucleoside: lacks 5′-phosphate group”
ucaacgaccgaccuugagg

SEQ ID NO: 98 - RNA, 19 nt, Artificial sequence
  Description of the artificial sequence: sense strand of dsRNA
  modified_base 1: /mod_base = “nucleoside: lacks 5′-phosphate group”
cauaagaggacucuuggac

SEQ ID NO: 99 - RNA, 19 nt, Artificial sequence
  Description of the artificial sequence: sense strand of dsRNA
  modified_base 1: /mod_base = “nucleoside: lacks 5′-phosphate group”
gucaacgaccgaccuugag

SEQ ID NO: 100 - RNA, 19 nt, Artificial sequence
  Description of the artificial sequence: sense strand of dsRNA
  modified_base 1: /mod_base = “nucleoside: lacks 5′-phosphate group”
auauucuugggaacaagag

SEQ ID NO: 101 - RNA, 19 nt, Artificial sequence
  Description of the artificial sequence: sense strand of dsRNA
  modified_base 1: /mod_base = “nucleoside: lacks 5′-phosphate group”
ugcucguguuacaggcggg

SEQ ID NO: 102 - RNA, 19 nt, Artificial sequence
  Description of the artificial sequence: sense strand of dsRNA
  modified_base 1: /mod_base = “nucleoside: lacks 5′-phosphate group”
caaucgccgcgucgcagaa

SEQ ID NO: 103 - RNA, 19 nt, Artificial sequence
  Description of the artificial sequence: sense strand of dsRNA
  modified_base 1: /mod_base = “nucleoside: lacks 5′-phosphate group”
acuguucaagccuccaagc

SEQ ID NO: 104 - RNA, 19 nt, Artificial sequence
  Description of the artificial sequence: sense strand of dsRNA
  modified_base 1: /mod_base = “nucleoside: lacks 5′-phosphate group”
cgccgcgucgcagaagauc

SEQ ID NO: 105 - RNA, 19 nt, Artificial sequence
  Description of the artificial sequence: sense strand of dsRNA
  modified_base 1: /mod_base = “nucleoside: lacks 5′-phosphate group”
cauuuguucagugguucgu

SEQ ID NO: 106 - RNA, 19 nt, Artificial sequence
  Description of the artificial sequence: sense strand of dsRNA
  modified_base 1: /mod_base = “nucleoside: lacks 5′-phosphate group”
cgcugaaucccgcggacga

SEQ ID NO: 107 - RNA, 19 nt, Artificial sequence
  Description of the artificial sequence: sense strand of dsRNA
  modified_base 1: /mod_base = “nucleoside: lacks 5′-phosphate group”
ugggucaccauauucuugg

SEQ ID NO: 108 - RNA, 19 nt, Artificial sequence
  Description of the artificial sequence: sense strand of dsRNA
  modified_base 1: /mod_base = “nucleoside: lacks 5′-phosphate group”
uccucugccgauccauacu

SEQ ID NO: 109 - RNA, 19 nt, Artificial sequence
  Description of the artificial sequence: sense strand of dsRNA
  modified_base 1: /mod_base = “nucleoside: lacks 5′-phosphate group”
augucaacgaccgaccuug

SEQ ID NO: 110 - RNA, 19 nt, Artificial sequence
  Description of the artificial sequence: sense strand of dsRNA
  modified_base 1: /mod_base = “nucleoside: lacks 5′-phosphate group”
ccucugccuaaucaucuca

SEQ ID NO: 111 - RNA, 19 nt, Artificial sequence
  Description of the artificial sequence: sense strand of dsRNA
  modified_base 1: /mod_base = “nucleoside: lacks 5′-phosphate group”
accgugugcacuucgcuuc

SEQ ID NO: 112 - RNA, 19 nt, Artificial sequence
  Description of the artificial sequence: sense strand of dsRNA
  modified_base 1: /mod_base = “nucleoside: lacks 5′-phosphate group”
ugccgauccauacugcgga

SEQ ID NO: 113 - RNA, 19 nt, Artificial sequence
  Description of the artificial sequence: sense strand of dsRNA
  modified_base 1: /mod_base = “nucleoside: lacks 5′-phosphate group”
cagagucuagacucguggu

SEQ ID NO: 114 - RNA, 19 nt, Artificial sequence
  Description of the artificial sequence: sense strand of dsRNA
  modified_base 1: /mod_base = “nucleoside: lacks 5′-phosphate group”
cuguucaagccuccaagcu

SEQ ID NO: 115 - RNA, 19 nt, Artificial sequence
  Description of the artificial sequence: sense strand of dsRNA
  modified_base 1: /mod_base = “nucleoside: lacks 5′-phosphate group”
ggaggcuguaggcauaaau

SEQ ID NO: 116 - RNA, 19 nt, Artificial sequence
  Description of the artificial sequence: sense strand of dsRNA
  modified_base 1: /mod_base = “nucleoside: lacks 5′-phosphate group”
aggaggcuguaggcauaaa

SEQ ID NO: 117 - RNA, 19 nt, Artificial sequence
  Description of the artificial sequence: sense strand of dsRNA
  modified_base 1: /mod_base = “nucleoside: lacks 5′-phosphate group”
gguggacuucucucaauuu

SEQ ID NO: 118 - RNA, 19 nt, Artificial sequence
  Description of the artificial sequence: sense strand of dsRNA
  modified_base 1: /mod_base = “nucleoside: lacks 5′-phosphate group”
gcaacuuuuucaccucugc

SEQ ID NO: 119 - RNA, 19 nt, Artificial sequence
  Description of the artificial sequence: sense strand of dsRNA
  modified_base 1: /mod_base = “nucleoside: lacks 5′-phosphate group”
cugcucguguuacaggcga

SEQ ID NO: 120 - RNA, 19 nt, Artificial sequence
  Description of the artificial sequence: sense strand of dsRNA
  modified_base 1: /mod_base = “nucleoside: lacks 5′-phosphate group”
cuagugccauuuguucagu

SEQ ID NO: 121 - RNA, 19 nt, Artificial sequence
  Description of the artificial sequence: sense strand of dsRNA
  modified_base 1: /mod_base = “nucleoside: lacks 5′-phosphate group”
cugccgauccauacugcgg

SEQ ID NO: 122 - RNA, 19 nt, Artificial sequence
  Description of the artificial sequence: sense strand of dsRNA
  modified_base 1: /mod_base = “nucleoside: lacks 5′-phosphate group”
gugugcacuucgcuucacc

SEQ ID NO: 123 - RNA, 19 nt, Artificial sequence
  Description of the artificial sequence: sense strand of dsRNA
  modified_base 1: /mod_base = “nucleoside: lacks 5′-phosphate group”
gcucguguuacaggcgggc

SEQ ID NO: 124 - RNA, 19 nt, Artificial sequence
  Description of the artificial sequence: sense strand of dsRNA
  modified_base 1: /mod_base = “nucleoside: lacks 5′-phosphate group”
ccuaucuuaucaacacuuc

SEQ ID NO: 125 - RNA, 19 nt, Artificial sequence
  Description of the artificial sequence: sense strand of dsRNA
  modified_base 1: /mod_base = “nucleoside: lacks 5′-phosphate group”
ucucaaucgccgcgucgca

SEQ ID NO: 126 - RNA, 19 nt, Artificial sequence
  Description of the artificial sequence: sense strand of dsRNA
  modified_base 1: /mod_base = “nucleoside: lacks 5′-phosphate group”
gcccgucugugccuucuca

SEQ ID NO: 127 - RNA, 19 nt, Artificial sequence
  Description of the artificial sequence: sense strand of dsRNA
  modified_base 1: /mod_base = “nucleoside: lacks 5′-phosphate group”
cuaucuuaucaacacuucc

SEQ ID NO: 128 - RNA, 19 nt, Artificial sequence
  Description of the artificial sequence: sense strand of dsRNA
  modified_base 1: /mod_base = “nucleoside: lacks 5′-phosphate group”
auguugcccguuuguccuc

SEQ ID NO: 129 - RNA, 19 nt, Artificial sequence
  Description of the artificial sequence: sense strand of dsRNA
  modified_base 1: /mod_base = “nucleoside: lacks 5′-phosphate group”
guauguugcccguuugucc

SEQ ID NO: 130 - RNA, 19 nt, Artificial sequence
  Description of the artificial sequence: sense strand of dsRNA
  modified_base 1: /mod_base = “nucleoside: lacks 5′-phosphate group”
cuucgcuucaccucugcac

SEQ ID NO: 131 - RNA, 19 nt, Artificial sequence
  Description of the artificial sequence: sense strand of dsRNA
  modified_base 1: /mod_base = “nucleoside: lacks 5′-phosphate group”
ugugcacuucgcuucaccu

SEQ ID NO: 132 - RNA, 19 nt, Artificial sequence
  Description of the artificial sequence: sense strand of dsRNA
  modified_base 1: /mod_base = “nucleoside: lacks 5′-phosphate group”
gccaaaauucgcagucccg

SEQ ID NO: 133 - RNA, 19 nt, Artificial sequence
  Description of the artificial sequence: sense strand of dsRNA
  modified_base 1: /mod_base = “nucleoside: lacks 5′-phosphate group”
ccugcucguguuacaggcg

SEQ ID NO: 134 - RNA, 19 nt, Artificial sequence
  Description of the artificial sequence: sense strand of dsRNA
  modified_base 1: /mod_base = “nucleoside: lacks 5′-phosphate group”
uggaguguggauucgcacu

SEQ ID NO: 135 - RNA, 19 nt, Artificial sequence
  Description of the artificial sequence: sense strand of dsRNA
  modified_base 1: /mod_base = “nucleoside: lacks 5′-phosphate group”
aacgaccgaccuugaggca

SEQ ID NO: 136 - RNA, 19 nt, Artificial sequence
  Description of the artificial sequence: sense strand of dsRNA
  modified_base 1: /mod_base = “nucleoside: lacks 5′-phosphate group”
acagagucuagacucgugg

SEQ ID NO: 137 - RNA, 19 nt, Artificial sequence
  Description of the artificial sequence: sense strand of dsRNA
  modified_base 1: /mod_base = “nucleoside: lacks 5′-phosphate group”
aaucgccgcgucgcagaag

SEQ ID NO: 138 - RNA, 19 nt, Artificial sequence
  Description of the artificial sequence: sense strand of dsRNA
  modified_base 1: /mod_base = “nucleoside: lacks 5′-phosphate group”
gguauguugcccguuuguc

SEQ ID NO: 139 - RNA, 19 nt, Artificial sequence
  Description of the artificial sequence: sense strand of dsRNA
  modified_base 1: /mod_base = “nucleoside: lacks 5′-phosphate group”
gccgauccauacugcggaa

SEQ ID NO: 140 - RNA, 19 nt, Artificial sequence
  Description of the artificial sequence: sense strand of dsRNA
  modified_base 1: /mod_base = “nucleoside: lacks 5′-phosphate group”
gcccuaucuuaucaacacu

SEQ ID NO: 141 - RNA, 19 nt, Artificial sequence
  Description of the artificial sequence: sense strand of dsRNA
  modified_base 1: /mod_base = “nucleoside: lacks 5′-phosphate group”
aguuuacuagugccauuug

SEQ ID NO: 142 - RNA, 19 nt, Artificial sequence
  Description of the artificial sequence: sense strand of dsRNA
  modified_base 1: /mod_base = “nucleoside: lacks 5′-phosphate group”
ugucaacgaccgaccuuga

SEQ ID NO: 143 - RNA, 19 nt, Artificial sequence
  Description of the artificial sequence: sense strand of dsRNA
  modified_base 1: /mod_base = “nucleoside: lacks 5′-phosphate group”
acuucucucaauuuucuag

SEQ ID NO: 144 - RNA, 19 nt, Artificial sequence
  Description of the artificial sequence: sense strand of dsRNA
  modified_base 1: /mod_base = “nucleoside: lacks 5′-phosphate group”
gcgcgggacguccuuuguc

SEQ ID NO: 145 - RNA, 19 nt, Artificial sequence
  Description of the artificial sequence: sense strand of dsRNA
  modified_base 1: /mod_base = “nucleoside: lacks 5′-phosphate group”
ucuagacucgugguggacu

SEQ ID NO: 146 - RNA, 19 nt, Artificial sequence
  Description of the artificial sequence: sense strand of dsRNA
  modified_base 1: /mod_base = “nucleoside: lacks 5′-phosphate group”
gauccauacugcggaacuc

SEQ ID NO: 147 - RNA, 19 nt, Artificial sequence
  Description of the artificial sequence: sense strand of dsRNA
  modified_base 1: /mod_base = “nucleoside: lacks 5′-phosphate group”
cucugccgauccauacugc

SEQ ID NO: 148 - RNA, 19 nt, Artificial sequence
  Description of the artificial sequence: sense strand of dsRNA
  modified_base 1: /mod_base = “nucleoside: lacks 5′-phosphate group”
ucugccgauccauacugcg

SEQ ID NO: 149 - RNA, 19 nt, Artificial sequence
  Description of the artificial sequence: sense strand of dsRNA
  modified_base 1: /mod_base = “nucleoside: lacks 5′-phosphate group”
ccucugccgauccauacug

SEQ ID NO: 150 - RNA, 19 nt, Artificial sequence
  Description of the artificial sequence: sense strand of dsRNA
  modified_base 1: /mod_base = “nucleoside: lacks 5′-phosphate group”
gcaccucucuuuacgcggu

SEQ ID NO: 151 - RNA, 19 nt, Artificial sequence
  Description of the artificial sequence: sense strand of dsRNA
  modified_base 1: /mod_base = “nucleoside: lacks 5′-phosphate group”
aagaacucccucgccucgc

SEQ ID NO: 152 - RNA, 19 nt, Artificial sequence
  Description of the artificial sequence: sense strand of dsRNA
  modified_base 1: /mod_base = “nucleoside: lacks 5′-phosphate group”
gaacucccucgccucgcag

SEQ ID NO: 153 - RNA, 19 nt, Artificial sequence
  Description of the artificial sequence: sense strand of dsRNA
  modified_base 1: /mod_base = “nucleoside: lacks 5′-phosphate group”
ucucucaauuuucuagggc

SEQ ID NO: 154 - RNA, 19 nt, Artificial sequence
  Description of the artificial sequence: sense strand of dsRNA
  modified_base 1: /mod_base = “nucleoside: lacks 5′-phosphate group”
gggcgcaccucucuuuacg

SEQ ID NO: 155 - RNA, 19 nt, Artificial sequence
  Description of the artificial sequence: sense strand of dsRNA
  modified_base 1: /mod_base = “nucleoside: lacks 5′-phosphate group”
ccgauccauacugcggaac

SEQ ID NO: 156 - RNA, 19 nt, Artificial sequence
  Description of the artificial sequence: sense strand of dsRNA
  modified_base 1: /mod_base = “nucleoside: lacks 5′-phosphate group”
aacucccucgccucgcaga

SEQ ID NO: 157 - RNA, 19 nt, Artificial sequence
  Description of the artificial sequence: sense strand of dsRNA
  modified_base 1: /mod_base = “nucleoside: lacks 5′-phosphate group”
cuccucugccgauccauac

SEQ ID NO: 158 - RNA, 19 nt, Artificial sequence
  Description of the artificial sequence: sense strand of dsRNA
  modified_base 1: /mod_base = “nucleoside: lacks 5′-phosphate group”
ggaguguggauucgcacuc

SEQ ID NO: 159 - RNA, 19 nt, Artificial sequence
  Description of the artificial sequence: sense strand of dsRNA
  modified_base 1: /mod_base = “nucleoside: lacks 5′-phosphate group”
cgggcgcaccucucuuuac

SEQ ID NO: 160 - RNA, 19 nt, Artificial sequence
  Description of the artificial sequence: sense strand of dsRNA
  modified_base 1: /mod_base = “nucleoside: lacks 5′-phosphate group”
gucucaaucgccgcgucgc

SEQ ID NO: 161 - RNA, 19 nt, Artificial sequence
  Description of the artificial sequence: sense strand of dsRNA
  modified_base 1: /mod_base = “nucleoside: lacks 5′-phosphate group”
auccauacugcggaacucc

SEQ ID NO: 162 - RNA, 19 nt, Artificial sequence
  Description of the artificial sequence: sense strand of dsRNA
  modified_base 1: /mod_base = “nucleoside: lacks 5′-phosphate group”
cgcaccucucuuuacgcgg

SEQ ID NO: 163 - RNA, 19 nt, Artificial sequence
  Description of the artificial sequence: sense strand of dsRNA
  modified_base 1: /mod_base = “nucleoside: lacks 5′-phosphate group”
caacgaccgaccuugaggc

SEQ ID NO: 164 - RNA, 19 nt, Artificial sequence
  Description of the artificial sequence: sense strand of dsRNA
  modified_base 1: /mod_base = “nucleoside: lacks 5′-phosphate group”
ccauacugcggaacuccua

SEQ ID NO: 165 - RNA, 19 nt, Artificial sequence
  Description of the artificial sequence: sense strand of dsRNA
  modified_base 1: /mod_base = “nucleoside: lacks 5′-phosphate group”
ugaaucccgcggacgaccc

SEQ ID NO: 166 - RNA, 19 nt, Artificial sequence
  Description of the artificial sequence: sense strand of dsRNA
  modified_base 1: /mod_base = “nucleoside: lacks 5′-phosphate group”
agaacucccucgccucgca

SEQ ID NO: 167 - RNA, 19 nt, Artificial sequence
  Description of the artificial sequence: sense strand of dsRNA
  modified_base 1: /mod_base = “nucleoside: lacks 5′-phosphate group”
ggcgcaccucucuuuacgc

SEQ ID NO: 168 - RNA, 19 nt, Artificial sequence
  Description of the artificial sequence: sense strand of dsRNA
  modified_base 1: /mod_base = “nucleoside: lacks 5′-phosphate group”
gcgcaccucucuuuacgcg

SEQ ID NO: 169 - RNA, 19 nt, Artificial sequence
  Description of the artificial sequence: sense strand of dsRNA
  modified_base 1: /mod_base = “nucleoside: lacks 5′-phosphate group”
gcugaaucccgcggacgac

SEQ ID NO: 170 - RNA, 19 nt, Artificial sequence
  Description of the artificial sequence: sense strand of dsRNA
  modified_base 1: /mod_base = “nucleoside: lacks 5′-phosphate group”
cacuucgcuucaccucugc

SEQ ID NO: 171 - RNA, 19 nt, Artificial sequence
  Description of the artificial sequence: sense strand of dsRNA
  modified_base 1: /mod_base = “nucleoside: lacks 5′-phosphate group”
cucaaucgccgcgucgcag

SEQ ID NO: 172 - RNA, 19 nt, Artificial sequence
  Description of the artificial sequence: sense strand of dsRNA
  modified_base 1: /mod_base = “nucleoside: lacks 5′-phosphate group”
ucccgucggcgcugaaucc

SEQ ID NO: 173 - RNA, 19 nt, Artificial sequence
  Description of the artificial sequence: sense strand of dsRNA
  modified_base 1: /mod_base = “nucleoside: lacks 5′-phosphate group”
cugaaucccgcggacgacc

SEQ ID NO: 174 - RNA, 19 nt, Artificial sequence
  Description of the artificial sequence: sense strand of dsRNA
  modified_base 1: /mod_base = “nucleoside: lacks 5′-phosphate group”
agagucuagacucguggug

SEQ ID NO: 175 - RNA, 19 nt, Artificial sequence
  Description of the artificial sequence: sense strand of dsRNA
  modified_base 1: /mod_base = “nucleoside: lacks 5′-phosphate group”
uccauacugcggaacuccu

SEQ ID NO: 176 - RNA, 19 nt, Artificial sequence
  Description of the artificial sequence: sense strand of dsRNA
  modified_base 1: /mod_base = “nucleoside: lacks 5′-phosphate group”
gcgcugaaucccgcggacg

SEQ ID NO: 177 - RNA, 19 nt, Artificial sequence
  Description of the artificial sequence: sense strand of dsRNA
  modified_base 1: /mod_base = “nucleoside: lacks 5′-phosphate group”
aguguggauucgcacuccu

SEQ ID NO: 178 - RNA, 19 nt, Artificial sequence
  Description of the artificial sequence: sense strand of dsRNA
  modified_base 1: /mod_base = “nucleoside: lacks 5′-phosphate group”
cccugcucguguuacaggc

SEQ ID NO: 179 - RNA, 19 nt, Artificial sequence
  Description of the artificial sequence: sense strand of dsRNA
  modified_base 1: /mod_base = “nucleoside: lacks 5′-phosphate group”
gaaucccgcggacgacccg

SEQ ID NO: 180 - RNA, 19 nt, Artificial sequence
  Description of the artificial sequence: sense strand of dsRNA
  modified_base 1: /mod_base = “nucleoside: lacks 5′-phosphate group”
aagcugugccuuggguggc

SEQ ID NO: 181 - RNA, 19 nt, Artificial sequence
  Description of the artificial sequence: sense strand of dsRNA
  modified_base 1: /mod_base = “nucleoside: lacks 5′-phosphate group”
gcccugcucguguuacagg

SEQ ID NO: 182 - RNA, 19 nt, Artificial sequence
  Description of the artificial sequence: sense strand of dsRNA
  modified_base 1: /mod_base = “nucleoside: lacks 5′-phosphate group”
gucccgucggcgcugaauc

SEQ ID NO: 183 - RNA, 19 nt, Artificial sequence
  Description of the artificial sequence: sense strand of dsRNA
  modified_base 1: /mod_base = “nucleoside: lacks 5′-phosphate group”
aucuuaucaacacuuccgg

SEQ ID NO: 184 - RNA, 19 nt, Artificial sequence
  Description of the artificial sequence: sense strand of dsRNA
  modified_base 1: /mod_base = “nucleoside: lacks 5′-phosphate group”
cuuaucaacacuuccggaa

SEQ ID NO: 185 - RNA, 19 nt, Artificial sequence
  Description of the artificial sequence: antisense strand of dsRNA
  modified_base 1: /mod_base = “nucleoside: lacks 5′-phosphate group”
aaacgggcaacauaccuug

SEQ ID NO: 186 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
taccaauuuaugccuacag

SEQ ID NO: 187 - RNA, 19 nt, Artificial sequence
  Description of the artificial sequence: antisense strand of dsRNA
  modified_base 1: /mod_base = “nucleoside: lacks 5′-phosphate group”
uaugauaaaacgccgcaga

SEQ ID NO: 188 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
taugauaaaacgccgcaga

SEQ ID NO: 189 - RNA, 19 nt, Artificial sequence
  Description of the artificial sequence: antisense strand of dsRNA
  modified_base 1: /mod_base = “nucleoside: lacks 5′-phosphate group”
gagaugauuaggcagaggu

SEQ ID NO: 190 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tacaaauggcacuaguaaa

SEQ ID NO: 191 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tagaugauuaggcagaggu

SEQ ID NO: 192 - RNA, 19 nt, Artificial sequence
  Description of the artificial sequence: antisense strand of dsRNA
  modified_base 1: /mod_base = “nucleoside: lacks 5′-phosphate group”
gaccaauuuaugccuacag

SEQ ID NO: 193 - RNA, 19 nt, Artificial sequence
  Description of the artificial sequence: antisense strand of dsRNA
  modified_base 1: /mod_base = “nucleoside: lacks 5′-phosphate group”
ugauaaaacgccgcagaca

SEQ ID NO: 194 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tgauaaaacgccgcagaca

SEQ ID NO: 195 - RNA, 19 nt, Artificial sequence
  Description of the artificial sequence: antisense strand of dsRNA
  modified_base 1: /mod_base = “nucleoside: lacks 5′-phosphate group”
ugaacaaauggcacuagua

SEQ ID NO: 196 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tgaacaaauggcacuagua

SEQ ID NO: 197 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tgcagaggugaaaaaguug

SEQ ID NO: 198 - RNA, 19 nt, Artificial sequence
  Description of the artificial sequence: antisense strand of dsRNA
  modified_base 1: /mod_base = “nucleoside: lacks 5′-phosphate group”
cgaaccacugaacaaaugg

SEQ ID NO: 199 - RNA, 19 nt, Artificial sequence
  Description of the artificial sequence: antisense strand of dsRNA
  modified_base 1: /mod_base = “nucleoside: lacks 5′-phosphate group”
ugcgucagcaaacacuugg

SEQ ID NO: 200 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tgcgucagcaaacacuugg

SEQ ID NO: 201 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tgaaccacugaacaaaugg

SEQ ID NO: 202 - RNA, 19 nt, Artificial sequence
  Description of the artificial sequence: antisense strand of dsRNA
  modified_base 1: /mod_base = “nucleoside: lacks 5′-phosphate group”
aacaaauggcacuaguaaa

SEQ ID NO: 203 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tgaugauuaggcagaggug

SEQ ID NO: 204 - RNA, 19 nt, Artificial sequence
  Description of the artificial sequence: antisense strand of dsRNA
  modified_base 1: /mod_base = “nucleoside: lacks 5′-phosphate group”
cacuaguaaacugagccag

SEQ ID NO: 205 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
taacgggcaacauaccuug

SEQ ID NO: 206 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tacuaguaaacugagccag

SEQ ID NO: 207 - RNA, 19 nt, Artificial sequence
  Description of the artificial sequence: antisense strand of dsRNA
  modified_base 1: /mod_base = “nucleoside: lacks 5′-phosphate group”
aauuuaugccuacagccuc

SEQ ID NO: 208 - RNA, 19 nt, Artificial sequence
  Description of the artificial sequence: antisense strand of dsRNA
  modified_base 1: /mod_base = “nucleoside: lacks 5′-phosphate group”
aaauggcacuaguaaacug

SEQ ID NO: 209 - RNA, 19 nt, Artificial sequence
  Description of the artificial sequence: antisense strand of dsRNA
  modified_base 1: /mod_base = “nucleoside: lacks 5′-phosphate group”
acaaacgggcaacauaccu

SEQ ID NO: 210 - RNA, 19 nt, Artificial sequence
  Description of the artificial sequence: antisense strand of dsRNA
  modified_base 1: /mod_base = “nucleoside: lacks 5′-phosphate group”
uggacaaacgggcaacaua

SEQ ID NO: 211 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tauuuaugccuacagccuc

SEQ ID NO: 212 - RNA, 19 nt, Artificial sequence
  Description of the artificial sequence: antisense strand of dsRNA
  modified_base 1: /mod_base = “nucleoside: lacks 5′-phosphate group”
augauaaaacgccgcagac

SEQ ID NO: 213 - RNA, 19 nt, Artificial sequence
  Description of the artificial sequence: antisense strand of dsRNA
  modified_base 1: /mod_base = “nucleoside: lacks 5′-phosphate group”
ggcagaggugaaaaaguug

SEQ ID NO: 214 - RNA, 19 nt, Artificial sequence
  Description of the artificial sequence: antisense strand of dsRNA
  modified_base 1: /mod_base = “nucleoside: lacks 5′-phosphate group”
ugaagcgaagugcacacgg

SEQ ID NO: 215 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tgaagcgaagugcacacgg

SEQ ID NO: 216 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tacgggcaacauaccuuga

SEQ ID NO: 217 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
taauggcacuaguaaacug

SEQ ID NO: 218 - RNA, 19 nt, Artificial sequence
  Description of the artificial sequence: antisense strand of dsRNA
  modified_base 1: /mod_base = “nucleoside: lacks 5′-phosphate group”
aauugagagaaguccacca

SEQ ID NO: 219 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tcaaacgggcaacauaccu

SEQ ID NO: 220 - RNA, 19 nt, Artificial sequence
  Description of the artificial sequence: antisense strand of dsRNA
  modified_base 1: /mod_base = “nucleoside: lacks 5′-phosphate group”
ccgccuguaacacgagcag

SEQ ID NO: 221 - RNA, 19 nt, Artificial sequence
  Description of the artificial sequence: antisense strand of dsRNA
  modified_base 1: /mod_base = “nucleoside: lacks 5′-phosphate group”
aggacaaacgggcaacaua

SEQ ID NO: 222 - RNA, 19 nt, Artificial sequence
  Description of the artificial sequence: antisense strand of dsRNA
  modified_base 1: /mod_base = “nucleoside: lacks 5′-phosphate group”
aacgggcaacauaccuuga

SEQ ID NO: 223 - RNA, 19 nt, Artificial sequence
  Description of the artificial sequence: antisense strand of dsRNA
  modified_base 1: /mod_base = “nucleoside: lacks 5′-phosphate group”
uccggaaguguugauaaga

SEQ ID NO: 224 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tccggaaguguugauaaga

SEQ ID NO: 225 - RNA, 19 nt, Artificial sequence
  Description of the artificial sequence: antisense strand of dsRNA
  modified_base 1: /mod_base = “nucleoside: lacks 5′-phosphate group”
agaugauuaggcagaggug

SEQ ID NO: 226 - RNA, 19 nt, Artificial sequence
  Description of the artificial sequence: antisense strand of dsRNA
  modified_base 1: /mod_base = “nucleoside: lacks 5′-phosphate group”
aguccaagaguccucuuau

SEQ ID NO: 227 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tugauaaaacgccgcagac

SEQ ID NO: 228 - RNA, 19 nt, Artificial sequence
  Description of the artificial sequence: antisense strand of dsRNA
  modified_base 1: /mod_base = “nucleoside: lacks 5′-phosphate group”
guccgcgggauucagcgcc

SEQ ID NO: 229 - RNA, 19 nt, Artificial sequence
  Description of the artificial sequence: antisense strand of dsRNA
  modified_base 1: /mod_base = “nucleoside: lacks 5′-phosphate group”
ugagaucuucugcgacgcg

SEQ ID NO: 230 - RNA, 19 nt, Artificial sequence
  Description of the artificial sequence: antisense strand of dsRNA
  modified_base 1: /mod_base = “nucleoside: lacks 5′-phosphate group”
aaggucggucguugacauu

SEQ ID NO: 231 - RNA, 19 nt, Artificial sequence
  Description of the artificial sequence: antisense strand of dsRNA
  modified_base 1: /mod_base = “nucleoside: lacks 5′-phosphate group”
uggcacuaguaaacugagc

SEQ ID NO: 232 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tauugagagaaguccacca

SEQ ID NO: 233 - RNA, 19 nt, Artificial sequence
  Description of the artificial sequence: antisense strand of dsRNA
  modified_base 1: /mod_base = “nucleoside: lacks 5′-phosphate group”
ucuucugcgacgcggcgau

SEQ ID NO: 234 - RNA, 19 nt, Artificial sequence
  Description of the artificial sequence: antisense strand of dsRNA
  modified_base 1: /mod_base = “nucleoside: lacks 5′-phosphate group”
gaaccacugaacaaauggc

SEQ ID NO: 235 - RNA, 19 nt, Artificial sequence
  Description of the artificial sequence: antisense strand of dsRNA
  modified_base 1: /mod_base = “nucleoside: lacks 5′-phosphate group”
aguuccgcaguauggaucg

SEQ ID NO: 236 - RNA, 19 nt, Artificial sequence
  Description of the artificial sequence: antisense strand of dsRNA
  modified_base 1: /mod_base = “nucleoside: lacks 5′-phosphate group”
gaugauuaggcagagguga

SEQ ID NO: 237 - RNA, 19 nt, Artificial sequence
  Description of the artificial sequence: antisense strand of dsRNA
  modified_base 1: /mod_base = “nucleoside: lacks 5′-phosphate group”
aaaauugagagaaguccac

SEQ ID NO: 238 - RNA, 19 nt, Artificial sequence
  Description of the artificial sequence: antisense strand of dsRNA
  modified_base 1: /mod_base = “nucleoside: lacks 5′-phosphate group”
cccaagaauauggugaccc

SEQ ID NO: 239 - RNA, 19 nt, Artificial sequence
  Description of the artificial sequence: antisense strand of dsRNA
  modified_base 1: /mod_base = “nucleoside: lacks 5′-phosphate group”
agaucuucugcgacgcggc

SEQ ID NO: 240 - RNA, 19 nt, Artificial sequence
  Description of the artificial sequence: antisense strand of dsRNA
  modified_base 1: /mod_base = “nucleoside: lacks 5′-phosphate group”
ucugcgacgcggcgauuga

SEQ ID NO: 241 - RNA, 19 nt, Artificial sequence
  Description of the artificial sequence: antisense strand of dsRNA
  modified_base 1: /mod_base = “nucleoside: lacks 5′-phosphate group”
aacgccgcagacacaucca

SEQ ID NO: 242 - RNA, 19 nt, Artificial sequence
  Description of the artificial sequence: antisense strand of dsRNA
  modified_base 1: /mod_base = “nucleoside: lacks 5′-phosphate group”
cuuggaggcuugaacagua

SEQ ID NO: 243 - RNA, 19 nt, Artificial sequence
  Description of the artificial sequence: antisense strand of dsRNA
  modified_base 1: /mod_base = “nucleoside: lacks 5′-phosphate group”
acaaauggcacuaguaaac

SEQ ID NO: 244 - RNA, 19 nt, Artificial sequence
  Description of the artificial sequence: antisense strand of dsRNA
  modified_base 1: /mod_base = “nucleoside: lacks 5′-phosphate group”
cugaacaaauggcacuagu

SEQ ID NO: 245 - RNA, 19 nt, Artificial sequence
  Description of the artificial sequence: antisense strand of dsRNA
  modified_base 1: /mod_base = “nucleoside: lacks 5′-phosphate group”
gagaucuucugcgacgcgg

SEQ ID NO: 246 - RNA, 19 nt, Artificial sequence
  Description of the artificial sequence: antisense strand of dsRNA
  modified_base 1: /mod_base = “nucleoside: lacks 5′-phosphate group”
cggaaguguugauaagaua

SEQ ID NO: 247 - RNA, 19 nt, Artificial sequence
  Description of the artificial sequence: antisense strand of dsRNA
  modified_base 1: /mod_base = “nucleoside: lacks 5′-phosphate group”
gggacugcgaauuuuggcc

SEQ ID NO: 248 - RNA, 19 nt, Artificial sequence
  Description of the artificial sequence: antisense strand of dsRNA
  modified_base 1: /mod_base = “nucleoside: lacks 5′-phosphate group”
augauuaggcagaggugaa

SEQ ID NO: 249 - RNA, 19 nt, Artificial sequence
  Description of the artificial sequence: antisense strand of dsRNA
  modified_base 1: /mod_base = “nucleoside: lacks 5′-phosphate group”
auggcacuaguaaacugag

SEQ ID NO: 250 - RNA, 19 nt, Artificial sequence
  Description of the artificial sequence: antisense strand of dsRNA
  modified_base 1: /mod_base = “nucleoside: lacks 5′-phosphate group”
agaggacaaacgggcaaca

SEQ ID NO: 251 - RNA, 19 nt, Artificial sequence
  Description of the artificial sequence: antisense strand of dsRNA
  modified_base 1: /mod_base = “nucleoside: lacks 5′-phosphate group”
cacugaacaaauggcacua

SEQ ID NO: 252 - RNA, 19 nt, Artificial sequence
  Description of the artificial sequence: antisense strand of dsRNA
  modified_base 1: /mod_base = “nucleoside: lacks 5′-phosphate group”
caauuuaugccuacagccu

SEQ ID NO: 253 - RNA, 19 nt, Artificial sequence
  Description of the artificial sequence: antisense strand of dsRNA
  modified_base 1: /mod_base = “nucleoside: lacks 5′-phosphate group”
uaaaacgccgcagacacau

SEQ ID NO: 254 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
taaaacgccgcagacacau

SEQ ID NO: 255 - RNA, 19 nt, Artificial sequence
  Description of the artificial sequence: antisense strand of dsRNA
  modified_base 1: /mod_base = “nucleoside: lacks 5′-phosphate group”
ugcagaggugaagcgaagu

SEQ ID NO: 256 - RNA, 19 nt, Artificial sequence
  Description of the artificial sequence: antisense strand of dsRNA
  modified_base 1: /mod_base = “nucleoside: lacks 5′-phosphate group”
gugaagcgaagugcacacg

SEQ ID NO: 257 - RNA, 19 nt, Artificial sequence
  Description of the artificial sequence: antisense strand of dsRNA
  modified_base 1: /mod_base = “nucleoside: lacks 5′-phosphate group”
auugagagaaguccaccac

SEQ ID NO: 258 - RNA, 19 nt, Artificial sequence
  Description of the artificial sequence: antisense strand of dsRNA
  modified_base 1: /mod_base = “nucleoside: lacks 5′-phosphate group”
auaaaacgccgcagacaca

SEQ ID NO: 259 - RNA, 19 nt, Artificial sequence
  Description of the artificial sequence: antisense strand of dsRNA
  modified_base 1: /mod_base = “nucleoside: lacks 5′-phosphate group”
caaacgggcaacauaccuu

SEQ ID NO: 260 - RNA, 19 nt, Artificial sequence
  Description of the artificial sequence: antisense strand of dsRNA
  modified_base 1: /mod_base = “nucleoside: lacks 5′-phosphate group”
ccucaaggucggucguuga

SEQ ID NO: 261 - RNA, 19 nt, Artificial sequence
  Description of the artificial sequence: antisense strand of dsRNA
  modified_base 1: /mod_base = “nucleoside: lacks 5′-phosphate group”
guccaagaguccucuuaug

SEQ ID NO: 262 - RNA, 19 nt, Artificial sequence
  Description of the artificial sequence: antisense strand of dsRNA
  modified_base 1: /mod_base = “nucleoside: lacks 5′-phosphate group”
cucaaggucggucguugac

SEQ ID NO: 263 - RNA, 19 nt, Artificial sequence
  Description of the artificial sequence: antisense strand of dsRNA
  modified_base 1: /mod_base = “nucleoside: lacks 5′-phosphate group”
cucuuguucccaagaauau

SEQ ID NO: 264 - RNA, 19 nt, Artificial sequence
  Description of the artificial sequence: antisense strand of dsRNA
  modified_base 1: /mod_base = “nucleoside: lacks 5′-phosphate group”
cccgccuguaacacgagca

SEQ ID NO: 265 - RNA, 19 nt, Artificial sequence
  Description of the artificial sequence: antisense strand of dsRNA
  modified_base 1: /mod_base = “nucleoside: lacks 5′-phosphate group”
uucugcgacgcggcgauug

SEQ ID NO: 266 - RNA, 19 nt, Artificial sequence
  Description of the artificial sequence: antisense strand of dsRNA
  modified_base 1: /mod_base = “nucleoside: lacks 5′-phosphate group”
gcuuggaggcuugaacagu

SEQ ID NO: 267 - RNA, 19 nt, Artificial sequence
  Description of the artificial sequence: antisense strand of dsRNA
  modified_base 1: /mod_base = “nucleoside: lacks 5′-phosphate group”
gaucuucugcgacgcggcg

SEQ ID NO: 268 - RNA, 19 nt, Artificial sequence
  Description of the artificial sequence: antisense strand of dsRNA
  modified_base 1: /mod_base = “nucleoside: lacks 5′-phosphate group”
acgaaccacugaacaaaug

SEQ ID NO: 269 - RNA, 19 nt, Artificial sequence
  Description of the artificial sequence: antisense strand of dsRNA
  modified_base 1: /mod_base = “nucleoside: lacks 5′-phosphate group”
ucguccgcgggauucagcg

SEQ ID NO: 270 - RNA, 19 nt, Artificial sequence
  Description of the artificial sequence: antisense strand of dsRNA
  modified_base 1: /mod_base = “nucleoside: lacks 5′-phosphate group”
ccaagaauauggugaccca

SEQ ID NO: 271 - RNA, 19 nt, Artificial sequence
  Description of the artificial sequence: antisense strand of dsRNA
  modified_base 1: /mod_base = “nucleoside: lacks 5′-phosphate group”
aguauggaucggcagagga

SEQ ID NO: 272 - RNA, 19 nt, Artificial sequence
  Description of the artificial sequence: antisense strand of dsRNA
  modified_base 1: /mod_base = “nucleoside: lacks 5′-phosphate group”
caaggucggucguugacau

SEQ ID NO: 273 - RNA, 19 nt, Artificial sequence
  Description of the artificial sequence: antisense strand of dsRNA
  modified_base 1: /mod_base = “nucleoside: lacks 5′-phosphate group”
ugagaugauuaggcagagg

SEQ ID NO: 274 - RNA, 19 nt, Artificial sequence
  Description of the artificial sequence: antisense strand of dsRNA
  modified_base 1: /mod_base = “nucleoside: lacks 5′-phosphate group”
gaagcgaagugcacacggu

SEQ ID NO: 275 - RNA, 19 nt, Artificial sequence
  Description of the artificial sequence: antisense strand of dsRNA
  modified_base 1: /mod_base = “nucleoside: lacks 5′-phosphate group”
uccgcaguauggaucggca

SEQ ID NO: 276 - RNA, 19 nt, Artificial sequence
  Description of the artificial sequence: antisense strand of dsRNA
  modified_base 1: /mod_base = “nucleoside: lacks 5′-phosphate group”
accacgagucuagacucug

SEQ ID NO: 277 - RNA, 19 nt, Artificial sequence
  Description of the artificial sequence: antisense strand of dsRNA
  modified_base 1: /mod_base = “nucleoside: lacks 5′-phosphate group”
agcuuggaggcuugaacag

SEQ ID NO: 278 - RNA, 19 nt, Artificial sequence
  Description of the artificial sequence: antisense strand of dsRNA
  modified_base 1: /mod_base = “nucleoside: lacks 5′-phosphate group”
auuuaugccuacagccucc

SEQ ID NO: 279 - RNA, 19 nt, Artificial sequence
  Description of the artificial sequence: antisense strand of dsRNA
  modified_base 1: /mod_base = “nucleoside: lacks 5′-phosphate group”
uuuaugccuacagccuccu

SEQ ID NO: 280 - RNA, 19 nt, Artificial sequence
  Description of the artificial sequence: antisense strand of dsRNA
  modified_base 1: /mod_base = “nucleoside: lacks 5′-phosphate group”
aaauugagagaaguccacc

SEQ ID NO: 281 - RNA, 19 nt, Artificial sequence
  Description of the artificial sequence: antisense strand of dsRNA
  modified_base 1: /mod_base = “nucleoside: lacks 5′-phosphate group”
gcagaggugaaaaaguugc

SEQ ID NO: 282 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tcgccuguaacacgagcag

SEQ ID NO: 283 - RNA, 19 nt, Artificial sequence
  Description of the artificial sequence: antisense strand of dsRNA
  modified_base 1: /mod_base = “nucleoside: lacks 5′-phosphate group”
acugaacaaauggcacuag

SEQ ID NO: 284 - RNA, 19 nt, Artificial sequence
  Description of the artificial sequence: antisense strand of dsRNA
  modified_base 1: /mod_base = “nucleoside: lacks 5′-phosphate group”
ccgcaguauggaucggcag

SEQ ID NO: 285 - RNA, 19 nt, Artificial sequence
  Description of the artificial sequence: antisense strand of dsRNA
  modified_base 1: /mod_base = “nucleoside: lacks 5′-phosphate group”
ggugaagcgaagugcacac

SEQ ID NO: 286 - RNA, 19 nt, Artificial sequence
  Description of the artificial sequence: antisense strand of dsRNA
  modified_base 1: /mod_base = “nucleoside: lacks 5′-phosphate group”
gcccgccuguaacacgagc

SEQ ID NO: 287 - RNA, 19 nt, Artificial sequence
  Description of the artificial sequence: antisense strand of dsRNA
  modified_base 1: /mod_base = “nucleoside: lacks 5′-phosphate group”
gaaguguugauaagauagg

SEQ ID NO: 288 - RNA, 19 nt, Artificial sequence
  Description of the artificial sequence: antisense strand of dsRNA
  modified_base 1: /mod_base = “nucleoside: lacks 5′-phosphate group”
ugcgacgcggcgauugaga

SEQ ID NO: 289 - RNA, 19 nt, Artificial sequence
  Description of the artificial sequence: antisense strand of dsRNA
  modified_base 1: /mod_base = “nucleoside: lacks 5′-phosphate group”
ugagaaggcacagacgggc

SEQ ID NO: 290 - RNA, 19 nt, Artificial sequence
  Description of the artificial sequence: antisense strand of dsRNA
  modified_base 1: /mod_base = “nucleoside: lacks 5′-phosphate group”
ggaaguguugauaagauag

SEQ ID NO: 291 - RNA, 19 nt, Artificial sequence
  Description of the artificial sequence: antisense strand of dsRNA
  modified_base 1: /mod_base = “nucleoside: lacks 5′-phosphate group”
gaggacaaacgggcaacau

SEQ ID NO: 292 - RNA, 19 nt, Artificial sequence
  Description of the artificial sequence: antisense strand of dsRNA
  modified_base 1: /mod_base = “nucleoside: lacks 5′-phosphate group”
ggacaaacgggcaacauac

SEQ ID NO: 293 - RNA, 19 nt, Artificial sequence
  Description of the artificial sequence: antisense strand of dsRNA
  modified_base 1: /mod_base = “nucleoside: lacks 5′-phosphate group”
gugcagaggugaagcgaag

SEQ ID NO: 294 - RNA, 19 nt, Artificial sequence
  Description of the artificial sequence: antisense strand of dsRNA
  modified_base 1: /mod_base = “nucleoside: lacks 5′-phosphate group”
aggugaagcgaagugcaca

SEQ ID NO: 295 - RNA, 19 nt, Artificial sequence
  Description of the artificial sequence: antisense strand of dsRNA
  modified_base 1: /mod_base = “nucleoside: lacks 5′-phosphate group”
cgggacugcgaauuuuggc

SEQ ID NO: 296 - RNA, 19 nt, Artificial sequence
  Description of the artificial sequence: antisense strand of dsRNA
  modified_base 1: /mod_base = “nucleoside: lacks 5′-phosphate group”
cgccuguaacacgagcagg

SEQ ID NO: 297 - RNA, 19 nt, Artificial sequence
  Description of the artificial sequence: antisense strand of dsRNA
  modified_base 1: /mod_base = “nucleoside: lacks 5′-phosphate group”
agugcgaauccacacucca

SEQ ID NO: 298 - RNA, 19 nt, Artificial sequence
  Description of the artificial sequence: antisense strand of dsRNA
  modified_base 1: /mod_base = “nucleoside: lacks 5′-phosphate group”
ugccucaaggucggucguu

SEQ ID NO: 299 - RNA, 19 nt, Artificial sequence
  Description of the artificial sequence: antisense strand of dsRNA
  modified_base 1: /mod_base = “nucleoside: lacks 5′-phosphate group”
ccacgagucuagacucugu

SEQ ID NO: 300 - RNA, 19 nt, Artificial sequence
  Description of the artificial sequence: antisense strand of dsRNA
  modified_base 1: /mod_base = “nucleoside: lacks 5′-phosphate group”
cuucugcgacgcggcgauu

SEQ ID NO: 301 - RNA, 19 nt, Artificial sequence
  Description of the artificial sequence: antisense strand of dsRNA
  modified_base 1: /mod_base = “nucleoside: lacks 5′-phosphate group”
gacaaacgggcaacauacc

SEQ ID NO: 302 - RNA, 19 nt, Artificial sequence
  Description of the artificial sequence: antisense strand of dsRNA
  modified_base 1: /mod_base = “nucleoside: lacks 5′-phosphate group”
uuccgcaguauggaucggc

SEQ ID NO: 303 - RNA, 19 nt, Artificial sequence
  Description of the artificial sequence: antisense strand of dsRNA
  modified_base 1: /mod_base = “nucleoside: lacks 5′-phosphate group”
aguguugauaagauagggc

SEQ ID NO: 304 - RNA, 19 nt, Artificial sequence
  Description of the artificial sequence: antisense strand of dsRNA
  modified_base 1: /mod_base = “nucleoside: lacks 5′-phosphate group”
caaauggcacuaguaaacu

SEQ ID NO: 305 - RNA, 19 nt, Artificial sequence
  Description of the artificial sequence: antisense strand of dsRNA
  modified_base 1: /mod_base = “nucleoside: lacks 5′-phosphate group”
ucaaggucggucguugaca

SEQ ID NO: 306 - RNA, 19 nt, Artificial sequence
  Description of the artificial sequence: antisense strand of dsRNA
  modified_base 1: /mod_base = “nucleoside: lacks 5′-phosphate group”
cuagaaaauugagagaagu

SEQ ID NO: 307 - RNA, 19 nt, Artificial sequence
  Description of the artificial sequence: antisense strand of dsRNA
  modified_base 1: /mod_base = “nucleoside: lacks 5′-phosphate group”
gacaaaggacgucccgcgc

SEQ ID NO: 308 - RNA, 19 nt, Artificial sequence
  Description of the artificial sequence: antisense strand of dsRNA
  modified_base 1: /mod_base = “nucleoside: lacks 5′-phosphate group”
aguccaccacgagucuaga

SEQ ID NO: 309 - RNA, 19 nt, Artificial sequence
  Description of the artificial sequence: antisense strand of dsRNA
  modified_base 1: /mod_base = “nucleoside: lacks 5′-phosphate group”
gaguuccgcaguauggauc

SEQ ID NO: 310 - RNA, 19 nt, Artificial sequence
  Description of the artificial sequence: antisense strand of dsRNA
  modified_base 1: /mod_base = “nucleoside: lacks 5′-phosphate group”
gcaguauggaucggcagag

SEQ ID NO: 311 - RNA, 19 nt, Artificial sequence
  Description of the artificial sequence: antisense strand of dsRNA
  modified_base 1: /mod_base = “nucleoside: lacks 5′-phosphate group”
cgcaguauggaucggcaga

SEQ ID NO: 312 - RNA, 19 nt, Artificial sequence
  Description of the artificial sequence: antisense strand of dsRNA
  modified_base 1: /mod_base = “nucleoside: lacks 5′-phosphate group”
caguauggaucggcagagg

SEQ ID NO: 313 - RNA, 19 nt, Artificial sequence
  Description of the artificial sequence: antisense strand of dsRNA
  modified_base 1: /mod_base = “nucleoside: lacks 5′-phosphate group”
accgcguaaagagaggugc

SEQ ID NO: 314 - RNA, 19 nt, Artificial sequence
  Description of the artificial sequence: antisense strand of dsRNA
  modified_base 1: /mod_base = “nucleoside: lacks 5′-phosphate group”
gcgaggcgagggaguucuu

SEQ ID NO: 315 - RNA, 19 nt, Artificial sequence
  Description of the artificial sequence: antisense strand of dsRNA
  modified_base 1: /mod_base = “nucleoside: lacks 5′-phosphate group”
cugcgaggcgagggaguuc

SEQ ID NO: 316 - RNA, 19 nt, Artificial sequence
  Description of the artificial sequence: antisense strand of dsRNA
  modified_base 1: /mod_base = “nucleoside: lacks 5′-phosphate group”
gcccuagaaaauugagaga

SEQ ID NO: 317 - RNA, 19 nt, Artificial sequence
  Description of the artificial sequence: antisense strand of dsRNA
  modified_base 1: /mod_base = “nucleoside: lacks 5′-phosphate group”
cguaaagagaggugcgccc

SEQ ID NO: 318 - RNA, 19 nt, Artificial sequence
  Description of the artificial sequence: antisense strand of dsRNA
  modified_base 1: /mod_base = “nucleoside: lacks 5′-phosphate group”
guuccgcaguauggaucgg

SEQ ID NO: 319 - RNA, 19 nt, Artificial sequence
  Description of the artificial sequence: antisense strand of dsRNA
  modified_base 1: /mod_base = “nucleoside: lacks 5′-phosphate group”
ucugcgaggcgagggaguu

SEQ ID NO: 320 - RNA, 19 nt, Artificial sequence
  Description of the artificial sequence: antisense strand of dsRNA
  modified_base 1: /mod_base = “nucleoside: lacks 5′-phosphate group”
guauggaucggcagaggag

SEQ ID NO: 321 - RNA, 19 nt, Artificial sequence
  Description of the artificial sequence: antisense strand of dsRNA
  modified_base 1: /mod_base = “nucleoside: lacks 5′-phosphate group”
gagugcgaauccacacucc

SEQ ID NO: 322 - RNA, 19 nt, Artificial sequence
  Description of the artificial sequence: antisense strand of dsRNA
  modified_base 1: /mod_base = “nucleoside: lacks 5′-phosphate group”
guaaagagaggugcgcccg

SEQ ID NO: 323 - RNA, 19 nt, Artificial sequence
  Description of the artificial sequence: antisense strand of dsRNA
  modified_base 1: /mod_base = “nucleoside: lacks 5′-phosphate group”
gcgacgcggcgauugagac

SEQ ID NO: 324 - RNA, 19 nt, Artificial sequence
  Description of the artificial sequence: antisense strand of dsRNA
  modified_base 1: /mod_base = “nucleoside: lacks 5′-phosphate group”
ggaguuccgcaguauggau

SEQ ID NO: 325 - RNA, 19 nt, Artificial sequence
  Description of the artificial sequence: antisense strand of dsRNA
  modified_base 1: /mod_base = “nucleoside: lacks 5′-phosphate group”
ccgcguaaagagaggugcg

SEQ ID NO: 326 - RNA, 19 nt, Artificial sequence
  Description of the artificial sequence: antisense strand of dsRNA
  modified_base 1: /mod_base = “nucleoside: lacks 5′-phosphate group”
gccucaaggucggucguug

SEQ ID NO: 327 - RNA, 19 nt, Artificial sequence
  Description of the artificial sequence: antisense strand of dsRNA
  modified_base 1: /mod_base = “nucleoside: lacks 5′-phosphate group”
uaggaguuccgcaguaugg

SEQ ID NO: 328 - RNA, 19 nt, Artificial sequence
  Description of the artificial sequence: antisense strand of dsRNA
  modified_base 1: /mod_base = “nucleoside: lacks 5′-phosphate group”
gggucguccgcgggauuca

SEQ ID NO: 329 - RNA, 19 nt, Artificial sequence
  Description of the artificial sequence: antisense strand of dsRNA
  modified_base 1: /mod_base = “nucleoside: lacks 5′-phosphate group”
ugcgaggcgagggaguucu

SEQ ID NO: 330 - RNA, 19 nt, Artificial sequence
  Description of the artificial sequence: antisense strand of dsRNA
  modified_base 1: /mod_base = “nucleoside: lacks 5′-phosphate group”
gcguaaagagaggugcgcc

SEQ ID NO: 331 - RNA, 19 nt, Artificial sequence
  Description of the artificial sequence: antisense strand of dsRNA
  modified_base 1: /mod_base = “nucleoside: lacks 5′-phosphate group”
cgcguaaagagaggugcgc

SEQ ID NO: 332 - RNA, 19 nt, Artificial sequence
  Description of the artificial sequence: antisense strand of dsRNA
  modified_base 1: /mod_base = “nucleoside: lacks 5′-phosphate group”
gucguccgcgggauucagc

SEQ ID NO: 333 - RNA, 19 nt, Artificial sequence
  Description of the artificial sequence: antisense strand of dsRNA
  modified_base 1: /mod_base = “nucleoside: lacks 5′-phosphate group”
gcagaggugaagcgaagug

SEQ ID NO: 334 - RNA, 19 nt, Artificial sequence
  Description of the artificial sequence: antisense strand of dsRNA
  modified_base 1: /mod_base = “nucleoside: lacks 5′-phosphate group”
cugcgacgcggcgauugag

SEQ ID NO: 335 - RNA, 19 nt, Artificial sequence
  Description of the artificial sequence: antisense strand of dsRNA
  modified_base 1: /mod_base = “nucleoside: lacks 5′-phosphate group”
ggauucagcgccgacggga

SEQ ID NO: 336 - RNA, 19 nt, Artificial sequence
  Description of the artificial sequence: antisense strand of dsRNA
  modified_base 1: /mod_base = “nucleoside: lacks 5′-phosphate group”
ggucguccgcgggauucag

SEQ ID NO: 337 - RNA, 19 nt, Artificial sequence
  Description of the artificial sequence: antisense strand of dsRNA
  modified_base 1: /mod_base = “nucleoside: lacks 5′-phosphate group”
caccacgagucuagacucu

SEQ ID NO: 338 - RNA, 19 nt, Artificial sequence
  Description of the artificial sequence: antisense strand of dsRNA
  modified_base 1: /mod_base = “nucleoside: lacks 5′-phosphate group”
aggaguuccgcaguaugga

SEQ ID NO: 339 - RNA, 19 nt, Artificial sequence
  Description of the artificial sequence: antisense strand of dsRNA
  modified_base 1: /mod_base = “nucleoside: lacks 5′-phosphate group”
cguccgcgggauucagcgc

SEQ ID NO: 340 - RNA, 19 nt, Artificial sequence
  Description of the artificial sequence: antisense strand of dsRNA
  modified_base 1: /mod_base = “nucleoside: lacks 5′-phosphate group”
aggagugcgaauccacacu

SEQ ID NO: 341 - RNA, 19 nt, Artificial sequence
  Description of the artificial sequence: antisense strand of dsRNA
  modified_base 1: /mod_base = “nucleoside: lacks 5′-phosphate group”
gccuguaacacgagcaggg

SEQ ID NO: 342 - RNA, 19 nt, Artificial sequence
  Description of the artificial sequence: antisense strand of dsRNA
  modified_base 1: /mod_base = “nucleoside: lacks 5′-phosphate group”
cgggucguccgcgggauuc

SEQ ID NO: 343 - RNA, 19 nt, Artificial sequence
  Description of the artificial sequence: antisense strand of dsRNA
  modified_base 1: /mod_base = “nucleoside: lacks 5′-phosphate group”
gccacccaaggcacagcuu

SEQ ID NO: 344 - RNA, 19 nt, Artificial sequence
  Description of the artificial sequence: antisense strand of dsRNA
  modified_base 1: /mod_base = “nucleoside: lacks 5′-phosphate group”
ccuguaacacgagcagggc

SEQ ID NO: 345 - RNA, 19 nt, Artificial sequence
  Description of the artificial sequence: antisense strand of dsRNA
  modified_base 1: /mod_base = “nucleoside: lacks 5′-phosphate group”
gauucagcgccgacgggac

SEQ ID NO: 346 - RNA, 19 nt, Artificial sequence
  Description of the artificial sequence: antisense strand of dsRNA
  modified_base 1: /mod_base = “nucleoside: lacks 5′-phosphate group”
ccggaaguguugauaagau

SEQ ID NO: 347 - RNA, 19 nt, Artificial sequence
  Description of the artificial sequence: antisense strand of dsRNA
  modified_base 1: /mod_base = “nucleoside: lacks 5′-phosphate group”
uuccggaaguguugauaag

SEQ ID NO: 348 - DNA, 19 bp, Artificial sequence
  Description of the artificial sequence: antisense strand of dsRNA
  modified_base 1: /mod_base = “nucleoside: lacks 5′-phosphate group”
  modified_base 2..19: /mod_base = “2′-hydroxy corresponding nucleoside”
tuccggaaguguugauaag

SEQ ID NO: 349 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 6, 8, 10, 11, 13, 14, 15, 17, 18, 19: /mod_base = “2′-O-methyl corresponding nucleoside”
  modified_base 21: /mod_base = “5′-phosphorothioate thymidine”
  modified_base 3, 4, 5, 7, 9, 12, 16: /mod_base = “2′-hydroxy corresponding nucleoside”
caagguauguugcccguuutt

SEQ ID NO: 350 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
cuguaggcauaaauugguat

SEQ ID NO: 351 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 8, 10, 11, 12, 13, 15, 16, 18: /mod_base = “2′-O-methyl corresponding nucleoside”
  modified_base 21: /mod_base = “5′-phosphorothioate thymidine”
  modified_base 4, 6, 7, 9, 14, 17, 19: /mod_base = “2′-hydroxy corresponding nucleoside”
ucugcggcguuuuaucauatt

SEQ ID NO: 352 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
ucugcggcguuuuaucauat

SEQ ID NO: 353 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 6, 8, 9, 10, 13, 14, 16, 17, 18, 19: /mod_base = “2′-O-methyl corresponding nucleoside”
  modified_base 21: /mod_base = “5′-phosphorothioate thymidine”
  modified_base 7, 11, 12, 15: /mod_base = “2′-hydroxy corresponding nucleoside”
accucugccuaaucaucuctt

SEQ ID NO: 354 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
uuuacuagugccauuuguat

SEQ ID NO: 355 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
accucugccuaaucaucuat

SEQ ID NO: 356 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 8, 10, 14, 15, 18, 19: /mod_base = “2′-O-methyl corresponding nucleoside”
  modified_base 21: /mod_base = “5′-phosphorothioate thymidine”
  modified_base 3, 5, 6, 7, 9, 11, 12, 13, 16, 17: /mod_base = “2′-hydroxy corresponding nucleoside”
cuguaggcauaaauugguctt

SEQ ID NO: 357 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 10, 12, 13, 14, 15, 17, 18: /mod_base = “2′-O-methyl corresponding nucleoside”
  modified_base 21: /mod_base = “5′-phosphorothioate thymidine”
  modified_base 6, 8, 9, 11, 16, 19: /mod_base = “2′-hydroxy corresponding nucleoside”
ugucugcggcguuuuaucatt

SEQ ID NO: 358 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
ugucugcggcguuuuaucat

SEQ ID NO: 359 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 7, 9, 10, 12, 13, 14, 16, 17, 18: /mod_base = “2′-O-methyl corresponding nucleoside”
  modified_base 21: /mod_base = “5′-phosphorothioate thymidine”
  modified_base 5, 6, 8, 11, 15, 19: /mod_base = “2′-hydroxy corresponding nucleoside”
uacuagugccauuuguucatt

SEQ ID NO: 360 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
uacuagugccauuuguucat

SEQ ID NO: 361 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
caacuuuuucaccucugcat

SEQ ID NO: 362 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 8, 9, 10, 13, 16, 17, 18: /mod_base = “2′-O-methyl corresponding nucleoside”
  modified_base 21: /mod_base = “5′-phosphorothioate thymidine”
  modified_base 3, 7, 11, 12, 14, 15, 19: /mod_base = “2′-hydroxy corresponding nucleoside”
ccauuuguucagugguucgtt

SEQ ID NO: 363 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 6, 8, 9, 10, 12, 13, 16, 18: /mod_base = “2′-O-methyl corresponding nucleoside”
  modified_base 21: /mod_base = “5′-phosphorothioate thymidine”
  modified_base 3, 4, 5, 7, 11, 14, 15, 17, 19: /mod_base = “2′-hydroxy corresponding nucleoside”
ccaaguguuugcugacgcatt

SEQ ID NO: 364 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
ccaaguguuugcugacgcat

SEQ ID NO: 365 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
ccauuuguucagugguucat

SEQ ID NO: 366 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 9, 11, 12, 14, 15, 16, 18, 19: /mod_base = “2′-O-methyl corresponding nucleoside”
  modified_base 21: /mod_base = “5′-phosphorothioate thymidine”
  modified_base 4, 7, 8, 10, 13, 17: /mod_base = “2′-hydroxy corresponding nucleoside”
uuuacuagugccauuuguutt

SEQ ID NO: 367 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
caccucugccuaaucaucat

SEQ ID NO: 368 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 6, 7, 10, 11, 12, 14, 15, 18: /mod_base = “2′-O-methyl corresponding nucleoside”
  modified_base 21: /mod_base = “5′-phosphorothioate thymidine”
  modified_base 3, 4, 8, 9, 13, 16, 17, 19: /mod_base = “2′-hydroxy corresponding nucleoside”
cuggcucaguuuacuagugtt

SEQ ID NO: 369 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
caagguauguugcccguuat

SEQ ID NO: 370 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
cuggcucaguuuacuaguat

SEQ ID NO: 371 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 6, 8, 12, 14, 18, 19: /mod_base = “2′-O-methyl corresponding nucleoside”
  modified_base 21: /mod_base = “5′-phosphorothioate thymidine”
  modified_base 3, 4, 7, 9, 10, 11, 13, 15, 16, 17: /mod_base = “2′-hydroxy corresponding nucleoside”
gaggcuguaggcauaaauutt

SEQ ID NO: 372 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 8, 9, 12, 14, 15, 17, 18, 19: /mod_base = “2′-O-methyl corresponding nucleoside”
  modified_base 21: /mod_base = “5′-phosphorothioate thymidine”
  modified_base 3, 7, 10, 11, 13, 16: /mod_base = “2′-hydroxy corresponding nucleoside”
caguuuacuagugccauuutt

SEQ ID NO: 373 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 8, 9, 11, 12, 13, 15, 16, 17, 19: /mod_base = “2′-O-methyl corresponding nucleoside”
  modified_base 21: /mod_base = “5′-phosphorothioate thymidine”
  modified_base 3, 5, 7, 10, 14, 18: /mod_base = “2′-hydroxy corresponding nucleoside”
agguauguugcccguuugutt

SEQ ID NO: 374 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
uauguugcccguuuguccat

SEQ ID NO: 375 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
gaggcuguaggcauaaauat

SEQ ID NO: 376 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 9, 11, 12, 13, 14, 16, 17, 19: /mod_base = “2′-O-methyl corresponding nucleoside”
  modified_base 21: /mod_base = “5′-phosphorothioate thymidine”
  modified_base 5, 7, 8, 10, 15, 18: /mod_base = “2′-hydroxy corresponding nucleoside”
gucugcggcguuuuaucautt

SEQ ID NO: 377 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 7, 8, 9, 10, 12, 13, 14, 15, 16, 18, 19: /mod_base = “2′-O-methyl corresponding nucleoside”
  modified_base 21: /mod_base = “5′-phosphorothioate thymidine”
  modified_base 3, 11, 17: /mod_base = “2′-hydroxy corresponding nucleoside”
caacuuuuucaccucugcctt

SEQ ID NO: 378 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 8, 10, 11, 12, 13, 15, 16, 17, 18: /mod_base = “2′-O-methyl corresponding nucleoside”
  modified_base 21: /mod_base = “5′-phosphorothioate thymidine”
  modified_base 3, 5, 7, 9, 14, 19: /mod_base = “2′-hydroxy corresponding nucleoside”
ccgugugcacuucgcuucatt

SEQ ID NO: 379 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
ccgugugcacuucgcuucat

SEQ ID NO: 380 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
ucaagguauguugcccguat

SEQ ID NO: 381 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
caguuuacuagugccauuat

SEQ ID NO: 382 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 8, 9, 10, 11, 12, 13, 14, 15, 18, 19: /mod_base = “2′-O-methyl corresponding nucleoside”
  modified_base 21: /mod_base = “5′-phosphorothioate thymidine”
  modified_base 3, 5, 6, 7, 16, 17: /mod_base = “2′-hydroxy corresponding nucleoside”
ugguggacuucucucaauutt

SEQ ID NO: 383 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
agguauguugcccguuugat

SEQ ID NO: 384 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 8, 10, 11, 13, 17: /mod_base = “2′-O-methyl corresponding nucleoside”
  modified_base 21: /mod_base = “5′-phosphorothioate thymidine”
  modified_base 3, 7, 9, 12, 14, 15, 16, 18, 19: /mod_base = “2′-hydroxy corresponding nucleoside”
cugcucguguuacaggcggtt

SEQ ID NO: 385 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 8, 9, 10, 12, 13, 14, 16, 17, 18, 19: /mod_base = “2′-O-methyl corresponding nucleoside”
  modified_base 21: /mod_base = “5′-phosphorothioate thymidine”
  modified_base 4, 7, 11, 15: /mod_base = “2′-hydroxy corresponding nucleoside”
uauguugcccguuuguccutt

SEQ ID NO: 386 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 7, 9, 11, 12, 14, 15, 16, 18, 19: /mod_base = “2′-O-methyl corresponding nucleoside”
  modified_base 21: /mod_base = “5′-phosphorothioate thymidine”
  modified_base 3, 4, 5, 6, 8, 10, 13, 17: /mod_base = “2′-hydroxy corresponding nucleoside”
ucaagguauguugcccguutt

SEQ ID NO: 387 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 10, 12, 13, 14, 15, 16: /mod_base = “2′-O-methyl corresponding nucleoside”
  modified_base 21: /mod_base = “5′-phosphorothioate thymidine”
  modified_base 5, 8, 9, 11, 17, 18, 19: /mod_base = “2′-hydroxy corresponding nucleoside”
ucuuaucaacacuuccggatt

SEQ ID NO: 388 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
ucuuaucaacacuuccggat

SEQ ID NO: 389 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 6, 7, 9, 10, 11, 14, 15, 17, 18, 19: /mod_base = “2′-O-methyl corresponding nucleoside”
  modified_base 21: /mod_base = “5′-phosphorothioate thymidine”
  modified_base 8, 12, 13, 16: /mod_base = “2′-hydroxy corresponding nucleoside”
caccucugccuaaucaucutt

SEQ ID NO: 390 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 10, 11, 12, 13, 14, 18, 19: /mod_base = “2′-O-methyl corresponding nucleoside”
  modified_base 21: /mod_base = “5′-phosphorothioate thymidine”
  modified_base 3, 4, 5, 6, 7, 8, 9, 15, 16, 17: /mod_base = “2′-hydroxy corresponding nucleoside”
auaagaggacucuuggacutt

SEQ ID NO: 391 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
gucugcggcguuuuaucaat

SEQ ID NO: 392 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 10, 11, 12, 13, 15, 19: /mod_base = “2′-O-methyl corresponding nucleoside”
  modified_base 21: /mod_base = “5′-phosphorothioate thymidine”
  modified_base 4, 7, 8, 9, 14, 16, 17, 18: /mod_base = “2′-hydroxy corresponding nucleoside”
ggcgcugaaucccgcggactt

SEQ ID NO: 393 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 8, 15, 16, 17, 18: /mod_base = “2′-O-methyl corresponding nucleoside”
  modified_base 21: /mod_base = “5′-phosphorothioate thymidine”
  modified_base 4, 7, 9, 10, 11, 12, 13, 14, 19: /mod_base = “2′-hydroxy corresponding nucleoside”
cgcgucgcagaagaucucatt

SEQ ID NO: 394 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 9, 12, 13, 16, 17, 18, 19: /mod_base = “2′-O-methyl corresponding nucleoside”
  modified_base 21: /mod_base = “5′-phosphorothioate thymidine”
  modified_base 4, 7, 8, 10, 11, 14, 15: /mod_base = “2′-hydroxy corresponding nucleoside”
aaugucaacgaccgaccuutt

SEQ ID NO: 395 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 7, 8, 9, 11, 12, 15, 17, 18: /mod_base = “2′-O-methyl corresponding nucleoside”
  modified_base 21: /mod_base = “5′-phosphorothioate thymidine”
  modified_base 5, 6, 10, 13, 14, 16, 19: /mod_base = “2′-hydroxy corresponding nucleoside”
gcucaguuuacuagugccatt

SEQ ID NO: 396 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
ugguggacuucucucaauat

SEQ ID NO: 397 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 8, 10, 11, 13: /mod_base = “2′-O-methyl corresponding nucleoside”
  modified_base 21: /mod_base = “5′-phosphorothioate thymidine”
  modified_base 4, 7, 9, 12, 14, 15, 16, 17, 18, 19: /mod_base = “2′-hydroxy corresponding nucleoside”
aucgccgcgucgcagaagatt

SEQ ID NO: 398 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 7, 9, 10, 11, 14, 17, 18, 19: /mod_base = “2′-O-methyl corresponding nucleoside”
  modified_base 21: /mod_base = “5′-phosphorothioate thymidine”
  modified_base 4, 8, 12, 13, 15, 16: /mod_base = “2′-hydroxy corresponding nucleoside”
gccauuuguucagugguuctt

SEQ ID NO: 399 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 8, 10, 11, 13, 18, 19: /mod_base = “2′-O-methyl corresponding nucleoside”
  modified_base 21: /mod_base = “5′-phosphorothioate thymidine”
  modified_base 3, 7, 9, 12, 14, 15, 16, 17: /mod_base = “2′-hydroxy corresponding nucleoside”
cgauccauacugcggaacutt

SEQ ID NO: 400 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 7, 8, 10, 11, 12, 15, 16, 18, 19: /mod_base = “2′-O-methyl corresponding nucleoside”
  modified_base 21: /mod_base = “5′-phosphorothioate thymidine”
  modified_base 3, 9, 13, 14, 17: /mod_base = “2′-hydroxy corresponding nucleoside”
ucaccucugccuaaucauctt

SEQ ID NO: 401 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 6, 7, 8, 9, 10, 11, 12, 13, 16, 17, 18, 19: /mod_base = “2′-O-methyl corresponding nucleoside”
  modified_base 21: /mod_base = “5′-phosphorothioate thymidine”
  modified_base 3, 4, 5, 14, 15: /mod_base = “2′-hydroxy corresponding nucleoside”
guggacuucucucaauuuutt

SEQ ID NO: 402 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 7, 8, 10, 12, 13, 14, 15, 16: /mod_base = “2′-O-methyl corresponding nucleoside”
  modified_base 21: /mod_base = “5′-phosphorothioate thymidine”
  modified_base 3, 6, 9, 11, 17, 18, 19: /mod_base = “2′-hydroxy corresponding nucleoside”
gggucaccauauucuugggtt

SEQ ID NO: 403 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 7, 8, 10, 17, 18, 19: /mod_base = “2′-O-methyl corresponding nucleoside”
  modified_base 21: /mod_base = “5′-phosphorothioate thymidine”
  modified_base 4, 6, 9, 11, 12, 13, 14, 15, 16: /mod_base = “2′-hydroxy corresponding nucleoside”
gccgcgucgcagaagaucutt

SEQ ID NO: 404 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 6, 8, 9, 11, 13, 14, 16: /mod_base = “2′-O-methyl corresponding nucleoside”
  modified_base 21: /mod_base = “5′-phosphorothioate thymidine”
  modified_base 3, 4, 7, 10, 12, 15, 17, 18, 19: /mod_base = “2′-hydroxy corresponding nucleoside”
ucaaucgccgcgucgcagatt

SEQ ID NO: 405 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 7, 9, 10, 11, 13, 16, 18, 19: /mod_base = “2′-O-methyl corresponding nucleoside”
  modified_base 21: /mod_base = “5′-phosphorothioate thymidine”
  modified_base 3, 4, 6, 8, 12, 14, 15, 17: /mod_base = “2′-hydroxy corresponding nucleoside”
uggaugugucugcggcguutt

SEQ ID NO: 406 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 8, 12, 13, 14, 15, 16: /mod_base = “2′-O-methyl corresponding nucleoside”
  modified_base 21: /mod_base = “5′-phosphorothioate thymidine”
  modified_base 5, 9, 10, 11, 17, 18, 19: /mod_base = “2′-hydroxy corresponding nucleoside”
uacuguucaagccuccaagtt

SEQ ID NO: 407 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 10, 12, 13, 15, 16, 17, 19: /mod_base = “2′-O-methyl corresponding nucleoside”
  modified_base 21: /mod_base = “5′-phosphorothioate thymidine”
  modified_base 5, 8, 9, 11, 14, 18: /mod_base = “2′-hydroxy corresponding nucleoside”
guuuacuagugccauuugutt

SEQ ID NO: 408 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 6, 8, 9, 11, 12, 13, 15, 16, 17: /mod_base = “2′-O-methyl corresponding nucleoside”
  modified_base 21: /mod_base = “5′-phosphorothioate thymidine”
  modified_base 4, 5, 7, 10, 14, 18, 19: /mod_base = “2′-hydroxy corresponding nucleoside”
acuagugccauuuguucagtt

SEQ ID NO: 409 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 7, 9, 16, 17, 18, 19: /mod_base = “2′-O-methyl corresponding nucleoside”
  modified_base 21: /mod_base = “5′-phosphorothioate thymidine”
  modified_base 3, 5, 8, 10, 11, 12, 13, 14, 15: /mod_base = “2′-hydroxy corresponding nucleoside”
ccgcgucgcagaagaucuctt

SEQ ID NO: 410 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 6, 8, 9, 12, 14, 15, 16, 17, 18: /mod_base = “2′-O-methyl corresponding nucleoside”
  modified_base 21: /mod_base = “5′-phosphorothioate thymidine”
  modified_base 7, 10, 11, 13, 19: /mod_base = “2′-hydroxy corresponding nucleoside”
uaucuuaucaacacuuccgtt

SEQ ID NO: 411 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 9, 10, 11, 13, 16, 17, 18, 19: /mod_base = “2′-O-methyl corresponding nucleoside”
  modified_base 21: /mod_base = “5′-phosphorothioate thymidine”
  modified_base 5, 6, 7, 8, 12, 14, 15: /mod_base = “2′-hydroxy corresponding nucleoside”
ggccaaaauucgcaguccctt

SEQ ID NO: 412 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 7, 8, 9, 11, 12, 13, 16, 17, 19: /mod_base = “2′-O-methyl corresponding nucleoside”
  modified_base 21: /mod_base = “5′-phosphorothioate thymidine”
  modified_base 4, 10, 14, 15, 18: /mod_base = “2′-hydroxy corresponding nucleoside”
uucaccucugccuaaucautt

SEQ ID NO: 413 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 6, 7, 8, 10, 11, 14, 16, 17, 19: /mod_base = “2′-O-methyl corresponding nucleoside”
  modified_base 21: /mod_base = “5′-phosphorothioate thymidine”
  modified_base 4, 5, 9, 12, 13, 15, 18: /mod_base = “2′-hydroxy corresponding nucleoside”
cucaguuuacuagugccautt

SEQ ID NO: 414 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 8, 10, 11, 12, 14, 15, 16, 17, 18, 19: /mod_base = “2′-O-methyl corresponding nucleoside”
  modified_base 21: /mod_base = “5′-phosphorothioate thymidine”
  modified_base 5, 9, 13: /mod_base = “2′-hydroxy corresponding nucleoside”
uguugcccguuuguccucutt

SEQ ID NO: 415 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 7, 9, 10, 11, 13, 14, 15, 18: /mod_base = “2′-O-methyl corresponding nucleoside”
  modified_base 21: /mod_base = “5′-phosphorothioate thymidine”
  modified_base 3, 5, 8, 12, 16, 17, 19: /mod_base = “2′-hydroxy corresponding nucleoside”
uagugccauuuguucagugtt

SEQ ID NO: 416 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 7, 11, 13, 17, 18: /mod_base = “2′-O-methyl corresponding nucleoside”
  modified_base 21: /mod_base = “5′-phosphorothioate thymidine”
  modified_base 3, 6, 8, 9, 10, 12, 14, 15, 16, 19: /mod_base = “2′-hydroxy corresponding nucleoside”
aggcuguaggcauaaauugtt

SEQ ID NO: 417 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 7, 8, 10, 13, 15, 16, 17, 18: /mod_base = “2′-O-methyl corresponding nucleoside”
  modified_base 21: /mod_base = “5′-phosphorothioate thymidine”
  modified_base 3, 5, 9, 11, 12, 14, 19: /mod_base = “2′-hydroxy corresponding nucleoside”
augugucugcggcguuuuatt

SEQ ID NO: 418 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
augugucugcggcguuuuat

SEQ ID NO: 419 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 8, 9, 10, 12, 13, 14, 15, 16, 18: /mod_base = “2′-O-methyl corresponding nucleoside”
  modified_base 21: /mod_base = “5′-phosphorothioate thymidine”
  modified_base 6, 11, 17, 19: /mod_base = “2′-hydroxy corresponding nucleoside”
acuucgcuucaccucugcatt

SEQ ID NO: 420 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 7, 9, 10, 11, 12, 14, 15, 16, 17, 19: /mod_base = “2′-O-methyl corresponding nucleoside”
  modified_base 21: /mod_base = “5′-phosphorothioate thymidine”
  modified_base 4, 6, 8, 13, 18: /mod_base = “2′-hydroxy corresponding nucleoside”
cgugugcacuucgcuucactt

SEQ ID NO: 421 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 9, 10, 11, 12, 13, 14, 15, 16, 19: /mod_base = “2′-O-methyl corresponding nucleoside”
  modified_base 21: /mod_base = “5′-phosphorothioate thymidine”
  modified_base 3, 4, 6, 7, 8, 17, 18: /mod_base = “2′-hydroxy corresponding nucleoside”
gugguggacuucucucaautt

SEQ ID NO: 422 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 7, 9, 12, 14, 15, 16, 17, 19: /mod_base = “2′-O-methyl corresponding nucleoside”
  modified_base 21: /mod_base = “5′-phosphorothioate thymidine”
  modified_base 4, 8, 10, 11, 13, 18: /mod_base = “2′-hydroxy corresponding nucleoside”
ugugucugcggcguuuuautt

SEQ ID NO: 423 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 7, 9, 10, 12, 13, 14, 16, 17, 18: /mod_base = “2′-O-methyl corresponding nucleoside”
  modified_base 21: /mod_base = “5′-phosphorothioate thymidine”
  modified_base 3, 4, 6, 8, 11, 15, 19: /mod_base = “2′-hydroxy corresponding nucleoside”
aagguauguugcccguuugtt

SEQ ID NO: 424 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 8, 9, 12, 13, 14, 15: /mod_base = “2′-O-methyl corresponding nucleoside”
  modified_base 21: /mod_base = “5′-phosphorothioate thymidine”
  modified_base 3, 4, 6, 7, 10, 11, 16, 17, 18, 19: /mod_base = “2′-hydroxy corresponding nucleoside”
ucaacgaccgaccuugaggtt

SEQ ID NO: 425 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 11, 12, 13, 14, 15, 19: /mod_base = “2′-O-methyl corresponding nucleoside”
  modified_base 21: /mod_base = “5′-phosphorothioate thymidine”
  modified_base 4, 5, 6, 7, 8, 9, 10, 16, 17, 18: /mod_base = “2′-hydroxy corresponding nucleoside”
cauaagaggacucuuggactt

SEQ ID NO: 426 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 6, 9, 10, 13, 14, 15, 16: /mod_base = “2′-O-methyl corresponding nucleoside”
  modified_base 21: /mod_base = “5′-phosphorothioate thymidine”
  modified_base 4, 5, 7, 8, 11, 12, 17, 18, 19: /mod_base = “2′-hydroxy corresponding nucleoside”
gucaacgaccgaccuugagtt

SEQ ID NO: 427 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 7, 8, 14: /mod_base = “2′-O-methyl corresponding nucleoside”
  modified_base 21: /mod_base = “5′-phosphorothioate thymidine”
  modified_base 3, 9, 10, 11, 12, 13, 15, 16, 17, 18, 19: /mod_base = “2′-hydroxy corresponding nucleoside”
auauucuugggaacaagagtt

SEQ ID NO: 428 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 9, 10, 12, 16: /mod_base = “2′-O-methyl corresponding nucleoside”
  modified_base 21: /mod_base = “5′-phosphorothioate thymidine”
  modified_base 6, 8, 11, 13, 14, 15, 17, 18, 19: /mod_base = “2′-hydroxy corresponding nucleoside”
ugcucguguuacaggcgggtt

SEQ ID NO: 429 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 7, 8, 10, 12, 13, 15: /mod_base = “2′-O-methyl corresponding nucleoside”
  modified_base 21: /mod_base = “5′-phosphorothioate thymidine”
  modified_base 3, 6, 9, 11, 14, 16, 17, 18, 19: /mod_base = “2′-hydroxy corresponding nucleoside”
caaucgccgcgucgcagaatt

SEQ ID NO: 430 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 7, 11, 12, 13, 14, 15, 19: /mod_base = “2′-O-methyl corresponding nucleoside”
  modified_base 21: /mod_base = “5′-phosphorothioate thymidine”
  modified_base 4, 8, 9, 10, 16, 17, 18: /mod_base = “2′-hydroxy corresponding nucleoside”
acuguucaagccuccaagctt

SEQ ID NO: 431 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 8, 9, 11, 18, 19: /mod_base = “2′-O-methyl corresponding nucleoside”
  modified_base 21: /mod_base = “5′-phosphorothioate thymidine”
  modified_base 5, 7, 10, 12, 13, 14, 15, 16, 17: /mod_base = “2′-hydroxy corresponding nucleoside”
cgccgcgucgcagaagauctt

SEQ ID NO: 432 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 8, 9, 12, 15, 16, 17, 19: /mod_base = “2′-O-methyl corresponding nucleoside”
  modified_base 21: /mod_base = “5′-phosphorothioate thymidine”
  modified_base 6, 10, 11, 13, 14, 18: /mod_base = “2′-hydroxy corresponding nucleoside”
cauuuguucagugguucgutt

SEQ ID NO: 433 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 8, 9, 10, 11, 13, 17: /mod_base = “2′-O-methyl corresponding nucleoside”
  modified_base 21: /mod_base = “5′-phosphorothioate thymidine”
  modified_base 5, 6, 7, 12, 14, 15, 16, 18, 19: /mod_base = “2′-hydroxy corresponding nucleoside”
cgcugaaucccgcggacgatt

SEQ ID NO: 434 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 6, 8, 9, 11, 13, 14, 15, 16, 17: /mod_base = “2′-O-methyl corresponding nucleoside”
  modified_base 21: /mod_base = “5′-phosphorothioate thymidine”
  modified_base 3, 4, 7, 10, 12, 18, 19: /mod_base = “2′-hydroxy corresponding nucleoside”
ugggucaccauauucuuggtt

SEQ ID NO: 435 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 6, 8, 9, 12, 13, 14, 16, 18, 19: /mod_base = “2′-O-methyl corresponding nucleoside”
  modified_base 21: /mod_base = “5′-phosphorothioate thymidine”
  modified_base 7, 10, 11, 15, 17: /mod_base = “2′-hydroxy corresponding nucleoside”
uccucugccgauccauacutt

SEQ ID NO: 436 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 8, 11, 12, 15, 16, 17, 18: /mod_base = “2′-O-methyl corresponding nucleoside”
  modified_base 21: /mod_base = “5′-phosphorothioate thymidine”
  modified_base 3, 6, 7, 9, 10, 13, 14, 19: /mod_base = “2′-hydroxy corresponding nucleoside”
augucaacgaccgaccuugtt

SEQ ID NO: 437 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 8, 9, 12, 13, 15, 16, 17, 18: /mod_base = “2′-O-methyl corresponding nucleoside”
  modified_base 21: /mod_base = “5′-phosphorothioate thymidine”
  modified_base 6, 10, 11, 14, 19: /mod_base = “2′-hydroxy corresponding nucleoside”
ccucugccuaaucaucucatt

SEQ ID NO: 438 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 7, 9, 11, 12, 13, 14, 16, 17, 18, 19: /mod_base = “2′-O-methyl corresponding nucleoside”
  modified_base 21: /mod_base = “5′-phosphorothioate thymidine”
  modified_base 4, 6, 8, 10, 15: /mod_base = “2′-hydroxy corresponding nucleoside”
accgugugcacuucgcuuctt

SEQ ID NO: 439 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 7, 8, 9, 11, 13, 14, 16: /mod_base = “2′-O-methyl corresponding nucleoside”
  modified_base 21: /mod_base = “5′-phosphorothioate thymidine”
  modified_base 5, 6, 10, 12, 15, 17, 18, 19: /mod_base = “2′-hydroxy corresponding nucleoside”
ugccgauccauacugcggatt

SEQ ID NO: 440 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 6, 7, 8, 12, 13, 14, 16, 19: /mod_base = “2′-O-methyl corresponding nucleoside”
  modified_base 21: /mod_base = “5′-phosphorothioate thymidine”
  modified_base 3, 4, 5, 9, 10, 11, 15, 17, 18: /mod_base = “2′-hydroxy corresponding nucleoside”
cagagucuagacucguggutt

SEQ ID NO: 441 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 10, 11, 12, 13, 14, 18, 19: /mod_base = “2′-O-methyl corresponding nucleoside”
  modified_base 21: /mod_base = “5′-phosphorothioate thymidine”
  modified_base 3, 7, 8, 9, 15, 16, 17: /mod_base = “2′-hydroxy corresponding nucleoside”
cuguucaagccuccaagcutt

SEQ ID NO: 442 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 6, 7, 9, 13, 15, 19: /mod_base = “2′-O-methyl corresponding nucleoside”
  modified_base 21: /mod_base = “5′-phosphorothioate thymidine”
  modified_base 3, 4, 5, 8, 10, 11, 12, 14, 16, 17, 18: /mod_base = “2′-hydroxy corresponding nucleoside”
ggaggcuguaggcauaaautt

SEQ ID NO: 443 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 7, 8, 10, 14, 16: /mod_base = “2′-O-methyl corresponding nucleoside”
  modified_base 21: /mod_base = “5′-phosphorothioate thymidine”
  modified_base 3, 4, 5, 6, 9, 11, 12, 13, 15, 17, 18, 19: /mod_base = “2′-hydroxy corresponding nucleoside”
aggaggcuguaggcauaaatt

SEQ ID NO: 444 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 7, 8, 9, 10, 11, 12, 13, 14, 17, 18, 19: /mod_base = “2′-O-methyl corresponding nucleoside”
  modified_base 21: /mod_base = “5′-phosphorothioate thymidine”
  modified_base 4, 5, 6, 15, 16: /mod_base = “2′-hydroxy corresponding nucleoside”
gguggacuucucucaauuutt

SEQ ID NO: 445 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 6, 7, 8, 9, 10, 11, 13, 14, 15, 16, 17, 19: /mod_base = “2′-O-methyl corresponding nucleoside”
  modified_base 21: /mod_base = “5′-phosphorothioate thymidine”
  modified_base 3, 4, 12, 18: /mod_base = “2′-hydroxy corresponding nucleoside”
gcaacuuuuucaccucugctt

SEQ ID NO: 446 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
cugcucguguuacaggcgat

SEQ ID NO: 447 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 7, 8, 10, 11, 12, 14, 15, 16, 19: /mod_base = “2′-O-methyl corresponding nucleoside”
  modified_base 21: /mod_base = “5′-phosphorothioate thymidine”
  modified_base 3, 4, 6, 9, 13, 17, 18: /mod_base = “2′-hydroxy corresponding nucleoside”
cuagugccauuuguucagutt

SEQ ID NO: 448 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 8, 9, 10, 12, 14, 15, 17: /mod_base = “2′-O-methyl corresponding nucleoside”
  modified_base 21: /mod_base = “5′-phosphorothioate thymidine”
  modified_base 3, 6, 7, 11, 13, 16, 18, 19: /mod_base = “2′-hydroxy corresponding nucleoside”
cugccgauccauacugcggtt

SEQ ID NO: 449 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 8, 9, 10, 11, 13, 14, 15, 16, 18, 19: /mod_base = “2′-O-methyl corresponding nucleoside”
  modified_base 21: /mod_base = “5′-phosphorothioate thymidine”
  modified_base 3, 5, 7, 12, 17: /mod_base = “2′-hydroxy corresponding nucleoside”
gugugcacuucgcuucacctt

SEQ ID NO: 450 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 8, 9, 11, 15, 19: /mod_base = “2′-O-methyl corresponding nucleoside”
  modified_base 21: /mod_base = “5′-phosphorothioate thymidine”
  modified_base 5, 7, 10, 12, 13, 14, 16, 17, 18: /mod_base = “2′-hydroxy corresponding nucleoside”
gcucguguuacaggcgggctt

SEQ ID NO: 451 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 7, 8, 10, 11, 14, 16, 17, 18, 19: /mod_base = “2′-O-methyl corresponding nucleoside”
  modified_base 21: /mod_base = “5′-phosphorothioate thymidine”
  modified_base 4, 9, 12, 13, 15: /mod_base = “2′-hydroxy corresponding nucleoside”
ccuaucuuaucaacacuuctt

SEQ ID NO: 452 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 7, 8, 10, 11, 13, 15, 16, 18: /mod_base = “2′-O-methyl corresponding nucleoside”
  modified_base 21: /mod_base = “5′-phosphorothioate thymidine”
  modified_base 5, 6, 9, 12, 14, 17, 19: /mod_base = “2′-hydroxy corresponding nucleoside”
ucucaaucgccgcgucgcatt

SEQ ID NO: 453 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 8, 10, 12, 13, 14, 15, 16, 17, 18: /mod_base = “2′-O-methyl corresponding nucleoside”
  modified_base 21: /mod_base = “5′-phosphorothioate thymidine”
  modified_base 5, 9, 11, 19: /mod_base = “2′-hydroxy corresponding nucleoside”
gcccgucugugccuucucatt

SEQ ID NO: 454 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 7, 9, 10, 13, 15, 16, 17, 18, 19: /mod_base = “2′-O-methyl corresponding nucleoside”
  modified_base 21: /mod_base = “5′-phosphorothioate thymidine”
  modified_base 3, 8, 11, 12, 14: /mod_base = “2′-hydroxy corresponding nucleoside”
cuaucuuaucaacacuucctt

SEQ ID NO: 455 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 7, 8, 9, 11, 12, 13, 15, 16, 17, 18, 19: /mod_base = “2′-O-methyl corresponding nucleoside”
  modified_base 21: /mod_base = “5′-phosphorothioate thymidine”
  modified_base 3, 6, 10, 14: /mod_base = “2′-hydroxy corresponding nucleoside”
auguugcccguuuguccuctt

SEQ ID NO: 456 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 7, 9, 10, 11, 13, 14, 15, 17, 18, 19: /mod_base = “2′-O-methyl corresponding nucleoside”
  modified_base 21: /mod_base = “5′-phosphorothioate thymidine”
  modified_base 3, 5, 8, 12, 16: /mod_base = “2′-hydroxy corresponding nucleoside”
guauguugcccguuugucctt

SEQ ID NO: 457 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 8, 9, 11, 12, 13, 14, 15, 17, 19: /mod_base = “2′-O-methyl corresponding nucleoside”
  modified_base 21: /mod_base = “5′-phosphorothioate thymidine”
  modified_base 5, 10, 16, 18: /mod_base = “2′-hydroxy corresponding nucleoside”
cuucgcuucaccucugcactt

SEQ ID NO: 458 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 7, 8, 9, 10, 12, 13, 14, 15, 17, 18, 19: /mod_base = “2′-O-methyl corresponding nucleoside”
  modified_base 21: /mod_base = “5′-phosphorothioate thymidine”
  modified_base 4, 6, 11, 16: /mod_base = “2′-hydroxy corresponding nucleoside”
ugugcacuucgcuucaccutt

SEQ ID NO: 459 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 8, 9, 10, 12, 15, 16, 17, 18: /mod_base = “2′-O-methyl corresponding nucleoside”
  modified_base 21: /mod_base = “5′-phosphorothioate thymidine”
  modified_base 4, 5, 6, 7, 11, 13, 14, 19: /mod_base = “2′-hydroxy corresponding nucleoside”
gccaaaauucgcagucccgtt

SEQ ID NO: 460 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 7, 9, 11, 12, 14, 18: /mod_base = “2′-O-methyl corresponding nucleoside”
  modified_base 21: /mod_base = “5′-phosphorothioate thymidine”
  modified_base 4, 8, 10, 13, 15, 16, 17, 19: /mod_base = “2′-hydroxy corresponding nucleoside”
ccugcucguguuacaggcgtt

SEQ ID NO: 461 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 6, 8, 12, 13, 14, 16, 18, 19: /mod_base = “2′-O-methyl corresponding nucleoside”
  modified_base 21: /mod_base = “5′-phosphorothioate thymidine”
  modified_base 3, 4, 5, 7, 9, 10, 11, 15, 17: /mod_base = “2′-hydroxy corresponding nucleoside”
uggaguguggauucgcacutt

SEQ ID NO: 462 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 6, 7, 10, 11, 12, 13, 18: /mod_base = “2′-O-methyl corresponding nucleoside”
  modified_base 21: /mod_base = “5′-phosphorothioate thymidine”
  modified_base 4, 5, 8, 9, 14, 15, 16, 17, 19: /mod_base = “2′-hydroxy corresponding nucleoside”
aacgaccgaccuugaggcatt

SEQ ID NO: 463 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 7, 8, 9, 13, 14, 15, 17: /mod_base = “2′-O-methyl corresponding nucleoside”
  modified_base 21: /mod_base = “5′-phosphorothioate thymidine”
  modified_base 3, 4, 5, 6, 10, 11, 12, 16, 18, 19: /mod_base = “2′-hydroxy corresponding nucleoside”
acagagucuagacucguggtt

SEQ ID NO: 464 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 9, 11, 12, 14: /mod_base = “2′-O-methyl corresponding nucleoside”
  modified_base 21: /mod_base = “5′-phosphorothioate thymidine”
  modified_base 5, 8, 10, 13, 15, 16, 17, 18, 19: /mod_base = “2′-hydroxy corresponding nucleoside”
aaucgccgcgucgcagaagtt

SEQ ID NO: 465 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 7, 8, 10, 11, 12, 14, 15, 16, 18, 19: /mod_base = “2′-O-methyl corresponding nucleoside”
  modified_base 21: /mod_base = “5′-phosphorothioate thymidine”
  modified_base 4, 6, 9, 13, 17: /mod_base = “2′-hydroxy corresponding nucleoside”
gguauguugcccguuuguctt

SEQ ID NO: 466 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 6, 7, 8, 10, 12, 13, 15: /mod_base = “2′-O-methyl corresponding nucleoside”
  modified_base 21: /mod_base = “5′-phosphorothioate thymidine”
  modified_base 4, 5, 9, 11, 14, 16, 17, 18, 19: /mod_base = “2′-hydroxy corresponding nucleoside”
gccgauccauacugcggaatt

SEQ ID NO: 467 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 8, 9, 10, 12, 13, 16, 18, 19: /mod_base = “2′-O-methyl corresponding nucleoside”
  modified_base 21: /mod_base = “5′-phosphorothioate thymidine”
  modified_base 6, 11, 14, 15, 17: /mod_base = “2′-hydroxy corresponding nucleoside”
gcccuaucuuaucaacacutt

SEQ ID NO: 468 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 8, 11, 13, 14, 16, 17, 18: /mod_base = “2′-O-methyl corresponding nucleoside”
  modified_base 21: /mod_base = “5′-phosphorothioate thymidine”
  modified_base 6, 9, 10, 12, 15, 19: /mod_base = “2′-hydroxy corresponding nucleoside”
aguuuacuagugccauuugtt

SEQ ID NO: 469 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 7, 10, 11, 14, 15, 16, 17: /mod_base = “2′-O-methyl corresponding nucleoside”
  modified_base 21: /mod_base = “5′-phosphorothioate thymidine”
  modified_base 5, 6, 8, 9, 12, 13, 18, 19: /mod_base = “2′-hydroxy corresponding nucleoside”
ugucaacgaccgaccuugatt

SEQ ID NO: 470 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 6, 7, 8, 9, 12, 13, 14, 15, 16, 17: /mod_base = “2′-O-methyl corresponding nucleoside”
  modified_base 21: /mod_base = “5′-phosphorothioate thymidine”
  modified_base 10, 11, 18, 19: /mod_base = “2′-hydroxy corresponding nucleoside”
acuucucucaauuuucuagtt

SEQ ID NO: 471 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 9, 11, 12, 13, 14, 15, 16, 18, 19: /mod_base = “2′-O-methyl corresponding nucleoside”
  modified_base 21: /mod_base = “5′-phosphorothioate thymidine”
  modified_base 3, 5, 6, 7, 8, 10, 17: /mod_base = “2′-hydroxy corresponding nucleoside”
gcgcgggacguccuuuguctt

SEQ ID NO: 472 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 7, 8, 9, 11, 14, 18, 19: /mod_base = “2′-O-methyl corresponding nucleoside”
  modified_base 21: /mod_base = “5′-phosphorothioate thymidine”
  modified_base 4, 5, 6, 10, 12, 13, 15, 16, 17: /mod_base = “2′-hydroxy corresponding nucleoside”
ucuagacucgugguggacutt

SEQ ID NO: 473 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 9, 10, 12, 17, 18, 19: /mod_base = “2′-O-methyl corresponding nucleoside”
  modified_base 21: /mod_base = “5′-phosphorothioate thymidine”
  modified_base 6, 8, 11, 13, 14, 15, 16: /mod_base = “2′-hydroxy corresponding nucleoside”
gauccauacugcggaacuctt

SEQ ID NO: 474 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 10, 11, 12, 14, 16, 17, 19: /mod_base = “2′-O-methyl corresponding nucleoside”
  modified_base 21: /mod_base = “5′-phosphorothioate thymidine”
  modified_base 5, 8, 9, 13, 15, 18: /mod_base = “2′-hydroxy corresponding nucleoside”
cucugccgauccauacugctt

SEQ ID NO: 475 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 9, 10, 11, 13, 15, 16, 18: /mod_base = “2′-O-methyl corresponding nucleoside”
  modified_base 21: /mod_base = “5′-phosphorothioate thymidine”
  modified_base 4, 7, 8, 12, 14, 17, 19: /mod_base = “2′-hydroxy corresponding nucleoside”
ucugccgauccauacugcgtt

SEQ ID NO: 476 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 8, 11, 12, 13, 15, 17, 18: /mod_base = “2′-O-methyl corresponding nucleoside”
  modified_base 21: /mod_base = “5′-phosphorothioate thymidine”
  modified_base 6, 9, 10, 14, 16, 19: /mod_base = “2′-hydroxy corresponding nucleoside”
ccucugccgauccauacugtt

SEQ ID NO: 477 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 7, 8, 9, 10, 11, 12, 14, 16, 19: /mod_base = “2′-O-methyl corresponding nucleoside”
  modified_base 21: /mod_base = “5′-phosphorothioate thymidine”
  modified_base 3, 13, 15, 17, 18: /mod_base = “2′-hydroxy corresponding nucleoside”
gcaccucucuuuacgcggutt

SEQ ID NO: 478 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 6, 7, 8, 9, 10, 11, 12, 14, 15, 16, 17, 19: /mod_base = “2′-O-methyl corresponding nucleoside”
  modified_base 21: /mod_base = “5′-phosphorothioate thymidine”
  modified_base 3, 4, 5, 13, 18: /mod_base = “2′-hydroxy corresponding nucleoside”
aagaacucccucgccucgctt

SEQ ID NO: 479 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 7, 8, 9, 10, 12, 13, 14, 15, 17: /mod_base = “2′-O-methyl corresponding nucleoside”
  modified_base 21: /mod_base = “5′-phosphorothioate thymidine”
  modified_base 3, 11, 16, 18, 19: /mod_base = “2′-hydroxy corresponding nucleoside”
gaacucccucgccucgcagtt

SEQ ID NO: 480 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 6, 9, 10, 11, 12, 13, 14, 19: /mod_base = “2′-O-methyl corresponding nucleoside”
  modified_base 21: /mod_base = “5′-phosphorothioate thymidine”
  modified_base 7, 8, 15, 16, 17, 18: /mod_base = “2′-hydroxy corresponding nucleoside”
ucucucaauuuucuagggctt

SEQ ID NO: 481 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 8, 9, 10, 11, 12, 13, 14, 15, 16, 18: /mod_base = “2′-O-methyl corresponding nucleoside”
  modified_base 21: /mod_base = “5′-phosphorothioate thymidine”
  modified_base 3, 5, 7, 17, 19: /mod_base = “2′-hydroxy corresponding nucleoside”
gggcgcaccucucuuuacgtt

SEQ ID NO: 482 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 6, 7, 9, 11, 12, 14, 19: /mod_base = “2′-O-methyl corresponding nucleoside”
  modified_base 21: /mod_base = “5′-phosphorothioate thymidine”
  modified_base 3, 4, 8, 10, 13, 15, 16, 17, 18: /mod_base = “2′-hydroxy corresponding nucleoside”
ccgauccauacugcggaactt

SEQ ID NO: 483 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 6, 7, 8, 9, 11, 12, 13, 14, 16: /mod_base = “2′-O-methyl corresponding nucleoside”
  modified_base 21: /mod_base = “5′-phosphorothioate thymidine”
  modified_base 10, 15, 17, 18, 19: /mod_base = “2′-hydroxy corresponding nucleoside”
aacucccucgccucgcagatt

SEQ ID NO: 484 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 6, 7, 9, 10, 13, 14, 15, 17, 19: /mod_base = “2′-O-methyl corresponding nucleoside”
  modified_base 21: /mod_base = “5′-phosphorothioate thymidine”
  modified_base 8, 11, 12, 16, 18: /mod_base = “2′-hydroxy corresponding nucleoside”
cuccucugccgauccauactt

SEQ ID NO: 485 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 7, 11, 12, 13, 15, 17, 18, 19: /mod_base = “2′-O-methyl corresponding nucleoside”
  modified_base 21: /mod_base = “5′-phosphorothioate thymidine”
  modified_base 3, 4, 6, 8, 9, 10, 14, 16: /mod_base = “2′-hydroxy corresponding nucleoside”
ggaguguggauucgcacuctt

SEQ ID NO: 486 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 7, 9, 10, 11, 12, 13, 14, 15, 16, 17, 19: /mod_base = “2′-O-methyl corresponding nucleoside”
  modified_base 21: /mod_base = “5′-phosphorothioate thymidine”
  modified_base 3, 4, 6, 8, 18: /mod_base = “2′-hydroxy corresponding nucleoside”
cgggcgcaccucucuuuactt

SEQ ID NO: 487 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 8, 9, 11, 12, 14, 16, 17, 19: /mod_base = “2′-O-methyl corresponding nucleoside”
  modified_base 21: /mod_base = “5′-phosphorothioate thymidine”
  modified_base 6, 7, 10, 13, 15, 18: /mod_base = “2′-hydroxy corresponding nucleoside”
gucucaaucgccgcgucgctt

SEQ ID NO: 488 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 8, 9, 11, 16, 17, 18, 19: /mod_base = “2′-O-methyl corresponding nucleoside”
  modified_base 21: /mod_base = “5′-phosphorothioate thymidine”
  modified_base 5, 7, 10, 12, 13, 14, 15: /mod_base = “2′-hydroxy corresponding nucleoside”
auccauacugcggaacucctt

SEQ ID NO: 489 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 7, 8, 9, 10, 11, 12, 13, 15, 17: /mod_base = “2′-O-methyl corresponding nucleoside”
  modified_base 21: /mod_base = “5′-phosphorothioate thymidine”
  modified_base 4, 14, 16, 18, 19: /mod_base = “2′-hydroxy corresponding nucleoside”
cgcaccucucuuuacgcggtt

SEQ ID NO: 490 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 7, 8, 11, 12, 13, 14, 19: /mod_base = “2′-O-methyl corresponding nucleoside”
  modified_base 21: /mod_base = “5′-phosphorothioate thymidine”
  modified_base 3, 5, 6, 9, 10, 15, 16, 17, 18: /mod_base = “2′-hydroxy corresponding nucleoside”
caacgaccgaccuugaggctt

SEQ ID NO: 491 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 7, 9, 14, 15, 16, 17, 18: /mod_base = “2′-O-methyl corresponding nucleoside”
  modified_base 21: /mod_base = “5′-phosphorothioate thymidine”
  modified_base 3, 5, 8, 10, 11, 12, 13, 19: /mod_base = “2′-hydroxy corresponding nucleoside”
ccauacugcggaacuccuatt

SEQ ID NO: 492 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 6, 7, 8, 10, 14, 17, 18, 19: /mod_base = “2′-O-methyl corresponding nucleoside”
  modified_base 21: /mod_base = “5′-phosphorothioate thymidine”
  modified_base 3, 4, 9, 11, 12, 13, 15, 16: /mod_base = “2′-hydroxy corresponding nucleoside”
ugaaucccgcggacgaccctt

SEQ ID NO: 493 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 6, 7, 8, 9, 10, 11, 13, 14, 15, 16, 18: /mod_base = “2′-O-methyl corresponding nucleoside”
  modified_base 21: /mod_base = “5′-phosphorothioate thymidine”
  modified_base 3, 4, 12, 17, 19: /mod_base = “2′-hydroxy corresponding nucleoside”
agaacucccucgccucgcatt

SEQ ID NO: 494 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 7, 8, 9, 10, 11, 12, 13, 14, 15, 17, 19: /mod_base = “2′-O-methyl corresponding nucleoside”
  modified_base 21: /mod_base = “5′-phosphorothioate thymidine”
  modified_base 4, 6, 16, 18: /mod_base = “2′-hydroxy corresponding nucleoside”
ggcgcaccucucuuuacgctt

SEQ ID NO: 495 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 6, 7, 8, 9, 10, 11, 12, 13, 14, 16, 18: /mod_base = “2′-O-methyl corresponding nucleoside”
  modified_base 21: /mod_base = “5′-phosphorothioate thymidine”
  modified_base 3, 5, 15, 17, 19: /mod_base = “2′-hydroxy corresponding nucleoside”
gcgcaccucucuuuacgcgtt

SEQ ID NO: 496 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 7, 8, 9, 10, 12, 16, 19: /mod_base = “2′-O-methyl corresponding nucleoside”
  modified_base 21: /mod_base = “5′-phosphorothioate thymidine”
  modified_base 4, 5, 6, 11, 13, 14, 15, 17, 18: /mod_base = “2′-hydroxy corresponding nucleoside”
gcugaaucccgcggacgactt

SEQ ID NO: 497 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 6, 8, 9, 10, 11, 13, 14, 15, 16, 17, 19: /mod_base = “2′-O-methyl corresponding nucleoside”
  modified_base 21: /mod_base = “5′-phosphorothioate thymidine”
  modified_base 7, 12, 18: /mod_base = “2′-hydroxy corresponding nucleoside”
cacuucgcuucaccucugctt

SEQ ID NO: 498 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 6, 7, 9, 10, 12, 14, 15, 17: /mod_base = “2′-O-methyl corresponding nucleoside”
  modified_base 21: /mod_base = “5′-phosphorothioate thymidine”
  modified_base 4, 5, 8, 11, 13, 16, 18, 19: /mod_base = “2′-hydroxy corresponding nucleoside”
cucaaucgccgcgucgcagtt

SEQ ID NO: 499 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 10, 12, 13, 17, 18, 19: /mod_base = “2′-O-methyl corresponding nucleoside”
  modified_base 21: /mod_base = “5′-phosphorothioate thymidine”
  modified_base 5, 8, 9, 11, 14, 15, 16: /mod_base = “2′-hydroxy corresponding nucleoside”
ucccgucggcgcugaaucctt

SEQ ID NO: 500 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 6, 7, 8, 9, 11, 15, 18, 19: /mod_base = “2′-O-methyl corresponding nucleoside”
  modified_base 21: /mod_base = “5′-phosphorothioate thymidine”
  modified_base 3, 4, 5, 10, 12, 13, 14, 16, 17: /mod_base = “2′-hydroxy corresponding nucleoside”
cugaaucccgcggacgacctt

SEQ ID NO: 501 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 5, 6, 7, 11, 12, 13, 15, 18: /mod_base = “2′-O-methyl corresponding n ucleoside”
  modified_base 21: /mod_base = “5′-phosphorothioate thymidine”
  modified_base 3, 4, 8, 9, 10, 14, 16, 17, 19: /mod_base = “2′-hydroxy corresponding nucleoside”
agagucuagacucguggugtt

SEQ ID NO: 502 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 7, 8, 10, 15, 16, 17, 18, 19: /mod_base = “2′-O-methyl corresponding nucleoside”
  modified_base 21: /mod_base = “5′-phosphorothioate thymidine”
  modified_base 4, 6, 9, 11, 12, 13, 14: /mod_base = “2′-hydroxy corresponding nucleoside”
uccauacugcggaacuccutt

SEQ ID NO: 503 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 9, 10, 11, 12, 14, 18: /mod_base = “2′-O-methyl corresponding nucleoside”
  modified_base 21: /mod_base = “5′-phosphorothioate thymidine”
  modified_base 3, 6, 7, 8, 13, 15, 16, 17, 19: /mod_base = “2′-hydroxy corresponding nucleoside”
gcgcugaaucccgcggacgtt

SEQ ID NO: 504 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 9, 10, 11, 13, 15, 16, 17, 18, 19: /mod_base = “2′-O-methyl corresponding nucleoside”
  modified_base 21: /mod_base = “5′-phosphorothioate thymidine”
  modified_base 4, 6, 7, 8, 12, 14: /mod_base = “2′-hydroxy corresponding nucleoside”
aguguggauucgcacuccutt

SEQ ID NO: 505 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 6, 7, 8, 10, 12, 13, 15, 19: /mod_base = “2′-O-methyl corresponding nucleoside”
  modified_base 21: /mod_base = “5′-phosphorothioate thymidine”
  modified_base 5, 9, 11, 14, 16, 17, 18: /mod_base = “2′-hydroxy corresponding nucleoside”
cccugcucguguuacaggctt

SEQ ID NO: 506 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 6, 7, 9, 13, 16, 17, 18: /mod_base = “2′-O-methyl corresponding nucleoside”
  modified_base 21: /mod_base = “5′-phosphorothioate thymidine”
  modified_base 3, 8, 10, 11, 12, 14, 15, 19: /mod_base = “2′-hydroxy corresponding nucleoside”
gaaucccgcggacgacccgtt

SEQ ID NO: 507 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 4, 5, 7, 9, 10, 11, 12, 16, 19: /mod_base = “2′-O-methyl corresponding nucleoside”
  modified_base 21: /mod_base = “5′-phosphorothioate thymidine”
  modified_base 3, 6, 8, 13, 14, 15, 17, 18: /mod_base = “2′-hydroxy corresponding nucleoside”
aagcugugccuuggguggctt

SEQ ID NO: 508 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 8, 9, 11, 13, 14, 16: /mod_base = “2′-O-methyl corresponding nucleoside”
  modified_base 21: /mod_base = “5′-phosphorothioate thymidine”
  modified_base 6, 10, 12, 15, 17, 18, 19: /mod_base = “2′-hydroxy corresponding nucleoside”
gcccugcucguguuacaggtt

SEQ ID NO: 509 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 8, 11, 13, 14, 18, 19: /mod_base = “2′-O-methyl corresponding nucleoside”
  modified_base 21: /mod_base = “5′-phosphorothioate thymidine”
  modified_base 6, 9, 10, 12, 15, 16, 17: /mod_base = “2′-hydroxy corresponding nucleoside”
gucccgucggcgcugaauctt

SEQ ID NO: 510 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 4, 5, 7, 8, 11, 13, 14, 15, 16, 17: /mod_base = “2′-O-methyl corresponding nucleoside”
  modified_base 21: /mod_base = “5′-phosphorothioate thymidine”
  modified_base 6, 9, 10, 12, 18, 19: /mod_base = “2′-hydroxy corresponding nucleoside”
aucuuaucaacacuuccggtt

SEQ ID NO: 511 - DNA, 21 bp, Artificial sequence
  Description of the artificial sequence: sense strand of dsRNA
  modified_base 1: /mod_base = “nucleoside: lacks 5′-phosphate group”
  modified_base 1, 2, 3, 5, 6, 9, 11, 12, 13, 14, 15: /mod_base = “2′-O-methyl corresponding nucleoside”
  modified_base 21: /mod_base = “5′-phosphorothioate thymidine”
  modified_base 4, 7, 8, 10, 16, 17, 18, 19: /mod_base = “2′-hydroxy corresponding nucleoside”
cuuaucaacacuuccggaatt

SEQ ID NO: 512 - DNA, 20 bp, Artificial sequence
  Description of the artificial sequence: sense strand of dsRNA
  modified_base 1: /mod_base = “nucleoside: lacks 5′-phosphate group”
  modified_base 2, 4, 6, 8, 10, 12, 14, 16, 18: /mod_base = “2′-O-methyl corresponding nucleoside”
  modified_base 1, 3, 5, 7, 9, 11, 13, 15, 17, 19: /mod_base = “2′-deoxy-2′-fluoro corresponding nucleoside”
  modified_base 20: /mod_base = “3′-3′-linked desoxythymidine”
cuuaucaacacuuccggaat

SEQ ID NO: 513 - DNA, 21 bp, Artificial sequence
  Description of the artificial sequence: antisense strand of dsRNA
  modified_base 1: /mod_base = “nucleoside: lacks 5′-phosphate group”
  modified_base 8, 11, 13: /mod_base = “2′-O-methyl corresponding nucleoside”
  modified_base 21: /mod_base = “5′-phosphorothioate thymidine”
  modified_base 1, 2, 3, 4, 5, 6, 7, 9, 10, 12, 14, 15, 16, 17, 18, 19: /mod_base = “2′-hydroxy corresponding nucleoside”
aaacgggcaacauaccuugtt

SEQ ID NO: 514 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
taccaauuuaugccuacagtt

SEQ ID NO: 515 - DNA, 21 bp, Artificial sequence
  Description of the artificial sequence: antisense strand of dsRNA
  modified_base 1: /mod_base = “nucleoside: lacks 5′-phosphate group”
  modified_base 1, 6, 16: /mod_base = “2′-O-methyl corresponding nucleoside”
  modified_base 21: /mod_base = “5′-phosphorothioate thymidine”
  modified_base 2, 3, 4, 5, 7, 8, 9, 10, 11, 12, 13, 14, 15, 17, 18, 19: /mod_base = “2′-hydroxy corresponding nucleoside”
uaugauaaaacgccgcagatt

SEQ ID NO: 516 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
taugauaaaacgccgcagatt

SEQ ID NO: 517 - DNA, 21 bp, Artificial sequence
  Description of the artificial sequence: antisense strand of dsRNA
  modified_base 1: /mod_base = “nucleoside: lacks 5′-phosphate group”
  modified_base 9, 13: /mod_base = “2′-O-methyl corresponding nucleoside”
  modified_base 21: /mod_base = “5′-phosphorothioate thymidine”
  modified_base 1, 2, 3, 4, 5, 6, 7, 8, 10, 11, 12, 14, 15, 16, 17, 18,19: /mod_base = “2′-hydroxy corresponding nucleoside”
gagaugauuaggcagaggutt

SEQ ID NO: 518 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tacaaauggcacuaguaaatt

SEQ ID NO: 519 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tagaugauuaggcagaggutt

SEQ ID NO: 520 - DNA, 21 bp, Artificial sequence
  Description of the artificial sequence: antisense strand of dsRNA
  modified_base 1: /mod_base = “nucleoside: lacks 5′-phosphate group”
  modified_base 4, 9, 15, 17: /mod_base = “2′-O-methyl corresponding nucleoside”
  modified_base 21: /mod_base = “5′-phosphorothioate thymidine”
  modified_base 1, 2, 3, 5, 6, 7, 8, 10, 11, 12, 13, 14, 16, 18, 19: /mod_base = “2′-hydroxy corresponding nucleoside”
gaccaauuuaugccuacagtt

SEQ ID NO: 521 - DNA, 21 bp, Artificial sequence
  Description of the artificial sequence: antisense strand of dsRNA
  modified_base 1: /mod_base = “nucleoside: lacks 5′-phosphate group”
  modified_base 4, 14, 18: /mod_base = “2′-O-methyl corresponding nucleoside”
  modified_base 21: /mod_base = “5′-phosphorothioate thymidine”
  modified_base 1, 2, 3, 5, 6, 7, 8, 9, 10, 11, 12, 13, 15, 16, 17, 19: /mod_base = “2′-hydroxy corresponding nucleoside”
ugauaaaacgccgcagacatt

SEQ ID NO: 522 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tgauaaaacgccgcagacatt

SEQ ID NO: 523 - DNA, 21 bp, Artificial sequence
  Description of the artificial sequence: antisense strand of dsRNA
  modified_base 1: /mod_base = “nucleoside: lacks 5′-phosphate group”
  modified_base 5, 12, 15, 18: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 11, 13, 14, 16, 17, 19: /mod_base = “2′-hydroxy corresponding nucleoside”
ugaacaaauggcacuaguatt

SEQ ID NO: 524 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tgaacaaauggcacuaguatt

SEQ ID NO: 525 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tgcagaggugaaaaaguugtt

SEQ ID NO: 526 - DNA, 21 bp, Artificial sequence
  Description of the artificial sequence: antisense strand of dsRNA
  modified_base 1: /mod_base = “nucleoside: lacks 5′-phosphate group”
  modified_base 6, 13: /mod_base = “2′-O-methyl corresponding nucleoside”
  modified_base 21: /mod_base = “5′-phosphorothioate thymidine”
  modified_base 1, 2, 3, 4, 5, 7, 8, 9, 10, 11, 12, 14, 15, 16, 17, 18,19: /mod_base = “2′-hydroxy corresponding nucleoside”
cgaaccacugaacaaauggtt

SEQ ID NO: 527 - DNA, 21 bp, Artificial sequence
  Description of the artificial sequence: antisense strand of dsRNA
  modified_base 1: /mod_base = “nucleoside: lacks 5′-phosphate group”
  modified_base 6, 9, 13: /mod_base = “2′-O-methyl corresponding nucleoside”
  modified_base 21: /mod_base = “5′-phosphorothioate thymidine”
  modified_base 1, 2, 3, 4, 5, 7, 8, 10, 11, 12, 14, 15, 16, 17, 18, 19: /mod_base = “2′-hydroxy corresponding nucleoside”
ugcgucagcaaacacuuggtt

SEQ ID NO: 528 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tgcgucagcaaacacuuggtt

SEQ ID NO: 529 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tgaaccacugaacaaauggtt

SEQ ID NO: 530 - DNA, 21 bp, Artificial sequence
  Description of the artificial sequence: antisense strand of dsRNA
  modified_base 1: /mod_base = “nucleoside: lacks 5′-phosphate group”
  modified_base 3, 10, 13, 16: /mod_base = “2′-O-methyl corresponding nucleoside”
  modified_base 21: /mod_base = “5′-phosphorothioate thymidine”
  modified_base 1, 2, 4, 5, 6, 7, 8, 9, 11, 12, 14, 15, 17, 18, 19: /mod_base = “2′-hydroxy corresponding nucleoside”
aacaaauggcacuaguaaatt

SEQ ID NO: 531 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tgaugauuaggcagaggugtt

SEQ ID NO: 532 - DNA, 21 bp, Artificial sequence
  Description of the artificial sequence: antisense strand of dsRNA
  modified_base 1: /mod_base = “nucleoside: lacks 5′-phosphate group”
  modified_base 1, 4, 7, 17: /mod_base = “2′-O-methyl corresponding nucleoside”
  modified_base 21: /mod_base = “5′-phosphorothioate thymidine”
  modified_base 2, 3, 5, 6, 8, 9, 10, 11, 12, 13, 14, 15, 16, 18, 19: /mod_base = “2′-hydroxy corresponding nucleoside”
cacuaguaaacugagccagtt

SEQ ID NO: 533 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
taacgggcaacauaccuugtt

SEQ ID NO: 534 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tacuaguaaacugagccagtt

SEQ ID NO: 535 - DNA, 21 bp, Artificial sequence
  Description of the artificial sequence: antisense strand of dsRNA
  modified_base 1: /mod_base = “nucleoside: lacks 5′-phosphate group”
  modified_base 5, 11, 13: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 12, 14, 15, 16, 17, 18, 19: /mod_base = “2′-hydroxy corresponding nucleoside”
aauuuaugccuacagccuctt

SEQ ID NO: 536 - DNA, 21 bp, Artificial sequence
  Description of the artificial sequence: antisense strand of dsRNA
  modified_base 1: /mod_base = “nucleoside: lacks 5′-phosphate group”
  modified_base 7, 10, 13: /mod_base = “2′-O-methyl corresponding nucleoside”
  modified_base 21: /mod_base = “5′-phosphorothioate thymidine”
  modified_base 1, 2, 3, 4, 5, 6, 8, 9, 11, 12, 14, 15, 16, 17, 18, 19: /mod_base = “2′-hydroxy corresponding nucleoside”
aaauggcacuaguaaacugtt

SEQ ID NO: 537 - DNA, 21 bp, Artificial sequence
  Description of the artificial sequence: antisense strand of dsRNA
  modified_base 1: /mod_base = “nucleoside: lacks 5′-phosphate group”
  modified_base 2, 10, 13, 15: /mod_base = “2′-O-methyl corresponding nucleoside”
  modified_base 21: /mod_base = “5′-phosphorothioate thymidine”
  modified_base 1, 3, 4, 5, 6, 7, 8, 9, 11, 12, 14, 16, 17, 18, 19: /mod_base = “2′-hydroxy corresponding nucleoside”
acaaacgggcaacauaccutt

SEQ ID NO: 538 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tggacaaacgggcaacauatt

SEQ ID NO: 539 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tauuuaugccuacagccuctt

SEQ ID NO: 540 - DNA, 21 bp, Artificial sequence
  Description of the artificial sequence: antisense strand of dsRNA
  modified_base 1: /mod_base = “nucleoside: lacks 5′-phosphate group”
  modified_base 5, 15: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 11, 12, 13, 14, 16, 17, 18,19: /mod_base = “2′-hydroxy corresponding nucleoside”
augauaaaacgccgcagactt

SEQ ID NO: 541 - DNA, 21 bp, Artificial sequence
  Description of the artificial sequence: antisense strand of dsRNA
  modified_base 1: /mod_base = “nucleoside: lacks 5′-phosphate group”
  modified_base 3: /mod_base = “2′-O-methyl corresponding nucleoside”
  modified_base 21: /mod_base = “5′-phosphorothioate thymidine”
  modified_base 1, 2, 4, 5, 6, 7, 8, 9, 10, 11, 12, 13, 14, 15, 16, 17,18, 19: /mod_base = “2′-hydroxy corresponding nucleoside”
ggcagaggugaaaaaguugtt

SEQ ID NO: 542 - DNA, 21 bp, Artificial sequence
  Description of the artificial sequence: antisense strand of dsRNA
  modified_base 1: /mod_base = “nucleoside: lacks 5′-phosphate group”
  modified_base 13, 15: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 14, 16, 17, 18,19: /mod_base = “2′-hydroxy corresponding nucleoside”
ugaagcgaagugcacacggtt

SEQ ID NO: 543 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tgaagcgaagugcacacggtt

SEQ ID NO: 544 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tacgggcaacauaccuugatt

SEQ ID NO: 545 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
taauggcacuaguaaacugtt

SEQ ID NO: 546 - DNA, 21 bp, Artificial sequence
  Description of the artificial sequence: antisense strand of dsRNA
  modified_base 1: /mod_base = “nucleoside: lacks 5′-phosphate group”
  modified_base 15, 18: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 16, 17,19: /mod_base = “2′-hydroxy corresponding nucleoside”
aauugagagaaguccaccatt

SEQ ID NO: 547 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tcaaacgggcaacauaccutt

SEQ ID NO: 548 - DNA, 21 bp, Artificial sequence
  Description of the artificial sequence: antisense strand of dsRNA
  modified_base 1: /mod_base = “nucleoside: lacks 5′-phosphate group”
  modified_base 8, 11, 17: /mod_base = “2′-O-methyl corresponding nucleoside”
  modified_base 21: /mod_base = “5′-phosphorothioate thymidine”
  modified_base 1, 2, 3, 4, 5, 6, 7, 9, 10, 12, 13, 14, 15, 16, 18, 19: /mod_base = “2′-hydroxy corresponding nucleoside”
ccgccuguaacacgagcagtt

SEQ ID NO: 549 - DNA, 21 bp, Artificial sequence
  Description of the artificial sequence: antisense strand of dsRNA
  modified_base 1: /mod_base = “nucleoside: lacks 5′-phosphate group”
  modified_base 5, 13, 16, 18: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 11, 12, 14, 15, 17, 19: /mod_base = “2′-hydroxy corresponding nucleoside”
aggacaaacgggcaacauatt

SEQ ID NO: 550 - DNA, 21 bp, Artificial sequence
  Description of the artificial sequence: antisense strand of dsRNA
  modified_base 1: /mod_base = “nucleoside: lacks 5′-phosphate group”
  modified_base 7, 10, 12: /mod_base = “2′-O-methyl corresponding nucleoside”
  modified_base 21: /mod_base = “5′-phosphorothioate thymidine”
  modified_base 1, 2, 3, 4, 5, 6, 8, 9, 11, 13, 14, 15, 16, 17, 18, 19: /mod_base = “2′-hydroxy corresponding nucleoside”
aacgggcaacauaccuugatt

SEQ ID NO: 551 - DNA, 21 bp, Artificial sequence
  Description of the artificial sequence: antisense strand of dsRNA
  modified_base 1: /mod_base = “nucleoside: lacks 5′-phosphate group”
  modified_base 15: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 16, 17,18, 19: /mod_base = “2′-hydroxy corresponding nucleoside”
uccggaaguguugauaagatt

SEQ ID NO: 552 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tccggaaguguugauaagatt

SEQ ID NO: 553 - DNA, 21 bp, Artificial sequence
  Description of the artificial sequence: antisense strand of dsRNA
  modified_base 1: /mod_base = “nucleoside: lacks 5′-phosphate group”
  modified_base 8, 12: /mod_base = “2′-O-methyl corresponding nucleoside”
  modified_base 21: /mod_base = “5′-phosphorothioate thymidine”
  modified_base 1, 2, 3, 4, 5, 6, 7, 9, 10, 11, 13, 14, 15, 16, 17, 18,19: /mod_base = “2′-hydroxy corresponding nucleoside”
agaugauuaggcagaggugtt

SEQ ID NO: 554 - DNA, 21 bp, Artificial sequence
  Description of the artificial sequence: antisense strand of dsRNA
  modified_base 1: /mod_base = “nucleoside: lacks 5′-phosphate group”
  modified_base 5, 17: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 11, 12, 13, 14, 15, 16, 18,19: /mod_base = “2′-hydroxy corresponding nucleoside”
aguccaagaguccucuuautt

SEQ ID NO: 555 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tugauaaaacgccgcagactt

SEQ ID NO: 556 - DNA, 21 bp, Artificial sequence
  Description of the artificial sequence: antisense strand of dsRNA
  modified_base 1: /mod_base = “nucleoside: lacks 5′-phosphate group”
  modified_base 13: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 14, 15, 16, 17,18, 19: /mod_base = “2′-hydroxy corresponding nucleoside”
guccgcgggauucagcgcctt

SEQ ID NO: 557 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
ugagaucuucugcgacgcgtt

SEQ ID NO: 558 - DNA, 21 bp, Artificial sequence
  Description of the artificial sequence: antisense strand of dsRNA
  modified_base 1: /mod_base = “nucleoside: lacks 5′-phosphate group”
  modified_base 16: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 17,18, 19: /mod_base = “2′-hydroxy corresponding nucleoside”
aaggucggucguugacauutt

SEQ ID NO: 559 - DNA, 21 bp, Artificial sequence
  Description of the artificial sequence: antisense strand of dsRNA
  modified_base 1: /mod_base = “nucleoside: lacks 5′-phosphate group”
  modified_base 4, 7, 10: /mod_base = “2′-O-methyl corresponding nucleoside”
  modified_base 21: /mod_base = “5′-phosphorothioate thymidine”
  modified_base 1, 2, 3, 5, 6, 8, 9, 11, 12, 13, 14, 15, 16, 17, 18, 19: /mod_base = “2′-hydroxy corresponding nucleoside”
uggcacuaguaaacugagctt

SEQ ID NO: 560 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tauugagagaaguccaccatt

SEQ ID NO: 561 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
ucuucugcgacgcggcgautt

SEQ ID NO: 562 - DNA, 21 bp, Artificial sequence
  Description of the artificial sequence: antisense strand of dsRNA
  modified_base 1: /mod_base = “nucleoside: lacks 5′-phosphate group”
  modified_base 5, 12: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 11, 13, 14, 15, 16, 17, 18,19: /mod_base = “2′-hydroxy corresponding nucleoside”
gaaccacugaacaaauggctt

SEQ ID NO: 563 - DNA, 21 bp, Artificial sequence
  Description of the artificial sequence: antisense strand of dsRNA
  modified_base 1: /mod_base = “nucleoside: lacks 5′-phosphate group”
  modified_base 8, 11: /mod_base = “2′-O-methyl corresponding nucleoside”
  modified_base 21: /mod_base = “5′-phosphorothioate thymidine”
  modified_base 1, 2, 3, 4, 5, 6, 7, 9, 10, 12, 13, 14, 15, 16, 17, 18,19: /mod_base = “2′-hydroxy corresponding nucleoside”
aguuccgcaguauggaucgtt

SEQ ID NO: 564 - DNA, 21 bp, Artificial sequence
  Description of the artificial sequence: antisense strand of dsRNA
  modified_base 1: /mod_base = “nucleoside: lacks 5′-phosphate group”
  modified_base 7, 11: /mod_base = “2′-O-methyl corresponding nucleoside”
  modified_base 21: /mod_base = “5′-phosphorothioate thymidine”
  modified_base 1, 2, 3, 4, 5, 6, 8, 9, 10, 12, 13, 14, 15, 16, 17, 18,19: /mod_base = “2′-hydroxy corresponding nucleoside”
gaugauuaggcagaggugatt

SEQ ID NO: 565 - DNA, 21 bp, Artificial sequence
  Description of the artificial sequence: antisense strand of dsRNA
  modified_base 1: /mod_base = “nucleoside: lacks 5′-phosphate group”
  modified_base 17: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 16,18, 19: /mod_base = “2′-hydroxy corresponding nucleoside”
aaaauugagagaaguccactt

SEQ ID NO: 566 - DNA, 21 bp, Artificial sequence
  Description of the artificial sequence: antisense strand of dsRNA
  modified_base 1: /mod_base = “nucleoside: lacks 5′-phosphate group”
  modified_base 3, 9: /mod_base = “2′-O-methyl corresponding nucleoside”
  modified_base 21: /mod_base = “5′-phosphorothioate thymidine”
  modified_base 1, 2, 4, 5, 6, 7, 8, 10, 11, 12, 13, 14, 15, 16, 17,18, 19: /mod_base = “2′-hydroxy corresponding nucleoside”
cccaagaauauggugaccctt

SEQ ID NO: 567 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
agaucuucugcgacgcggctt

SEQ ID NO: 568 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
ucugcgacgcggcgauugatt

SEQ ID NO: 569 - DNA, 21 bp, Artificial sequence
  Description of the artificial sequence: antisense strand of dsRNA
  modified_base 1: /mod_base = “nucleoside: lacks 5′-phosphate group”
  modified_base 8, 12, 14, 18: /mod_base = “2′-O-methyl corresponding nucleoside”
  modified_base 21: /mod_base = “5′-phosphorothioate thymidine”
  modified_base 1, 2, 3, 4, 5, 6, 7, 9, 10, 11, 13, 15, 16, 17, 19: /mod_base = “2′-hydroxy corresponding nucleoside”
aacgccgcagacacauccatt

SEQ ID NO: 570 - DNA, 21 bp, Artificial sequence
  Description of the artificial sequence: antisense strand of dsRNA
  modified_base 1: /mod_base = “nucleoside: lacks 5′-phosphate group”
  modified_base 15, 18: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 16, 17,19: /mod_base = “2′-hydroxy corresponding nucleoside”
cuuggaggcuugaacaguatt

SEQ ID NO: 571 - DNA, 21 bp, Artificial sequence
  Description of the artificial sequence: antisense strand of dsRNA
  modified_base 1: /mod_base = “nucleoside: lacks 5′-phosphate group”
  modified_base 2, 9, 12, 15: /mod_base = “2′-O-methyl corresponding nucleoside”
  modified_base 21: /mod_base = “5′-phosphorothioate thymidine”
  modified_base 1, 3, 4, 5, 6, 7, 8, 10, 11, 13, 14, 16, 17, 18, 19: /mod_base = “2′-hydroxy corresponding nucleoside”
acaaauggcacuaguaaactt

SEQ ID NO: 572 - DNA, 21 bp, Artificial sequence
  Description of the artificial sequence: antisense strand of dsRNA
  modified_base 1: /mod_base = “nucleoside: lacks 5′-phosphate group”
  modified_base 6, 13, 16: /mod_base = “2′-O-methyl corresponding nucleoside”
  modified_base 21: /mod_base = “5′-phosphorothioate thymidine”
  modified_base 1, 2, 3, 4, 5, 7, 8, 9, 10, 11, 12, 14, 15, 17, 18, 19: /mod_base = “2′-hydroxy corresponding nucleoside”
cugaacaaauggcacuagutt

SEQ ID NO: 573 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
gagaucuucugcgacgcggtt

SEQ ID NO: 574 - DNA, 21 bp, Artificial sequence
  Description of the artificial sequence: antisense strand of dsRNA
  modified_base 1: /mod_base = “nucleoside: lacks 5′-phosphate group”
  modified_base 13, 18: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 14, 15, 16, 17,19: /mod_base = “2′-hydroxy corresponding nucleoside”
cggaaguguugauaagauatt

SEQ ID NO: 575 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
gggacugcgaauuuuggcctt

SEQ ID NO: 576 - DNA, 21 bp, Artificial sequence
  Description of the artificial sequence: antisense strand of dsRNA
  modified_base 1: /mod_base = “nucleoside: lacks 5′-phosphate group”
  modified_base 6, 10: /mod_base = “2′-O-methyl corresponding nucleoside”
  modified_base 21: /mod_base = “5′-phosphorothioate thymidine”
  modified_base 1, 2, 3, 4, 5, 7, 8, 9, 11, 12, 13, 14, 15, 16, 17, 18,19: /mod_base = “2′-hydroxy corresponding nucleoside”
augauuaggcagaggugaatt

SEQ ID NO: 577 - DNA, 21 bp, Artificial sequence
  Description of the artificial sequence: antisense strand of dsRNA
  modified_base 1: /mod_base = “nucleoside: lacks 5′-phosphate group”
  modified_base 5, 8, 11: /mod_base = “2′-O-methyl corresponding nucleoside”
  modified_base 21: /mod_base = “5′-phosphorothioate thymidine”
  modified_base 1, 2, 3, 4, 6, 7, 9, 10, 12, 13, 14, 15, 16, 17, 18, 19: /mod_base = “2′-hydroxy corresponding nucleoside”
auggcacuaguaaacugagtt

SEQ ID NO: 578 - DNA, 21 bp, Artificial sequence
  Description of the artificial sequence: antisense strand of dsRNA
  modified_base 1: /mod_base = “nucleoside: lacks 5′-phosphate group”
  modified_base 7, 15, 18: /mod_base = “2′-O-methyl corresponding nucleoside”
  modified_base 21: /mod_base = “5′-phosphorothioate thymidine”
  modified_base 1, 2, 3, 4, 5, 6, 8, 9, 10, 11, 12, 13, 14, 16, 17, 19: /mod_base = “2′-hydroxy corresponding nucleoside”
agaggacaaacgggcaacatt

SEQ ID NO: 579 - DNA, 21 bp, Artificial sequence
  Description of the artificial sequence: antisense strand of dsRNA
  modified_base 1: /mod_base = “nucleoside: lacks 5′-phosphate group”
  modified_base 1, 8, 15, 18: /mod_base = “2′-O-methyl corresponding nucleoside”
  modified_base 21: /mod_base = “5′-phosphorothioate thymidine”
  modified_base 2, 3, 4, 5, 6, 7, 9, 10, 11, 12, 13, 14, 16, 17, 19: /mod_base = “2′-hydroxy corresponding nucleoside”
cacugaacaaauggcacuatt

SEQ ID NO: 580 - DNA, 21 bp, Artificial sequence
  Description of the artificial sequence: antisense strand of dsRNA
  modified_base 1: /mod_base = “nucleoside: lacks 5′-phosphate group”
  modified_base 1, 6, 12, 14: /mod_base = “2′-O-methyl corresponding nucleoside”
  modified_base 21: /mod_base = “5′-phosphorothioate thymidine”
  modified_base 2, 3, 4, 5, 7, 8, 9, 10, 11, 13, 15, 16, 17, 18, 19: /mod_base = “2′-hydroxy corresponding nucleoside”
caauuuaugccuacagccutt

SEQ ID NO: 581 - DNA, 21 bp, Artificial sequence
  Description of the artificial sequence: antisense strand of dsRNA
  modified_base 1: /mod_base = “nucleoside: lacks 5′-phosphate group”
  modified_base 1, 11, 15, 17: /mod_base = “2′-O-methyl corresponding nucleoside”
  modified_base 21: /mod_base = “5′-phosphorothioate thymidine”
  modified_base 2, 3, 4, 5, 6, 7, 8, 9, 10, 12, 13, 14, 16, 18, 19: /mod_base = “2′-hydroxy corresponding nucleoside”
uaaaacgccgcagacacautt

SEQ ID NO: 582 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
taaaacgccgcagacacautt

SEQ ID NO: 583 - DNA, 21 bp, Artificial sequence
  Description of the artificial sequence: antisense strand of dsRNA
  modified_base 1: /mod_base = “nucleoside: lacks 5′-phosphate group”
  modified_base 3: /mod_base = “2′-O-methyl corresponding nucleoside”
  modified_base 21: /mod_base = “5′-phosphorothioate thymidine”
  modified_base 1, 2, 4, 5, 6, 7, 8, 9, 10, 11, 12, 13, 14, 15, 16, 17,18, 19: /mod_base = “2′-hydroxy corresponding nucleoside”
ugcagaggugaagcgaagutt

SEQ ID NO: 584 - DNA, 21 bp, Artificial sequence
  Description of the artificial sequence: antisense strand of dsRNA
  modified_base 1: /mod_base = “nucleoside: lacks 5′-phosphate group”
  modified_base 14, 16: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 15, 17, 18,19: /mod_base = “2′-hydroxy corresponding nucleoside”
gugaagcgaagugcacacgtt

SEQ ID NO: 585 - DNA, 21 bp, Artificial sequence
  Description of the artificial sequence: antisense strand of dsRNA
  modified_base 1: /mod_base = “nucleoside: lacks 5′-phosphate group”
  modified_base 14, 17: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 15, 16, 18,19: /mod_base = “2′-hydroxy corresponding nucleoside”
auugagagaaguccaccactt

SEQ ID NO: 586 - DNA, 21 bp, Artificial sequence
  Description of the artificial sequence: antisense strand of dsRNA
  modified_base 1: /mod_base = “nucleoside: lacks 5′-phosphate group”
  modified_base 2, 12, 16, 18: /mod_base = “2′-O-methyl corresponding nucleoside”
  modified_base 21: /mod_base = “5′-phosphorothioate thymidine”
  modified_base 1, 3, 4, 5, 6, 7, 8, 9, 10, 11, 13, 14, 15, 17, 19: /mod_base = “2′-hydroxy corresponding nucleoside”
auaaaacgccgcagacacatt

SEQ ID NO: 587 - DNA, 21 bp, Artificial sequence
  Description of the artificial sequence: antisense strand of dsRNA
  modified_base 1: /mod_base = “nucleoside: lacks 5′-phosphate group”
  modified_base 1, 9, 12, 14: /mod_base = “2′-O-methyl corresponding nucleoside”
  modified_base 21: /mod_base = “5′-phosphorothioate thymidine”
  modified_base 2, 3, 4, 5, 6, 7, 8, 10, 11, 13, 15, 16, 17, 18, 19: /mod_base = “2′-hydroxy corresponding nucleoside”
caaacgggcaacauaccuutt

SEQ ID NO: 588 - DNA, 21 bp, Artificial sequence
  Description of the artificial sequence: antisense strand of dsRNA
  modified_base 1: /mod_base = “nucleoside: lacks 5′-phosphate group”
  modified_base 4: /mod_base = “2′-O-methyl corresponding nucleoside”
  modified_base 21: /mod_base = “5′-phosphorothioate thymidine”
  modified_base 1, 2, 3, 5, 6, 7, 8, 9, 10, 11, 12, 13, 14, 15, 16, 17,18, 19: /mod_base = “2′-hydroxy corresponding nucleoside”
ccucaaggucggucguugatt

SEQ ID NO: 589 - DNA, 21 bp, Artificial sequence
  Description of the artificial sequence: antisense strand of dsRNA
  modified_base 1: /mod_base = “nucleoside: lacks 5′-phosphate group”
  modified_base 4, 16: /mod_base = “2′-O-methyl corresponding nucleoside”
  modified_base 21: /mod_base = “5′-phosphorothioate thymidine”
  modified_base 1, 2, 3, 5, 6, 7, 8, 9, 10, 11, 12, 13, 14, 15, 17, 18,19: /mod_base = “2′-hydroxy corresponding nucleoside”
guccaagaguccucuuaugtt

SEQ ID NO: 590 - DNA, 21 bp, Artificial sequence
  Description of the artificial sequence: antisense strand of dsRNA
  modified_base 1: /mod_base = “nucleoside: lacks 5′-phosphate group”
  modified_base 3: /mod_base = “2′-O-methyl corresponding nucleoside”
  modified_base 21: /mod_base = “5′-phosphorothioate thymidine”
  modified_base 1, 2, 4, 5, 6, 7, 8, 9, 10, 11, 12, 13, 14, 15, 16, 17,18, 19: /mod_base = “2′-hydroxy corresponding nucleoside”
cucaaggucggucguugactt

SEQ ID NO: 591 - DNA, 21 bp, Artificial sequence
  Description of the artificial sequence: antisense strand of dsRNA
  modified_base 1: /mod_base = “nucleoside: lacks 5′-phosphate group”
  modified_base 11, 17: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 12, 13, 14, 15, 16, 18,19: /mod_base = “2′-hydroxy corresponding nucleoside”
cucuuguucccaagaauautt

SEQ ID NO: 592 - DNA, 21 bp, Artificial sequence
  Description of the artificial sequence: antisense strand of dsRNA
  modified_base 1: /mod_base = “nucleoside: lacks 5′-phosphate group”
  modified_base 9, 12, 18: /mod_base = “2′-O-methyl corresponding nucleoside”
  modified_base 21: /mod_base = “5′-phosphorothioate thymidine”
  modified_base 1, 2, 3, 4, 5, 6, 7, 8, 10, 11, 13, 14, 15, 16, 17, 19: /mod_base = “2′-hydroxy corresponding nucleoside”
cccgccuguaacacgagcatt

SEQ ID NO: 593 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
uucugcgacgcggcgauugtt

SEQ ID NO: 594 - DNA, 21 bp, Artificial sequence
  Description of the artificial sequence: antisense strand of dsRNA
  modified_base 1: /mod_base = “nucleoside: lacks 5′-phosphate group”
  modified_base 16: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 17,18, 19: /mod_base = “2′-hydroxy corresponding nucleoside”
gcuuggaggcuugaacagutt

SEQ ID NO: 595 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
gaucuucugcgacgcggcgtt

SEQ ID NO: 596 - DNA, 21 bp, Artificial sequence
  Description of the artificial sequence: antisense strand of dsRNA
  modified_base 1: /mod_base = “nucleoside: lacks 5′-phosphate group”
  modified_base 7, 14: /mod_base = “2′-O-methyl corresponding nucleoside”
  modified_base 21: /mod_base = “5′-phosphorothioate thymidine”
  modified_base 1, 2, 3, 4, 5, 6, 8, 9, 10, 11, 12, 13, 15, 16, 17, 18,19: /mod_base = “2′-hydroxy corresponding nucleoside”
acgaaccacugaacaaaugtt

SEQ ID NO: 597 - DNA, 21 bp, Artificial sequence
  Description of the artificial sequence: antisense strand of dsRNA
  modified_base 1: /mod_base = “nucleoside: lacks 5′-phosphate group”
  modified_base 15: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 16, 17,18, 19: /mod_base = “2′-hydroxy corresponding nucleoside”
ucguccgcgggauucagcgtt

SEQ ID NO: 598 - DNA, 21 bp, Artificial sequence
  Description of the artificial sequence: antisense strand of dsRNA
  modified_base 1: /mod_base = “nucleoside: lacks 5′-phosphate group”
  modified_base 2, 8, 18: /mod_base = “2′-O-methyl corresponding nucleoside”
  modified_base 21: /mod_base = “5′-phosphorothioate thymidine”
  modified_base 1, 3, 4, 5, 6, 7, 9, 10, 11, 12, 13, 14, 15, 16, 17, 19: /mod_base = “2′-hydroxy corresponding nucleoside”
ccaagaauauggugacccatt

SEQ ID NO: 599 - DNA, 21 bp, Artificial sequence
  Description of the artificial sequence: antisense strand of dsRNA
  modified_base 1: /mod_base = “nucleoside: lacks 5′-phosphate group”
  modified_base 3, 13: /mod_base = “2′-O-methyl corresponding nucleoside”
  modified_base 21: /mod_base = “5′-phosphorothioate thymidine”
  modified_base 1, 2, 4, 5, 6, 7, 8, 9, 10, 11, 12, 14, 15, 16, 17, 18,19: /mod_base = “2′-hydroxy corresponding nucleoside”
aguauggaucggcagaggatt

SEQ ID NO: 600 - DNA, 21 bp, Artificial sequence
  Description of the artificial sequence: antisense strand of dsRNA
  modified_base 1: /mod_base = “nucleoside: lacks 5′-phosphate group”
  modified_base 1, 17: /mod_base = “2′-O-methyl corresponding nucleoside”
  modified_base 21: /mod_base = “5′-phosphorothioate thymidine”
  modified_base 2, 3, 4, 5, 6, 7, 8, 9, 10, 11, 12, 13, 14, 15, 16, 18,19: /mod_base = “2′-hydroxy corresponding nucleoside”
caaggucggucguugacautt

SEQ ID NO: 601 - DNA, 21 bp, Artificial sequence
  Description of the artificial sequence: antisense strand of dsRNA
  modified_base 1: /mod_base = “nucleoside: lacks 5′-phosphate group”
  modified_base 10, 14: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 11, 12, 13, 15, 16, 17, 18,19: /mod_base = “2′-hydroxy corresponding nucleoside”
ugagaugauuaggcagaggtt

SEQ ID NO: 602 - DNA, 21 bp, Artificial sequence
  Description of the artificial sequence: antisense strand of dsRNA
  modified_base 1: /mod_base = “nucleoside: lacks 5′-phosphate group”
  modified_base 12, 14: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 13, 15, 16, 17, 18,19: /mod_base = “2′-hydroxy corresponding nucleoside”
gaagcgaagugcacacggutt

SEQ ID NO: 603 - DNA, 21 bp, Artificial sequence
  Description of the artificial sequence: antisense strand of dsRNA
  modified_base 1: /mod_base = “nucleoside: lacks 5′-phosphate group”
  modified_base 5, 8, 18: /mod_base = “2′-O-methyl corresponding nucleoside”
  modified_base 21: /mod_base = “5′-phosphorothioate thymidine”
  modified_base 1, 2, 3, 4, 6, 7, 9, 10, 11, 12, 13, 14, 15, 16, 17, 19: /mod_base = “2′-hydroxy corresponding nucleoside”
uccgcaguauggaucggcatt

SEQ ID NO: 604 - DNA, 21 bp, Artificial sequence
  Description of the artificial sequence: antisense strand of dsRNA
  modified_base 1: /mod_base = “nucleoside: lacks 5′-phosphate group”
  modified_base 3, 11: /mod_base = “2′-O-methyl corresponding nucleoside”
  modified_base 21: /mod_base = “5′-phosphorothioate thymidine”
  modified_base 1, 2, 4, 5, 6, 7, 8, 9, 10, 12, 13, 14, 15, 16, 17, 18,19: /mod_base = “2′-hydroxy corresponding nucleoside”
accacgagucuagacucugtt

SEQ ID NO: 605 - DNA, 21 bp, Artificial sequence
  Description of the artificial sequence: antisense strand of dsRNA
  modified_base 1: /mod_base = “nucleoside: lacks 5′-phosphate group”
  modified_base 17: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 16,18, 19: /mod_base = “2′-hydroxy corresponding nucleoside”
agcuuggaggcuugaacagtt

SEQ ID NO: 606 - DNA, 21 bp, Artificial sequence
  Description of the artificial sequence: antisense strand of dsRNA
  modified_base 1: /mod_base = “nucleoside: lacks 5′-phosphate group”
  modified_base 4, 10, 12: /mod_base = “2′-O-methyl corresponding nucleoside”
  modified_base 21: /mod_base = “5′-phosphorothioate thymidine”
  modified_base 1, 2, 3, 5, 6, 7, 8, 9, 11, 13, 14, 15, 16, 17, 18, 19: /mod_base = “2′-hydroxy corresponding nucleoside”
auuuaugccuacagccucctt

SEQ ID NO: 607 - DNA, 21 bp, Artificial sequence
  Description of the artificial sequence: antisense strand of dsRNA
  modified_base 1: /mod_base = “nucleoside: lacks 5′-phosphate group”
  modified_base 3, 9, 11: /mod_base = “2′-O-methyl corresponding nucleoside”
  modified_base 21: /mod_base = “5′-phosphorothioate thymidine”
  modified_base 1, 2, 4, 5, 6, 7, 8, 10, 12, 13, 14, 15, 16, 17, 18, 19: /mod_base = “2′-hydroxy corresponding nucleoside”
uuuaugccuacagccuccutt

SEQ ID NO: 608 - DNA, 21 bp, Artificial sequence
  Description of the artificial sequence: antisense strand of dsRNA
  modified_base 1: /mod_base = “nucleoside: lacks 5′-phosphate group”
  modified_base 16: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 17,18, 19: /mod_base = “2′-hydroxy corresponding nucleoside”
aaauugagagaaguccacctt

SEQ ID NO: 609 - DNA, 21 bp, Artificial sequence
  Description of the artificial sequence: antisense strand of dsRNA
  modified_base 1: /mod_base = “nucleoside: lacks 5′-phosphate group”
  modified_base 2: /mod_base = “2′-O-methyl corresponding nucleoside”
  modified_base 21: /mod_base = “5′-phosphorothioate thymidine”
  modified_base 1, 3, 4, 5, 6, 7, 8, 9, 10, 11, 12, 13, 14, 15, 16, 17,18, 19: /mod_base = “2′-hydroxy corresponding nucleoside”
gcagaggugaaaaaguugctt

SEQ ID NO: 610 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tcgccuguaacacgagcagtt

SEQ ID NO: 611 - DNA, 21 bp, Artificial sequence
  Description of the artificial sequence: antisense strand of dsRNA
  modified_base 1: /mod_base = “nucleoside: lacks 5′-phosphate group”
  modified_base 7, 14, 17: /mod_base = “2′-O-methyl corresponding nucleoside”
  modified_base 21: /mod_base = “5′-phosphorothioate thymidine”
  modified_base 1, 2, 3, 4, 5, 6, 8, 9, 10, 11, 12, 13, 15, 16, 18, 19: /mod_base = “2′-hydroxy corresponding nucleoside”
acugaacaaauggcacuagtt

SEQ ID NO: 612 - DNA, 21 bp, Artificial sequence
  Description of the artificial sequence: antisense strand of dsRNA
  modified_base 1: /mod_base = “nucleoside: lacks 5′-phosphate group”
  modified_base 4, 7, 17: /mod_base = “2′-O-methyl corresponding nucleoside”
  modified_base 21: /mod_base = “5′-phosphorothioate thymidine”
  modified_base 1, 2, 3, 5, 6, 8, 9, 10, 11, 12, 13, 14, 15, 16, 18,19: /mod_base = “2′-hydroxy corresponding nucleoside”
ccgcaguauggaucggcagtt

SEQ ID NO: 613 - DNA, 21 bp, Artificial sequence
  Description of the artificial sequence: antisense strand of dsRNA
  modified_base 1: /mod_base = “nucleoside: lacks 5′-phosphate group”
  modified_base 15, 17: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 16, 18,19: /mod_base = “2′-hydroxy corresponding nucleoside”
ggugaagcgaagugcacactt

SEQ ID NO: 614 - DNA, 21 bp, Artificial sequence
  Description of the artificial sequence: antisense strand of dsRNA
  modified_base 1: /mod_base = “nucleoside: lacks 5′-phosphate group”
  modified_base 10, 13: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 11, 12, 14, 15, 16, 17, 18,19: /mod_base = “2′-hydroxy corresponding nucleoside”
gcccgccuguaacacgagctt

SEQ ID NO: 615 - DNA, 21 bp, Artificial sequence
  Description of the artificial sequence: antisense strand of dsRNA
  modified_base 1: /mod_base = “nucleoside: lacks 5′-phosphate group”
  modified_base 11, 16: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 12, 13, 14, 15, 17, 18,19: /mod_base = “2′-hydroxy corresponding nucleoside”
gaaguguugauaagauaggtt

SEQ ID NO: 616 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
ugcgacgcggcgauugagatt

SEQ ID NO: 617 - DNA, 21 bp, Artificial sequence
  Description of the artificial sequence: antisense strand of dsRNA
  modified_base 1: /mod_base = “nucleoside: lacks 5′-phosphate group”
  modified_base 9, 11: /mod_base = “2′-O-methyl corresponding nucleoside”
  modified_base 21: /mod_base = “5′-phosphorothioate thymidine”
  modified_base 1, 2, 3, 4, 5, 6, 7, 8, 10, 12, 13, 14, 15, 16, 17, 18,19: /mod_base = “2′-hydroxy corresponding nucleoside”
ugagaaggcacagacgggctt

SEQ ID NO: 618 - DNA, 21 bp, Artificial sequence
  Description of the artificial sequence: antisense strand of dsRNA
  modified_base 1: /mod_base = “nucleoside: lacks 5′-phosphate group”
  modified_base 12, 17: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 13, 14, 15, 16, 18,19: /mod_base = “2′-hydroxy corresponding nucleoside”
ggaaguguugauaagauagtt

SEQ ID NO: 619 - DNA, 21 bp, Artificial sequence
  Description of the artificial sequence: antisense strand of dsRNA
  modified_base 1: /mod_base = “nucleoside: lacks 5′-phosphate group”
  modified_base 6, 14, 17: /mod_base = “2′-O-methyl corresponding nucleoside”
  modified_base 21: /mod_base = “5′-phosphorothioate thymidine”
  modified_base 1, 2, 3, 4, 5, 7, 8, 9, 10, 11, 12, 13, 15, 16, 18, 19: /mod_base = “2′-hydroxy corresponding nucleoside”
gaggacaaacgggcaacautt

SEQ ID NO: 620 - DNA, 21 bp, Artificial sequence
  Description of the artificial sequence: antisense strand of dsRNA
  modified_base 1: /mod_base = “nucleoside: lacks 5′-phosphate group”
  modified_base 4, 12, 15, 17: /mod_base = “2′-O-methyl corresponding nucleoside”
  modified_base 21: /mod_base = “5′-phosphorothioate thymidine”
  modified_base 1, 2, 3, 5, 6, 7, 8, 9, 10, 11, 13, 14, 16, 18, 19: /mod_base = “2′-hydroxy corresponding nucleoside”
ggacaaacgggcaacauactt

SEQ ID NO: 621 - DNA, 21 bp, Artificial sequence
  Description of the artificial sequence: antisense strand of dsRNA
  modified_base 1: /mod_base = “nucleoside: lacks 5′-phosphate group”
  modified_base 4: /mod_base = “2′-O-methyl corresponding nucleoside”
  modified_base 21: /mod_base = “5′-phosphorothioate thymidine”
  modified_base 1, 2, 3, 5, 6, 7, 8, 9, 10, 11, 12, 13, 14, 15, 16, 17,18, 19: /mod_base = “2′-hydroxy corresponding nucleoside”
gugcagaggugaagcgaagtt

SEQ ID NO: 622 - DNA, 21 bp, Artificial sequence
  Description of the artificial sequence: antisense strand of dsRNA
  modified_base 1: /mod_base = “nucleoside: lacks 5′-phosphate group”
  modified_base 16, 18: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 17,19: /mod_base = “2′-hydroxy corresponding nucleoside”
aggugaagcgaagugcacatt

SEQ ID NO: 623 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
cgggacugcgaauuuuggctt

SEQ ID NO: 624 - DNA, 21 bp, Artificial sequence
  Description of the artificial sequence: antisense strand of dsRNA
  modified_base 1: /mod_base = “nucleoside: lacks 5′-phosphate group”
  modified_base 7, 10, 16: /mod_base = “2′-O-methyl corresponding nucleoside”
  modified_base 21: /mod_base = “5′-phosphorothioate thymidine”
  modified_base 1, 2, 3, 4, 5, 6, 8, 9, 11, 12, 13, 14, 15, 17, 18, 19: /mod_base = “2′-hydroxy corresponding nucleoside”
cgccuguaacacgagcaggtt

SEQ ID NO: 625 - DNA, 21 bp, Artificial sequence
  Description of the artificial sequence: antisense strand of dsRNA
  modified_base 1: /mod_base = “nucleoside: lacks 5′-phosphate group”
  modified_base 11, 13, 18: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 12, 14, 15, 16, 17, 19: /mod_base = “2′-hydroxy corresponding nucleoside”
agugcgaauccacacuccatt

SEQ ID NO: 626 - DNA, 21 bp, Artificial sequence
  Description of the artificial sequence: antisense strand of dsRNA
  modified_base 1: /mod_base = “nucleoside: lacks 5′-phosphate group”
  modified_base 6: /mod_base = “2′-O-methyl corresponding nucleoside”
  modified_base 21: /mod_base = “5′-phosphorothioate thymidine”
  modified_base 1, 2, 3, 4, 5, 7, 8, 9, 10, 11, 12, 13, 14, 15, 16, 17,18, 19: /mod_base = “2′-hydroxy corresponding nucleoside”
ugccucaaggucggucguutt

SEQ ID NO: 627 - DNA, 21 bp, Artificial sequence
  Description of the artificial sequence: antisense strand of dsRNA
  modified_base 1: /mod_base = “nucleoside: lacks 5′-phosphate group”
  modified_base 2, 10: /mod_base = “2′-O-methyl corresponding nucleoside”
  modified_base 21: /mod_base = “5′-phosphorothioate thymidine”
  modified_base 1, 3, 4, 5, 6, 7, 8, 9, 11, 12, 13, 14, 15, 16, 17, 18,19: /mod_base = “2′-hydroxy corresponding nucleoside”
ccacgagucuagacucugutt

SEQ ID NO: 628 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
cuucugcgacgcggcgauutt

SEQ ID NO: 629 - DNA, 21 bp, Artificial sequence
  Description of the artificial sequence: antisense strand of dsRNA
  modified_base 1: /mod_base = “nucleoside: lacks 5′-phosphate group”
  modified_base 3, 11, 14, 16: /mod_base = “2′-O-methyl corresponding nucleoside”
  modified_base 21: /mod_base = “5′-phosphorothioate thymidine”
  modified_base 1, 2, 4, 5, 6, 7, 8, 9, 10, 12, 13, 15, 17, 18, 19: /mod_base = “2′-hydroxy corresponding nucleoside”
gacaaacgggcaacauacctt

SEQ ID NO: 630 - DNA, 21 bp, Artificial sequence
  Description of the artificial sequence: antisense strand of dsRNA
  modified_base 1: /mod_base = “nucleoside: lacks 5′-phosphate group”
  modified_base 6, 9: /mod_base = “2′-O-methyl corresponding nucleoside”
  modified_base 21: /mod_base = “5′-phosphorothioate thymidine”
  modified_base 1, 2, 3, 4, 5, 7, 8, 10, 11, 12, 13, 14, 15, 16, 17,18, 19: /mod_base = “2′-hydroxy corresponding nucleoside”
uuccgcaguauggaucggctt

SEQ ID NO: 631 - DNA, 21 bp, Artificial sequence
  Description of the artificial sequence: antisense strand of dsRNA
  modified_base 1: /mod_base = “nucleoside: lacks 5′-phosphate group”
  modified_base 9, 14: /mod_base = “2′-O-methyl corresponding nucleoside”
  modified_base 21: /mod_base = “5′-phosphorothioate thymidine”
  modified_base 1, 2, 3, 4, 5, 6, 7, 8, 10, 11, 12, 13, 15, 16, 17, 18,19: /mod_base = “2′-hydroxy corresponding nucleoside”
aguguugauaagauagggctt

SEQ ID NO: 632 - DNA, 21 bp, Artificial sequence
  Description of the artificial sequence: antisense strand of dsRNA
  modified_base 1: /mod_base = “nucleoside: lacks 5′-phosphate group”
  modified_base 1, 8, 11, 14: /mod_base = “2′-O-methyl corresponding nucleoside”
  modified_base 21: /mod_base = “5′-phosphorothioate thymidine”
  modified_base 2, 3, 4, 5, 6, 7, 9, 10, 12, 13, 15, 16, 17, 18, 19: /mod_base = “2′-hydroxy corresponding nucleoside”
caaauggcacuaguaaacutt

SEQ ID NO: 633 - DNA, 21 bp, Artificial sequence
  Description of the artificial sequence: antisense strand of dsRNA
  modified_base 1: /mod_base = “nucleoside: lacks 5′-phosphate group”
  modified_base 2, 18: /mod_base = “2′-O-methyl corresponding nucleoside”
  modified_base 21: /mod_base = “5′-phosphorothioate thymidine”
  modified_base 1, 3, 4, 5, 6, 7, 8, 9, 10, 11, 12, 13, 14, 15, 16, 17,19: /mod_base = “2′-hydroxy corresponding nucleoside”
ucaaggucggucguugacatt

SEQ ID NO: 634 - DNA, 21 bp, Artificial sequence
  Description of the artificial sequence: antisense strand of dsRNA
  modified_base 1: /mod_base = “nucleoside: lacks 5′-phosphate group”
  modified_base 2: /mod_base = “2′-O-methyl corresponding nucleoside”
  modified_base 21: /mod_base = “5′-phosphorothioate thymidine”
  modified_base 1, 3, 4, 5, 6, 7, 8, 9, 10, 11, 12, 13, 14, 15, 16, 17,18, 19: /mod_base = “2′-hydroxy corresponding nucleoside”
cuagaaaauugagagaagutt

SEQ ID NO: 635 - DNA, 21 bp, Artificial sequence
  Description of the artificial sequence: antisense strand of dsRNA
  modified_base 1: /mod_base = “nucleoside: lacks 5′-phosphate group”
  modified_base 3: /mod_base = “2′-O-methyl corresponding nucleoside”
  modified_base 21: /mod_base = “5′-phosphorothioate thymidine”
  modified_base 1, 2, 4, 5, 6, 7, 8, 9, 10, 11, 12, 13, 14, 15, 16, 17,18, 19: /mod_base = “2′-hydroxy corresponding nucleoside”
gacaaaggacgucccgcgctt

SEQ ID NO: 636 - DNA, 21 bp, Artificial sequence
  Description of the artificial sequence: antisense strand of dsRNA
  modified_base 1: /mod_base = “nucleoside: lacks 5′-phosphate group”
  modified_base 5, 8, 16: /mod_base = “2′-O-methyl corresponding nucleoside”
  modified_base 21: /mod_base = “5′-phosphorothioate thymidine”
  modified_base 1, 2, 3, 4, 6, 7, 9, 10, 11, 12, 13, 14, 15, 17, 18, 19: /mod_base = “2′-hydroxy corresponding nucleoside”
aguccaccacgagucuagatt

SEQ ID NO: 637 - DNA, 21 bp, Artificial sequence
  Description of the artificial sequence: antisense strand of dsRNA
  modified_base 1: /mod_base = “nucleoside: lacks 5′-phosphate group”
  modified_base 9, 12: /mod_base = “2′-O-methyl corresponding nucleoside”
  modified_base 21: /mod_base = “5′-phosphorothioate thymidine”
  modified_base 1, 2, 3, 4, 5, 6, 7, 8, 10, 11, 13, 14, 15, 16, 17, 18,19: /mod_base = “2′-hydroxy corresponding nucleoside”
gaguuccgcaguauggauctt

SEQ ID NO: 638 - DNA, 21 bp, Artificial sequence
  Description of the artificial sequence: antisense strand of dsRNA
  modified_base 1: /mod_base = “nucleoside: lacks 5′-phosphate group”
  modified_base 2, 5, 15: /mod_base = “2′-O-methyl corresponding nucleoside”
  modified_base 21: /mod_base = “5′-phosphorothioate thymidine”
  modified_base 1, 3, 4, 6, 7, 8, 9, 10, 11, 12, 13, 14, 16, 17, 18, 19: /mod_base = “2′-hydroxy corresponding nucleoside”
gcaguauggaucggcagagtt

SEQ ID NO: 639 - DNA, 21 bp, Artificial sequence
  Description of the artificial sequence: antisense strand of dsRNA
  modified_base 1: /mod_base = “nucleoside: lacks 5′-phosphate group”
  modified_base 3, 6, 16: /mod_base = “2′-O-methyl corresponding nucleoside”
  modified_base 21: /mod_base = “5′-phosphorothioate thymidine”
  modified_base 1, 2, 4, 5, 7, 8, 9, 10, 11, 12, 13, 14, 15, 17, 18, 19: /mod_base = “2′-hydroxy corresponding nucleoside”
cgcaguauggaucggcagatt

SEQ ID NO: 640 - DNA, 21 bp, Artificial sequence
  Description of the artificial sequence: antisense strand of dsRNA
  modified_base 1: /mod_base = “nucleoside: lacks 5′-phosphate group”
  modified_base 1, 4, 14: /mod_base = “2′-O-methyl corresponding nucleoside”
  modified_base 21: /mod_base = “5′-phosphorothioate thymidine”
  modified_base 2, 3, 5, 6, 7, 8, 9, 10, 11, 12, 13, 15, 16, 17, 18, 19: /mod_base = “2′-hydroxy corresponding nucleoside”
caguauggaucggcagaggtt

SEQ ID NO: 641 - DNA, 21 bp, Artificial sequence
  Description of the artificial sequence: antisense strand of dsRNA
  modified_base 1: /mod_base = “nucleoside: lacks 5′-phosphate group”
  modified_base 7: /mod_base = “2′-O-methyl corresponding nucleoside”
  modified_base 21: /mod_base = “5′-phosphorothioate thymidine”
  modified_base 1, 2, 3, 4, 5, 6, 8, 9, 10, 11, 12, 13, 14, 15, 16, 17,18, 19: /mod_base = “2′-hydroxy corresponding nucleoside”
accgcguaaagagaggugctt

SEQ ID NO: 642 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
gcgaggcgagggaguucuutt

SEQ ID NO: 643 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
cugcgaggcgagggaguuctt

SEQ ID NO: 644 - DNA, 21 bp, Artificial sequence
  Description of the artificial sequence: antisense strand of dsRNA
  modified_base 1: /mod_base = “nucleoside: lacks 5′-phosphate group”
  modified_base 5: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 11, 12, 13, 14, 15, 16, 17,18, 19: /mod_base = “2′-hydroxy corresponding nucleoside”
gcccuagaaaauugagagatt

SEQ ID NO: 645 - DNA, 21 bp, Artificial sequence
  Description of the artificial sequence: antisense strand of dsRNA
  modified_base 1: /mod_base = “nucleoside: lacks 5′-phosphate group”
  modified_base 3: /mod_base = “2′-O-methyl corresponding nucleoside”
  modified_base 21: /mod_base = “5′-phosphorothioate thymidine”
  modified_base 1, 2, 4, 5, 6, 7, 8, 9, 10, 11, 12, 13, 14, 15, 16, 17,18, 19: /mod_base = “2′-hydroxy corresponding nucleoside”
cguaaagagaggugcgccctt

SEQ ID NO: 646 - DNA, 21 bp, Artificial sequence
  Description of the artificial sequence: antisense strand of dsRNA
  modified_base 1: /mod_base = “nucleoside: lacks 5′-phosphate group”
  modified_base 7, 10: /mod_base = “2′-O-methyl corresponding nucleoside”
  modified_base 21: /mod_base = “5′-phosphorothioate thymidine”
  modified_base 1, 2, 3, 4, 5, 6, 8, 9, 11, 12, 13, 14, 15, 16, 17, 18,19: /mod_base = “2′-hydroxy corresponding nucleoside”
guuccgcaguauggaucggtt

SEQ ID NO: 647 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
ucugcgaggcgagggaguutt

SEQ ID NO: 648 - DNA, 21 bp, Artificial sequence
  Description of the artificial sequence: antisense strand of dsRNA
  modified_base 1: /mod_base = “nucleoside: lacks 5′-phosphate group”
  modified_base 2, 12: /mod_base = “2′-O-methyl corresponding nucleoside”
  modified_base 21: /mod_base = “5′-phosphorothioate thymidine”
  modified_base 1, 3, 4, 5, 6, 7, 8, 9, 10, 11, 13, 14, 15, 16, 17, 18,19: /mod_base = “2′-hydroxy corresponding nucleoside”
guauggaucggcagaggagtt

SEQ ID NO: 649 - DNA, 21 bp, Artificial sequence
  Description of the artificial sequence: antisense strand of dsRNA
  modified_base 1: /mod_base = “nucleoside: lacks 5′-phosphate group”
  modified_base 12, 14: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 13, 15, 16, 17, 18,19: /mod_base = “2′-hydroxy corresponding nucleoside”
gagugcgaauccacacucctt

SEQ ID NO: 650 - DNA, 21 bp, Artificial sequence
  Description of the artificial sequence: antisense strand of dsRNA
  modified_base 1: /mod_base = “nucleoside: lacks 5′-phosphate group”
  modified_base 2: /mod_base = “2′-O-methyl corresponding nucleoside”
  modified_base 21: /mod_base = “5′-phosphorothioate thymidine”
  modified_base 1, 3, 4, 5, 6, 7, 8, 9, 10, 11, 12, 13, 14, 15, 16, 17,18, 19: /mod_base = “2′-hydroxy corresponding nucleoside”
guaaagagaggugcgcccgtt

SEQ ID NO: 651 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
gcgacgcggcgauugagactt

SEQ ID NO: 652 - DNA, 21 bp, Artificial sequence
  Description of the artificial sequence: antisense strand of dsRNA
  modified_base 1: /mod_base = “nucleoside: lacks 5′-phosphate group”
  modified_base 10, 13: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 11, 12, 14, 15, 16, 17,18, 19: /mod_base = “2′-hydroxy corresponding nucleoside”
ggaguuccgcaguauggautt

SEQ ID NO: 653 - DNA, 21 bp, Artificial sequence
  Description of the artificial sequence: antisense strand of dsRNA
  modified_base 1: /mod_base = “nucleoside: lacks 5′-phosphate group”
  modified_base 6: /mod_base = “2′-O-methyl corresponding nucleoside”
  modified_base 21: /mod_base = “5′-phosphorothioate thymidine”
  modified_base 1, 2, 3, 4, 5, 7, 8, 9, 10, 11, 12, 13, 14, 15, 16, 17,18, 19: /mod_base = “2′-hydroxy corresponding nucleoside”
ccgcguaaagagaggugcgtt

SEQ ID NO: 654 - DNA, 21 bp, Artificial sequence
  Description of the artificial sequence: antisense strand of dsRNA
  modified_base 1: /mod_base = “nucleoside: lacks 5′-phosphate group”
  modified_base 5: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 11, 12, 13, 14, 15, 16, 17,18, 19: /mod_base = “2′-hydroxy corresponding nucleoside”
gccucaaggucggucguugtt

SEQ ID NO: 655 - DNA, 21 bp, Artificial sequence
  Description of the artificial sequence: antisense strand of dsRNA
  modified_base 1: /mod_base = “nucleoside: lacks 5′-phosphate group”
  modified_base 1, 12, 15: /mod_base = “2′-O-methyl corresponding nucleoside”
  modified_base 21: /mod_base = “5′-phosphorothioate thymidine”
  modified_base 2, 3, 4, 5, 6, 7, 8, 9, 10, 11, 13, 14, 16, 17, 18, 19: /mod_base = “2′-hydroxy corresponding nucleoside”
uaggaguuccgcaguauggtt

SEQ ID NO: 656 - DNA, 21 bp, Artificial sequence
  Description of the artificial sequence: antisense strand of dsRNA
  modified_base 1: /mod_base = “nucleoside: lacks 5′-phosphate group”
  modified_base 18: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 16,17, 19: /mod_base = “2′-hydroxy corresponding nucleoside”
gggucguccgcgggauucatt

SEQ ID NO: 657 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
ugcgaggcgagggaguucutt

SEQ ID NO: 658 - DNA, 21 bp, Artificial sequence
  Description of the artificial sequence: antisense strand of dsRNA
  modified_base 1: /mod_base = “nucleoside: lacks 5′-phosphate group”
  modified_base 4: /mod_base = “2′-O-methyl corresponding nucleoside”
  modified_base 21: /mod_base = “5′-phosphorothioate thymidine”
  modified_base 1, 2, 3, 5, 6, 7, 8, 9, 10, 11, 12, 13, 14, 15, 16, 17,18, 19: /mod_base = “2′-hydroxy corresponding nucleoside”
gcguaaagagaggugcgcctt

SEQ ID NO: 659 - DNA, 21 bp, Artificial sequence
  Description of the artificial sequence: antisense strand of dsRNA
  modified_base 1: /mod_base = “nucleoside: lacks 5′-phosphate group”
  modified_base 5: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 11, 12, 13, 14, 15, 16, 17,18, 19: /mod_base = “2′-hydroxy corresponding nucleoside”
cgcguaaagagaggugcgctt

SEQ ID NO: 660 - DNA, 21 bp, Artificial sequence
  Description of the artificial sequence: antisense strand of dsRNA
  modified_base 1: /mod_base = “nucleoside: lacks 5′-phosphate group”
  modified_base 16: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 17,18, 19: /mod_base = “2′-hydroxy corresponding nucleoside”
gucguccgcgggauucagctt

SEQ ID NO: 661 - DNA, 21 bp, Artificial sequence
  Description of the artificial sequence: antisense strand of dsRNA
  modified_base 1: /mod_base = “nucleoside: lacks 5′-phosphate group”
  modified_base 2: /mod_base = “2′-O-methyl corresponding nucleoside”
  modified_base 21: /mod_base = “5′-phosphorothioate thymidine”
  modified_base 1, 3, 4, 5, 6, 7, 8, 9, 10, 11, 12, 13, 14, 15, 16, 17,18, 19: /mod_base = “2′-hydroxy corresponding nucleoside”
gcagaggugaagcgaagugtt

SEQ ID NO: 662 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
cugcgacgcggcgauugagtt

SEQ ID NO: 663 - DNA, 21 bp, Artificial sequence
  Description of the artificial sequence: antisense strand of dsRNA
  modified_base 1: /mod_base = “nucleoside: lacks 5′-phosphate group”
  modified_base 6: /mod_base = “2′-O-methyl corresponding nucleoside”
  modified_base 21: /mod_base = “5′-phosphorothioate thymidine”
  modified_base 1, 2, 3, 4, 5, 7, 8, 9, 10, 11, 12, 13, 14, 15, 16, 17,18, 19: /mod_base = “2′-hydroxy corresponding nucleoside”
ggauucagcgccgacgggatt

SEQ ID NO: 664 - DNA, 21 bp, Artificial sequence
  Description of the artificial sequence: antisense strand of dsRNA
  modified_base 1: /mod_base = “nucleoside: lacks 5′-phosphate group”
  modified_base 17: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 16,18, 19: /mod_base = “2′-hydroxy corresponding nucleoside”
ggucguccgcgggauucagtt

SEQ ID NO: 665 - DNA, 21 bp, Artificial sequence
  Description of the artificial sequence: antisense strand of dsRNA
  modified_base 1: /mod_base = “nucleoside: lacks 5′-phosphate group”
  modified_base 1, 4, 12: /mod_base = “2′-O-methyl corresponding nucleoside”
  modified_base 21: /mod_base = “5′-phosphorothioate thymidine”
  modified_base 2, 3, 5, 6, 7, 8, 9, 10, 11, 13, 14, 15, 16, 17, 18, 19: /mod_base = “2′-hydroxy corresponding nucleoside”
caccacgagucuagacucutt

SEQ ID NO: 666 - DNA, 21 bp, Artificial sequence
  Description of the artificial sequence: antisense strand of dsRNA
  modified_base 1: /mod_base = “nucleoside: lacks 5′-phosphate group”
  modified_base 11, 14: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 12, 13, 15, 16, 17, 18,19: /mod_base = “2′-hydroxy corresponding nucleoside”
aggaguuccgcaguauggatt

SEQ ID NO: 667 - DNA, 21 bp, Artificial sequence
  Description of the artificial sequence: antisense strand of dsRNA
  modified_base 1: /mod_base = “nucleoside: lacks 5′-phosphate group”
  modified_base 14: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 15, 16, 17,18, 19: /mod_base = “2′-hydroxy corresponding nucleoside”
cguccgcgggauucagcgctt

SEQ ID NO: 668 - DNA, 21 bp, Artificial sequence
  Description of the artificial sequence: antisense strand of dsRNA
  modified_base 1: /mod_base = “nucleoside: lacks 5′-phosphate group”
  modified_base 14, 16: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 15, 17, 18,19: /mod_base = “2′-hydroxy corresponding nucleoside”
aggagugcgaauccacacutt

SEQ ID NO: 669 - DNA, 21 bp, Artificial sequence
  Description of the artificial sequence: antisense strand of dsRNA
  modified_base 1: /mod_base = “nucleoside: lacks 5′-phosphate group”
  modified_base 6, 9, 15: /mod_base = “2′-O-methyl corresponding nucleoside”
  modified_base 21: /mod_base = “5′-phosphorothioate thymidine”
  modified_base 1, 2, 3, 4, 5, 7, 8, 10, 11, 12, 13, 14, 16, 17, 18, 19: /mod_base = “2′-hydroxy corresponding nucleoside”
gccuguaacacgagcagggtt

SEQ ID NO: 670 - DNA, 21 bp, Artificial sequence
  Description of the artificial sequence: antisense strand of dsRNA
  modified_base 1: /mod_base = “nucleoside: lacks 5′-phosphate group”
  modified_base 21: /mod_base = “5′-phosphorothioate thymidine”
  modified_base 1..19: /mod_base = “2′-hydroxy corresponding nucleoside”
cgggucguccgcgggauuctt

SEQ ID NO: 671 - DNA, 21 bp, Artificial sequence
  Description of the artificial sequence: antisense strand of dsRNA
  modified_base 1: /mod_base = “nucleoside: lacks 5′-phosphate group”
  modified_base 3, 7, 12, 14: /mod_base = “2′-O-methyl corresponding nucleoside”
  modified_base 21: /mod_base = “5′-phosphorothioate thymidine”
  modified_base 1, 2, 4, 5, 6, 8, 9, 10, 11, 13, 15, 16, 17, 18, 19: /mod_base = “2′-hydroxy corresponding nucleoside”
gccacccaaggcacagcuutt

SEQ ID NO: 672 - DNA, 21 bp, Artificial sequence
  Description of the artificial sequence: antisense strand of dsRNA
  modified_base 1: /mod_base = “nucleoside: lacks 5′-phosphate group”
  modified_base 5, 8, 14: /mod_base = “2′-O-methyl corresponding nucleoside”
  modified_base 21: /mod_base = “5′-phosphorothioate thymidine”
  modified_base 1, 2, 3, 4, 6, 7, 9, 10, 11, 12, 13, 15, 16, 17, 18, 19: /mod_base = “2′-hydroxy corresponding nucleoside”
ccuguaacacgagcagggctt

SEQ ID NO: 673 - DNA, 21 bp, Artificial sequence
  Description of the artificial sequence: antisense strand of dsRNA
  modified_base 1: /mod_base = “nucleoside: lacks 5′-phosphate group”
  modified_base 5: /mod_base = “2′-O-methyl corresponding nucleoside”
  modified_base 21: /mod_base = “5′-phosphorothioate thymidine”
  modified_base 1, 2, 3, 4, 6, 7, 8, 9, 10, 11, 12, 13, 14, 15, 16, 17,18, 19: /mod_base = “2′-hydroxy corresponding nucleoside”
gauucagcgccgacgggactt

SEQ ID NO: 674 - DNA, 21 bp, Artificial sequence
  Description of the artificial sequence: antisense strand of dsRNA
  modified_base 1: /mod_base = “nucleoside: lacks 5′-phosphate group”
  modified_base 14: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 15, 16, 17,18, 19: /mod_base = “2′-hydroxy corresponding nucleoside”
ccggaaguguugauaagautt

SEQ ID NO: 675 - DNA, 21 bp, Artificial sequence
  Description of the artificial sequence: antisense strand of dsRNA
  modified_base 1: /mod_base = “nucleoside: lacks 5′-phosphate group”
  modified_base 16: /mod_base = “2′-O-methyl corresponding nucleoside”
  modified_base 21: /mod_base = “5′-phosphorothioate thymidine”
  modified_base 1, 2, 3, 4, 5, 6, 7, 8, 9, 10, 11, 12, 13, 14, 15, 17,18, 19: /mod_base = “2′-hydroxy corresponding nucleoside”
uuccggaaguguugauaagtt

SEQ ID NO: 676 - DNA, 21 bp, Artificial sequence
  Description of the artificial sequence: antisense strand of dsRNA
  modified_base 3, 5, 7, 9, 11, 13, 15, 17, 19: /mod_base = “2′-O-methyl corresponding nucleoside”
  modified_base 2, 4, 6, 8, 10, 12, 14, 16, 18: /mod_base = “2′-deoxy-2′-fluoro corresponding nucleoside”
  modified_base 21: /mod_base = “5′-phosphorothioate thymidine”
tuccggaaguguugauaagtt

SEQ ID NO: 677 - DNA, 19 bp, Artificial sequence
  HBV cDNA target sequence
gtggtggacttctctcaat

SEQ ID NO: 678 - DNA, 19 bp, Artificial sequence
  HBV cDNA target sequence
tggtggacttctctcaatt

SEQ ID NO: 679 - DNA, 19 bp, Artificial sequence
  HBV cDNA target sequence
ggacttctctcaattttct

SEQ ID NO: 680 - DNA, 19 bp, Artificial sequence
  HBV cDNA target sequence
gctgtaggcataaattggt

SEQ ID NO: 681 - DNA, 19 bp, Artificial sequence
  HBV cDNA target sequence
ctgtaggcataaattggtc

SEQ ID NO: 682 - RNA, 19 nt, Artificial sequence
  HBV RNAi agent antisense strand core stretch sequence
auugagagaaguccaccac

SEQ ID NO: 683 - RNA, 19 nt, Artificial sequence
  HBV RNAi agent antisense strand core stretch sequence
uuugagagaaguccaccac

SEQ ID NO: 684 - RNA, 19 nt, Artificial sequence
  HBV RNAi agent antisense strand core stretch sequence
  modified_base 19: n = any nucleotide
auugagagaaguccaccan

SEQ ID NO: 685 - RNA, 19 nt, Artificial sequence
  HBV RNAi agent antisense strand core stretch sequence
  modified_base 19: n = any nucleotide
uuugagagaaguccaccan

SEQ ID NO: 686 - RNA, 19 nt, Artificial sequence
  HBV RNAi agent antisense strand core stretch sequence
  modified_base 1, 19: n = any nucleotide
nuugagagaaguccaccan

SEQ ID NO: 687 - RNA, 19 nt, Artificial sequence
  HBV RNAi agent antisense strand core stretch sequence
aauugagagaaguccacca

SEQ ID NO: 688 - RNA, 19 nt, Artificial sequence
  HBV RNAi agent antisense strand core stretch sequence
uauugagagaaguccacca

SEQ ID NO: 689 - RNA, 19 nt, Artificial sequence
  HBV RNAi agent antisense strand core stretch sequence
  modified_base 19: n = any nucleotide
aauugagagaaguccaccn

SEQ ID NO: 690 - RNA, 19 nt, Artificial sequence
  HBV RNAi agent antisense strand core stretch sequence
  modified_base 19: n = any nucleotide
uauugagagaaguccaccn

SEQ ID NO: 691 - RNA, 19 nt, Artificial sequence
  HBV RNAi agent antisense strand core stretch sequence
  modified_base 1, 19: n = any nucleotide
nauugagagaaguccaccn

SEQ ID NO: 692 - RNA, 19 nt, Artificial sequence
  HBV RNAi agent antisense strand core stretch sequence
agaaaauugagagaagucc

SEQ ID NO: 693 - RNA, 19 nt, Artificial sequence
  HBV RNAi agent antisense strand core stretch sequence
ugaaaauugagagaagucc

SEQ ID NO: 694 - RNA, 19 nt, Artificial sequence
  HBV RNAi agent antisense strand core stretch sequence
  modified_base 19: n = any nucleotide
agaaaauugagagaagucn

SEQ ID NO: 695 - RNA, 19 nt, Artificial sequence
  HBV RNAi agent antisense strand core stretch sequence
  modified_base 19: n = any nucleotide
ugaaaauugagagaagucn

SEQ ID NO: 696 - RNA, 19 nt, Artificial sequence
  HBV RNAi agent antisense strand core stretch sequence
  modified_base 1, 19: n = any nucleotide
ngaaaauugagagaagucn

SEQ ID NO: 697 - RNA, 19 nt, Artificial sequence
  HBV RNAi agent antisense strand core stretch sequence
accaauuuaugccuacagc

SEQ ID NO: 698 - RNA, 19 nt, Artificial sequence
  HBV RNAi agent antisense strand core stretch sequence
uccaauuuaugccuacagc

SEQ ID NO: 699 - RNA, 19 nt, Artificial sequence
  HBV RNAi agent antisense strand core stretch sequence
  modified_base 19: n = any nucleotide
accaauuuaugccuacagn

SEQ ID NO: 700 - RNA, 19 nt, Artificial sequence
  HBV RNAi agent antisense strand core stretch sequence
  modified_base 19: n = any nucleotide
uccaauuuaugccuacagn

SEQ ID NO: 701 - RNA, 19 nt, Artificial sequence
  HBV RNAi agent antisense strand core stretch sequence
  modified_base 1,19: n = any nucleotide
nccaauuuaugccuacagn

SEQ ID NO: 702 - RNA, 19 nt, Artificial sequence
  HBV RNAi agent antisense strand core stretch sequence
gaccaauuuaugccuacag

SEQ ID NO: 703 - RNA, 19 nt, Artificial sequence
  HBV RNAi agent antisense strand core stretch sequence
aaccaauuuaugccuacag

SEQ ID NO: 704 - RNA, 19 nt, Artificial sequence
  HBV RNAi agent antisense strand core stretch sequence
uaccaauuuaugccuacag

SEQ ID NO: 705 - RNA, 19 nt, Artificial sequence
  HBV RNAi agent antisense strand core stretch sequence
  modified_base 19: n = any nucleotide
gaccaauuuaugccuacan

SEQ ID NO: 706 - RNA, 19 nt, Artificial sequence
  HBV RNAi agent antisense strand core stretch sequence
  modified_base 19: n = any nucleotide
aaccaauuuaugccuacan

SEQ ID NO: 707 - RNA, 19 nt, Artificial sequence
  HBV RNAi agent antisense strand core stretch sequence
  modified_base 19: n = any nucleotide
uaccaauuuaugccuacan

SEQ ID NO: 708 - RNA, 19 nt, Artificial sequence
  HBV RNAi agent antisense strand core stretch sequence
  modified_base 1, 19: n = any nucleotide
naccaauuuaugccuacan

SEQ ID NO: 709 - DNA, 19 bp, Artificial sequence
  HBV RNAi agent sense strand core stretch sequence
gugguggacuucucucaau

SEQ ID NO: 710 - RNA, 19 nt, Artificial sequence
  HBV RNAi agent sense strand core stretch sequence
gugguggacuucucucaaa

SEQ ID NO: 711 - RNA, 19 nt, Artificial sequence
  HBV RNAi agent sense strand core stretch sequence
  modified_base 1: n = any nucleotide
nugguggacuucucucaau

SEQ ID NO: 712 - RNA, 19 nt, Artificial sequence
  HBV RNAi agent sense strand core stretch sequence
  modified_base 1: n = any nucleotide
nugguggacuucucucaaa

SEQ ID NO: 713 - RNA, 19 nt, Artificial sequence
  HBV RNAi agent sense strand core stretch sequence
  modified_base 1, 19: n = any nucleotide
nugguggacuucucucaan

SEQ ID NO: 714 - RNA, 19 nt, Artificial sequence
  HBV RNAi agent sense strand core stretch sequence
ugguggacuucucucaauu

SEQ ID NO: 715 - RNA, 19 nt, Artificial sequence
  HBV RNAi agent sense strand core stretch sequence
ugguggacuucucucaaua

SEQ ID NO: 716 - RNA, 19 nt, Artificial sequence
  HBV RNAi agent sense strand core stretch sequence
  modified_base 1: n = any nucleotide
ngguggacuucucucaauu

SEQ ID NO: 717 - RNA, 19 nt, Artificial sequence
  HBV RNAi agent sense strand core stretch sequence
  modified_base 1: n = any nucleotide
ngguggacuucucucaaua

SEQ ID NO: 718 - RNA, 19 nt, Artificial sequence
  HBV RNAi agent sense strand core stretch sequence
  modified_base 1, 19: n = any nucleotide
ngguggacuucucucaaun

SEQ ID NO: 719 - RNA, 19 nt, Artificial sequence
  HBV RNAi agent sense strand core stretch sequence
ggacuucucucaauuuucu

SEQ ID NO: 720 - RNA, 19 nt, Artificial sequence
  HBV RNAi agent sense strand core stretch sequence
ggacuucucucaauuuuca

SEQ ID NO: 721 - RNA, 19 nt, Artificial sequence
  HBV RNAi agent sense strand core stretch sequence
  modified_base 1: n = any nucleotide
ngacuucucucaauuuucu

SEQ ID NO: 722 - RNA, 19 nt, Artificial sequence
  HBV RNAi agent sense strand core stretch sequence
  modified_base 1: n = any nucleotide
ngacuucucucaauuuuca

SEQ ID NO: 723 - RNA, 19 nt, Artificial sequence
  HBV RNAi agent sense strand core stretch sequence
  modified_base 1, 19: n = any nucleotide
ngacuucucucaauuuucn

SEQ ID NO: 724 - RNA, 19 nt, Artificial sequence
  HBV RNAi agent sense strand core stretch sequence
gcuguaggcauaaauuggu

SEQ ID NO: 725 - RNA, 19 nt, Artificial sequence
  HBV RNAi agent sense strand core stretch sequence
gcuguaggcauaaauugga

SEQ ID NO: 726 - RNA, 19 nt, Artificial sequence
  HBV RNAi agent sense strand core stretch sequence
  modified_base 1: n = any nucleotide
ncuguaggcauaaauuggu

SEQ ID NO: 727 - RNA, 19 nt, Artificial sequence
  HBV RNAi agent sense strand core stretch sequence
  modified_base 1: n = any nucleotide
ncuguaggcauaaauugga

SEQ ID NO: 728 - RNA, 19 nt, Artificial sequence
  HBV RNAi agent sense strand core stretch sequence
  modified_base 1, 19: n = any nucleotide
ncuguaggcauaaauuggn

SEQ ID NO: 729 - RNA, 19 nt, Artificial sequence
  HBV RNAi agent sense strand core stretch sequence
cuguaggcauaaauugguc

SEQ ID NO: 730 - RNA, 19 nt, Artificial sequence
  HBV RNAi agent sense strand core stretch sequence
cuguaggcauaaauugguu

SEQ ID NO: 731 - RNA, 19 nt, Artificial sequence
  HBV RNAi agent sense strand core stretch sequence
cuguaggcauaaauuggua

SEQ ID NO: 732 - RNA, 19 nt, Artificial sequence
  HBV RNAi agent sense strand core stretch sequence
  modified_base 1: n = any nucleotide
nuguaggcauaaauugguc

SEQ ID NO: 733 - RNA, 19 nt, Artificial sequence
  HBV RNAi agent sense strand core stretch sequence
  modified_base 1: n = any nucleotide
nuguaggcauaaauugguu

SEQ ID NO: 734 - RNA, 19 nt, Artificial sequence
  HBV RNAi agent sense strand core stretch sequence
  modified_base 1: n = any nucleotide
nuguaggcauaaauuggua

SEQ ID NO: 735 - RNA, 19 nt, Artificial sequence
  HBV RNAi agent sense strand core stretch sequence
  modified_base 1, 19: n = any nucleotide
nuguaggcauaaauuggun

SEQ ID NO: 736 - RNA, 26 nt, Artificial sequence
  HBV RNAi Agent antisense strand modified sequence
uaccaauuuaugccuacaggccuuau

SEQ ID NO: 737 - RNA, 23 nt, Artificial sequence
  HBV RNAi Agent antisense strand modified sequence
uaccaauuuaugccuacaggccu

SEQ ID NO: 738 - RNA, 23 nt, Artificial sequence
  HBV RNAi Agent antisense strand modified sequence
uaccaauuuaugccuacaggccu

SEQ ID NO: 739 - RNA, 21 nt, Artificial sequence
  HBV RNAi Agent antisense strand modified sequence
uaccaauuuaugccuacaggc

SEQ ID NO: 740 - RNA, 21 nt, Artificial sequence
  HBV RNAi Agent antisense strand modified sequence
ugugaagcgaagugcacacuu

SEQ ID NO: 741 - RNA, 26 nt, Artificial sequence
  HBV RNAi Agent antisense strand modified sequence
uaccaauuuaugccuacagccuccgc

SEQ ID NO: 742 - RNA, 26 nt, Artificial sequence
  HBV RNAi Agent antisense strand modified sequence
uaccaauuuaugccuacagccuccgc

SEQ ID NO: 743 - RNA, 21 nt, Artificial sequence
  HBV RNAi Agent antisense strand modified sequence
uaccaauuuaugccuacaguu

SEQ ID NO: 744 - RNA, 21 nt, Artificial sequence
  HBV RNAi Agent antisense strand modified sequence
uaccaauuuaugccuacaggc

SEQ ID NO: 745 - RNA, 21 nt, Artificial sequence
  HBV RNAi Agent antisense strand modified sequence
uaccaauuuaugccuacaggc

SEQ ID NO: 746 - RNA, 21 nt, Artificial sequence
  HBV RNAi Agent antisense strand modified sequence
uaccaauuuaugccuacaguu

SEQ ID NO: 747 - RNA, 21 nt, Artificial sequence
  HBV RNAi Agent antisense strand modified sequence
auugagagaaguccaccacga

SEQ ID NO: 748 - RNA, 21 nt, Artificial sequence
  HBV RNAi Agent antisense strand modified sequence
auugagagaaguccaccacga

SEQ ID NO: 749 - RNA, 21 nt, Artificial sequence
  HBV RNAi Agent antisense strand modified sequence
auugagagaaguccaccacuu

SEQ ID NO: 750 - RNA, 21 nt, Artificial sequence
  HBV RNAi Agent antisense strand modified sequence
uuugagagaaguccaccacga

SEQ ID NO: 751 - RNA, 21 nt, Artificial sequence
  HBV RNAi Agent antisense strand modified sequence
aauugagagaaguccaccacg

SEQ ID NO: 752 - RNA, 21 nt, Artificial sequence
  HBV RNAi Agent antisense strand modified sequence
aauugagagaaguccaccacg

SEQ ID NO: 753 - RNA, 21 nt, Artificial sequence
  HBV RNAi Agent antisense strand modified sequence
aauugagagaaguccaccauu

SEQ ID NO: 754 - RNA, 21 nt, Artificial sequence
  HBV RNAi Agent antisense strand modified sequence
uauugagagaaguccaccacg

SEQ ID NO: 755 - RNA, 21 nt, Artificial sequence
  HBV RNAi Agent antisense strand modified sequence
uaccaauuuaugccuacaggu

SEQ ID NO: 756 - RNA, 21 nt, Artificial sequence
  HBV RNAi Agent antisense strand modified sequence
uaccaauuuaugccuacaguu

SEQ ID NO: 757 - RNA, 21 nt, Artificial sequence
  HBV RNAi Agent antisense strand modified sequence
uaccaauuuaugccuacagcc

SEQ ID NO: 758 - RNA, 23 nt, Artificial sequence
  HBV RNAi Agent antisense strand modified sequence
uaccaauuuaugccuacagccuu

SEQ ID NO: 759 - RNA, 23 nt, Artificial sequence
  HBV RNAi Agent antisense strand modified sequence
uaccaauuuaugccuacagccuc

SEQ ID NO: 760 - RNA, 21 nt, Artificial sequence
  HBV RNAi Agent antisense strand modified sequence
uaccaauuuaugccuacagcc

SEQ ID NO: 761 - RNA, 23 nt, Artificial sequence
  HBV RNAi Agent antisense strand modified sequence
uaccaauuuaugccuacagccuu

SEQ ID NO: 762 - RNA, 23 nt, Artificial sequence
  HBV RNAi Agent antisense strand modified sequence
uaccaauuuaugccuacagccuc

SEQ ID NO: 763 - RNA, 21 nt, Artificial sequence
  HBV RNAi Agent antisense strand modified sequence
auugagagaaguccaccacuu

SEQ ID NO: 764 - RNA, 21 nt, Artificial sequence
  HBV RNAi Agent antisense strand modified sequence
uuugagagaaguccaccacuu

SEQ ID NO: 765 - RNA, 23 nt, Artificial sequence
  HBV RNAi Agent antisense strand modified sequence
auugagagaaguccaccacgguu

SEQ ID NO: 766 - RNA, 23 nt, Artificial sequence
  HBV RNAi Agent antisense strand modified sequence
auugagagaaguccaccacgguu

SEQ ID NO: 767 - RNA, 23 nt, Artificial sequence
  HBV RNAi Agent antisense strand modified sequence
auugagagaaguccaccacgagu

SEQ ID NO: 768 - RNA, 21 nt, Artificial sequence
  HBV RNAi Agent antisense strand modified sequence
uauugagagaaguccaccacg

SEQ ID NO: 769 - RNA, 21 nt, Artificial sequence
  HBV RNAi Agent antisense strand modified sequence
uauugagagaaguccaccacg

SEQ ID NO: 770 - RNA, 23 nt, Artificial sequence
  HBV RNAi Agent antisense strand modified sequence
uauugagagaaguccaccacguu

SEQ ID NO: 771 - RNA, 23 nt, Artificial sequence
  HBV RNAi Agent antisense strand modified sequence
uauugagagaaguccaccacgag

SEQ ID NO: 772 - RNA, 23 nt, Artificial sequence
  HBV RNAi Agent antisense strand modified sequence
uauugagagaaguccaccacgag

SEQ ID NO: 773 - RNA, 22 nt, Artificial sequence
  HBV RNAi Agent antisense strand modified sequence
uauugagagaaguccaccacga

SEQ ID NO: 774 - RNA, 22 nt, Artificial sequence
  HBV RNAi Agent antisense strand modified sequence
uauugagagaaguccaccacga

SEQ ID NO: 775 - RNA, 21 nt, Artificial sequence
  HBV RNAi Agent antisense strand modified sequence
agaaaauugagagaaguccac

SEQ ID NO: 776 - RNA, 21 nt, Artificial sequence
  HBV RNAi Agent antisense strand modified sequence
uaccaauuuaugccuacaguu

SEQ ID NO: 777 - RNA, 21 nt, Artificial sequence
  HBV RNAi Agent antisense strand modified sequence
uaccaauuuaugccuacagcc

SEQ ID NO: 778 - RNA, 22 nt, Artificial sequence
  HBV RNAi Agent antisense strand modified sequence
uaccaauuuaugccuacagcuu

SEQ ID NO: 779 - RNA, 22 nt, Artificial sequence
  HBV RNAi Agent antisense strand modified sequence
uaccaauuuaugccuacagccu

SEQ ID NO: 780 - RNA, 23 nt, Artificial sequence
  HBV RNAi Agent antisense strand modified sequence
uaccaauuuaugccuacagccuu

SEQ ID NO: 781 - RNA, 23 nt, Artificial sequence
  HBV RNAi Agent antisense strand modified sequence
uaccaauuuaugccuacagccuc

SEQ ID NO: 782 - RNA, 21 nt, Artificial sequence
  HBV RNAi Agent antisense strand modified sequence
uaccaauuuaugccuacaguu

SEQ ID NO: 783 - RNA, 21 nt, Artificial sequence
  HBV RNAi Agent antisense strand modified sequence
uauugagagaaguccaccacg

SEQ ID NO: 784 - RNA, 21 nt, Artificial sequence
  HBV RNAi Agent antisense strand modified sequence
uauugagagaaguccaccauu

SEQ ID NO: 785 - RNA, 21 nt, Artificial sequence
  HBV RNAi Agent antisense strand modified sequence
uauugagagaaguccaccacg

SEQ ID NO: 786 - RNA, 22 nt, Artificial sequence
  HBV RNAi Agent antisense strand modified sequence
uauugagagaaguccaccacuu

SEQ ID NO: 787 - RNA, 22 nt, Artificial sequence
  HBV RNAi Agent antisense strand modified sequence
uauugagagaaguccaccacga

SEQ ID NO: 788 - RNA, 21 nt, Artificial sequence
  HBV RNAi Agent antisense strand modified sequence
uauugagagaaguccaccacg

SEQ ID NO: 789 - RNA, 21 nt, Artificial sequence
  HBV RNAi Agent antisense strand modified sequence
uuugagagaaguccaccacuu

SEQ ID NO: 790 - RNA, 21 nt, Artificial sequence
  HBV RNAi Agent antisense strand modified sequence
uuugagagaaguccaccacga

SEQ ID NO: 791 - RNA, 22 nt, Artificial sequence
  HBV RNAi Agent antisense strand modified sequence
uuugagagaaguccaccacguu

SEQ ID NO: 792 - RNA, 22 nt, Artificial sequence
  HBV RNAi Agent antisense strand modified sequence
uuugagagaaguccaccacgag

SEQ ID NO: 793 - RNA, 21 nt, Artificial sequence
  HBV RNAi Agent antisense strand modified sequence
uuugagagaaguccaccacuu

SEQ ID NO: 794 - RNA, 22 nt, Artificial sequence
  HBV RNAi Agent antisense strand modified sequence
uauugagagaaguccaccacuu

SEQ ID NO: 795 - RNA, 22 nt, Artificial sequence
  HBV RNAi Agent antisense strand modified sequence
uauugagagaaguccaccacuu

SEQ ID NO: 796 - RNA, 23 nt, Artificial sequence
  HBV RNAi Agent antisense strand modified sequence
uauugagagaaguccaccacguu

SEQ ID NO: 797 - RNA, 22 nt, Artificial sequence
  HBV RNAi Agent antisense strand modified sequence
uauugagagaaguccaccacuu

SEQ ID NO: 798 - RNA, 23 nt, Artificial sequence
  HBV RNAi Agent antisense strand modified sequence
uauugagagaaguccaccacgag

SEQ ID NO: 799 - RNA, 22 nt, Artificial sequence
  HBV RNAi Agent antisense strand modified sequence
uauugagagaaguccaccacuu

SEQ ID NO: 800 - RNA, 21 nt, Artificial sequence
  HBV RNAi Agent antisense strand modified sequence
agaaaauugagagaaguccuu

SEQ ID NO: 801 - RNA, 21 nt, Artificial sequence
  HBV RNAi Agent antisense strand modified sequence
agaaaauugagagaaguccac

SEQ ID NO: 802 - RNA, 23 nt, Artificial sequence
  HBV RNAi Agent antisense strand modified sequence
agaaaauugagagaaguccacuu

SEQ ID NO: 803 - RNA, 22 nt, Artificial sequence
  HBV RNAi Agent antisense strand modified sequence
agaaaauugagagaaguccacc

SEQ ID NO: 804 - RNA, 21 nt, Artificial sequence
  HBV RNAi Agent antisense strand modified sequence
ugaaaauugagagaaguccuu

SEQ ID NO: 805 - RNA, 21 nt, Artificial sequence
  HBV RNAi Agent antisense strand modified sequence
ugaaaauugagagaaguccac

SEQ ID NO: 806 - RNA, 21 nt, Artificial sequence
  HBV RNAi Agent antisense strand modified sequence
accaauuuaugccuacagcuu

SEQ ID NO: 807 - RNA, 22 nt, Artificial sequence
  HBV RNAi Agent antisense strand modified sequence
accaauuuaugccuacagccuu

SEQ ID NO: 808 - RNA, 22 nt, Artificial sequence
  HBV RNAi Agent antisense strand modified sequence
accaauuuaugccuacagccuc

SEQ ID NO: 809 - RNA, 21 nt, Artificial sequence
  HBV RNAi Agent antisense strand modified sequence
uccaauuuaugccuacagcuu

SEQ ID NO: 810 - RNA, 22 nt, Artificial sequence
  HBV RNAi Agent antisense strand modified sequence
uccaauuuaugccuacagccuu

SEQ ID NO: 811 - RNA, 21 nt, Artificial sequence
  HBV RNAi Agent antisense strand modified sequence
uaccaauuuaugccuacagcc

SEQ ID NO: 812 - RNA, 21 nt, Artificial sequence
  HBV RNAi Agent antisense strand modified sequence
uaccaauuuaugccuacagcc

SEQ ID NO: 813 - RNA, 21 nt, Artificial sequence
  HBV RNAi Agent antisense strand modified sequence
uaccaauuuaugccuacagcc

SEQ ID NO: 814 - RNA, 21 nt, Artificial sequence
  HBV RNAi Agent antisense strand modified sequence
uaccaauuuaugccuacagcu

SEQ ID NO: 815 - RNA, 21 nt, Artificial sequence
  HBV RNAi Agent antisense strand modified sequence
uaccaauuuaugccuacagcg

SEQ ID NO: 816 - RNA, 21 nt, Artificial sequence
  HBV RNAi Agent antisense strand modified sequence
aaccaauuuaugccuacagcc

SEQ ID NO: 817 - RNA, 21 nt, Artificial sequence
  HBV RNAi Agent antisense strand modified sequence
uaccaauuuaugccuacaguu

SEQ ID NO: 818 - RNA, 21 nt, Artificial sequence
  HBV RNAi Agent antisense strand modified sequence
uaccaauuuaugccuacagcc

SEQ ID NO: 819 - RNA, 21 nt, Artificial sequence
  HBV RNAi Agent antisense strand modified sequence
accaauuuaugccuacagccu

SEQ ID NO: 820 - RNA, 21 nt, Artificial sequence
  HBV RNAi Agent antisense strand modified sequence
uccaauuuaugccuacagccu

SEQ ID NO: 821 - RNA, 21 nt, Artificial sequence
  HBV RNAi Agent antisense strand modified sequence
accaauuuaugccuacagccg

SEQ ID NO: 822 - RNA, 21 nt, Artificial sequence
  HBV RNAi Agent antisense strand modified sequence
uccaauuuaugccuacagccg

SEQ ID NO: 823 - RNA, 21 nt, Artificial sequence
  HBV RNAi Agent antisense strand modified sequence
uaccaauuuaugccuacaggg

SEQ ID NO: 824 - RNA, 26 nt, Artificial sequence
  HBV RNAi Agent antisense strand unmodified sequence
uaccaauuuaugccuacaggccuuau

SEQ ID NO: 825 - RNA, 23 nt, Artificial sequence
  HBV RNAi Agent antisense strand unmodified sequence
uaccaauuuaugccuacaggccu

SEQ ID NO: 826 - RNA, 21 nt, Artificial sequence
  HBV RNAi Agent antisense strand unmodified sequence
uaccaauuuaugccuacaggc

SEQ ID NO: 827 - RNA, 21 nt, Artificial sequence
  HBV RNAi Agent antisense strand unmodified sequence
ugugaagcgaagugcacacuu

SEQ ID NO: 828 - RNA, 26 nt, Artificial sequence
  HBV RNAi Agent antisense strand unmodified sequence
uaccaauuuaugccuacagccuccgc

SEQ ID NO: 829 - RNA, 21 nt, Artificial sequence
  HBV RNAi Agent antisense strand unmodified sequence
uaccaauuuaugccuacaguu

SEQ ID NO: 830 - RNA, 21 nt, Artificial sequence
  HBV RNAi Agent antisense strand unmodified sequence
auugagagaaguccaccacga

SEQ ID NO: 831 - RNA, 21 nt, Artificial sequence
  HBV RNAi Agent antisense strand unmodified sequence
auugagagaaguccaccacuu

SEQ ID NO: 832 - RNA, 21 nt, Artificial sequence
  HBV RNAi Agent antisense strand unmodified sequence
uuugagagaaguccaccacga

SEQ ID NO: 833 - RNA, 21 nt, Artificial sequence
  HBV RNAi Agent antisense strand unmodified sequence
aauugagagaaguccaccacg

SEQ ID NO: 834 - RNA, 21 nt, Artificial sequence
  HBV RNAi Agent antisense strand unmodified sequence
aauugagagaaguccaccauu

SEQ ID NO: 835 - RNA, 21 nt, Artificial sequence
  HBV RNAi Agent antisense strand unmodified sequence
uauugagagaaguccaccacg

SEQ ID NO: 836 - RNA, 21 nt, Artificial sequence
  HBV RNAi Agent antisense strand unmodified sequence
uaccaauuuaugccuacaggu

SEQ ID NO: 837 - RNA, 21 nt, Artificial sequence
  HBV RNAi Agent antisense strand unmodified sequence
uaccaauuuaugccuacagcc

SEQ ID NO: 838 - RNA, 23 nt, Artificial sequence
  HBV RNAi Agent antisense strand unmodified sequence
uaccaauuuaugccuacagccuu

SEQ ID NO: 839 - RNA, 23 nt, Artificial sequence
  HBV RNAi Agent antisense strand unmodified sequence
uaccaauuuaugccuacagccuc

SEQ ID NO: 840 - RNA, 21 nt, Artificial sequence
  HBV RNAi Agent antisense strand unmodified sequence
uuugagagaaguccaccacuu

SEQ ID NO: 841 - RNA, 23 nt, Artificial sequence
  HBV RNAi Agent antisense strand unmodified sequence
auugagagaaguccaccacgguu

SEQ ID NO: 842 - RNA, 23 nt, Artificial sequence
  HBV RNAi Agent antisense strand unmodified sequence
auugagagaaguccaccacgagu

SEQ ID NO: 843 - RNA, 23 nt, Artificial sequence
  HBV RNAi Agent antisense strand unmodified sequence
uauugagagaaguccaccacguu

SEQ ID NO: 844 - RNA, 23 nt, Artificial sequence
  HBV RNAi Agent antisense strand unmodified sequence
uauugagagaaguccaccacgag

SEQ ID NO: 845 - RNA, 22 nt, Artificial sequence
  HBV RNAi Agent antisense strand unmodified sequence
uauugagagaaguccaccacga

SEQ ID NO: 846 - RNA, 21 nt, Artificial sequence
  HBV RNAi Agent antisense strand unmodified sequence
agaaaauugagagaaguccac

SEQ ID NO: 847 - RNA, 22 nt, Artificial sequence
  HBV RNAi Agent antisense strand unmodified sequence
uaccaauuuaugccuacagcuu

SEQ ID NO: 848 - RNA, 22 nt, Artificial sequence
  HBV RNAi Agent antisense strand unmodified sequence
uaccaauuuaugccuacagccu

SEQ ID NO: 849 - RNA, 21 nt, Artificial sequence
  HBV RNAi Agent antisense strand unmodified sequence
uauugagagaaguccaccauu

SEQ ID NO: 850 - RNA, 22 nt, Artificial sequence
  HBV RNAi Agent antisense strand unmodified sequence
uauugagagaaguccaccacuu

SEQ ID NO: 851 - RNA, 22 nt, Artificial sequence
  HBV RNAi Agent antisense strand unmodified sequence
uuugagagaaguccaccacguu

SEQ ID NO: 852 - RNA, 22 nt, Artificial sequence
  HBV RNAi Agent antisense strand unmodified sequence
uuugagagaaguccaccacgag

SEQ ID NO: 853 - RNA, 21 nt, Artificial sequence
  HBV RNAi Agent antisense strand unmodified sequence
agaaaauugagagaaguccuu

SEQ ID NO: 854 - RNA, 23 nt, Artificial sequence
  HBV RNAi Agent antisense strand unmodified sequence
agaaaauugagagaaguccacuu

SEQ ID NO: 855 - RNA, 22 nt, Artificial sequence
  HBV RNAi Agent antisense strand unmodified sequence
agaaaauugagagaaguccacc

SEQ ID NO: 856 - RNA, 21 nt, Artificial sequence
  HBV RNAi Agent antisense strand unmodified sequence
ugaaaauugagagaaguccac

SEQ ID NO: 857 - RNA, 21 nt, Artificial sequence
  HBV RNAi Agent antisense strand unmodified sequence
accaauuuaugccuacagcuu

SEQ ID NO: 858 - RNA, 22 nt, Artificial sequence
  HBV RNAi Agent antisense strand unmodified sequence
accaauuuaugccuacagccuu

SEQ ID NO: 859 - RNA, 22 nt, Artificial sequence
  HBV RNAi Agent antisense strand unmodified sequence
accaauuuaugccuacagccuc

SEQ ID NO: 860 - RNA, 21 nt, Artificial sequence
  HBV RNAi Agent antisense strand unmodified sequence
uccaauuuaugccuacagcuu

SEQ ID NO: 861 - RNA, 22 nt, Artificial sequence
  HBV RNAi Agent antisense strand unmodified sequence
uccaauuuaugccuacagccuu

SEQ ID NO: 862 - RNA, 21 nt, Artificial sequence
  HBV RNAi Agent antisense strand unmodified sequence
uaccaauuuaugccuacagcu

SEQ ID NO: 863 - RNA, 21 nt, Artificial sequence
  HBV RNAi Agent antisense strand unmodified sequence
uaccaauuuaugccuacagcg

SEQ ID NO: 864 - RNA, 21 nt, Artificial sequence
  HBV RNAi Agent antisense strand unmodified sequence
aaccaauuuaugccuacagcc

SEQ ID NO: 865 - RNA, 21 nt, Artificial sequence
  HBV RNAi Agent antisense strand unmodified sequence
accaauuuaugccuacagccu

SEQ ID NO: 866 - RNA, 21 nt, Artificial sequence
  HBV RNAi Agent antisense strand unmodified sequence
uccaauuuaugccuacagccu

SEQ ID NO: 867 - RNA, 21 nt, Artificial sequence
  HBV RNAi Agent antisense strand unmodified sequence
accaauuuaugccuacagccg

SEQ ID NO: 868 - RNA, 21 nt, Artificial sequence
  HBV RNAi Agent antisense strand unmodified sequence
uccaauuuaugccuacagccg

SEQ ID NO: 869 - RNA, 21 nt, Artificial sequence
  HBV RNAi Agent antisense strand unmodified sequence
uaccaauuuaugccuacaggg

SEQ ID NO: 870 - DNA, 25 bp, Artificial sequence
  HBV RNAi Agent sense strand modified sequence
uugccuguaggcauaaauugguaut

SEQ ID NO: 871 - RNA, 26 nt, Artificial sequence
  HBV RNAi Agent sense strand modified sequence
uauaugccuguaggcauaaauuggua

SEQ ID NO: 872 - DNA, 26 bp, Artificial sequence
  HBV RNAi Agent sense strand modified sequence
gcggaggcuguaggcauaaauuggta

SEQ ID NO: 873 - RNA, 21 nt, Artificial sequence
  HBV RNAi Agent sense strand modified sequence
cuguaggcauaaauugguauu

SEQ ID NO: 874 - RNA, 21 nt, Artificial sequence
  HBV RNAi Agent sense strand modified sequence
gccuguaggcauaaauuggua

SEQ ID NO: 875 - RNA, 21 nt, Artificial sequence
  HBV RNAi Agent sense strand modified sequence
gccuguaggcauaaauuggua

SEQ ID NO: 876 - RNA, 21 nt, Artificial sequence
  HBV RNAi Agent sense strand modified sequence
gccuguaggcauaaauuggua

SEQ ID NO: 877 - DNA, 21 bp, Artificial sequence
  HBV RNAi Agent sense strand modified sequence
gccuguaggcauaaauuggta

SEQ ID NO: 878 - RNA, 21 nt, Artificial sequence
  HBV RNAi Agent sense strand modified sequence
aacuguaggcauaaauuggua

SEQ ID NO: 879 - RNA, 21 nt, Artificial sequence
  HBV RNAi Agent sense strand modified sequence
ucgugguggacuucucucaau

SEQ ID NO: 880 - RNA, 21 nt, Artificial sequence
  HBV RNAi Agent sense strand modified sequence
aagugguggacuucucucaau

SEQ ID NO: 881 - DNA, 21 bp, Artificial sequence
  HBV RNAi Agent sense strand modified sequence
ucgugguggacuucucucaat

SEQ ID NO: 882 - RNA, 21 nt, Artificial sequence
  HBV RNAi Agent sense strand modified sequence
cgugguggacuucucucaauu

SEQ ID NO: 883 - RNA, 21 nt, Artificial sequence
  HBV RNAi Agent sense strand modified sequence
aaugguggacuucucucaauu

SEQ ID NO: 884 - DNA, 21 bp, Artificial sequence
  HBV RNAi Agent sense strand modified sequence
cgugguggacuucucucaatt

SEQ ID NO: 885 - RNA, 21 nt, Artificial sequence
  HBV RNAi Agent sense strand modified sequence
ggacuucucucaauuuucuaa

SEQ ID NO: 886 - RNA, 21 nt, Artificial sequence
  HBV RNAi Agent sense strand modified sequence
cgugguggacuucucucaaua

SEQ ID NO: 887 - RNA, 21 nt, Artificial sequence
  HBV RNAi Agent sense strand modified sequence
ucgugguggacuucucucaaa

SEQ ID NO: 888 - RNA, 21 nt, Artificial sequence
  HBV RNAi Agent sense strand modified sequence
accuguaggcauaaauuggua

SEQ ID NO: 889 - RNA, 19 nt, Artificial sequence
  HBV RNAi Agent sense strand modified sequence
cuguaggcauaaauuggua

SEQ ID NO: 890 - RNA, 19 nt, Artificial sequence
  HBV RNAi Agent sense strand modified sequence
cuguaggcauaaauuggua

SEQ ID NO: 891 - RNA, 19 nt, Artificial sequence
  HBV RNAi Agent sense strand modified sequence
cuguaggcauaaauuggua

SEQ ID NO: 892 - RNA, 20 nt, Artificial sequence
  HBV RNAi Agent sense strand modified sequence
acuguaggcauaaauuggua

SEQ ID NO: 893 - RNA, 21 nt, Artificial sequence
  HBV RNAi Agent sense strand modified sequence
ggcuguaggcauaaauuggua

SEQ ID NO: 894 - RNA, 21 nt, Artificial sequence
  HBV RNAi Agent sense strand modified sequence
aagugguggacuucucucaau

SEQ ID NO: 895 - RNA, 21 nt, Artificial sequence
  HBV RNAi Agent sense strand modified sequence
aagugguggacuucucucaaa

SEQ ID NO: 896 - RNA, 21 nt, Artificial sequence
  HBV RNAi Agent sense strand modified sequence
ccgugguggacuucucucaau

SEQ ID NO: 897 - RNA, 21 nt, Artificial sequence
  HBV RNAi Agent sense strand modified sequence
ccgugguggacuucucucaau

SEQ ID NO: 898 - RNA, 22 nt, Artificial sequence
  HBV RNAi Agent sense strand modified sequence
cucgugguggacuucucucaau

SEQ ID NO: 899 - RNA, 19 nt, Artificial sequence
  HBV RNAi Agent sense strand modified sequence
gugguggacuucucucaau

SEQ ID NO: 900 - RNA, 21 nt, Artificial sequence
  HBV RNAi Agent sense strand modified sequence
gugguggacuucucucaauuu

SEQ ID NO: 901 - RNA, 22 nt, Artificial sequence
  HBV RNAi Agent sense strand modified sequence
ucgugguggacuucucucaauu

SEQ ID NO: 902 - RNA, 19 nt, Artificial sequence
  HBV RNAi Agent sense strand modified sequence
ugguggacuucucucaauu

SEQ ID NO: 903 - RNA, 19 nt, Artificial sequence
  HBV RNAi Agent sense strand modified sequence
ugguggacuucucucaauu

SEQ ID NO: 904 - RNA, 21 nt, Artificial sequence
  HBV RNAi Agent sense strand modified sequence
guggacuucucucaauuuucu

SEQ ID NO: 905 - RNA, 21 nt, Artificial sequence
  HBV RNAi Agent sense strand modified sequence
cuguaggcauaaauugguauu

SEQ ID NO: 906 - RNA, 22 nt, Artificial sequence
  HBV RNAi Agent sense strand modified sequence
gcuguaggcauaaauugguauu

SEQ ID NO: 907 - RNA, 23 nt, Artificial sequence
  HBV RNAi Agent sense strand modified sequence
ggcuguaggcauaaauugguauu

SEQ ID NO: 908 - RNA, 23 nt, Artificial sequence
  HBV RNAi Agent sense strand modified sequence
aacuguaggcauaaauugguauu

SEQ ID NO: 909 - RNA, 21 nt, Artificial sequence
  HBV RNAi Agent sense strand modified sequence
ugguggacuucucucaauauu

SEQ ID NO: 910 - RNA, 22 nt, Artificial sequence
  HBV RNAi Agent sense strand modified sequence
gugguggacuucucucaauauu

SEQ ID NO: 911 - RNA, 23 nt, Artificial sequence
  HBV RNAi Agent sense strand modified sequence
aaugguggacuucucucaauauu

SEQ ID NO: 912 - RNA, 23 nt, Artificial sequence
  HBV RNAi Agent sense strand modified sequence
cgugguggacuucucucaauauu

SEQ ID NO: 913 - RNA, 21 nt, Artificial sequence
  HBV RNAi Agent sense strand modified sequence
cgugguggacuucucucaaua

SEQ ID NO: 914 - RNA, 21 nt, Artificial sequence
  HBV RNAi Agent sense strand modified sequence
aagugguggacuucucucaau

SEQ ID NO: 915 - RNA, 21 nt, Artificial sequence
  HBV RNAi Agent sense strand modified sequence
gugguggacuucucucaaauu

SEQ ID NO: 916 - RNA, 22 nt, Artificial sequence
  HBV RNAi Agent sense strand modified sequence
cgugguggacuucucucaaauu

SEQ ID NO: 917 - RNA, 23 nt, Artificial sequence
  HBV RNAi Agent sense strand modified sequence
aagugguggacuucucucaaauu

SEQ ID NO: 918 - RNA, 23 nt, Artificial sequence
  HBV RNAi Agent sense strand modified sequence
ucgugguggacuucucucaaauu

SEQ ID NO: 919 - RNA, 22 nt, Artificial sequence
  HBV RNAi Agent sense strand modified sequence
gugguggacuucucucaauauu

SEQ ID NO: 920 - RNA, 23 nt, Artificial sequence
  HBV RNAi Agent sense strand modified sequence
cgugguggacuucucucaauauu

SEQ ID NO: 921 - RNA, 23 nt, Artificial sequence
  HBV RNAi Agent sense strand modified sequence
cucgugguggacuucucucaaua

SEQ ID NO: 922 - RNA, 25 nt, Artificial sequence
  HBV RNAi Agent sense strand modified sequence
cucgugguggacuucucucaauauu

SEQ ID NO: 923 - RNA, 21 nt, Artificial sequence
  HBV RNAi Agent sense strand modified sequence
ggcuguaggcauaaauuggua

SEQ ID NO: 924 - RNA, 23 nt, Artificial sequence
  HBV RNAi Agent sense strand modified sequence
gaggcuguaggcauaaauuggua

SEQ ID NO: 925 - RNA, 25 nt, Artificial sequence
  HBV RNAi Agent sense strand modified sequence
gaggcuguaggcauaaauugguauu

SEQ ID NO: 926 - RNA, 19 nt, Artificial sequence
  HBV RNAi Agent sense strand modified sequence
ggacuucucucaauuuucu

SEQ ID NO: 927 - RNA, 21 nt, Artificial sequence
  HBV RNAi Agent sense strand modified sequence
guggacuucucucaauuuucu

SEQ ID NO: 928 - RNA, 22 nt, Artificial sequence
  HBV RNAi Agent sense strand modified sequence
gguggacuucucucaauuuucu

SEQ ID NO: 929 - RNA, 19 nt, Artificial sequence
  HBV RNAi Agent sense strand modified sequence
ggacuucucucaauuuuca

SEQ ID NO: 930 - RNA, 19 nt, Artificial sequence
  HBV RNAi Agent sense strand modified sequence
ggacuucucucaauuuuca

SEQ ID NO: 931 - RNA, 19 nt, Artificial sequence
  HBV RNAi Agent sense strand modified sequence
gcuguaggcauaaauuggu

SEQ ID NO: 932 - RNA, 20 nt, Artificial sequence
  HBV RNAi Agent sense strand modified sequence
ggcuguaggcauaaauuggu

SEQ ID NO: 933 - RNA, 22 nt, Artificial sequence
  HBV RNAi Agent sense strand modified sequence
gaggcuguaggcauaaauuggu

SEQ ID NO: 934 - RNA, 19 nt, Artificial sequence
  HBV RNAi Agent sense strand modified sequence
gcuguaggcauaaauugga

SEQ ID NO: 935 - RNA, 20 nt, Artificial sequence
  HBV RNAi Agent sense strand modified sequence
ggcuguaggcauaaauugga

SEQ ID NO: 936 - RNA, 21 nt, Artificial sequence
  HBV RNAi Agent sense strand modified sequence
agcuguaggcauaaauuggua

SEQ ID NO: 937 - RNA, 21 nt, Artificial sequence
  HBV RNAi Agent sense strand modified sequence
cgcuguaggcauaaauuggua

SEQ ID NO: 938 - RNA, 21 nt, Artificial sequence
  HBV RNAi Agent sense strand modified sequence
ggcuguaggcauaaauugguu

SEQ ID NO: 939 - RNA, 21 nt, Artificial sequence
  HBV RNAi Agent sense strand modified sequence
cuguaggcauaaauugguauu

SEQ ID NO: 940 - RNA, 21 nt, Artificial sequence
  HBV RNAi Agent sense strand modified sequence
cuguaggcauaaauugguauu

SEQ ID NO: 941 - RNA, 21 nt, Artificial sequence
  HBV RNAi Agent sense strand modified sequence
aggcuguaggcauaaauuggu

SEQ ID NO: 942 - RNA, 21 nt, Artificial sequence
  HBV RNAi Agent sense strand modified sequence
aggcuguaggcauaaauugga

SEQ ID NO: 943 - RNA, 21 nt, Artificial sequence
  HBV RNAi Agent sense strand modified sequence
cggcuguaggcauaaauuggu

SEQ ID NO: 944 - RNA, 21 nt, Artificial sequence
  HBV RNAi Agent sense strand modified sequence
cggcuguaggcauaaauugga

SEQ ID NO: 945 - RNA, 21 nt, Artificial sequence
  HBV RNAi Agent sense strand modified sequence
cccuguaggcauaaauuggua

SEQ ID NO: 946 - RNA, 21 nt, Artificial sequence
  HBV RNAi Agent sense strand modified sequence
cgcuguaggcauaaauuggua

SEQ ID NO: 947 - RNA, 21 nt, Artificial sequence
  HBV RNAi Agent sense strand modified sequence
cccuguaggcauaaauuggua

SEQ ID NO: 948 - RNA, 21 nt, Artificial sequence
  HBV RNAi Agent sense strand modified sequence
guggacuucucucaauuuucu

SEQ ID NO: 949 - RNA, 21 nt, Artificial sequence
  HBV RNAi Agent sense strand modified sequence
cgcuguaggcauaaauuggua

SEQ ID NO: 950 - DNA, 25 bp, Artificial sequence
  HBV RNAi Agent sense strand unmodified sequence
uugccuguaggcauaaauugguaut

SEQ ID NO: 951 - RNA, 26 nt, Artificial sequence
  HBV RNAi Agent sense strand unmodified sequence
uauaugccuguaggcauaaauuggua

SEQ ID NO: 952 - DNA, 26 bp, Artificial sequence
  HBV RNAi Agent sense strand unmodified sequence
gcggaggcuguaggcauaaauuggta

SEQ ID NO: 953 - RNA, 21 nt, Artificial sequence
  HBV RNAi Agent sense strand unmodified sequence
cuguaggcauaaauugguauu

SEQ ID NO: 954 - RNA, 21 nt, Artificial sequence
  HBV RNAi Agent sense strand unmodified sequence
gccuguaggcauaaauuggua

SEQ ID NO: 955 - DNA, 21 bp, Artificial sequence
  HBV RNAi Agent sense strand unmodified sequence
gccuguaggcauaaauuggta

SEQ ID NO: 956 - RNA, 21 nt, Artificial sequence
  HBV RNAi Agent sense strand unmodified sequence
aacuguaggcauaaauuggua

SEQ ID NO: 957 - RNA, 21 nt, Artificial sequence
  HBV RNAi Agent sense strand unmodified sequence
ucgugguggacuucucucaau

SEQ ID NO: 958 - RNA, 21 nt, Artificial sequence
  HBV RNAi Agent sense strand unmodified sequence
aagugguggacuucucucaau

SEQ ID NO: 959 - DNA, 21 bp, Artificial sequence
  HBV RNAi Agent sense strand unmodified sequence
ucgugguggacuucucucaat

SEQ ID NO: 960 - RNA, 21 nt, Artificial sequence
  HBV RNAi Agent sense strand unmodified sequence
cgugguggacuucucucaauu

SEQ ID NO: 961 - RNA, 21 nt, Artificial sequence
  HBV RNAi Agent sense strand unmodified sequence
aaugguggacuucucucaauu

SEQ ID NO: 962 - DNA, 21 bp, Artificial sequence
  HBV RNAi Agent sense strand unmodified sequence
cgugguggacuucucucaatt

SEQ ID NO: 963 - RNA, 21 nt, Artificial sequence
  HBV RNAi Agent sense strand unmodified sequence
ggacuucucucaauuuucuaa

SEQ ID NO: 964 - RNA, 21 nt, Artificial sequence
  HBV RNAi Agent sense strand unmodified sequence
cgugguggacuucucucaaua

SEQ ID NO: 965 - RNA, 21 nt, Artificial sequence
  HBV RNAi Agent sense strand unmodified sequence
ucgugguggacuucucucaaa

SEQ ID NO: 966 - RNA, 21 nt, Artificial sequence
  HBV RNAi Agent sense strand unmodified sequence
accuguaggcauaaauuggua

SEQ ID NO: 967 - RNA, 19 nt, Artificial sequence
  HBV RNAi Agent sense strand unmodified sequence
cuguaggcauaaauuggua

SEQ ID NO: 968 - RNA, 20 nt, Artificial sequence
  HBV RNAi Agent sense strand unmodified sequence
acuguaggcauaaauuggua

SEQ ID NO: 969 - RNA, 21 nt, Artificial sequence
  HBV RNAi Agent sense strand unmodified sequence
ggcuguaggcauaaauuggua

SEQ ID NO: 970 - RNA, 21 nt, Artificial sequence
  HBV RNAi Agent sense strand unmodified sequence
aagugguggacuucucucaaa

SEQ ID NO: 971 - RNA, 21 nt, Artificial sequence
  HBV RNAi Agent sense strand unmodified sequence
ccgugguggacuucucucaau

SEQ ID NO: 972 - RNA, 22 nt, Artificial sequence
  HBV RNAi Agent sense strand unmodified sequence
cucgugguggacuucucucaau

SEQ ID NO: 973 - RNA, 19 nt, Artificial sequence
  HBV RNAi Agent sense strand unmodified sequence
gugguggacuucucucaau

SEQ ID NO: 974 - RNA, 21 nt, Artificial sequence
  HBV RNAi Agent sense strand unmodified sequence
gugguggacuucucucaauuu

SEQ ID NO: 975 - RNA, 22 nt, Artificial sequence
  HBV RNAi Agent sense strand unmodified sequence
ucgugguggacuucucucaauu

SEQ ID NO: 976 - RNA, 19 nt, Artificial sequence
  HBV RNAi Agent sense strand unmodified sequence
ugguggacuucucucaauu

SEQ ID NO: 977 - RNA, 21 nt, Artificial sequence
  HBV RNAi Agent sense strand unmodified sequence
guggacuucucucaauuuucu

SEQ ID NO: 978 - RNA, 22 nt, Artificial sequence
  HBV RNAi Agent sense strand unmodified sequence
gcuguaggcauaaauugguauu

SEQ ID NO: 979 - RNA, 23 nt, Artificial sequence
  HBV RNAi Agent sense strand unmodified sequence
ggcuguaggcauaaauugguauu

SEQ ID NO: 980 - RNA, 23 nt, Artificial sequence
  HBV RNAi Agent sense strand unmodified sequence
aacuguaggcauaaauugguauu

SEQ ID NO: 981 - RNA, 21 nt, Artificial sequence
  HBV RNAi Agent sense strand unmodified sequence
ugguggacuucucucaauauu

SEQ ID NO: 982 - RNA, 22 nt, Artificial sequence
  HBV RNAi Agent sense strand unmodified sequence
gugguggacuucucucaauauu

SEQ ID NO: 983 - RNA, 23 nt, Artificial sequence
  HBV RNAi Agent sense strand unmodified sequence
aaugguggacuucucucaauauu

SEQ ID NO: 984 - RNA, 23 nt, Artificial sequence
  HBV RNAi Agent sense strand unmodified sequence
cgugguggacuucucucaauauu

SEQ ID NO: 985 - RNA, 21 nt, Artificial sequence
  HBV RNAi Agent sense strand unmodified sequence
gugguggacuucucucaaauu

SEQ ID NO: 986 - RNA, 22 nt, Artificial sequence
  HBV RNAi Agent sense strand unmodified sequence
cgugguggacuucucucaaauu

SEQ ID NO: 987 - RNA, 23 nt, Artificial sequence
  HBV RNAi Agent sense strand unmodified sequence
aagugguggacuucucucaaauu

SEQ ID NO: 988 - RNA, 23 nt, Artificial sequence
  HBV RNAi Agent sense strand unmodified sequence
ucgugguggacuucucucaaauu

SEQ ID NO: 989 - RNA, 23 nt, Artificial sequence
  HBV RNAi Agent sense strand unmodified sequence
cucgugguggacuucucucaaua

SEQ ID NO: 990 - RNA, 25 nt, Artificial sequence
  HBV RNAi Agent sense strand unmodified sequence
cucgugguggacuucucucaauauu

SEQ ID NO: 991 - RNA, 23 nt, Artificial sequence
  HBV RNAi Agent sense strand unmodified sequence
gaggcuguaggcauaaauuggua

SEQ ID NO: 992 - RNA, 25 nt, Artificial sequence
  HBV RNAi Agent sense strand unmodified sequence
gaggcuguaggcauaaauugguauu

SEQ ID NO: 993 - RNA, 19 nt, Artificial sequence
  HBV RNAi Agent sense strand unmodified sequence
ggacuucucucaauuuucu

SEQ ID NO: 994 - RNA, 22 nt, Artificial sequence
  HBV RNAi Agent sense strand unmodified sequence
gguggacuucucucaauuuucu

SEQ ID NO: 995 - RNA, 19 nt, Artificial sequence
  HBV RNAi Agent sense strand unmodified sequence
ggacuucucucaauuuuca

SEQ ID NO: 996 - RNA, 21 nt, Artificial sequence
  HBV RNAi Agent sense strand unmodified sequence
guggacuucucucaauuuuca

SEQ ID NO: 997 - RNA, 19 nt, Artificial sequence
  HBV RNAi Agent sense strand unmodified sequence
gcuguaggcauaaauuggu

SEQ ID NO: 998 - RNA, 20 nt, Artificial sequence
  HBV RNAi Agent sense strand unmodified sequence
ggcuguaggcauaaauuggu

SEQ ID NO: 999 - RNA, 22 nt, Artificial sequence
  HBV RNAi Agent sense strand unmodified sequence
gaggcuguaggcauaaauuggu

SEQ ID NO: 1000 - RNA, 19 nt, Artificial sequence
  HBV RNAi Agent sense strand unmodified sequence
gcuguaggcauaaauugga

SEQ ID NO: 1001 - RNA, 20 nt, Artificial sequence
  HBV RNAi Agent sense strand unmodified sequence
ggcuguaggcauaaauugga

SEQ ID NO: 1002 - RNA, 21 nt, Artificial sequence
  HBV RNAi Agent sense strand unmodified sequence
agcuguaggcauaaauuggua

SEQ ID NO: 1003 - RNA, 21 nt, Artificial sequence
  HBV RNAi Agent sense strand unmodified sequence
cgcuguaggcauaaauuggua

SEQ ID NO: 1004 - RNA, 21 nt, Artificial sequence
  HBV RNAi Agent sense strand unmodified sequence
ggcuguaggcauaaauugguu

SEQ ID NO: 1005 - RNA, 21 nt, Artificial sequence
  HBV RNAi Agent sense strand unmodified sequence
aggcuguaggcauaaauuggu

SEQ ID NO: 1006 - RNA, 21 nt, Artificial sequence
  HBV RNAi Agent sense strand unmodified sequence
aggcuguaggcauaaauugga

SEQ ID NO: 1007 - RNA, 21 nt, Artificial sequence
  HBV RNAi Agent sense strand unmodified sequence
cggcuguaggcauaaauuggu

SEQ ID NO: 1008 - RNA, 21 nt, Artificial sequence
  HBV RNAi Agent sense strand unmodified sequence
cggcuguaggcauaaauugga

SEQ ID NO: 1009 - RNA, 21 nt, Artificial sequence
  HBV RNAi Agent sense strand unmodified sequence
cccuguaggcauaaauuggua